(19) United States
(12) Patent Application Publication
(10) Pub. No.: US 2026/0042851 A1
(43) Pub. Date: Feb. 12, 2026

(54) BINDING AGENTS AND USES THEREOF

(71) Applicant: EpiBiologics, Inc., San Mateo, CA (US)

(72) Inventors: Shyra Jane Gardai, San Mateo, CA (US); Hai Tran, San Mateo, CA (US); Andrew Christopher Goodrich, San Mateo, CA (US); Lisa Marshall, San Mateo, CA (US); Isaac J. Rondon, San Mateo, CA (US); Shruti Yadav, San Mateo, CA (US); Jonathan Sitrin, San Mateo, CA (US); Fortunato Ferrara, Santa Fe, NM (US)

(21) Appl. No.: 19/354,455

(22) Filed: Oct. 9, 2025

Related U.S. Application Data
(63) Continuation of application No. PCT/US2025/036166, filed on Jul. 1, 2025.
(60) Provisional application No. 63/667,038, filed on Jul. 2, 2024.
(60) Provisional application No. 63/700,559, filed on Sep. 27, 2024.
(60) Provisional application No. 63/721,252, filed on Nov. 15, 2024.
(60) Provisional application No. 63/763,713, filed on Feb. 26, 2025.

(51) Int. Cl. C07K 16/28; A61K 39/00; A61K 47/68; A61P 35/00

(52) U.S. Cl. CPC C07K 16/2863; A61K 47/6879; A61P 35/00; C07K 16/2839; A61K 2039/505; C07K 2317/31; C07K 2317/565

(57) ABSTRACT

The present disclosure provides methods of degrading an EGFR protein on a target cell. The present disclosure further discloses antigen binding molecules that bind to an EGFR protein and a membrane-associated internalizing protein, such as ITGB6.

### CROSS-REFERENCE

This application is a continuation of International Patent Application No. PCT/US2025/036166 filed on Jul. 1, 2025, which claims the benefit of U.S. Provisional Patent Application No. 63/667,038 filed on Jul. 2, 2024, U.S. Provisional Patent Application No. 63/700,559 filed on Sep. 27, 2024, U.S. Provisional Patent Application No. 63/721,252 filed on Nov. 15, 2024, and U.S. Provisional Patent Application No. 63/763,713 filed on Feb. 26, 2025, each of which is incorporated by reference in its entirety.

### SEQUENCE LISTING

The instant application contains a Sequence Listing which has been submitted electronically in XML format and is hereby incorporated by reference in its entirety. Said XML copy, created on Aug. 1, 2025, is named 61563-717_301_SL.xml and is 718,694 bytes in size.

### BACKGROUND

Standard binding-based small molecule inhibitors rely on sustained, occupancy-driven pharmacology, necessitating high affinity binders capable of abrogating catalytic or binding functions. Inhibiting protein-protein interactions or scaffolding function has been extremely challenging for standard binding-based small molecules. In contrast, protein degraders can be catalytic and utilize event-driven pharmacology, alleviating the need for high affinity binders, and durably abrogate all protein functions at once. To date, most degraders are heterobifunctional small molecules that recruit intracellular E3 ubiquitin ligases to an intracellular target of interest, which induces ubiquitination of the target protein. Most degrader technologies, including PROTACs, utilize an intracellular mechanism of action and have thus been largely limited to targeting proteins with cytoplasmic domains. However, recent approaches, such as LYTACs have been described for specifically degrading cell surface proteins. These utilize recycling glycan receptors such as the mannose-6-phosphate receptor (M6PR) or asialoglycoprotein receptor (ASGR) to target proteins for internalization. Hybrid antibody-based approaches, such as PROTACs (AbTACs), utilize an IgG bispecific antibody format to bring a cell surface E3 ligase (RNF43) into proximity of a membrane protein of interest (POI) to mediate its degradation through the lysosomal or proteasomal pathway.

### SUMMARY

Provided herein, in some aspects, are antigen binding molecule, comprising: (i) a first antigen binding domain that binds to an epidermal growth factor receptor (EGFR); and (ii) a second antigen binding domain that binds to an integrin subunit beta 6 (ITGB6), wherein the first antigen binding domain comprises a heavy chain variable region (VH) and a light chain variable region (VL), wherein the VH comprises: (a) a heavy chain complementarity determining region 1 (HCDR1) amino acid sequence of SEQ ID NO: 1; (b) a heavy chain complementarity determining region 2 (HCDR2) amino acid sequence of SEQ ID NO: 2; and (c) a heavy chain complementarity determining region 3 (HCDR3) amino acid sequence of SEQ ID NO: 3. In some embodiments, the VL of the first antigen binding domain comprises: (a) a light chain complementarity determining region 1 (LCDR1) amino acid sequence of SEQ ID NO: 4; (b) a light chain complementarity determining region 2 (LCDR2) amino acid sequence of SEQ ID NO: 5; and (c) a light chain complementarity determining region 3 (LCDR3) amino acid sequence of SEQ ID NO: 6. Further provided herein, in some aspects, are antigen binding molecules, comprising: (i) a first antigen binding domain that binds to an epidermal growth factor receptor (EGFR); and (ii) a second antigen binding domain that binds to an integrin subunit beta 6 (ITGB6), wherein the second antigen binding domain comprises a heavy chain variable region (VH) and a light chain variable region (VL), wherein the VH comprises: (a) a heavy chain complementarity determining region 1 (HCDR1) amino acid sequence of SEQ ID NO: 7; (b) a heavy chain complementarity determining region 2 (HCDR2) amino acid sequence of SEQ ID NO: 8; and (c) a heavy chain complementarity determining region 3 (HCDR3) amino acid sequence of SEQ ID NO: 9. In some embodiments, the VL of the second antigen binding domain comprises: (a) a light chain complementarity determining region 1 (LCDR1) amino acid sequence of SEQ ID NO: 10; (b) a light chain complementarity determining region 2 (LCDR2) amino acid sequence of SEQ ID NO: 11; and (c) a light chain complementarity determining region 3 (LCDR3) amino acid sequence of SEQ ID NO: 12. In some embodiments, the first antigen binding domain comprises a heavy chain variable region (VH) comprising: (a) a HCDR1 amino acid sequence selected from: DYGMH (SEQ ID NO: 16), and NQGIS (SEQ ID NO: 25); (b) a HCDR2 amino acid sequence selected from: AIDAGGSTDYADSVEG (SEQ ID NO: 17) and GFDPDAGETIYAQKFQG (SEQ ID NO: 26); or (c) a HCDR3 amino acid sequence selected from: DLEAGYYAPDV (SEQ ID NO: 18) and GVDSYGYGRYNWFDP (SEQ ID NO: 27). In some embodiments, the first antigen binding domain comprises a light chain variable region (VL) comprising: (a) a LCDR1 amino acid sequence selected from: RASQDIGRFLA (SEQ ID NO: 31), and RASQDIRHYLA (SEQ ID NO: 37); (b) a LCDR2 amino acid sequence selected from: AVSNLQS (SEQ ID NO: 32) and DTFNRAT (SEQ ID NO: 38); or (c) a LCDR3 amino acid sequence selected from: QQYSTSVYT (SEQ ID NO: 33) and QQYHNLPYS (SEQ ID NO: 39). In some embodiments, the second antigen binding domain comprises a heavy chain variable region (VH) comprising: (a) a HCDR1 amino acid sequence selected from: NDLIE (SEQ ID NO: 58), and NYLIE (SEQ ID NO: 67); (b) a HCDR2 amino acid sequence selected from: VINPGSGRTNYAQKFQG (SEQ ID NO: 59) and VISPGSGIINYAQKFQG (SEQ ID NO: 68); or (c) a HCDR3 amino acid sequence selected from: IYYGPHSYAMDY (SEQ ID NO: 60) and IDYSGPYAVDD (SEQ ID NO: 69). In some embodiments, the second antigen binding domain comprises a light chain variable region (VL) comprising: (a) a LCDR1 amino acid sequence selected from: KASLDVRTAVA (SEQ ID NO: 73), and KASQAVNTAVA (SEQ ID NO: 79); (b) a LCDR2 amino acid sequence selected from: SASYRYT (SEQ ID NO: 74) and SASYGYT (SEQ ID NO: 80); or (c) a LCDR3 amino acid sequence selected from: QQHYGIPWT (SEQ ID NO: 75) and QHHYGVPWT (SEQ ID NO: 81) In some embodiments, the first antigen binding domain comprises a heavy chain variable region (VH) comprising: (a) a HCDR1 amino acid sequence of DYGMH (SEQ ID NO: 16); (b) a HCDR2 amino acid sequence of AIDAGGSTDYADSVEG (SEQ ID NO: 17); and (c) a HCDR3 amino acid sequence of DLEAGYYAPDV (SEQ ID NO: 18). In some embodiments, the first antigen binding domain comprises a light chain variable region (VL) comprising: (a) a LCDR1 amino acid sequence of RASQDIGRFLA (SEQ ID NO: 31); (b) a LCDR2 amino acid sequence of AVSNLQS (SEQ ID NO: 32); and (c) a LCDR3 amino acid sequence of QQYSTSVYT (SEQ ID NO: 33). In some embodiments, the first antigen binding domain comprises: (a) a VL comprising a LCDR1 amino acid sequence of RASQDIGRFLA (SEQ ID NO: 31), a LCDR2 amino acid sequence of AVSNLQS (SEQ ID NO: 32), and a LCDR3 amino acid sequence of QQYSTSVYT (SEQ ID NO: 33); and (b) a VH comprising a HCDR1 amino acid sequence of DYGMH (SEQ ID NO: 16), a HCDR2 amino acid sequence of AIDAGGSTDYADSVEG (SEQ ID NO: 17), and a HCDR3 amino acid sequence of DLEAGYYAPDV (SEQ ID NO: 18). In some embodiments, the first antigen binding domain comprises a VL comprising a sequence having at least 75%, at least 80%, at least 85%, at least 90%, at least 95%, or at least 99% sequence identity to the sequence of SEQ ID NO: 49. In some embodiments, the first antigen binding domain comprises a VL of SEQ ID NO: 49. In some embodiments, the first antigen binding domain comprises a VH comprising a sequence having at least 75%, at least 80%, at least 85%, at least 90%, at least 95%, or at least 99% sequence identity to the sequence of SEQ ID NO: 43. In some embodiments, the first antigen binding domain comprises a VH of SEQ ID NO: 43. In some embodiments, the second antigen binding domain comprises a heavy chain variable region (VH) comprising: (a) a HCDR1 amino acid sequence of NDLIE (SEQ ID NO: 58); (b) a HCDR2 amino acid sequence of VINPGSGRTNYAQKFQG (SEQ ID NO: 59); and (c) a HCDR3 amino acid sequence of 1YYGPHSYAMDY (SEQ ID NO: 60). In some embodiments, the second antigen binding domain comprises a light chain variable region (VL) comprising: (a) a LCDR1 amino acid sequence of KASLDVRTAVA (SEQ ID NO: 73); (b) a LCDR2 amino acid sequence of SASYRYT (SEQ ID NO: 74); and (c) a LCDR3 amino acid sequence of QQHYGIPWT (SEQ ID NO: 75). In some embodiments, the second antigen binding domain comprises: (a) a VL comprising a LCDR1 amino acid sequence of KASLDVRTAVA (SEQ ID NO: 73), a LCDR2 amino acid sequence of SASYRYT (SEQ ID NO: 74), and a LCDR3 amino acid sequence of QQHYGIPWT (SEQ ID NO: 75); and (b) a VH comprising a HCDR1 amino acid sequence of NDLIE (SEQ ID NO: 58), a HCDR2 amino acid sequence of VINPGSGRTNYAQKFQG (SEQ ID NO: 59), and a HCDR3 amino acid sequence of IYYGPHSYAMDY (SEQ ID NO: 60). In some embodiments, the second antigen binding domain comprises a VL comprising a sequence having at least 75%, at least 80%, at least 85%, at least 90%, at least 95%, or at least 99% sequence identity to the sequence of SEQ ID NO: 91. In some embodiments, the second antigen binding domain comprises a VL of SEQ ID NO: 91. In some embodiments, the second antigen binding domain comprises a VH comprising a sequence having at least 75%, at least 80%, at least 85%, at least 90%, at least 95%, or at least 99% sequence identity to the sequence of SEQ ID NO: 85. In some embodiments, the second antigen binding domain comprises a VH of SEQ ID NO: 85. In some embodiments, the second antigen binding domain comprises a heavy chain variable region (VH) comprising: (a) a HCDR1 amino acid sequence of NYLIE (SEQ ID NO: 67); (b) a HCDR2 amino acid sequence of VISPGSGIINYAQKFQG (SEQ ID NO: 68); and (c) a HCDR3 amino acid sequence of IDYSGPYAVDD (SEQ ID NO: 69). In some embodiments, the second antigen binding domain comprises a light chain variable region (VL) comprising: (a) a LCDR1 amino acid sequence of KASQAVNTAVA (SEQ ID NO: 79); (b) a LCDR2 amino acid sequence of SASYGYT (SEQ ID NO: 80); and (c) a LCDR3 amino acid sequence of QHHYGVPWT (SEQ ID NO: 81). In some embodiments, the second antigen binding domain comprises: (a) a VL comprising a LCDR1 amino acid sequence of KASQAVNTAVA (SEQ ID NO: 79), a LCDR2 amino acid sequence of SASYGYT (SEQ ID NO: 80), and a LCDR3 amino acid sequence of QHHYGVPWT (SEQ ID NO: 81); and (b) a VH comprising a HCDR1 amino acid sequence of NYLIE (SEQ ID NO: 67), a HCDR2 amino acid sequence of VISPGSGIINYAQKFQG (SEQ ID NO: 68), and a HCDR3 amino acid sequence of IDYSGPYAVDD (SEQ ID NO: 69). In some embodiments, the second antigen binding domain comprises a VL comprising a sequence having at least 75%, at least 80%, at least 85%, at least 90%, at least 95%, or at least 99% sequence identity to the sequence of SEQ ID NO: 94. In some embodiments, the second antigen binding domain comprises a VL comprising the sequence of SEQ ID NO: 94. In some embodiments, the second antigen binding domain comprises a VH comprising a sequence having at least 75%, at least 80%, at least 85%, at least 90%, at least 95%, or at least 99% sequence identity to the sequence of SEQ ID NO: 88. In some embodiments, the second antigen binding domain comprises a VH comprising the sequence of SEQ ID NO: 88. In some embodiments, the second antigen binding domain comprises a VL comprising a sequence having at least 75%, at least 80%, at least 85%, at least 90%, at least 95%, or at least 99% sequence identity to the sequence of SEQ ID NO: 94 and a VH comprising a sequence having at least 75%, at least 80%, at least 85%, at least 90%, at least 95%, or at least 99% sequence identity to the sequence of SEQ ID NO: 88. In some embodiments, the second antigen binding domain comprises a VL comprising the sequence of SEQ ID NO: 94 and a VH comprising the sequence of SEQ ID NO: 88. In some embodiments, the first antigen binding domain comprises a heavy chain variable region (VH) comprising: (a) a HCDR1 amino acid sequence of NQGIS (SEQ ID NO: 25); (b) a HCDR2 amino acid sequence of GFDPDAGETIYAQKFQG (SEQ ID NO: 26); and (c) a HCDR3 amino acid sequence of GVDSYGYGRYNWFDP (SEQ ID NO: 27). In some embodiments, the first antigen binding domain comprises a light chain variable region (VL) comprising: (a) a LCDR1 amino acid sequence of RASQDIRHYLA (SEQ ID NO: 37); (b) a LCDR2 amino acid sequence of DTFNRAT (SEQ ID NO: 38); and (c) a LCDR3 amino acid sequence of QQYHNLPYS (SEQ ID NO: 39). In some embodiments, the first antigen binding domain comprises: (a) a VL comprising a LCDR1 amino acid sequence of RASQDIRHYLA (SEQ ID NO: 37), a LCDR2 amino acid sequence of DTFNRAT (SEQ ID NO: 38), and a LCDR3 amino acid sequence of QQYHNLPYS (SEQ ID NO: 39); and (b) a VH comprising a HCDR1 amino acid sequence of NQGIS (SEQ ID NO: 25), a HCDR2 amino acid sequence of GFDPDAGETIYAQKFQG (SEQ ID NO: 26), and a HCDR3 amino acid sequence of GVDSYGYGRYNWFDP (SEQ ID NO: 27). In some embodiments, the first antigen binding domain comprises a VL comprising a sequence having at least 75%, at least 80%, at least 85%, at least 90%, at least 95%, or at least 99% sequence identity to the sequence of SEQ ID NO: 52. In some embodiments, the first antigen binding domain comprises a VL of SEQ ID NO: 52. In some embodiments, the first antigen binding domain comprises a VH comprising a sequence having at least 75%, at least 80%, at least 85%, at least 90%, at least 95%, or at least 99% sequence identity to the sequence of SEQ ID NO: 46. In some embodiments, the first antigen binding domain comprises a VH of SEQ ID NO: 46. In some embodiments, the first antigen binding domain comprises a Fab or a scFv. In some embodiments, the second antigen binding domain comprises a Fab or a scFv. In some embodiments, the first antigen binding domain comprises a Fab with a first light chain constant region and a first heavy chain region. In some embodiments, the second antigen binding domain comprises a Fab with a second light chain constant region and a second heavy chain region. In some embodiments, the first light chain constant region and the second light chain constant region are independently selected from a kappa light chain constant region or functional fragment thereof, and a lambda light chain constant region or functional fragment thereof. In some embodiments, the first light chain constant region is a kappa light chain constant region. In some embodiments, the second light chain constant region is a kappa light chain constant region. In some embodiments, the first heavy chain constant region and the second heavy chain constant region are independently selected from an IgG1 heavy chain constant region or functional fragment thereof, an IgG2 heavy chain constant region or functional fragment thereof, an IgG3 heavy chain constant region or functional fragment thereof, an IgGA1 heavy chain constant region or functional fragment thereof, an IgGA2 heavy chain constant region or functional fragment thereof, an IgG4 heavy chain constant region or functional fragment thereof, an IgJ heavy chain constant region or functional fragment thereof, an IgM heavy chain constant region or functional fragment thereof, an IgD heavy chain constant region or functional fragment thereof, and an IgE heavy chain constant region or functional fragment thereof. In some embodiments, the first heavy chain constant region is an IgG1 heavy chain constant region. In some embodiments, the second heavy chain constant region is an IgG1 heavy chain constant region. In some embodiments, the antigen binding molecule comprises a first polypeptide and a second polypeptide, wherein the first polypeptide and the second polypeptide are non-contiguous, wherein: (a) the first polypeptide comprises the VL of the first antigen binding domain and a first Light Chain Constant Region (CL), wherein the first CL is linked to the VL of the first antigen binding domain; and (b) the second polypeptide comprises the VH of the first antigen binding domain and a first immunoglobulin constant region (Fc region), wherein the first Fc region is linked to the VH of the first antigen binding domain. In some embodiments, the antigen binding molecule comprises a third polypeptide and a fourth polypeptide, wherein the third polypeptide and the fourth polypeptide are non-contiguous, wherein: (a) the third polypeptide comprises the VL of the second antigen binding domain and a second Light Chain Constant Region (CL), wherein the second CL is linked to the VL of the second antigen binding domain; and (b) the fourth polypeptide comprises the VH of the second antigen binding domain and a second immunoglobulin constant region (Fc region), wherein the second Fc region is linked to the VH of the second antigen binding domain. In some embodiments, the second polypeptide further comprises a first heavy chain constant region (CH) linked to the VH of the first antigen binding domain.

In some embodiments, the fourth polypeptide further comprises a second heavy chain constant region (CH) linked to the VH of the second antigen binding domain. In some embodiments, the first Fc region and the second Fc region are independently selected from an IgG1 Fc region or a functional fragment thereof, an IgG2 Fc region or a functional fragment thereof, an IgG3 Fc region or a functional fragment thereof, an IgGA1 Fc region or a functional fragment thereof, an IgGA2 Fc region or a functional fragment thereof, an IgG4 Fc region or a functional fragment thereof, an IgJ Fc region or a functional fragment thereof, an IgM Fc region or a functional fragment thereof, an IgD Fc region or a functional fragment thereof, and an IgE Fc region or a functional fragment thereof. In some embodiments, the first Fc region is an IgG1 Fc region or a functional fragment thereof. In some embodiments, the second Fc region is an IgG1 Fc region or a functional fragment thereof. In some embodiments, the antigen binding molecule is a multispecific antibody, a bispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab. In some embodiments, the antigen binding molecule is a bispecific antibody. In some embodiments, the first CL comprises a sequence having at least 75%, at least 80%, at least 85%, at least 90%, at least 95%, or at least 99% sequence identity to the sequence of any one of SEQ ID NO: 51 or 54. In some embodiments, the first CL comprises a sequence of any one of SEQ ID NO: 51 or 54. In some embodiments, the first CH comprises a sequence having at least 75%, at least 80%, at least 85%, at least 90%, at least 95%, or at least 99% sequence identity to the sequence of any one of SEQ ID NO: 45 or 48. In some embodiments, the first CH comprises a sequence of any one of SEQ ID NO: 45 or 48. In some embodiments, the first CL comprises a sequence having at least 75%, at least 80%, at least 85%, at least 90%, at least 95%, or at least 99% sequence identity to the sequence of any one of SEQ ID NOs: 51 or 54; and the first CH comprises a sequence having at least 75%, at least 80%, at least 85%, at least 90%, at least 95%, or at least 99% sequence identity to the sequence of any one of SEQ ID NOs: 45 or 48. In some embodiments, the first CL comprises a sequence of any one of SEQ ID NOs: 51 or 54; and the first CH comprises a sequence of any one of SEQ ID NOs: 45 or 48. In some embodiments, the second CL comprises a sequence having at least 75%, at least 80%, at least 85%, at least 90%, at least 95%, or at least 99% sequence identity to the sequence of any one of SEQ ID NOs: 93 or 96. In some embodiments, the second CL comprises a sequence of any one of SEQ ID NOs: 93 or 96. In some embodiments, the second CH comprises a sequence having at least 75%, at least 80%, at least 85%, at least 90%, at least 95%, or at least 99% sequence identity to the sequence of any one of SEQ ID NOs: 87 or 90. In some embodiments, the second CH comprises a sequence of any one of SEQ ID NOs: 87 or 90. In some embodiments, the second CL comprises a sequence having at least 75%, at least 80%, at least 85%, at least 90%, at least 95%, or at least 99% sequence identity to the sequence of any one of SEQ ID NOs: 93 or 96; and the second CH comprises a sequence having at least 75%, at least 80%, at least 85%, at least 90%, at least 95%, or at least 99% sequence identity to the sequence of any one of SEQ ID NOs: 87 or 90. In some embodiments, the second CL comprises a sequence of any one of SEQ ID NOs: 93 or 96; and the second CH comprises a sequence of any one of SEQ ID NOs: 87 or 90. In some embodiments, the second polypeptide comprises a sequence having at least 75%, at least 80%, at least 85%, at least 90%, at least 95%, or at least 99% sequence identity to the sequence of any one of SEQ ID NOs: 44 or 47. In some embodiments, the second polypeptide comprises the sequence of any one of SEQ ID NOs: 44 or 47. In some embodiments, the fourth polypeptide comprises a sequence having at least 75%, at least 80%, at least 85%, at least 90%, at least 95%, or at least 99% sequence identity to the sequence of any one of SEQ ID NOs: 86 or 89. In some embodiments, the fourth polypeptide comprises the sequence of any one of SEQ ID NOs: 86 or 89. In some embodiments, the first polypeptide comprises a sequence having at least 75%, at least 80%, at least 85%, at least 90%, at least 95%, or at least 99% sequence identity to the sequence of any one of SEQ ID NOs: 50 or 53. In some embodiments, the first polypeptide comprises the sequence of any one of SEQ ID NOs: 50 or 53. In some embodiments, the third polypeptide comprises a sequence having at least 75%, at least 80%, at least 85%, at least 90%, at least 95%, or at least 99% sequence identity to the sequence of any one of SEQ ID NOs: 92 or 95. In some embodiments, third polypeptide comprises the sequence of any one of SEQ ID NOs: 92 or 95. In some embodiments, the first polypeptide comprises a sequence having at least 75%, at least 80%, at least 85%, at least 90%, at least 95%, or at least 99% sequence identity to the sequence of any one of SEQ ID NOs: 50 or 53; and the second polypeptide a sequence having at least 75%, at least 80%, at least 85%, at least 90%, at least 95%, or at least 99% sequence identity to the sequence of any one of SEQ ID NOs: 44 or 47. In some embodiments, the first polypeptide comprises the sequence of any one of SEQ ID NOs: 50 or 53; and the second polypeptide comprises the sequence of any one of SEQ ID NOs: 44 or 47. In some embodiments, the third polypeptide comprises a sequence having at least 75%, at least 80%, at least 85%, at least 90%, at least 95%, or at least 99% sequence identity to the sequence of any one of SEQ ID NOs: 92 or 95; and the fourth polypeptide comprises a sequence having at least 75%, at least 80%, at least 85%, at least 90%, at least 95%, or at least 99% sequence identity to the sequence of any one of SEQ ID NOs: 86 or 89. In some embodiments, the third polypeptide comprises the sequence of any one of SEQ ID NOs: 92 or 95; and the fourth polypeptide comprises the sequence of any one of SEQ ID NOs: 86 or 89. In some embodiments, the antigen binding molecule comprises: (a) the first polypeptide, wherein the first polypeptide comprises a sequence having at least 75%, at least 80%, at least 85%, at least 90%, at least 95%, or at least 99% sequence identity to the sequence of any one of SEQ ID NOs: 50 or 53; (b) the second polypeptide, wherein the second polypeptide comprises a sequence having at least 75%, at least 80%, at least 85%, at least 90%, at least 95%, or at least 99% sequence identity to the sequence of any one of SEQ ID NOs: 44 or 47; (c) the third polypeptide, wherein the third polypeptide comprises a sequence having at least 75%, at least 80%, at least 85%, at least 90%, at least 95%, or at least 99% sequence identity to the sequence of any one of SEQ ID NOs: 92 or 95; and (d) the fourth polypeptide, wherein the fourth polypeptide comprises a sequence having at least 75%, at least 80%, at least 85%, at least 90%, at least 95%, or at least 99% sequence identity to the sequence of any one of SEQ ID NOs: 86 or 89. In some embodiments, the antigen binding molecule comprises: (a) the first polypeptide, wherein the first polypeptide comprises the sequence of any one of SEQ ID NOs: 50 or 53; (b) the second polypeptide, wherein the second polypeptide comprises the sequence of any one of SEQ ID NOs: 44 or 47; (c) the third polypeptide, wherein the third polypeptide comprises the sequence of any one of SEQ ID NOs: 92 or 95; and (d) the fourth polypeptide, wherein the fourth polypeptide comprises the sequence of any one of SEQ ID NOs: 86 or 89. In some embodiments, the antigen binding molecule comprises: (a) the first polypeptide, wherein the first polypeptide comprises the sequence of SEQ ID NO: 50; (b) the second polypeptide, wherein the second polypeptide comprises the sequence of SEQ ID NO: 44; (c) the third polypeptide, wherein the third polypeptide comprises the sequence of any one of SEQ ID NO: 92; and (d) the fourth polypeptide, wherein the fourth polypeptide comprises the sequence of SEQ ID NO: 86. In some embodiments, the antigen binding molecule comprises: (a) the first polypeptide, wherein the first polypeptide comprises the sequence of SEQ ID NO: 53; (b) the second polypeptide, wherein the second polypeptide comprises the sequence of SEQ ID NO: 47; (c) the third polypeptide, wherein the third polypeptide comprises the sequence of SEQ ID NO: 95; and (d) the fourth polypeptide, wherein the fourth polypeptide comprises the sequence of SEQ ID NO: 89. In some embodiments, the antigen binding molecule or fragment thereof is conjugated or linked to a cytotoxic agent. In some embodiments, the antigen binding molecule or fragment thereof is conjugated or linked to a small molecule.

Provided herein, in some aspects, are antibodies or an antigen-binding portion thereof, wherein the antibody or an antigen-binding portion thereof competes with and/or binds the same epitope as a reference antibody, wherein the reference antibody comprises: (a) a heavy chain variable region (VH) comprising the amino acid sequence set forth in SEQ ID NO: 43; and (b) a light chain variable region (VL) comprising the amino acid sequence set forth in SEQ ID NO: 49. In some embodiments, the Kd of the antibody or an antigen-binding portion thereof to EGFR is within +/−10%, +/−20%, or +/−30% of the binding affinity of the reference antibody to EGFR. In some embodiments, binding of the antibody or an antigen-binding portion thereof to EGFR is configured to block the binding of epidermal growth factor (EGF). In some embodiments, the antibody or an antigen-binding portion thereof is configured to bind an epitope that overlaps with a cetuximab epitope.

Provided herein, in some aspects, are antibodies or an antigen-binding portion thereof, wherein the antibody or an antigen-binding portion thereof competes with and/or binds the same epitope as a reference antibody, wherein the reference antibody comprises: (a) a heavy chain variable region (VH) comprising the amino acid sequence set forth in SEQ ID NO: 85; and (b) a light chain variable region (VL) comprising the amino acid sequence set forth in SEQ ID NO: 91. In some embodiments, the Kd of the antibody or an antigen-binding portion thereof to ITGB6 is within +/−10%, +/−20%, or +/−30% of the binding affinity of the reference antibody to ITGB6. In some embodiments, the antibody or an antigen-binding portion thereof is configured to bind to an epitope of ITGB6 on a target cell, wherein the epitope does not comprise an epitope to which latency-associated peptide (LAP) binds.

Provided herein, in some aspects, are recombinant polynucleotides molecule comprising the polynucleotide sequences encoding the antigen binding molecule disclosed herein. In some embodiments, the recombinant polynucleotide molecule is an isolated recombinant polynucleotide molecule.

Provided herein, in some aspects, are vectors comprising the recombinant polynucleotide molecule disclosed herein.

Provided herein, in some aspects, are cells comprising the recombinant polynucleotide molecule disclosed herein, or the vector disclosed herein.

Provided herein, in some aspects, are pharmaceutical compositions comprising the antigen binding molecule disclosed herein, the recombinant polynucleotide disclosed herein, the vector disclosed herein, or the cell disclosed herein, and a pharmaceutically acceptable carrier, excipient, or diluent.

Provided herein, in some aspects, are methods of treating a condition or disease in a subject in need thereof, comprising administering to the subject a therapeutically effective amount of the antigen binding molecule disclosed herein, the recombinant polynucleotide disclosed herein, the vector disclosed herein, the cell disclosed herein, the pharmaceutical composition disclosed herein, or any combination thereof, thereby treating the condition or disease in the subject. In some embodiments, the condition or disease is cancer.

Provided herein, in some aspects, are methods of degrading EGFR on the surface of a cancer cell comprising, contacting the cell with an EGFR×ITGB6 bispecific antibody that competes with and/or binds the same epitope as a reference antibody, wherein the reference antibody comprises: (a) a heavy chain variable region (VH) comprising the amino acid sequence set forth in SEQ ID NO: 43; and (b) a light chain variable region (VL) comprising the amino acid sequence set forth in SEQ ID NO: 49. Also provided herein, in some aspects, are methods of degrading EGFR on the surface of a cancer cell comprising, contacting the cell with an EGFR×ITGB6 bispecific antibody that competes with and/or binds the same epitope as a reference antibody, wherein the reference antibody comprises: (a) a heavy chain variable region (VH) comprising the amino acid sequence set forth in SEQ ID NO: 85; and (b) a light chain variable region (VL) comprising the amino acid sequence set forth in SEQ ID NO: 91. Provided herein, in some aspects, are methods of selectively killing an EFGR expressing cancer cell comprising, contacting the cell with an EGFR×ITGB6 bispecific antibody that competes with and/or binds the same epitope as a reference antibody, wherein the reference antibody comprises: (a) a heavy chain variable region (VH) comprising the amino acid sequence set forth in SEQ ID NO: 43; and (b) a light chain variable region (VL) comprising the amino acid sequence set forth in SEQ ID NO: 49. Further provided herein, in some aspects, are methods of selectively killing an EFGR expressing cancer cell comprising, contacting the cell with an EGFR×ITGB6 bispecific antibody that competes with and/or binds the same epitope as a reference antibody, wherein the reference antibody comprises: (a) a heavy chain variable region (VH) comprising the amino acid sequence set forth in SEQ ID NO: 85; and (b) a light chain variable region (VL) comprising the amino acid sequence set forth in SEQ ID NO: 91. In some embodiments, the cancer cell is a non-small cell lung cancer (NSCLC) cell, a colorectal cancer (CRC) cell, or a squamous cell carcinoma (HNSCC) cell. In some embodiments, the cancer cell is a NSCLC cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “figure” and “FIG.” herein), of which:

FIG. 1 depicts a method of the present disclosure in which degradation of a target protein 112 (e.g., EGFR) on a surface of a target cell 111 is mediated by binding of an endogenous internalizing receptor 113 (e.g., ITGB6) and the target protein with an antigen binding molecule 101 disclosed herein.

FIG. 2A shows a graph depicting group mean tumor volume growth kinetics and bispecific antibody-mediated tumor growth suppression in a non-small cell lung cancer model. EGFR-ESTT corresponds to SEQ ID NOs: 126, 181, 236, 291, 346, 401, 456, 511, 566, and 621. ITGB6(2A1) corresponds to SEQ ID NOs: 177, 232, 287, 342, 397, 452, 507, 562, 617, and 672. ITGB6(2G2) corresponds to SEQ ID NOs: 179, 234, 289, 344, 399, 454, 509, 564, 619, and 674.

FIG. 2B shows a graph depicting group mean and individual animal tumor volumes at day 14 of bispecific antibody-mediated tumor growth suppression.

FIG. 3 shows a process for discovery of human-mouse cross-reactive EGFR antibodies.

FIG. 4A shows kinetic profiles of anti-EGFR antibodies binding to human EGFR analyte measured by surface plasmon resonance. Depicted antibodies: ES11 (SEQ ID NOs: 126, 181, 236, 291, 346, 401, 456, 511, 566, and 621), ES_11v23 (SEQ ID NOs: 148, 203, 258, 313, 368, 423, 478, 533, 588, and 643), ES 11v37 (SEQ ID NOs: 162, 217, 272, 327, 382, 437, 492, 547, 602, and 657), ES_11v38 (SEQ ID NOs: 163, 218, 273, 328, 383, 438, 493, 548, 603, and 658), ES_20 (SEQ ID NOs: 174, 229, 284, 339, 394, 449, 504, 559, 614, and 669), ES_21 (SEQ ID NOs: 175, 230, 285, 340, 395, 450, 505, 560, 615, and 670), ES_30 (SEQ ID NOs: 176, 231, 286, 341, 396, 451, 506, 561, 616, and 671).

FIG. 4B shows a dose-dependent binding of single arm EGFR binders on EGFR-expressing tumor cells. Depicted antibodies: ES 11 (SEQ ID NOs: 126, 181, 236, 291, 346, 401, 456, 511, 566, and 621), ES_11v23 (SEQ ID NOs: 148, 203, 258, 313, 368, 423, 478, 533, 588, and 643), ES_11v37 (SEQ ID NOs: 162, 217, 272, 327, 382, 437, 492, 547, 602, and 657), ES_11v38 (SEQ ID NOs: 163, 218, 273, 328, 383, 438, 493, 548, 603, and 658), ES_20 (SEQ ID NOs: 174, 229, 284, 339, 394, 449, 504, 559, 614, and 669), ES_21 (SEQ ID NOs: 175, 230, 285, 340, 395, 450, 505, 560, 615, and 670), ES_30 (SEQ ID NOs: 176, 231, 286, 341, 396, 451, 506, 561, 616, and 671).

FIG. 5A shows the EGF/EGFR signaling pathway in INDIGO's reporter cells that contain the luciferase reporter gene linked to an upstream promoter STAT3, which upon activation drives the Luc gene expression.

FIGS. 5B and 5C shows EGF/EGFR blocking potency of single arm anti-EGFR antibodies (B) or bispecific antibodies with ITGB6 and EGFR binding arms (C) at various concentrations. EGFR-ES11v23 corresponds to SEQ ID NOs: 148, 203, 258, 313, 368, 423, 478, 533, 588, and 643. h2A1 corresponds to SEQ ID NOs: 178, 233, 288, 343, 398, 453, 508, 563, 618, and 673. EGFR-ES11v37 corresponds to SEQ ID NOs: 162, 217, 272, 327, 382, 437, 492, 547, 602, and 657. EGFR-ES11v38 corresponds to SEQ ID NOs: 163, 218, 273, 328, 383, 438, 493, 548, 603, and 658. EGFR-ES20 corresponds to SEQ ID NOs: 174, 229, 284, 339, 394, 449, 504, 559, 614, and 669. EGFR-ES21 corresponds to SEQ ID NOs: 175, 230, 285, 340, 395, 450, 505, 560, 615, and 670. EGFR-ES11v30 corresponds to SEQ ID NOs: 155, 210, 265, 320, 375, 430, 485, 540, 595, and 650. EGFR-ES30 corresponds to SEQ ID NOs: 176, 231, 286, 341, 396, 451, 506, 561, 616, and 671.

FIG. 6 shows a western blot of EGFR degradation and phospho-EGFR levels in primary epidermal keratinocytes collected 48 hours after a single dose of single arm anti-EGFR antibodies (50 and 500 nM).

FIG. 7 shows a graph depicting group mean serum concentrations of monovalent anti-EGFR antibodies in mice following a single intravenous dose at either 3 mg/kg or 15 mg/kg.

FIGS. 8A-G show flow cytometry plots of A375 cells which overexpress ITGB6, ITGB8, or neither incubated with anti-ITGB6 antibodies and fluorescently labeled with secondary antibodies that bind to the anti-ITGB6 antibodies. Depicted antibodies: ITGB6 (2A1) (SEQ ID NOs: 177, 232, 287, 342, 397, 452, 507, 562, 617, 672), ITGB6 (2G2) (SEQ ID NOs: 179, 234, 289, 344, 399, 454, 509, 564, 619, 674).

FIG. 9 shows a graph depicting group mean serum concentrations of bivalent anti-ITGB6 (2A1 and 2G2) parent antibodies and Cetuximab in mice following a single intravenous dose at either 3 mg/kg or 15 mg/kg.

FIG. 10A shows SPR kinetic profiles of anti-ITGB6 antibody h2A1_H5 binding to uV06 ECD analyte in the absence and presence of 10-fold excess Latency Associated Peptide (LAP).

FIG. 10B shows EGFR degradation in NCIH1975 non-small cell lung cancer (NSCLC) cells treated with EGFRxITGB6 or control antibodies.

FIG. 11A shows expression of EGFR and Beta6 on primary Keratinocytes.

FIG. 11B shows a western blot of EGFR degradation and phospho-EGFR levels in primary epidermal keratinocytes collected 48 hours after a single dose of EGFRxITGB6 antibodies (50 nM and 500 nM).

FIG. 12 shows cytokine levels in primary human skin treated with standard of care antibodies or EGFRxITGB6 antibodies.

FIG. 13 shows EGFR cell surface removal on NCIH1975 cells 72 hours after treatment with single arm anti-EGFR antibodies and EGFRxITGB6 antibodies. Depicted antibodies: ES11 (SEQ ID NOs: 126, 181, 236, 291, 346, 401, 456, 511, 566, and 621), ES11v23 (SEQ ID NOs: 148, 203, 258, 313, 368, 423, 478, 533, 588, and 643), ES11v37 (SEQ ID NOs: 162, 217, 272, 327, 382, 437, 492, 547, 602, and 657), ESI1v38 (SEQ ID NOs: 163, 218, 273, 328, 383, 438, 493, 548, 603, and 658), ES20 (SEQ ID NOs: 174, 229, 284, 339, 394, 449, 504, 559, 614, and 669), ES21 (SEQ ID NOs: 175, 230, 285, 340, 395, 450, 505, 560, 615, and 670), ES30 (SEQ ID NOs: 176, 231, 286, 341, 396, 451, 506, 561, 616, and 671).

FIG. 14 shows EGFR degradation, pEGFR reduction, and downstream pERK signaling inhibition in NCIH1975 cells treated with single arm anti-EGFR antibodies and EGFRxITGB6 antibodies for 48 hours.

FIG. 15 shows mean tumor cell killing, as measured by GFP signal, for single arm EGFR antibodies and EGFRxITGB6 antibodies. Depicted antibodies: RSV×EGFR-ES11v23, RSV×EGFR-ES11v37, RSV×EGFR-ES11v38, RSV×EGFR-ES20.

FIGS. 16A-B show mean tumor volume growth kinetics of mice treated with EGFRxITGB6 antibodies or single arm EGFR antibodies. The insert panel in FIG. 16A shows individual animal tumor volume growth kinetics.

FIGS. 17A-C show % EGFR cell surface removal, % EGFR degradation, and % pEGFR degradation for various bispecific antibodies with bYlok®.

FIGS. 18A-B shows structural models of the human C_{H}1 domain (FIG. 18A left), human CL domain (FIG. 18A middle), shark C_{H}1 domain (FIG. 18B left), and shark C_{L} domain (FIG. 18B middle). On the C_{H}1 domains, Cluster 1 and Cluster 2 residues (highlighted in black) appear on the right and left side of the structure, respectively, while on the CL domain Cluster 1 residues appear in the middle of the domain and Cluster 2 residues appear at the top of the domain. The rightmost panels (FIGS. 18A-B) show the direct interaction between each cluster across the CH1-CL interface in the human (FIG. 18A right) and shark (FIG. 18B right) Fabs.

FIGS. 19A-C shows binding of EPI4004 (FIG. 19A, top panel) and charge-pair variants EPI4439 (FIG. 19A, middle panel) and EPI4629 (FIG. 19A, bottom panel) to EGFR and the integrin alpha V/beta 6 heterodimer (FIG. 19B) as measured on the Octet, along with associated equilibrium binding constants and kinetic rates (FIG. 19C). EPI4004 (EGFR×ITGB6) corresponds to SEQ ID NOs: 13-21, 31-36, 43-45, 49-51, 55-63, 73-78, 85-87, and 91-93).

FIG. 20 shows the western blot measurements of known standard of care molecules, and monovalent and bispecific EpiTAC-mediated phospho-EGFR levels in NCI-1975 NSCLC tumor cells collected 48 hrs following a single dose of mAbs. Exemplary antibody/antibody arm “ES_11v38/ByLok h2A1_H5_4×2 shark” comprises an EGFRxITGB6 bispecific with F116T, F118M, N137D, and N138D Light Chain mutations in an EGFR arm; L124S, G141L, H172K and T192K Heavy Chain mutations in the EGFR arm; N137K and N138K Light Chain mutations in an ITGB6 arm; H172D and T192D Heavy Chain mutations in the ITGB6 arm; and the ITGB6 arm further comprises bYlok® mutations. Exemplary antibody/antibody arm “ES_11v38/ByLok h2A1_H5_2×2 shark” comprises an EGFRxITGB6 bispecific with N137D and N138D Light Chain mutations in an EGFR arm; H172K and T192K Heavy Chain mutations in the EGFR arm; N137K and N138K Light Chain mutations in an ITGB6 arm; H172D and T192D Heavy Chain mutations in the ITGB6 arm; and the ITGB6 arm further comprises bYlok® mutations.

FIGS. 21A-B shows the binding affinities of the standard of care molecules and EpiTACs to human EGFR and EGFR blocking potency in EGFR reporter cell assay (FIG. 21A) and western blot measurements of known standard of care molecules, and monovalent and bispecific EpiTAC-mediated phospho-EGFR levels in primary epidermal keratinocytes collected 48 hrs following a single dose of mAbs at 500 and 50 nM concentration (FIG. 21B). Exemplary antibody/antibody arm “ES_11v38×2A1” comprises an EGFRxITGB6 bispecific with F116T, F118M, N137D, and N138D Light Chain mutations in an EGFR arm; L124S, G141L, H172K and T192K Heavy Chain mutations in the EGFR arm; N137K and N138K Light Chain mutations in an ITGB6 arm; H172D and T192D Heavy Chain mutations in the ITGB6 arm; and the ITGB6 arm further comprises bYlok® mutations.

FIGS. 22A-B shows the quantification of western blot measurements of monovalent (single-arm EGFR antibody) and EPI4004 mediated EGFR levels collected 48 hrs following a single dose of mAbs at 200 nM concentration (FIG. 22A) and the mean tumor volume growth kinetics in EGFR mutant (EGFR exon 19 deletion) xenograft models treated with EPI4004 (exemplary EGFRxITGB6 bispecific disclosed herein), or Osimertinib (FIG. 22B). Exemplary antibody “EP14004” comprises an EGFRxITGB6 bispecific with F116T, and F118M Light Chain mutations in an EGFR arm; L124S, and G141L Heavy Chain mutations in the EGFR arm; an ITGB6 arm comprises bYlok® mutations.

FIGS. 23A-C shows a schematic of an exemplary EGFR signaling cascade (e.g., binding affinities of the standard of care molecules and EpiTACs to human EGFR) (FIG. 23A); the quantification of western blot measurements of monovalent (e.g., single-arm EGFR antibody) and EP14004 mediated phospho-protein levels collected 48 hrs following a single dose of mAbs at 200 nM concentration (FIG. 23B); and immunohistopathology (IHC) staining for total EGFR and p-EGFR following treatment with EP14004 (exemplary EGFRxITGB6 bispecific disclosed herein) (FIG. 23C).

FIGS. 24A-C shows the mean tumor volume growth kinetics of EGFR mutant tumor-bearing mice treated with EP14004 and monovalent (e.g., single-arm EGFR and single-arm ITGB6) binder control mAbs (FIG. 24A); quantification of the total EGFR and pEGFR western blot protein signal from tumors collected 72 hrs after a single dose of EP13473 and monovalent (e.g., single-arm EGFR and single-arm ITGB6) binder control mAbs (FIG. 24B); and the mean tumor volume growth kinetics of EGFR L858R/T790M xenograft tumor models treated with an exemplary EGFRxITGB6 bispecific disclosed herein or Osimertinib (FIG. 24C). Exemplary antibody “EP14004 CC” comprises EP14004 with charge complimentary mutations. Exemplary antibody “EPI3473” comprises an EGFRxITGB6 bispecific with F116T, and F118M Light Chain mutations in an EGFR arm; L124S, and G141L Heavy Chain mutations in the EGFR arm.

FIGS. 25A-E shows the extended tumor suppression and survival kinetics of EGFR mutant tumor-bearing mice from multiple studies. Studies were conducted with and without Osimertinib resistance mutations (C797S). The mean tumor volume growth kinetics of Osimertinib responsive models NCI-H1975(L858R/T790M), treated with EGFRxITGB6 mAbs including EP14004 or Osimertinib (FIG. 25A). The mean tumor volume growth kinetics of resistant mice treated with EPI1550 or Osimertinib in the NCI-H1975(L858R/T790M/C797S) model (FIG. 25B). The mean tumor volume of mice from both models treated with a monovalent binder (e.g., single-arm EGFR), EPI4004, or Osimertinib (FIG. 25C). The mean tumor volume growth kinetics (FIG. 25D) and the survival kinetics (FIG. 25E) of Osimertinib-resistant EGFR L858R/T790M/C797S mutant xenograft NSCLC tumor models treated with an exemplary EGFRxITGB6 bispecific disclosed herein or Osimertinib. Exemplary antibody “EPIl550” comprises an EGFRxITGB6 bispecific.

FIG. 26 shows exemplary expression (e.g., RNA levels) of a degrader protein (e.g., ITGB6) on various tissue types (e.g., normal skin, normal colon, NSCLC, head and neck squamous cell carcinoma (HNSCC), esophageal tumor, bladder tumor, colorectal tumor) and indicates that degrader receptor expression may localize activity and drive degradation to tumors expressing distinct oncogenic forms of EGFR.

FIGS. 27A-B shows a graph depicting group mean serum concentrations of EPI4004 and EPI4629 in tumor-free mice following a single intravenous dose at either 3 mg/kg or 10 mg/kg (FIG. 27A) and a graph depicting group mean serum concentrations of exemplary EGFRxITGB6 bispecifics disclosed herein in non-human primate (NHP, cynomolgus macaques) following a single intravenous dose at 3 dose levels over a period of a week (FIG. 27B). Exemplary antibody “EPI4629” comprises an EGFRxITGB6 bispecific with F116T, F118M, N137D, and N138D Light Chain mutations in an EGFR arm; L124S, G141L, H172K and T192K Heavy Chain mutations in the EGFR arm; N137K and N138K Light Chain mutations in an ITGB6 arm; H172D and T192D Heavy Chain mutations in the ITGB6 arm; and the ITGB6 arm further comprises bYlok® mutations.

FIG. 28 shows the quantification of western blot measurements of IgG control, monovalent (e.g., single-arm EGFR), EP14004, and EP14629 mediated EGFR levels collected 48 hrs following a single dose of mAbs at 200 nM concentration.

FIG. 29 depicts mean tumor volume growth kinetics and tumor growth suppression in an NCIH1975 NSCLC xenograft tumor model treated with an exemplary EGFRxITGB6 bispecific antibody (EP14004).

FIG. 30 depicts total tumor EGFR degradation during a 1-week period in an NCIH1975 NSCLC xenograft tumor model following treatment with an exemplary EGFRxITGB6 bispecific antibody (EP14004) via a single intravenous dose.

FIG. 31 shows exemplary western blot gel images measuring total tumor EGFR on day 7 in an NCIH1975 NSCLC xenograft tumor model following treatment with an exemplary EGFRxITGB6 bispecific antibody (EP14004) via a single intravenous dose (see FIG. 30).

FIG. 32 depicts mean tumor volume growth kinetics in an NCIH1975 NSCLC xenograft tumor model following treatment with an exemplary EGFRxITGB6 bispecific antibody (EP14004) at a dose of 15 mg/kg over an extended dosing schedule.

FIG. 33 depicts mean tumor volume growth kinetics and tumor growth suppression in a Detroit562 HNSCC xenograft tumor model following weekly treatment with an exemplary EGFRxITGB6 bispecific antibody (EP14004) at a dose of 15 mg/kg.

FIG. 34 depicts mean tumor volume growth kinetics and tumor growth suppression in a CTG-0149 Patient-Derived HNSCC xenograft tumor model following weekly treatment with an exemplary EGFRxITGB6 bispecific antibody (EP14004) at a dose of 15 mg/kg.

FIG. 35 depicts mean tumor volume growth kinetics and tumor growth suppression in a CR5030 Patient-Derived Colorectal Cancer xenograft tumor model following weekly treatment with an exemplary EGFRxITGB6 bispecific antibody (EP14004) at a dose of 15 mg/kg.

FIG. 36 depicts mean tumor volume growth kinetics and tumor growth suppression in a CR9510 Patient-Derived Colorectal Cancer xenograft tumor model following weekly treatment with an exemplary EGFRxITGB6 bispecific antibody (EP14004) at a dose of 15 mg/kg.

FIG. 37. shows the tumor suppression in NCI-H1975(L858R/T790M) mouse model treated with an isotype control, EGFRxITGB6 bispecific antibody (EP14004), or an scFVxFab bispecific antibody (scFV: EGFR; Fab: ITGB6).

### DETAILED DESCRIPTION

### Overview

Targeted protein degradation is a promising therapeutic strategy compared to conventional inhibition-based therapeutics. For example, targeted protein degradation allows for the elimination of disease-causing proteins that were previously considered “undruggable” using conventional inhibitors. It also offers the potential for increased selectivity and reduced off-target effects, as the degradation process specifically targets the disease-associated protein. While targeted protein degradation therapies have shown great promise in treating various diseases, there can be challenges associated with achieving cell specificity. For example, targeted protein degradation therapies may act on several cell types, leading to off-target effects which can result in adverse reactions and interfere with normal cellular functions. This can be particularly problematic when treating cancers where treatments for cancers which lack cell-type specificity often result in killing many fast-growing cells, not just cancer cells. Consequently, cancer treatments which lack cell-type specificity can have significant side effects and increase the risk of infections. Thus, improving the selectivity of targeted protein degradation therapies, and developing strategies to target specific cell-types within heterogeneous populations may be crucial for the successful development of targeted protein degradation therapies for the treatment of cancers.

The methods and compositions described herein can overcome the drawbacks of the currently available targeted protein degradation therapies. The present disclosure generally relates to antigen binding molecules, which bind to both a target protein, and a membrane-associated internalizing protein and/or a membrane-associated degrading protein present on the surface of a target cell. In some embodiments, the present disclosure provides an antigen binding molecule (e.g., bispecific antibody) that binds a target protein and a membrane-associated internalizing protein, leading to cellular internalization of the target protein and subsequent degradation of the target protein. In other embodiments, the present disclosure provides methods of degrading a target protein comprising contacting the target protein with an antigen binding molecule that binds a membrane-associated degrading protein, leading to degradation of the target protein.

A common challenge of producing bispecific antigen binding molecules (e.g., bispecific antibodies) is the potential heterogeneity that can arise from the presence of two different binding arms. Several purification strategies designed for bispecific antibodies have been developed. For example, one approach involves the use of dual-column chromatography systems, where two separate affinity chromatography columns are used in tandem. Each column is packed with a specific ligand that can selectively bind to an arm of the antibody. However, these methods can be challenging with bispecific antibodies due to the multiplicity of structures generated during the production process (e.g., mispairing of heavy and light chains). Since the variants generated during the production process often copurify with the desired bispecific antibody product. Moreover, affinity chromatography purification of bispecific antibodies is often a two-step process (e.g., Protein G purification and Protein A purification), which can present certain manufacturing challenges. For example, a two-step purification process may be more time-consuming, result in increased production costs, lead to reduced overall yield and lower productivity, and can affect the quality and stability of the final product. Thus, there is a need for more efficient production strategies for bispecific antibodies. The methods and compositions described herein can overcome the drawbacks of the currently available production strategies for bispecific antibodies. The present disclosure generally relates to antigen binding molecules, which bind to both a target protein, and an internalizing protein and/or degrading protein, wherein the antigen binding molecules comprise one or more mutations that promote the correct heterodimerization of the bispecific antigen binding molecules disclosed herein.

### Binding Agents

The present disclosure provides binding agents. The binding agent can be an antigen binding molecule, such as a bispecific antibody. For example, the antigen binding molecule can comprise at least two binding domains: one specific for a membrane-associated internalizing protein and/or a membrane-associated degrading protein, such as ITGB6, and the other specific for an EGFR protein. The antigen binding molecules of the present disclosure comprise: (i) a first antigen binding domain that binds to an epidermal growth factor receptor (EGFR); and (ii) a second antigen binding domain that binds to an integrin subunit beta 6 (ITGB6).

Epidermal Growth Factor Receptor (EGFR) is a transmembrane protein that is a receptor for extracellular protein ligands of the epidermal growth factor family (EGF family). EGFR is activated by binding of these specific ligands, including epidermal growth factor (EGF) and transforming growth factor a (TGFα). Aberrant EGFR function and/or expression is implicated in cancer, where it causes enhanced cell growth and division and drives tumor growth and invasion.

Mutations that lead to EGFR overexpression (known as upregulation or amplification) have been associated with a number of cancers, including adenocarcinoma of the lung cancer, anal cancers, glioblastoma and epithelial tumors of the head and neck. Mutations, amplifications or mis-regulations of EGFR or family members are implicated in about 30% of all epithelial cancers. Many of these somatic mutations involving EGFR lead to its constant activation, which produces uncontrolled cell division. Therefore, the degradation of EGFR in cancer is a promising treatment modality for cancer.

ITGB6, also known as Integrin Subunit Beta 6, is a protein that is part of the integrin family of cell surface receptors. Integrins play crucial roles in cell adhesion, migration, and signaling. ITGB6 specifically forms a heterodimeric complex with the alpha-v integrin subunit, resulting in the formation of the integrin avP6. ITGB6 can be expressed in epithelial cells, including cells in the respiratory tract, gastrointestinal tract, and skin. Its expression is often low or absent in normal adult tissues but can be upregulated in response to injury or during certain pathological conditions, including inflammation and cancer.

ITGB6 can mediate cell adhesion and regulate cell behavior. It interacts with specific extracellular matrix proteins, such as fibronectin and tenascin-C, as well as soluble ligands like latent transforming growth factor-beta (TGF-0). The binding of ITGB6 to these ligands can trigger intracellular signaling pathways, leading to various cellular responses.

ITGB6 can play a role in TGF-β activation. The binding of ITGB6 to latent TGF-β complexes on the cell surface induces a conformational change, leading to the release and activation of TGF-β, which is a potent regulator of cell growth, differentiation, and tissue repair. Additionally, ITGB6 has been implicated in various biological processes and diseases. It is associated with tissue remodeling, wound healing, immune responses, and cancer progression. Dysregulation of ITGB6 expression or function has been observed in certain cancers, including pancreatic, lung, and oral squamous cell carcinomas, where it can contribute to tumor growth, invasion, and metastasis.

Antigen binding molecules of the disclosure include, without limitation, agents wherein the ITGB6 binding domain and the EGFR binding domain are each independently selected from an antibody (or half of an antibody), a nanobody, or a minibody, a Fab fragment, a single chain variable fragment (scFv), and a single domain antibody (sdAb), or a functional fragment thereof. These two binding domains can be the same type of molecule, or different. For example, antigen binding molecules of the disclosure include, without limitation, multispecific antigen binding molecules having an IgG that binds a membrane-associated internalizing or degrading protein, and an scFv domain that binds EGFR. The binding domains of the multispecific antigen binding molecule can be connected through covalent bonds, non-covalent interactions, or a combination thereof.

The antigen binding molecule can generally take the form of a protein, glycoprotein, lipoprotein, phosphoprotein, and the like. Some antigen binding molecules of the disclosure take the form of multispecific antibodies, bispecific antibodies, antibody-drug conjugates (ADCs), or antibody derivatives. In some embodiments, the antigen binding molecule comprises an antibody. In some embodiments, the antigen binding molecule comprises a multispecific antibody. In some embodiments, the antigen binding molecule comprises a bispecific antibody. In some embodiments, the antigen binding molecule comprises an IgG antibody. In some embodiments, the antigen binding molecule comprises a multispecific IgG antibody. In some embodiments, the antigen binding molecule comprises a knob and hole bispecific IgG. In some embodiments, the antigen binding molecule comprises an ADC. In some embodiments, the antigen binding molecule comprises a T cell engager. In some embodiments, the antigen binding molecule comprises a bispecific antigen binding molecule. In some embodiments, the antigen binding molecule comprises a bispecific antibody. In some embodiments, the antigen binding molecule comprises a bispecific diabody. In some embodiments, the antigen binding molecule comprises a bispecific Fab2. In some embodiments, the antigen binding molecule comprises a bispecific camelid antibody. In some embodiments, the antigen binding molecule comprises a bispecific peptibody scFv-Fc. In some embodiments, the antigen binding molecule comprises Fc-Fab. In some embodiments, the antigen binding molecule comprises a knob and hole bispecific Fc-Fab. In some embodiments, the target protein binding domain is selected from the group consisting of a half antibody, a nanobody, or a minibody, a F(ab′)2 fragment, a Fab fragment, a single chain variable fragment (scFv), and a single domain antibody (sdAb), or a functional fragment thereof. The binding domains may together take the form of a bispecific antibody, a bispecific diabody, a bispecific camelid antibody or a bispecific peptibody, and the like. Antibody derivatives need not be derived from a specific wild type antibody. For example, one can employ known techniques such as phage display to generate and select for small proteins having a binding domain similar to an antibody complementarity-determining region (CDR). In some embodiments, the antigen-binding moiety includes an scFv. The binding domain can also be derived from a natural or synthetic ligand or receptor, whether soluble or membrane-bound, that specifically binds to the EGFR protein. The binding domain can also be derived from a natural or synthetic ligand or receptor, whether soluble or membrane-bound, that specifically binds to the ITGB6 protein.

Multispecific antibodies can be prepared by known methods. Embodiments of the disclosure include “knob-into-hole” bispecific antibodies, wherein the otherwise symmetric dimerization region of a bispecific antigen binding molecule is altered so that it is asymmetric. For example, a knob-into-hole bispecific IgG that is specific for antigens A and B can be altered so that the Fc portion of the A-binding chain has one or more protrusions (“knobs”), and the Fc portion of the B-binding chain has one or more hollows (“holes”), where the knobs and holes are arranged to interact. This reduces the homodimerization (A-A and B-B antibodies) and promotes the heterodimerization desired for a bispecific antigen binding molecule. See, e.g., Y. Xu et al., mAbs (2015) 7(1):231-42. In some embodiments, the bispecific antigen binding molecule has a knob-into-hole design. In some embodiments, the “knob” comprises a T336W alteration of the CH3 domain, i.e., the threonine at position 336 is replaced by a tryptophan. In some embodiments, the “hole” comprises one or a combination of T366S, L368A, and Y407V. In some embodiments, the “hole” comprises T366S, L368A, and Y407V.

In some embodiments, the multispecific antigen binding molecule comprises an FcRn receptor recognition domain, to promote return of the bispecific antigen binding molecule to the extracellular space if the bispecific antigen binding molecule is internalized.

In some embodiments, “complementarity-determining region” or “CDR” can refer to variable regions of either H (heavy) or L (light) chains (e.g., VH and VL, respectively) and can contain the amino acid sequences capable of specifically binding to antigenic targets. For example, the CDR regions can account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have three CDR regions, each non-contiguous with the others (termed LCDR1, LCDR2, LCDR3, HCDR1, HCDR2, HCDR3) for the respective light (L) and heavy (H) chains. In some embodiments, nanobodies can comprise a single amino acid chain that can be considered to comprise four “framework sequences or regions” or FRs and three complementarity-determining regions” or CDRs. The nanobodies have three CDR regions, each non-contiguous with the others (termed CDR1, CDR2, CDR3). The delineation of the FR and CDR sequences is based on the IMGT unique numbering system for V-domains and V-like domains.

The present disclosure provides a bispecific anti-EGFR and anti-ITGB6 antigen binding molecule (e.g., a bispecific antibody). In some embodiments, the bispecific antigen binding molecule disclosed herein can bind to epidermal growth factor receptor (EGFR) and block the binding of epidermal growth factor (EGF). In some cases, the bispecific antigen binding molecule disclosed herein binds an epitope that overlaps with cetuximab. Cetuximab is an antibody that binds to a specific region on EGFR, inhibiting the interaction of EGF and other ligands with the receptor. By overlapping with the cetuximab epitope, a bispecific antibody can essentially compete with cetuximab for binding to EGFR, thereby blocking the binding of EGF. For example, the bispecific antigen binding molecule disclosed herein may bind to an epitope, wherein the epitope is present on human and murine EGFR (e.g., an epitope that overlaps with a murine epitope). In some cases, the bispecific antigen binding molecule (e.g., a bispecific antibody) may recognize a specific region on EGFR that is both different from the cetuximab binding site and similar to an EGFR murine binding site. The design and development of the bispecific antigen binding molecules disclosed herein targeting EGFR with an overlapping epitope to cetuximab can effectively bind to EGFR and simultaneously prevent EGF from interacting with the receptor. By targeting this distinct epitope, the bispecific antibody can effectively block the interaction between EGF and EGFR, further preventing downstream signaling events. This inhibition of the EGFR signaling pathway can have multiple potential benefits. For example, EGFR signaling plays a critical role in cell proliferation, survival, and angiogenesis, and dysregulation of this pathway is commonly observed in various cancers. Thus, by blocking EGF binding to EGFR, the bispecific antigen binding molecules disclosed herein can hinder tumor growth, invasion, and metastasis.

In some embodiments, the bispecific antigen binding molecule disclosed herein can bind to epidermal growth factor receptor (EGFR) and not block the binding of epidermal growth factor (EGF). In some embodiments, the bispecific antigen binding molecules disclosed herein targeting EGFR comprise an overlapping epitope to cetuximab and allow EGF to interact with the receptor. As described in Examples 9 and 14, the antigen binding molecules disclosed herein can effectively allow the interaction between EGF and EGFR, further enabling downstream signaling events. Maintaining the EGFR signaling pathway can have multiple potential benefits. For example, EGFR signaling plays a critical role in cell proliferation, and survival, thus enabling downstream signaling events to occur can help to maintain cellular homeostasis.

In some embodiments, the bispecific antigen binding molecule disclosed herein degrades EGFR when the second portion (e.g., anti-ITGB6 second binding arm) is present. For example, when the antigen binding molecule lacks the anti-ITGB6 binding arm EGFR is degraded less when compared to a corresponding anti-EGFR monovalent antigen binding molecule (e.g., a one-armed monovalent antibody). This design can offer several advantages, for example, it can enhance specificity by selectively targeting EGFR only in the presence of the desired target molecule, such as ITGB6, reducing the risk of off-target effects and potential toxicity. It can also help to localize the therapeutic action to the specific tumor site or microenvironment, improving the precision of treatment.

In some embodiments, the bispecific antigen binding molecule disclosed herein selectively binds to integrin subunit beta 6 (ITGB6) without blocking latent-associated peptide (LAP) binding. In some embodiments, the bispecific antigen binding molecule disclosed herein selectively binds to integrin subunit beta 6 (ITGB6) without blocking transforming growth factor-beta (TGF-0) activation. For example, unlike traditional therapeutic approaches that aim to block ITGB6 or TGF-0 signaling, the bispecific antigen binding molecule disclosed herein takes a more nuanced approach by selectively targeting ITGB6 without interfering with the important regulatory functions of TGF-0. LAP, a protein component of the latent TGF-0 complex, plays a crucial role in maintaining TGF-0 in an inactive form. It is important to preserve this interaction to allow for TGF-0 activation when needed. By ensuring that the second binding arm (e.g., anti-ITGB6 portion) of the bispecific antigen binding molecule disclosed herein does not block LAP binding, the bispecific antigen binding molecule can specifically bind to ITGB6 without affecting the latent TGF-0 complex. Additionally, by not interfering with LAP binding or TGF-0 activation, the bispecific antigen binding molecule disclosed herein can avoid disrupting the important functions of the TGF-0 pathway, including tissue homeostasis, immune regulation, and wound healing.

In some embodiments, the bispecific antigen binding molecule disclosed herein specifically targets integrin subunit beta 6 (ITGB6) without binding to other beta integrins. Integrin proteins play essential roles in cell adhesion, migration, and signaling. ITGB6 is a specific subunit that forms heterodimeric complexes with alpha integrins, particularly alpha-v integrins, contributing to the functional diversity of integrin receptors. However, the challenge lies in developing a bispecific antibody that recognizes ITGB6 exclusively without binding to other beta integrins. By avoiding interactions with other beta integrins, the bispecific antigen binding molecule disclosed herein can specifically modulate signaling pathways associated with ITGB6. This selective modulation may be crucial in conditions where ITGB6 plays a distinct role, such as in tumor progression, epithelial-mesenchymal transition, or tissue fibrosis.

In some embodiments, the antigen binding molecules disclosed herein are cross-reactive, wherein the antigen binding molecules can bind to targets (e.g., EGFR and/or ITGB6) in different species, such as human, murine (Mus musculus), and cynomolgus (non-human primate). As described in Example 4, this cross-species reactivity enables the study of molecules, pathways, and therapeutic candidates in various animal models and facilitates the translation of findings from preclinical studies to human clinical trials. In therapeutic applications, a cross-reactive bispecific antibody (e.g., the antigen binding molecules disclosed herein) that recognizes multiple species may offer advantages in early-stage development and clinical trials. It can aid in toxicity assessments and pharmacokinetic evaluations, helping to predict potential adverse effects and optimize dosage regimens in humans based on data obtained in relevant animal models. In some embodiments, the antigen binding molecules disclosed herein binds to an epitope of a human EGFR. In some embodiments, the antigen binding molecules disclosed herein binds to an epitope of a murine EGFR. In some embodiments, the antigen binding molecules disclosed herein binds to an epitope of a cynomolgus EGFR.

Provided herein are antibodies or an antigen-binding portion thereof, wherein the antibody or an antigen-binding portion thereof competes with and/or binds the same epitope as a reference antibody. In some embodiments, the reference antibody comprises: a heavy chain variable region (VH) comprising the amino acid sequence set forth in SEQ ID NO: 43; and a light chain variable region (VL) comprising the amino acid sequence set forth in SEQ ID NO: 49. In some embodiments, the Kd of the antibody or an antigen-binding portion thereof to EGFR is within +/−10%, +/−20%, or +/−30% of the binding affinity of the reference antibody to EGFR. In some embodiments, binding of the antibody or an antigen-binding portion thereof to EGFR is configured to block the binding of epidermal growth factor (EGF). In some embodiments, the antibody or an antigen-binding portion thereof is configured to bind an epitope that overlaps with a cetuximab epitope. In some embodiments, the reference antibody comprises: a heavy chain variable region (VH) comprising the amino acid sequence set forth in SEQ ID NO: 85; and a light chain variable region (VL) comprising the amino acid sequence set forth in SEQ ID NO: 91. In some embodiments, the Kd of the antibody or an antigen-binding portion thereof to ITGB6 is within +/−10%, +/−20%, or +/−30% of the binding affinity of the reference antibody to ITGB6. In some embodiments, the antibody or an antigen-binding portion thereof is configured to bind to an epitope of ITGB6 on a target cell, wherein the epitope does not comprise an epitope to which latency-associated peptide (LAP) binds.

The present disclosure provides an antigen binding molecule, comprising a first polypeptide, a second polypeptide, a third polypeptide and/or a fourth polypeptide. The first polypeptide, the second polypeptide, the third polypeptide and/or the fourth polypeptide can comprise an “arm” of an antibody (e.g., an antigen binding molecule disclosed herein). In some embodiments, the first polypeptide and the second polypeptide comprises an anti-EGFR arm of the antigen binding molecule disclosed herein. In some embodiments, the third polypeptide and the fourth polypeptide comprises an anti-ITGB6 arm of the antigen binding molecule disclosed herein. In the context of bispecific antibodies (e.g., antigen binding molecules disclosed herein), an arm of an antibody, such as an arm of the antigen binding molecules disclosed herein, can refer to one of the two binding specificities incorporated into the antibody molecule/antigen binding molecule. For example, bispecific antibodies are engineered to simultaneously target two different antigens, unlike traditional antibodies that typically recognize a single antigen, and each antibody arm of a bispecific antibody is designed to bind to a specific antigen. For instance, if we consider a bispecific antibody with one arm targeting antigen A (e.g., EGFR) and the other arm targeting antigen B (e.g., ITGB6), each arm will have its own antigen-binding site. This allows the bispecific antibody (e.g., bispecific anti-EGFR and anti-ITGB6 antigen binding molecules disclosed herein) to simultaneously bind to both antigens, likely bringing them in close proximity to each other.

In some embodiments, the first polypeptide comprises a first light chain constant region. In some embodiments, the third polypeptide comprises a second light chain constant region. In some embodiments, the first light chain constant region or the second light chain constant region, or a combination thereof comprises a kappa light chain constant region or functional fragment thereof, a lambda light chain constant region or functional fragment thereof, or a combination thereof. In some embodiments, the first polypeptide, the second polypeptide, the third polypeptide and/or the fourth polypeptide comprises a Fab or a scFv.

In some embodiments, the second polypeptide comprises one or more heavy chain constant regions. In some embodiments, the third polypeptide comprises one or more heavy chain constant regions. In some embodiments, the one or more heavy chain constant regions selected from the group consisting of IgG1 heavy chain constant region or functional fragment thereof, IgG2 heavy chain constant region or functional fragment thereof, IgG3 heavy chain constant region or functional fragment thereof, IgGA1 heavy chain constant region or functional fragment thereof, IgGA2 heavy chain constant region or functional fragment thereof, IgG4 heavy chain constant region or functional fragment thereof, IgJ heavy chain constant region or functional fragment thereof, IgM heavy chain constant region or functional fragment thereof, IgD heavy chain constant region or functional fragment thereof, and IgE heavy chain constant region or functional fragment thereof. In some embodiments, the one or more heavy chain constant regions is an IgG1 heavy chain constant region or functional fragment thereof.

In some embodiments, the second polypeptide comprises a first immunoglobulin constant region (Fc region). In some embodiments, the fourth polypeptide comprises a second Fc region. In some embodiments, the first Fc region, the second Fc region, or a combination thereof is selected from the group consisting of an IgG1 Fc region or a functional fragment thereof, an IgG2 Fc region or a functional fragment thereof, an IgG3 Fc region or a functional fragment thereof, an IgGA1 Fc region or a functional fragment thereof, an IgGA2 Fc region or a functional fragment thereof, an IgG4 Fc region or a functional fragment thereof, an IgJ Fc region or a functional fragment thereof, an IgM Fc region or a functional fragment thereof, an IgD Fc region or a functional fragment thereof, and an IgE Fc region or a functional fragment thereof. In some embodiments, the first Fc region and/or the second Fc region is an IgG1 Fc region or a functional fragment thereof. In some embodiments, the IgG1 Fc region or a functional fragment thereof comprises a backbone mutation, wherein the backbone mutation results in a change to FcγR binding and/or effector function. In some embodiments, the IgG1 Fc region or a functional fragment thereof comprises a Fc mutation. In some embodiments, the IgG1 Fc region or a functional fragment thereof comprises a deletion (such as removal) of a core fucose glycan.

In some embodiments, the antigen binding molecule is a multispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab.

In some embodiments, the antigen binding molecule comprises a first polypeptide and a second polypeptide, wherein the first polypeptide and the second polypeptide are non-contiguous, wherein: the first polypeptide comprises the VL of the first antigen binding domain and a first Light Chain Constant Region (CL), wherein the first CL is linked to the VL of the first antigen binding domain; and the second polypeptide comprises the VH of the first antigen binding domain and a first immunoglobulin constant region (Fc region), wherein the first Fc region is linked to the VH of the first antigen binding domain.

In some embodiments, the antigen binding molecule comprises a third polypeptide and a fourth polypeptide, wherein the third polypeptide and the fourth polypeptide are non-contiguous, wherein: the third polypeptide comprises the VL of the second antigen binding domain and a second Light Chain Constant Region (CL), wherein the second CL is linked to the VL of the second antigen binding domain; and the fourth polypeptide comprises the VH of the second antigen binding domain and a second immunoglobulin constant region (Fc region), wherein the second Fc region is linked to the VH of the second antigen binding domain.

In some embodiments, the VH of the first antigen binding domain comprises a dimerization domain; the VL of the first antigen binding domain comprises a dimerization domain; the VH of the second antigen binding domain comprises a dimerization domain; the VL of the second antigen binding domain comprises a dimerization domain; the CH of the second polypeptide comprises a dimerization domain; the CL of the first polypeptide comprises a dimerization domain; the CH of the fourth polypeptide comprises a dimerization domain; the CL of the third polypeptide comprises a dimerization domain; or a combination thereof. In some embodiments, the VH and VL of the first antigen binding domain are dimerized; the VH and VL of the second antigen binding domain are dimerized; the CH of the fourth polypeptide and the CL of the third polypeptide are dimerized; the CH of the second polypeptide and CL of the first polypeptide are dimerized; the CH of the second polypeptide and the CH of the fourth polypeptide are dimerized, or a combination thereof. In some embodiments, the dimerization domain comprises a disulfide bond.

In some embodiments, the antigen binding molecule comprises an anti-EGFR arm.

In some embodiments, the antigen binding molecule comprises an anti-ITGB6 arm. As described in Examples 15 and 18, in some embodiments, the first polypeptide, the second polypeptide, the third polypeptide and/or the fourth polypeptide of the antigen binding molecules disclosed herein are humanized. In some embodiments, the antigen binding molecules disclosed herein comprise a knob and hole bispecific antibody, wherein the anti-EGFR arm comprises a knob portion and the anti-ITGB6 arm comprises a hole portion.

In some embodiments, the antigen binding molecules disclosed herein targeting EGFR and/or ITGB6 may bind with a similar affinity as any one of the sequences listed in Table 1, Table 2 and/or Table 5. In certain embodiments, the antigen binding molecules have a Kd less than, more than, within 10%, within 20%, within 30%, within 40%, within 50%, withing 75%, or within 100% of the binding affinity of a monovalent EGFR or ITGB6 antigen binding molecule. For example, the binding affinity of a monovalent EGFR or ITGB6 antigen binding molecule may have a Kd of between 0.1 nM and 100 nM. When incorporated into the antigen binding molecule, disclosed herein, the Kd may be within the same range. Alternatively, the binding affinity may be slightly greater than, but within two-fold of the monovalent EGFR or ITGB6 binding affinity. The binding affinity may be within three-fold of the monovalent binding affinity.

As described in Examples 5 and 9, the antigen binding molecules disclosed herein display a “Goldilocks” binding affinity for EGFR. The term “Goldilocks” can refer to an antibody (e.g., the antigen binding molecules disclosed herein) that exhibits just the right balance in binding affinity, neither too strong nor too weak, to avoid harming healthy cells while effectively targeting and killing cancer cells. Achieving this balance requires careful consideration of binding affinity, target selection, and pharmacokinetics and can help to maximize therapeutic benefits while minimizing potential off-target effects. Binding affinity, such as the strength of binding between an antibody and its target, is essential to optimize to ensure specific and effective binding to cancer cells while minimizing binding to healthy cells. If the binding affinity is too weak, a bispecific antibody may not effectively target and eliminate cancer cells, possibly reducing therapeutic potential. Conversely, if the binding affinity is too strong, there is an increased risk of non-specific binding to healthy cells, which can lead to off-target toxicities. The selection of target antigens can also be important in achieving the desired “Goldilocks” balance. Bispecific antibodies can target antigens that are primarily expressed or overexpressed on cancer cells, while having limited or no expression on healthy cells. This selective targeting can help minimize damage to healthy tissues and reduce potential side effects. In some embodiments, the antigen binding molecules disclosed herein have increased EGFR degradation on a target cancer cell compared to EGFR degradation on a corresponding non-cancerous target cell. In some embodiments, the antigen binding molecules disclosed herein targeting EGFR and/or ITGB6 have a Kd less than the binding affinity of cetuximab.

In some embodiments, the antigen binding molecules, disclosed herein, targeting EGFR and ITGB6 comprise a sequence listed Table 1, Table 2 and/or Table 5. In some embodiments, the antigen binding molecules, disclosed herein, targeting EGFR and ITGB6 comprise a sequence listed Table 1, Table 2 and/or Table 5. In some embodiments, the antigen binding molecules, disclosed herein, targeting EGFR and ITGB6 comprise at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to a sequence listed Table 1, Table 2 and/or Table 5.

In some embodiments, the antigen binding molecules disclosed herein comprise one or more sequences having at least 75%, at least 80%, at least 85%, at least 90%, at least 95%, or at least 99% sequence identity to one or more sequences of SEQ ID NOs: 13-21, 31-36, 43-45, 49-51, 55-63, 73-78, 85-87, and 91-93. In some embodiments, the antigen binding molecules disclosed herein comprise one or more sequences having at least 75%, at least 80%, at least 85%, at least 90%, at least 95%, or at least 99% sequence identity to one or more sequences of SEQ ID NOs: 22-30, 37-42, 46-48, 52-54, 55-63, 73-78, 85-87, and 91-93.

In some embodiments, the antigen binding molecules disclosed herein comprise one or more sequences having at least 75%, at least 80%, at least 85%, at least 90%, at least 95%, or at least 99% sequence identity to one or more sequences of SEQ ID NOs: 13-21, 31-36, 43-45, 49-51, 64-72, 79-84, 88-90, and 94-96. In some embodiments, the antigen binding molecules disclosed herein comprise one or more sequences having at least 75%, at least 80%, at least 85%, at least 90%, at least 95%, or at least 99% sequence identity to one or more sequences of SEQ ID NOs: 22-30, 37-42, 46-48, 52-54, 64-72, 79-84, 88-90, and 94-96.

In some embodiments, the first polypeptide comprises a sequence having at least 75%, at least 80%, at least 85%, at least 90%, at least 95%, or at least 99% sequence identity to the sequence of any one of SEQ ID NO: 51 or 54. In some embodiments, the first polypeptide comprises a sequence of any one of SEQ ID NO: 51 or 54. In some embodiments, the second polypeptide comprises a sequence having at least 75%, at least 80%, at least 85%, at least 90%, at least 95%, or at least 99% sequence identity to the sequence of any one of SEQ ID NO: 45 or 48. In some embodiments, the second polypeptide comprises a sequence of any one of SEQ ID NO: 45 or 48. In some embodiments, the first polypeptide comprises a sequence having at least 75%, at least 80%, at least 85%, at least 90%, at least 95%, or at least 99% sequence identity to the sequence of any one of SEQ ID NOs: 51 or 54; and the second polypeptide comprises a sequence having at least 75%, at least 80%, at least 85%, at least 90%, at least 95%, or at least 99% sequence identity to the sequence of any one of SEQ ID NOs: 45 or 48. In some embodiments, the first polypeptide comprises a sequence of any one of SEQ ID NOs: 51 or 54; and the second polypeptide comprises a sequence of any one of SEQ ID NOs: 45 or 48.

In some embodiments, the third polypeptide comprises a sequence having at least 75%, at least 80%, at least 85%, at least 90%, at least 95%, or at least 99% sequence identity to the sequence of any one of SEQ ID NOs: 93 or 96. In some embodiments, the third polypeptide comprises a sequence of any one of SEQ ID NOs: 93 or 96. In some embodiments, the fourth polypeptide comprises a sequence having at least 75%, at least 80%, at least 85%, at least 90%, at least 95%, or at least 99% sequence identity to the sequence of any one of SEQ ID NOs: 87 or 90. In some embodiments, the fourth polypeptide comprises a sequence of any one of SEQ ID NOs: 87 or 90. In some embodiments, the third polypeptide comprises a sequence having at least 75%, at least 80%, at least 85%, at least 90%, at least 95%, or at least 99% sequence identity to the sequence of any one of SEQ ID NOs: 93 or 96; and the fourth polypeptide comprises a sequence having at least 75%, at least 80%, at least 85%, at least 90%, at least 95%, or at least 99% sequence identity to the sequence of any one of SEQ ID NOs: 87 or 90. In some embodiments, the third polypeptide comprises a sequence of any one of SEQ ID NOs: 93 or 96; and the fourth polypeptide comprises a sequence of any one of SEQ ID NOs: 87 or 90.

In some embodiments, the antigen binding molecule comprises the CH of the second polypeptide linked to the VH of the first antigen binding domain, wherein the CH further comprises a C_{H}1. In some embodiments, the antigen binding molecule comprises the CH of the fourth polypeptide linked to the VH of the second antigen binding domain, wherein the CH further comprises a C_{H}1. In some embodiments, the C_{H}1 is linked to the C-terminus of the VH.

In some embodiments, the second polypeptide comprises a Heavy Chain (VH-CH) comprising a sequence having at least 75%, at least 80%, at least 85%, at least 90%, at least 95%, or at least 99% sequence identity to the sequence of any one of SEQ ID NOs: 44 or 47.

In some embodiments, the second polypeptide comprises a VH-CH comprising the sequence of any one of SEQ ID NOs: 44 or 47. In some embodiments, the fourth polypeptide comprises a Heavy Chain (VH-CH) comprising a sequence having at least 75%, at least 80%, at least 85%, at least 90%, at least 95%, or at least 99% sequence identity to the sequence of any one of SEQ ID NOs: 86 or 89. In some embodiments, the fourth polypeptide comprises a VH-CH comprising the sequence of any one of SEQ ID NOs: 86 or 89.

In some embodiments, the first polypeptide comprises the CL of the first antigen binding domain linked to the VL of the first antigen binding domain. In some embodiments, the third polypeptide comprises the CL of the second antigen binding domain linked to the VL of the second antigen binding domain. In some embodiments, the CL is linked to a C-terminus of the VL.

In some embodiments, the first polypeptide comprises a Light Chain (VL-CL) comprising a sequence having at least 75%, at least 80%, at least 85%, at least 90%, at least 95%, or at least 99% sequence identity to the sequence of any one of SEQ ID NOs: 50 or 53.

In some embodiments, the first polypeptide comprises a VL-CL comprising the sequence of any one of SEQ ID NOs: 50 or 53. In some embodiments, the third polypeptide comprises a Light Chain (VL-CL) comprising a sequence having at least 75%, at least 80%, at least 85%, at least 90%, at least 95%, or at least 99% sequence identity to the sequence of any one of SEQ ID NOs: 92 or 95. In some embodiments, the third polypeptide comprises a VL-CL comprising the sequence of any one of SEQ ID NOs: 92 or 95.

In some embodiments, the first polypeptide comprises a VL-CL comprising a sequence having at least 75%, at least 80%, at least 85%, at least 90%, at least 95%, or at least 99% sequence identity to the sequence of any one of SEQ ID NOs: 50 or 53; and the second polypeptide comprises a VH-CH comprising a sequence having at least 75%, at least 80%, at least 85%, at least 90%, at least 95%, or at least 99% sequence identity to the sequence of any one of SEQ ID NOs: 44 or 47. In some embodiments, the first polypeptide comprises a VL-CL comprising the sequence of any one of SEQ ID NOs: 50 or 53; and the second polypeptide comprises a VH-CH comprising the sequence of any one of SEQ ID NOs: 44 or 47.

In some embodiments, the third polypeptide comprises a VL-CL comprising a sequence having at least 75%, at least 80%, at least 85%, at least 90%, at least 95%, or at least 99% sequence identity to the sequence of any one of SEQ ID NOs: 92 or 95; and the fourth polypeptide comprises a VH-CH comprising a sequence having at least 75%, at least 80%, at least 85%, at least 90%, at least 95%, or at least 99% sequence identity to the sequence of any one of SEQ ID NOs: 86 or 89. In some embodiments, the third polypeptide comprises a VL-CL comprising the sequence of any one of SEQ ID NOs: 92 or 95; and the fourth polypeptide comprises a VH-CH comprising the sequence of any one of SEQ ID NOs: 86 or 89.

In some embodiments, the first polypeptide comprises a VL-CL comprising a sequence having at least 75%, at least 80%, at least 85%, at least 90%, at least 95%, or at least 99% sequence identity to the sequence of any one of SEQ ID NOs: 50 or 53; the second polypeptide comprises a VH-CH comprising a sequence having at least 75%, at least 80%, at least 85%, at least 90%, at least 95%, or at least 99% sequence identity to the sequence of any one of SEQ ID NOs: 44 or 47; the third polypeptide comprises a VL-CL comprising a sequence having at least 75%, at least 80%, at least 85%, at least 90%, at least 95%, or at least 99% sequence identity to the sequence of any one of SEQ ID NOs: 92 or 95; and the fourth polypeptide comprises a VH-CH comprising a sequence having at least 75%, at least 80%, at least 85%, at least 90%, at least 95%, or at least 99% sequence identity to the sequence of any one of SEQ ID NOs: 86 or 89. In some embodiments, the first polypeptide comprises a VL-CL comprising a sequence of any one of SEQ ID NOs: 50 or 53; the second polypeptide comprises a VH-CH comprising a sequence of any one of SEQ ID NOs: 44 or 47; the third polypeptide comprises a VL-CL comprising a sequence of any one of SEQ ID NOs: 92 or 95; and the fourth polypeptide comprises a VH-CH comprising a sequence of any one of SEQ ID NOs: 86 or 89.

In some embodiments, the antigen binding molecule disclosed herein may comprise post-translational modifications. For example, the post-translational modification can include one or more additional amino acid residues incorporated at the c-terminus of the first polypeptide, the second polypeptide, the third polypeptide, and/or the fourth polypeptide. In some embodiments, the first polypeptide comprises a VL-CL comprising a sequence of any one of SEQ ID NOs: 50 or 53; the second polypeptide comprises a VH-CH comprising a sequence of:

(SEQ ID NO: 676)
EVQLVESGGGLVQPGRSLRLSCAASGFTFSDYGMHWVRQAPGKGLEWVSAIDAGGS
TDYADSVEGRFTISRDNAKNSLYLQMNSLRAEDTAVYYCAKDLEAGYYAPDVWGK
GTTVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISK
AKGQPREPQVYTLPPSRDELTKNQVSLWCLVKGFYPSDIAVEWESNGQPENNYKTTP
PVLDSDGSFFLYSKLTVDKSRWQQGNVFSCSVMHEALHNHYTQKSLSLSPGK;
the third polypeptide comprises a VL-CL comprising a
sequence of any one of SEQ ID NOs: 92 or 95; and the
fourth polypeptide comprises a VH-CH comprising a sequence of:
(SEQ ID NO: 677)
QVQLVQSGAEVKKPGASVKVSCKASGYDFNNDLIEWVRQAPGQCLEWMAVINPGS
GRTNYAQKFQGRVTMTADKSTSTVYMELSSLRSEDTAVYYCAMIYYGPHSYAMDY
WGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPKSAD
KTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVYTLPPSRDELTKNQVSLSCAVKGFYPSDIAVEWESNGQPENNY
KTTPPVLDSDGSFFLVSKLTVDKSRWQQGNVFSCSVMHEALHNHYTQKSLSLSPGK.

In some embodiments, the HCDR1 (Kabat) of the first antigen binding domain comprises the amino acid sequence of DX₁×₂MH, wherein X₁ is Y/A, and X₂ is G/A. In some embodiments, the HCDR2 (Kabat) of the first antigen binding domain comprises the amino acid sequence of AIDX₁GGX₂×₃×₄YADSVEG (SEQ ID NO: 678), wherein X₁ is A/R, X₂ is S/A, X₃ is T/A, and X₄ is A/D/G/Y. In some embodiments, the HCDR3 (Kabat) of the first antigen binding domain comprises the amino acid sequence of DLX₁×₂GX₃YX₄PDV (SEQ ID NO: 679), wherein X₁ is A/E, X₂ is A/S, X₃ is Y/A, and X₄ is A/G. In some embodiments, the LCDR1 (Kabat) of the first antigen binding domain comprises the amino acid sequence of RASQDIX₁×₂×₃LA (SEQ ID NO: 4), wherein X₁ is G/R, X₂ is R/H, and X₃ is F/Y. In some embodiments, the LCDR2 (Kabat) of the first antigen binding domain comprises the amino acid sequence of AX₁×₂×₃NLQS (SEQ ID NO: 680), wherein X₁ is A/V, X₂ is S/A, and X₃ is N/T/A. In some embodiments, the LCDR3 (Kabat) of the first antigen binding domain comprises the amino acid sequence of QQYX₁×₂×₃×₄YX₅ (SEQ ID NO: 6), wherein X₁ is S/H, X₂ is T/N, X₃ is S/L, X₄ is V/P, X₅ is T/S.

In some embodiments, the antigen binding molecules of the present disclosure take the form of an immunoconjugate or a portion thereof (e.g., a monoclonal antibody conjugated to a cytotoxic drug payload, an Antibody-Drug Conjugate, an antibody-small molecule conjugate, etc.). In some embodiments, the antigen binding molecules provided herein can be selected from the group consisting of an antibody-small molecule conjugates (ASCs), an antibody-drug conjugate (ADC), a Targeted Drug Conjugate (TDC), an Antibody-Drug Bioconjugate, an Antibody Payload Conjugate (APC), an Antibody-Cytotoxic Conjugate, a Chemotherapeutic Antibody Conjugate, an Antibody-Linked Drug (ALD), a Conjugated Monoclonal Antibody (cMAB), a Bioconjugated Antibody, Precision Medicines with Antibodies, Hybrid Biologics, or any variations thereof.

Provided herein, in some aspects, is an antigen binding molecule comprising: (i) a first antigen binding domain that binds to an epidermal growth factor receptor (EGFR); and (ii) a second antigen binding domain that binds to an integrin subunit beta 6 (ITGB6), wherein the antigen binding molecule is conjugated or linked to a cytotoxic payload or a therapeutic agent. Provided herein, in some aspects, is an antigen binding molecule or an antigen-binding portion thereof, wherein the antigen binding molecule or an antigen-binding portion thereof competes with and/or binds the same epitope as a reference antibody, wherein the reference antibody comprises: (a) a heavy chain variable region (VH) comprising the amino acid sequence set forth in SEQ ID NO: 43; and (b) a light chain variable region (VL) comprising the amino acid sequence set forth in SEQ ID NO: 49, wherein the antigen binding molecule is conjugated or linked to a cytotoxic payload or a therapeutic agent. Provided herein, in some aspects, is an antigen binding molecule or an antigen-binding portion thereof, wherein the antigen binding molecule or an antigen-binding portion thereof competes with and/or binds the same epitope as a reference antibody, wherein the reference antibody comprises: (a) a heavy chain variable region (VH) comprising the amino acid sequence set forth in SEQ ID NO: 85; and (b) a light chain variable region (VL) comprising the amino acid sequence set forth in SEQ ID NO: 91, wherein the antigen binding molecule is conjugated or linked to a cytotoxic payload or a therapeutic agent. In some embodiments, the antigen binding molecule disclosed herein, or a functional fragment thereof, is conjugated or linked to a cytotoxic agent. In some embodiments, the antigen binding molecule disclosed herein, or a functional fragment thereof, can be an ADC. In some embodiments, the antigen binding molecule disclosed herein, or a functional fragment thereof, is conjugated or linked to a small molecule. In some embodiments, the antigen binding molecule disclosed herein, or a functional fragment thereof, can be an ASC. Various classes of cytotoxic drugs can be used as drug payloads. For example, traditional chemotherapeutic agents, small-molecule inhibitors, radioisotopes, or biologics such as toxins or enzymes. The selection of a drug payload/small molecule can depend on factors such as a target cancer type, desired mechanism of action, toxicity profile, and drug release kinetics. The selection of a drug payload/small molecule can be based on potency and dose selection. For example, the drug payload/small molecule should be potent enough to induce cell death at low concentrations, ensuring efficacy against cancer cells while minimizing systemic toxicity to normal tissues. The dose of the drug payload/small molecule, including the amount of drug payload/small molecule attached to the antigen binding molecule, can be optimized to achieve the desired therapeutic effect and balance between efficacy and safety.

### First Antigen Binding Domain

The present disclosure provides an antigen binding molecule, comprising: (i) a first antigen binding domain that binds to an epidermal growth factor receptor (EGFR); and (ii) a second antigen binding domain that binds to an integrin subunit beta 6 (ITGB6). In some embodiments, the first antigen binding domain (e.g., the EGFR binding domain) comprises an EGFR binding domain derived from an anti-EGFR antibody (e.g., a CDR that specifically binds to EGFR). Such antibodies are known to those skilled in the art and can be incorporated into methods and bispecific antigen binding molecules of the present disclosure. Antibodies targeting EGFR are known in the art, and include, for example, the following anti-EGFR antibodies: (i) cetuximab, described in, for example, P. Kirkpatrick, et al., “Cetuximab.” Nature Reviews Drug Discovery, 3(7) (2004): 549; (ii) panitumumab, described in, for example, L. Saltz, et al., “Panitumamab.” Nature Reviews Drug Discovery, 5(12) (2006): 987; (iii) nimotuzumab, described in, for example, M.S. Ramakrishnan, “Nimotuzumab, a promising therapeutic monoclonal for treatment of tumors of epithelial origin.” mAbs 1(1) (2009):41; and (iv) necitumumab, described in, for example, D.R. Tabemero, “Necitumumab, a fully human IgG1 mAb directed against the EGFR for the potential treatment of cancer.” Current Opinions in Investigational Drugs, 11(12) (2000): 1434.

The first antigen binding domain can comprise an “arm” of an antibody (e.g., an antigen binding molecule disclosed herein). In some embodiments, the first antigen binding domain comprises an anti-EGFR arm of the antigen binding molecule disclosed herein. In some embodiments, the first antigen binding domain binds to a mutant EGFR protein. In some embodiments, the first antigen binding domain selectively binds to a mutant EGFR protein.

In some embodiments, the first antigen binding domain binds to an epitope of EGFR on a target cell, wherein the epitope comprises at least 70% sequence identity to an epitope to which Cetuximab binds. In some embodiments, the first antigen binding domain binds to an epitope of EGFR on the target cell, wherein the epitope comprises at least 80% sequence identity to an epitope to which Cetuximab binds. In some cases, the first antigen binding domain binds to an epitope of EGFR on the target cell, wherein the epitope comprises at least 90% sequence identity to the epitope to which Cetuximab binds. In some embodiments, the first antigen binding domain binds to an epitope of EGFR on the target cell, wherein the epitope comprises at least 95% sequence identity to an epitope to which Cetuximab binds. In some embodiments, the first antigen binding domain binds to domain III of the EGFR extracellular domain. In some embodiments, the first antigen binding domain binds an epitope that cross-competes with cetuximab. In some embodiments, the first antigen binding domain binds an epitope that cross-competes with an EGF ligand.

In some embodiments, the first antigen binding domain binds to an epitope of EGFR on the target cell that does not include any of the amino acids from the epitope to which Cetuximab binds. In some embodiments, the first antigen binding domain binds to an epitope of EGFR on the target cell that includes one, two, three, four, five, or six of the amino acids from the epitope to which Cetuximab binds. In some embodiments, the first antigen binding domain binds to an epitope of EGFR on the target cell that includes one or more of the amino acids from the epitope to which Cetuximab binds. In some embodiments, the first antigen binding domain binds to an epitope of EGFR on the target cell that includes two or more of the amino acids from the epitope to which Cetuximab binds. In some embodiments, the first antigen binding domain binds to an epitope of EGFR on the target cell that includes three or more of the amino acids from the epitope to which Cetuximab binds. In some embodiments, the first antigen binding domain binds to an epitope of EGFR on the target cell that includes four or more of the amino acids from the epitope to which Cetuximab binds.

In some embodiments, the first antigen binding domain binds to EGFR on the target cell, wherein the epitope comprises at least 70% sequence identity to an epitope to which Mav2 binds. In some embodiments, the first antigen binding domain binds to an epitope of EGFR on the target cell, wherein the epitope comprises at least 80% sequence identity to an epitope to which Mav2 binds. In some cases, the first antigen binding domain binds to an epitope of EGFR on the target cell, wherein the epitope comprises at least 90% sequence identity to the epitope to which Mav2 binds. In some embodiments, the first antigen binding domain binds to an epitope of EGFR on the target cell, wherein the epitope comprises at least 95% sequence identity to an epitope to which Mav2 binds.

In some embodiments, the first antigen binding domain binds to an epitope of EGFR on the target cell that does not include any of the amino acids from the epitope to which Mav2 binds. In some embodiments, the first antigen binding domain binds to an epitope of EGFR on the target cell that includes one, two, three, four, five, or six of the amino acids from the epitope to which Mav2 binds. In some embodiments, the first antigen binding domain binds to an epitope of EGFR on the target cell that includes one or more of the amino acids from the epitope to which Mav2 binds. In some embodiments, the first antigen binding domain binds to an epitope of EGFR on the target cell that includes two or more of the amino acids from the epitope to which Mav2 binds. In some embodiments, the first antigen binding domain binds to an epitope of EGFR on the target cell that includes three or more of the amino acids from the epitope to which Mav2 binds. In some embodiments, the first antigen binding domain binds to an epitope of EGFR on the target cell that includes four or more of the amino acids from the epitope to which Mav2 binds.

In some embodiments, the epitope of EGFR comprises the following amino acids of human EGFR (UniProt ID: P00533): P373, R377, L406, Q407, Q432, H433, Q435, F436, V441, S442, 1462, S464, G465, K467, K489, I490, I491, S492, N493, G495, and N497. The antigen binding molecules of the present disclosure targeting EGFR may target the epitope comprising the amino acids P373, R377, L406, Q407, Q432, H433, Q435, F436, V441, S442, 1462, S464, G465, K467, K489, I490, I491, S492, N493, G495, and N497 of human EGFR. In some embodiments, the antibody targeting the amino acids P373, R377, L406, Q407, Q432, H433, Q435, F436, V441, S442, 1462, S464, G465, K467, K489, I490, I491, S492, N493, G495, and N497 of human EGFR comprises Cetuximab. In some embodiments, the epitope of EGFR comprises the following amino acids of human EGFR: L349, H370, L372, P373, V374, R377, D379, F381, T382, Q408, H433, S442. The antigen binding molecules of the present disclosure targeting EGFR may target the epitope comprising the amino acids L349, H370, L372, P373, V374, R377, D379, F381, T382, Q408, H433, and S442 of human EGFR. In some embodiments, the antibody targeting the amino acids L349, H370, L372, P373, V374, R377, D379, F381, T382, Q408, H433, and S442 of human EGFR comprises Mav2 (h7D12 hIgG1). In some embodiments, the epitope of EGFR comprises amino acids of Mus musculus EGFR (UniProt ID: Q5SVE7).

The antigen binding molecules of the present disclosure targeting EGFR may bind to an epitope that comprises about 70% sequence identity to the epitope to which Cetuximab or Mav2 (h7D12 hIgG1) binds. The antigen binding molecules of the present disclosure targeting EGFR may bind to an epitope that comprises about 75% sequence identity to the epitope to which Cetuximab or Mav2 (h7D12 hIgG1) binds. The antigen binding molecules of the present disclosure targeting EGFR may bind to an epitope that comprises about 80% sequence identity to the epitope to which Cetuximab or Mav2 (h7D12 hIgG1) binds. The antigen binding molecules of the present disclosure targeting EGFR may bind to an epitope that comprises about 85% sequence identity to the epitope to which Cetuximab or Mav2 (h7D12 hIgG1) binds. The antigen binding molecules of the present disclosure targeting EGFR may bind to an epitope that comprises about 90% sequence identity to the epitope to which Cetuximab or Mav2 (h7D12 hIgG1) binds. The antigen binding molecules of the present disclosure targeting EGFR may bind to an epitope that comprises about 95% sequence identity to the epitope to which Cetuximab or Mav2 (h7D12 hIgG1) binds. The antigen binding molecules of the present disclosure targeting EGFR may bind to an epitope that comprises about 99% sequence identity to the epitope to which Cetuximab or Mav2 (h7D12 hIgG1) binds.

The antigen binding molecules of the present disclosure targeting EGFR may bind to an epitope that comprises a different epitope than the epitope to which Cetuximab or Mav2 (h7D12 hIgG1) binds. The antigen binding molecules of the present disclosure targeting EGFR may bind to an epitope that comprises a different epitope than the epitope to which Cetuximab or Mav2 (h7D12 hIgG1) binds, wherein the epitopes do not bind to any of the same amino acids on EGFR. The antigen binding molecules of the present disclosure targeting EGFR may bind to an epitope that comprises a different epitope than the epitope to which Cetuximab or Mav2 (h7D12 hIgG1) binds, wherein the epitopes bind to any one or more of the same amino acids on EGFR. The antigen binding molecules of the present disclosure targeting EGFR may bind to an epitope that comprises a different epitope than the epitope to which Cetuximab or Mav2 (h7D12 hIgG1) binds, wherein the epitopes bind to any two or more of the same amino acids on EGFR. The antigen binding molecules of the present disclosure targeting EGFR may bind to an epitope that comprises a different epitope than the epitope to which Cetuximab or Mav2 (h7D12 hIgG1) binds, wherein the epitopes bind to any three or more of the same amino acids on EGFR. The antigen binding molecules of the present disclosure targeting EGFR may bind to an epitope that comprises a different epitope than the epitope to which Cetuximab or Mav2 (h7D12 hIgG1) binds, wherein the epitopes bind to any four or more of the same amino acids on EGFR. The antigen binding molecules of the present disclosure targeting EGFR may bind to an epitope that comprises a different epitope than the epitope to which Cetuximab or Mav2 (h7D12 hIgG1) binds, wherein the epitopes bind to any five or more of the same amino acids on EGFR. The antigen binding molecules of the present disclosure targeting EGFR may bind to an epitope that comprises a different epitope than the epitope to which Cetuximab or Mav2 (h7D12 hIgG1) binds, wherein the epitopes bind to any six or more of the same amino acids on EGFR. The antigen binding molecules of the present disclosure targeting EGFR may bind to an epitope that comprises a different epitope than the epitope to which Cetuximab or Mav2 (h7D12 hIgG1) binds, wherein the epitopes bind to any seven or more of the same amino acids on EGFR. The antigen binding molecules of the present disclosure targeting EGFR may bind to an epitope that comprises a different epitope than the epitope to which Cetuximab or Mav2 (h7D12 hIgG1) binds, wherein the epitopes bind to any eight or more of the same amino acids on EGFR. The antigen binding molecules of the present disclosure targeting EGFR may bind to an epitope that comprises a different epitope than the epitope to which Cetuximab or Mav2 (h7D12 hIgG1) binds, wherein the epitopes bind to any nine or more of the same amino acids on EGFR. The antigen binding molecules of the present disclosure targeting EGFR may bind to an epitope that comprises a different epitope than the epitope to which Cetuximab or Mav2 (h7D12 hIgG1) binds, wherein the epitopes bind to any ten or more of the same amino acids on EGFR.

In some cases, the antigen binding molecules disclosed herein targeting EGFR may bind the same epitope as any one of the sequences listed in Table 1 (e.g., SEQ ID NOs: 1-6, and/or 13-54) and/or Table 2 (e.g., SEQ ID NOs: 103-107, and/or SEQ ID NOs: 110-111). The antigen binding molecules disclosed herein targeting EGFR may bind to an epitope that comprises about 70% sequence identity to the epitope to which any one of the sequences listed in Table 1 (e.g., SEQ ID NOs: 1-6, and/or 13-54) and/or Table 2 (e.g., SEQ ID NOs: 103-107, and/or SEQ ID NOs: 110-111) binds. The antigen binding molecules disclosed herein targeting EGFR may bind to an epitope that comprises about 75% sequence identity to the epitope to which any one of the sequences listed in Table 1 (e.g., SEQ ID NOs: 1-6, and/or 13-54) and/or Table 2 (e.g., SEQ ID NOs: 103-107, and/or SEQ ID NOs: 110-111) binds. The antigen binding molecules disclosed herein targeting EGFR may bind to an epitope that comprises about 80% sequence identity to the epitope to which any one of the sequences listed in Table 1 (e.g., SEQ ID NOs: 1-6, and/or 13-54) and/or Table 2 (e.g., SEQ ID NOs: 103-107, and/or SEQ ID NOs: 110-111) binds. The antigen binding molecules disclosed herein targeting EGFR may bind to an epitope that comprises about 85% sequence identity to the epitope to which any one of the sequences listed in Table 1 (e.g., SEQ ID NOs: 1-6, and/or 13-54) and/or Table 2 (e.g., SEQ ID NOs: 103-107, and/or SEQ ID NOs: 110-111) binds. The antigen binding molecules disclosed herein targeting EGFR may bind to an epitope that comprises about 90% sequence identity to the epitope to which any one of the sequences listed in Table 1 (e.g., SEQ ID NOs: 1-6, and/or 13-54) and/or Table 2 (e.g., SEQ ID NOs: 103-107, and/or SEQ ID NOs: 110-111) binds. The antigen binding molecules disclosed herein targeting EGFR may bind to an epitope that comprises about 95% sequence identity to the epitope to which any one of the sequences listed in Table 1 (e.g., SEQ ID NOs: 1-6, and/or 13-54) and/or Table 2 (e.g., SEQ ID NOs: 103-107, and/or SEQ ID NOs: 110-111) binds. The antigen binding molecules disclosed herein targeting EGFR may bind to an epitope that comprises about 99% sequence identity to the epitope to which any one of the sequences listed in Table 1 (e.g., SEQ ID NOs: 1-6, and/or 13-54) and/or Table 2 (e.g., SEQ ID NOs: 103-107, and/or SEQ ID NOs: 110-111) binds. The antigen binding molecules disclosed herein targeting EGFR may bind to an epitope that comprises a different epitope than the epitope to which any one of the sequences listed in Table 1 (e.g., SEQ ID NOs: 1-6, and/or 13-54) and/or Table 2 (e.g., SEQ ID NOs: 103-107, and/or SEQ ID NOs: 110-111) binds.

In some embodiments, the antigen binding molecules disclosed herein targeting EGFR may bind with a similar affinity as any one of the sequences listed in Table 1 (e.g., SEQ ID NOs: 1-6, and/or 13-54) and/or Table 2 (e.g., SEQ ID NOs: 103-107, and/or SEQ ID NOs: 110-111). In certain embodiments, the antigen binding molecules have a Kd less than, more than, within 10%, within 20%, within 30%, within 40%, within 50%, withing 75%, or within 100% of the binding affinity of a monovalent antigen binding molecule (e.g., cetuximab). For example, the binding affinity of a monovalent antigen binding molecule may have a Kd of between 0.1 nM and 100 nM. When incorporated into the antigen binding molecule disclosed herein the Kd may be within the same range. Alternatively, the binding affinity may be slightly greater than, but within two-fold of the monovalent binding affinity (e.g., cetuximab). The binding affinity may be within three-fold of the monovalent binding affinity (e.g., cetuximab).

In some embodiments, the antigen binding molecules disclosed herein targeting EGFR comprise a sequence listed Table 1 (e.g., SEQ ID NOs: 1-6, and/or 13-54) and/or Table 2 (e.g., SEQ ID NOs: 103-107, and/or SEQ ID NOs: 110-111). In some embodiments, the antigen binding molecules disclosed herein targeting EGFR comprise a sequence listed Table 1 (e.g., SEQ ID NOs: 1-6, and/or 13-54) and/or Table 2 (e.g., SEQ ID NOs: 103-107, and/or SEQ ID NOs: 110-111). In some embodiments, the antigen binding molecules, disclosed herein, targeting EGFR comprise at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to a sequence listed Table 1 (e.g., SEQ ID NOs: 1-6, and/or 13-54) and/or Table 2 (e.g., SEQ ID NOs: 103-107, and/or SEQ ID NOs: 110-111).

In some embodiments, the first antigen binding domain comprises a heavy chain variable region (VH) comprising: (a) a heavy chain complementarity determining region 1 (HCDR1) amino acid sequence of SEQ ID NO: 1; (b) a heavy chain complementarity determining region 2 (HCDR2) amino acid sequence of SEQ ID NO: 2; and (c) a heavy chain complementarity determining region 3 (HCDR3) amino acid sequence of SEQ ID NO: 3. In some embodiments, the first antigen binding domain comprises a light chain variable region (VL) comprising: (a) a light chain complementarity determining region 1 (LCDR1) amino acid sequence of SEQ ID NO: 4; (b) a light chain complementarity determining region 2 (LCDR2) amino acid sequence of SEQ ID NO: 5; and (c) a light chain complementarity determining region 3 (LCDR3) amino acid sequence of SEQ ID NO: 6.

In some embodiments, the first antigen binding domain comprises a heavy chain variable region (VH) comprising a heavy chain complementarity determining region 1 (HCDR1) amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to any one of SEQ ID NOs: 16 or 25. In some embodiments, the first antigen binding domain comprises a heavy chain variable region (VH) comprising a heavy chain complementarity determining region 2 (HCDR2) amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to any one of SEQ ID NOs: 17 or 26. In some embodiments, the first antigen binding domain comprises a heavy chain variable region (VH) comprising a heavy chain complementarity determining region 3 (HCDR3) amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to any one of SEQ ID NOs: 18 or 27.

In some embodiments, the first antigen binding domain comprises a heavy chain variable region (VH) comprising a heavy chain complementarity determining region 1 (HCDR1) amino acid sequence of any one of SEQ ID NOs: 16 or 25. In some embodiments, the first antigen binding domain comprises a heavy chain variable region (VH) comprising a heavy chain complementarity determining region 1 (HCDR1) amino acid sequence of SEQ ID NO: 16. In some embodiments, the first antigen binding domain comprises a heavy chain variable region (VH) comprising a heavy chain complementarity determining region 1 (HCDR1) amino acid sequence of SEQ ID NO: 25. In some embodiments, the first antigen binding domain comprises a heavy chain variable region (VH) comprising a heavy chain complementarity determining region 2 (HCDR2) amino acid sequence of any one of SEQ ID NOs: 17 or 26. In some embodiments, the first antigen binding domain comprises a heavy chain variable region (VH) comprising a heavy chain complementarity determining region 2 (HCDR2) amino acid sequence of SEQ ID NO: 17. In some embodiments, the first antigen binding domain comprises a heavy chain variable region (VH) comprising a heavy chain complementarity determining region 2 (HCDR2) amino acid sequence of SEQ ID NO: 26. In some embodiments, the first antigen binding domain comprises a heavy chain variable region (VH) comprising a heavy chain complementarity determining region 3 (HCDR3) amino acid sequence of any one of SEQ ID NOs: 18 or 27. In some embodiments, the first antigen binding domain comprises a heavy chain variable region (VH) comprising a heavy chain complementarity determining region 3 (HCDR3) amino acid sequence of SEQ ID NO: 18. In some embodiments, the first antigen binding domain comprises a heavy chain variable region (VH) comprising a heavy chain complementarity determining region 3 (HCDR3) amino acid sequence of SEQ ID NO: 27.

In some embodiments, the first antigen binding domain comprises a light chain variable region (VL) comprising a light chain complementarity determining region 1 (LCDR1) amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to any one of SEQ ID NOs: 31 or 37. In some embodiments, the first antigen binding domain comprises a light chain variable region (VL) comprising a light chain complementarity determining region 2 (LCDR2) amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to any one of SEQ ID NOs: 32 or 38. In some embodiments, the first antigen binding domain comprises a light chain variable region (VL) comprising a light chain complementarity determining region 3 (LCDR3) amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to any one of SEQ ID NOs: 33 or 39.

In some embodiments, the first antigen binding domain comprises a light chain variable region (VL) comprising a light chain complementarity determining region 1 (LCDR1) amino acid sequence of any one of SEQ ID NOs: 31 or 37. In some embodiments, the first antigen binding domain comprises a light chain variable region (VL) comprising a light chain complementarity determining region 1 (LCDR1) amino acid sequence of SEQ ID NO: 31. In some embodiments, the first antigen binding domain comprises a light chain variable region (VL) comprising a light chain complementarity determining region 1 (LCDR1) amino acid sequence of SEQ ID NO: 37. In some embodiments, the first antigen binding domain comprises a light chain variable region (VL) comprising a light chain complementarity determining region 2 (LCDR2) amino acid sequence of any one of SEQ ID NOs: 32 or 38. In some embodiments, the first antigen binding domain comprises a light chain variable region (VL) comprising a light chain complementarity determining region 2 (LCDR2) amino acid sequence of SEQ ID NO: 32. In some embodiments, the first antigen binding domain comprises a light chain variable region (VL) comprising a light chain complementarity determining region 2 (LCDR2) amino acid sequence of SEQ ID NO: 38. In some embodiments, the first antigen binding domain comprises a light chain variable region (VL) comprising a light chain complementarity determining region 3 (LCDR3) amino acid sequence of any one of SEQ ID NOs: 33 or 39. In some embodiments, the first antigen binding domain comprises a light chain variable region (VL) comprising a light chain complementarity determining region 3 (LCDR3) amino acid sequence of SEQ ID NO: 33. In some embodiments, the first antigen binding domain comprises a light chain variable region (VL) comprising a light chain complementarity determining region 3 (LCDR3) amino acid sequence of SEQ ID NO: 39.

In some embodiments, the first antigen binding domain comprises a VL comprising a sequence having at least 75%, at least 80%, at least 85%, at least 90%, at least 95%, or at least 99% sequence identity to the sequence of SEQ ID NO: 49. In some embodiments, the first antigen binding domain comprises a VL comprising the sequence of SEQ ID NO: 49. In some embodiments, the first antigen binding domain comprises a VL comprising a sequence having at least 75%, at least 80%, at least 85%, at least 90%, at least 95%, or at least 99% sequence identity to the sequence of SEQ ID NO: 52. In some embodiments, the first antigen binding domain comprises a VL comprising the sequence of SEQ ID NO: 52.

In some embodiments, the first antigen binding domain comprises a VH comprising a sequence having at least 75%, at least 80%, at least 85%, at least 90%, at least 95%, or at least 99% sequence identity to the sequence of SEQ ID NO: 43. In some embodiments, the first antigen binding domain comprises a VH comprising the sequence of SEQ ID NO: 43. In some embodiments, the first antigen binding domain comprises a VH comprising a sequence having at least 75%, at least 80%, at least 85%, at least 90%, at least 95%, or at least 99% sequence identity to the sequence of SEQ ID NO: 46. In some embodiments, the first antigen binding domain comprises a VH comprising the sequence of SEQ ID NO: 46.

TABLE 1
A summary of exemplary amino acid sequences

| SEQ ID | | | |
|---|---|---|---|
| NO | Antigen | Domain | Sequence |
| | EGFR | HCDR1 (Kabat) | X₁X₂GX₃X₄, |
| | | | wherein X₁ is D/N, X₂ is Y/Q, X₃ is M/I, and |
| | | | X₄ is H/S |
| 2 | EGFR | HCDR2 (Kabat) | X₁X₂DX₃X₄X₅GX₆TX₇YAX₈X₉X₁₀X₁₁G, |
| | | | wherein X₁ is A/G, X₂ is F/I, X₃ is P/-, X₄ is |
| | | | A/D, X₅ is G/A, X₆ is S/E, X₇ is D/I, X₈ is |
| | | | D/Q, X₉ is S/K, X₁₀ is V/F, and X₁₁ is E/Q |
| | EGFR | HCDR3 (Kabat) | X₁X₂X₃X₄X₅X₆YX₇X₈X₉X₁₀X₁₁X₁₂DX₁₃, |
| | | | wherein X₁ is D/G, X₂ is L/V, X₃ is E/D, X₄ |
| | | | is A/S, X₅ is G/Y, X₆ is G/Y, X₇ is A/G, X₈ |
| | | | is P/R, X₉ is Y/-, X₁₀ is N/-, X₁₁ is W/-, X₁₂ |
| | | | is F/-, and X₁₃ is V/P |
| 4 | EGFR | LCDR1 (Kabat) | RASQDIX₁X₂X₃LA, |
| | | | wherein X₁ is G/R, X₂ is R/H, and X₃ is F/Y |
| | EGFR | LCDR2 (Kabat) | X₁X₂X₃NX₄X₅X₆, |
| | | | wherein X₁ is A/D, X₂ is V/T, X₃ is S/F, X₄ |
| | | | is L/R, X₅ is Q/A, and X₆ is S/T |
| 6 | EGFR | LCDR3 (Kabat) | QQYX₁X₂X₃X₄YX₅, |
| | | | wherein X₁ is S/H, X₂ is T/N, X₃ is S/L, X₄ is |
| | | | V/P, X₅ is T/S |
| 7 | ITGB6 | HCDR1 (Kabat) | NXILIE, |
| | | | wherein X₁ is D/Y |
| 8 | ITGB6 | HCDR2 (Kabat) | VIX₁PGSGX₂X₃NYAQKFQG, |
| | | | wherein X₁ is N/S, X₂ is R/I, and X₃ is T/I |
| | ITGB6 | HCDR3 (Kabat) | IX₁YX₂X₃X₄X₅X₆X₇X₈DX₉, |
| | | | wherein X₁ is Y/D, X₂ is G/S, X₃ is P/G, X₄ |
| | | | is H/P, X₅ is S/Y, X₆ is Y/A, X₇ is A/V, X₈ is |
| | | | M/-, and X₉ is Y/D |
| 10 | ITGB6 | LCDR1 (Kabat) | KASX₁X₂VX₃TAVA, |
| | | | wherein X₁ is L/Q, X₂ is D/A, and X₃ is R/N |
| 11 | ITGB6 | LCDR2 (Kabat) | SASYX₁YT, |
| | | | wherein X1 is R/G |
| 12 | ITGB6 | LCDR3 (Kabat) | QX₁HYGX₂PWT, |
| | | | wherein X₁ is Q/H, and X₂ is I/V |
| 13 | EGFR | HCDR1 | GFTFSDYGMH |
| | | (Combined) | |
| 14 | EGFR | HCDR2 | AIDAGGSTDYADSVEG |
| | | (Combined) | |
| 15 | EGFR | HCDR3 | DLEAGYYAPDV |
| | | (Combined) | |
| 16 | EGFR | HCDR1 (Kabat) | DYGMH |
| 17 | EGFR | HCDR2 (Kabat) | AIDAGGSTDYADSVEG |
| 18 | EGFR | HCDR3 (Kabat) | DLEAGYYAPDV |
| 19 | EGFR | HCDR1 | GFTFSDY |
| | | (Chothia) | |
| 20 | EGFR | HCDR2 | DAGGS |
| | | (Chothia) | |
| 21 | EGFR | HCDR3 | DLEAGYYAPDV |
| | | (Chothia) | |
| 22 | EGFR | HCDR1 | GDTFSNQGIS |
| | | (Combined) | |
| 23 | EGFR | HCDR2 | GFDPDAGETIYAQKFQG |
| | | (Combined) | |
| 24 | EGFR | HCDR3 | GVDSYGYGRYNWFDP |
| | | (Combined) | |
| 25 | EGFR | HCDR1 (Kabat) | NQGIS |
| 26 | EGFR | HCDR2 (Kabat) | GFDPDAGETIYAQKFQG |
| 27 | EGFR | HCDR3 (Kabat) | GVDSYGYGRYNWFDP |
| 28 | EGFR | HCDR1 | GDTFSNQ |
| | | (Chothia) | |
| 29 | EGFR | HCDR2 | DPDAGE |
| | | (Chothia) | |
| 30 | EGFR | HCDR3 | GVDSYGYGRYNWFDP |
| | | (Chothia) | |
| 31 | EGFR | LCDR1 (Kabat) | RASQDIGRFLA |
| 32 | EGFR | LCDR2 (Kabat) | AVSNLQS |
| 33 | EGFR | LCDR3 (Kabat) | QQYSTSVYT |
| 34 | EGFR | LCDR1 | RASQDIGRFLA |
| | | (Chothia) | |
| 35 | EGFR | LCDR2 | AVSNLQS |
| | | (Chothia) | |
| 36 | EGFR | LCDR3 | QQYSTSVYT |
| | | (Chothia) | |
| 37 | EGFR | LCDR1 (Kabat) | RASQDIRHYLA |
| 38 | EGFR | LCDR2 (Kabat) | DTFNRAT |
| 39 | EGFR | LCDR3 (Kabat) | QQYHNLPYS |
| 40 | EGFR | LCDR1 | RASQDIRHYLA |
| | | (Chothia) | |
| 41 | EGFR | LCDR2 | DTFNRAT |
| | | (Chothia) | |
| 42 | EGFR | LCDR3 | QQYHNLPYS |
| | | (Chothia) | |
| 43 | EGFR | VH | EVQLVESGGGLVQPGRSLRLSCAASGF |
| | | | TFSDYGMHWVRQAPGKGLEWVSAIDA |
| | | | GGSTDYADSVEGRFTISRDNAKNSLYL |
| | | | QMNSLRAEDTAVYYCAKDLEAGYYAP |
| | | | DVWGKGTTVTVSS |
| 44 | EGFR | Heavy Chain | EVQLVESGGGLVQPGRSLRLSCAASGF |
| | | (VH-CH) | TFSDYGMHWVRQAPGKGLEWVSAIDA |
| | | | GGSTDYADSVEGRFTISRDNAKNSLYL |
| | | | QMNSLRAEDTAVYYCAKDLEAGYYAP |
| | | | DVWGKGTTVTVSSASTKGPSVFPLAPS |
| | | | SKSTSGGTAALGCLVKDYFPEPVTVSW |
| | | | NSGALTSGVHTFPAVLQSSGLYSLSSVV |
| | | | TVPSSSLGTQTYICNVNHKPSNTKVDKK |
| | | | VEPKSCDKTHTCPPCPAPELLGGPSVFL |
| | | | FPPKPKDTLMISRTPEVTCVVVDVSHED |
| | | | PEVKFNWYVDGVEVHNAKTKPREEQY |
| | | | NSTYRVVSVLTVLHQDWLNGKEYKCK |
| | | | VSNKALPAPIEKTISKAKGQPREPQVYT |
| | | | LPPSRDELTKNQVSLWCLVKGFYPSDIA |
| | | | VEWESNGQPENNYKTTPPVLDSDGSFF |
| | | | LYSKLTVDKSRWQQGNVFSCSVMHEA |
| | | | LHNHYTQKSLSLSPG |
| 45 | | Heavy Chain | ASTKGPSVFPLAPSSKSTSGGTAALGCL |
| | | Constant Region | VKDYFPEPVTVSWNSGALTSGVHTFPA |
| | | (CH) | VLQSSGLYSLSSVVTVPSSSLGTQTYIC |
| | | | NVNHKPSNTKVDKKVEPKSCDKTHTCP |
| | | | PCPAPELLGGPSVFLFPPKPKDTLMISRT |
| | | | PEVTCVVVDVSHEDPEVKFNWYVDGV |
| | | | EVHNAKTKPREEQYNSTYRVVSVLTVL |
| | | | HQDWLNGKEYKCKVSNKALPAPIEKTI |
| | | | SKAKGQPREPQVYTLPPSRDELTKNQV |
| | | | SLWCLVKGFYPSDIAVEWESNGQPENN |
| | | | YKTTPPVLDSDGSFFLYSKLTVDKSRW |
| | | | QQGNVFSCSVMHEALHNHYTQKSLSLS |
| | | | PG |
| 46 | EGFR | VH | QVQLVQSGAEVKKPGASVKVSCKVSG |
| | | | DTFSNQGISWVRQAPGKGLEWMGGFD |
| | | | PDAGETIYAQKFQGRVTMTEDTSTDTA |
| | | | YMELSSLKSEDTAVYYCARGVDSYGY |
| | | | GRYNWFDPWGQGTLVTVSS |
| 47 | EGFR | Heavy Chain | QVQLVQSGAEVKKPGASVKVSCKVSG |
| | | (VH-CH) | DTFSNQGISWVRQAPGKGLEWMGGFD |
| | | | PDAGETIYAQKFQGRVTMTEDTSTDTA |
| | | | YMELSSLKSEDTAVYYCARGVDSYGY |
| | | | GRYNWFDPWGQGTLVTVSSASTKGPS |
| | | | VFPLAPSSKSTSGGTAALGCLVKDYFPE |
| | | | PVTVSWNSGALTSGVHTFPAVLQSSGL |
| | | | YSLSSVVTVPSSSLGTQTYICNVNHKPS |
| | | | NTKVDKKVEPKSCDKTHTCPPCPAPEL |
| | | | LGGPSVFLFPPKPKDTLMISRTPEVTCV |
| | | | VVDVSHEDPEVKFNWYVDGVEVHNAK |
| | | | TKPREEQYNSTYRVVSVLTVLHQDWLN |
| | | | GKEYKCKVSNKALPAPIEKTISKAKGQP |
| | | | REPQVYTLPPSRDELTKNQVSLWCLVK |
| | | | GFYPSDIAVEWESNGQPENNYKTTPPVL |
| | | | DSDGSFFLYSKLTVDKSRWQQGNVFSC |
| | | | SVMHEALHNHYTQKSLSLSPG |
| 48 | | Heavy Chain | ASTKGPSVFPLAPSSKSTSGGTAALGCL |
| | | Constant Region | VKDYFPEPVTVSWNSGALTSGVHTFPA |
| | | (CH) | VLQSSGLYSLSSVVTVPSSSLGTQTYIC |
| | | | NVNHKPSNTKVDKKVEPKSCDKTHTCP |
| | | | PCPAPELLGGPSVFLFPPKPKDTLMISRT |
| | | | PEVTCVVVDVSHEDPEVKFNWYVDGV |
| | | | EVHNAKTKPREEQYNSTYRVVSVLTVL |
| | | | HQDWLNGKEYKCKVSNKALPAPIEKTI |
| | | | SKAKGQPREPQVYTLPPSRDELTKNQV |
| | | | SLWCLVKGFYPSDIAVEWESNGQPENN |
| | | | YKTTPPVLDSDGSFFLYSKLTVDKSRW |
| | | | QQGNVFSCSVMHEALHNHYTQKSLSLS |
| | | | PG |
| 49 | EGFR | VL | DIQMTQSPSSLSASVGDRVTITCRASQDI |
| | | | GRFLAWYQQKPGKAPKLLIYAVSNLQS |
| | | | GVPSRFSGSGSGTDFTLTISSLQPEDVAT |
| | | | YYCQQYSTSVYTFGGGTKVEIK |
| 50 | EGFR | Light Chain | DIQMTQSPSSLSASVGDRVTITCRASQDI |
| | | (VL-CL) | GRFLAWYQQKPGKAPKLLIYAVSNLQS |
| | | | GVPSRFSGSGSGTDFTLTISSLQPEDVAT |
| | | | YYCQQYSTSVYTFGGGTKVEIKRTVAA |
| | | | PSVFIFPPSDEQLKSGTASVVCLLNNFYP |
| | | | REAKVQWKVDNALQSGNSQESVTEQD |
| | | | SKDSTYSLSSTLTLSKADYEKHKVYAC |
| | | | EVTHQGLSSPVTKSFNRGEC |
| 51 | | Light Chain | RTVAAPSVFIFPPSDEQLKSGTASVVCL |
| | | Constant Region | LNNFYPREAKVQWKVDNALQSGNSQE |
| | | (CL) | SVTEQDSKDSTYSLSSTLTLSKADYEKH |
| | | | KVYACEVTHQGLSSPVTKSFNRGEC |
| 52 | EGFR | VL | DIQMTQSPSSVSASVGDRVTITCRASQD |
| | | | IRHYLAWYQQKPGKAPKLLIYDTFNRA |
| | | | TGVPSRFSGSGSGTDFTLTISSLQPEDFA |
| | | | NYYCQQYHNLPYSFGGGTKVEIK |
| 53 | EGFR | Light Chain | DIQMTQSPSSVSASVGDRVTITCRASQD |
| | | (VL-CL) | IRHYLAWYQQKPGKAPKLLIYDTFNRA |
| | | | TGVPSRFSGSGSGTDFTLTISSLQPEDFA |
| | | | NYYCQQYHNLPYSFGGGTKVEIKRTVA |
| | | | APSVFIFPPSDEQLKSGTASVVCLLNNF |
| | | | YPREAKVQWKVDNALQSGNSQESVTE |
| | | | QDSKDSTYSLSSTLTLSKADYEKHKVY |
| | | | ACEVTHQGLSSPVTKSFNRGEC |
| 54 | | Light Chain | RTVAAPSVFIFPPSDEQLKSGTASVVCL |
| | | Constant Region | LNNFYPREAKVQWKVDNALQSGNSQE |
| | | (CL) | SVTEQDSKDSTYSLSSTLTLSKADYEKH |
| | | | KVYACEVTHQGLSSPVTKSFNRGEC |
| 55 | ITGB6 | HCDR1 | GYDFNNDLIE |
| | | (Combined) | |
| 56 | ITGB6 | HCDR2 | VINPGSGRTNYAQKFQG |
| | | (Combined) | |
| 57 | ITGB6 | HCDR3 | IYYGPHSYAMDY |
| | | (Combined) | |
| 58 | ITGB6 | HCDR1 (Kabat) | NDLIE |
| 59 | ITGB6 | HCDR2 (Kabat) | VINPGSGRTNYAQKFQG |
| 60 | ITGB6 | HCDR3 (Kabat) | IYYGPHSY AMDY |
| 61 | ITGB6 | HCDR1 | GYDFNND |
| | | (Chothia) | |
| 62 | ITGB6 | HCDR2 | NPGSGR |
| | | (Chothia) | |
| 63 | ITGB6 | HCDR3 | IYYGPHSYAMDY |
| | | (Chothia) | |
| 64 | ITGB6 | HCDR1 | GYAFTNYLIE |
| | | (Combined) | |
| 65 | ITGB6 | HCDR2 | VISPGSGIINYAQKFQG |
| | | (Combined) | |
| 66 | ITGB6 | HCDR3 | IDYSGPYAVDD |
| | | (Combined) | |
| 67 | ITGB6 | HCDR1 (Kabat) | NYLIE |
| 68 | ITGB6 | HCDR2 (Kabat) | VISPGSGIINYAQKFQG |
| 69 | ITGB6 | HCDR3 (Kabat) | IDYSGPYAVDD |
| 70 | ITGB6 | HCDR1 | GYAFTNY |
| | | (Chothia) | |
| 71 | ITGB6 | HCDR2 | SPGSGI |
| | | (Chothia) | |
| 72 | ITGB6 | HCDR3 | IDYSGPYAVDD |
| | | (Chothia) | |
| 73 | ITGB6 | LCDR1 (Kabat) | KASLDVRTAVA |
| 74 | ITGB6 | LCDR2 (Kabat) | SASYRYT |
| 75 | ITGB6 | LCDR3 (Kabat) | QQHYGIPWT |
| 76 | ITGB6 | LCDR1 | KASLDVRTAVA |
| | | (Chothia) | |
| 77 | ITGB6 | LCDR2 | SASYRYT |
| | | (Chothia) | |
| 78 | ITGB6 | LCDR3 | QQHYGIPWT |
| | | (Chothia) | |
| 79 | ITGB6 | LCDR1 (Kabat) | KASQAVNTAVA |
| 80 | ITGB6 | LCDR2 (Kabat) | SASYGYT |
| 81 | ITGB6 | LCDR3 (Kabat) | QHHYGVPWT |
| 82 | ITGB6 | LCDR1 | KASQAVNTAVA |
| | | (Chothia) | |
| 83 | ITGB6 | LCDR2 | SASYGYT |
| | | (Chothia) | |
| 84 | ITGB6 | LCDR3 | QHHYGVPWT |
| | | (Chothia) | |
| 85 | ITGB6 | VH | QVQLVQSGAEVKKPGASVKVSCKASG |
| | | | YDFNNDLIEWVRQAPGQCLEWMAVIN |
| | | | PGSGRTNYAQKFQGRVTMTADKSTSTV |
| | | | YMELSSLRSEDTAVYYCAMIYYGPHSY |
| | | | AMDYWGQGTLVTVSS |
| 86 | ITGB6 | Heavy Chain | QVQLVQSGAEVKKPGASVKVSCKASG |
| | | (VH-CH) | YDFNNDLIEWVRQAPGQCLEWMAVIN |
| | | | PGSGRTNYAQKFQGRVTMTADKSTSTV |
| | | | YMELSSLRSEDTAVYYCAMIYYGPHSY |
| | | | AMDYWGQGTLVTVSSASTKGPSVFPLA |
| | | | PSSKSTSGGTAALGCLVKDYFPEPVTVS |
| | | | WNSGALTSGVHTFPAVLQSSGLYSLSS |
| | | | VVTVPSSSLGTQTYICNVNHKPSNTKVD |
| | | | KKVEPKSADKTHTCPPCPAPELLGGPSV |
| | | | FLFPPKPKDTLMISRTPEVTCVVVDVSH |
| | | | EDPEVKFNWYVDGVEVHNAKTKPREE |
| | | | QYNSTYRVVSVLTVLHQDWLNGKEYK |
| | | | CKVSNKALPAPIEKTISKAKGQPREPQV |
| | | | YTLPPSRDELTKNQVSLSCAVKGFYPSD |
| | | | IAVEWESNGQPENNYKTTPPVLDSDGSF |
| | | | FLVSKLTVDKSRWQQGNVFSCSVMHE |
| | | | ALHNHYTQKSLSLSPG |
| 87 | | Heavy Chain | ASTKGPSVFPLAPSSKSTSGGTAALGCL |
| | | Constant | VKDYFPEPVTVSWNSGALTSGVHTFPA |
| | | Region (CH) | VLQSSGLYSLSSVVTVPSSSLGTQTYIC |
| | | | NVNHKPSNTKVDKKVEPKSADKTHTCP |
| | | | PCPAPELLGGPSVFLFPPKPKDTLMISRT |
| | | | PEVTCVVVDVSHEDPEVKFNWYVDGV |
| | | | EVHNAKTKPREEQYNSTYRVVSVLTVL |
| | | | HQDWLNGKEYKCKVSNKALPAPIEKTI |
| | | | SKAKGQPREPQVYTLPPSRDELTKNQV |
| | | | SLSCAVKGFYPSDIAVEWESNGQPENN |
| | | | YKTTPPVLDSDGSFFLVSKLTVDKSRW |
| | | | QQGNVFSCSVMHEALHNHYTQKSLSLS |
| | | | PG |
| 88 | ITGB6 | VH | QVQLVQSGAEVKKPGSSVKVSCKASGY |
| | | | AFTNYLIEWVRQAPGQCLEWMGVISPG |
| | | | SGIINYAQKFQGRATLTADKSTSTAYME |
| | | | LSSLRSEDTAVYYCAAIDYSGPYAVDD |
| | | | WGQGTLVTVSS |
| 89 | ITGB6 | Heavy Chain | QVQLVQSGAEVKKPGSSVKVSCKASGY |
| | | (VH-CH) | AFTNYLIEWVRQAPGQCLEWMGVISPG |
| | | | SGIINYAQKFQGRATLTADKSTSTAYME |
| | | | LSSLRSEDTAVYYCAAIDYSGPYAVDD |
| | | | WGQGTLVTVSSASTKGPSVFPLAPSSKS |
| | | | TSGGTAALGCLVKDYFPEPVTVSWNSG |
| | | | ALTSGVHTFPAVLQSSGLYSLSSVVTVP |
| | | | SSSLGTQTYICNVNHKPSNTKVDKKVEP |
| | | | KSADKTHTCPPCPAPELLGGPSVFLFPP |
| | | | KPKDTLMISRTPEVTCVVVDVSHEDPE |
| | | | VKFNWYVDGVEVHNAKTKPREEQYNS |
| | | | TYRVVSVLTVLHQDWLNGKEYKCKVS |
| | | | NKALPAPIEKTISKAKGQPREPQVYTLP |
| | | | PSRDELTKNQVSLSCAVKGFYPSDIAVE |
| | | | WESNGQPENNYKTTPPVLDSDGSFFLV |
| | | | SKLTVDKSRWQQGNVFSCSVMHEALH |
| | | | NHYTQKSLSLSPG |
| 90 | | Heavy Chain | ASTKGPSVFPLAPSSKSTSGGTAALGCL |
| | | Constant | VKDYFPEPVTVSWNSGALTSGVHTFPA |
| | | Region (CH) | VLQSSGLYSLSSVVTVPSSSLGTQTYIC |
| | | | NVNHKPSNTKVDKKVEPKSADKTHTCP |
| | | | PCPAPELLGGPSVFLFPPKPKDTLMISRT |
| | | | PEVTCVVVDVSHEDPEVKFNWYVDGV |
| | | | EVHNAKTKPREEQYNSTYRVVSVLTVL |
| | | | HQDWLNGKEYKCKVSNKALPAPIEKTI |
| | | | SKAKGQPREPQVYTLPPSRDELTKNQV |
| | | | SLSCAVKGFYPSDIAVEWESNGQPENN |
| | | | YKTTPPVLDSDGSFFLVSKLTVDKSRW |
| | | | QQGNVFSCSVMHEALHNHYTQKSLSLS |
| | | | PG |
| 91 | ITGB6 | VL | DIQMTQSPSSLSASVGDRVTITCKASLD |
| | | | VRTAVAWYQQKPGKAPKLLIYSASYRY |
| | | | TGVPSRFSGSGSGTDFTFTISSLQPEDIA |
| | | | TYYCQQHYGIPWTFGCGTKVEIK |
| 92 | ITGB6 | Light Chain | DIQMTQSPSSLSASVGDRVTITCKASLD |
| | | (VL-CL) | VRTAVAWYQQKPGKAPKLLIYSASYRY |
| | | | TGVPSRFSGSGSGTDFTFTISSLQPEDIA |
| | | | TYYCQQHYGIPWTFGCGTKVEIKRTVA |
| | | | APSVFIFPPSDEQLKSGTASVVCLLNNF |
| | | | YPREAKVQWKVDNALQSGNSQESVTE |
| | | | QDSKDSTYSLSSTLTLSKADYEKHKVY |
| | | | ACEVTHQGLSSPVTKSFNRGE |
| 93 | | Light Chain | RTVAAPSVFIFPPSDEQLKSGTASVVCL |
| | | Constant Region | LNNFYPREAKVQWKVDNALQSGNSQE |
| | | (CL) | SVTEQDSKDSTYSLSSTLTLSKADYEKH |
| | | | KVYACEVTHQGLSSPVTKSFNRGE |
| 94 | ITGB6 | VL | DIQMTQSPSSLSASVGDRVTITCKASQA |
| | | | VNTAVAWYQQKPGKAPKLLIYSASYG |
| | | | YTGVPSRFSGSGSGTDFTLTISSLQPEDF |
| | | | ATYYCQHHYGVPWTFGCGTKLEIK |
| 95 | ITGB6 | Light Chain | DIQMTQSPSSLSASVGDRVTITCKASQA |
| | | (VL-CL) | VNTAVAWYQQKPGKAPKLLIYSASYG |
| | | | YTGVPSRFSGSGSGTDFTLTISSLQPEDF |
| | | | ATYYCQHHYGVPWTFGCGTKLEIKRTV |
| | | | AAPSVFIFPPSDEQLKSGTASVVCLLNN |
| | | | FYPREAKVQWKVDNALQSGNSQESVT |
| | | | EQDSKDSTYSLSSTLTLSKADYEKHKV |
| | | | YACEVTHQGLSSPVTKSFNRGE |
| 96 | | Light Chain | RTVAAPSVFIFPPSDEQLKSGTASVVCL |
| | | Constant Region | LNNFYPREAKVQWKVDNALQSGNSQE |
| | | (CL) | SVTEQDSKDSTYSLSSTLTLSKADYEKH |
| | | | KVYACEVTHQGLSSPVTKSFNRGE |

In some embodiments, the sequences listed in Table 1 (SEQ ID NOs: 1-96) are amino acid molecules. In some embodiments, the sequences listed in Table 1 (SEQ ID NOs: 1-96) are amino acid molecules that are synthetic constructs. In some embodiments, the sequences listed in Table 1 (SEQ ID NOs: 1-96) for CH sequences (constant heavy chain), VH sequence (variable heavy chain sequence), CL sequences (constant light chain), VL sequence (variable light chain sequence) are amino acid molecules that are synthetic constructs.

### Second Antigen Binding Domain

The present disclosure provides an antigen binding molecule, comprising: (i) a first antigen binding domain that binds to an epidermal growth factor receptor (EGFR); and (ii) a second antigen binding domain that binds to an integrin subunit beta 6 (ITGB6). In some embodiments, the second antigen binding domain comprises a membrane-associated internalizing proteins, such as ITGB6. In some embodiments, the membrane-associated internalizing protein internalizes and/or degrades the EGFR protein. The present disclosure utilizes the innate function of membrane-associated internalizing proteins, such as ITGB6, to internalize upon binding of an antigen binding molecule to the protein. By simultaneously binding to EGFR using the first binding domain and binding to a membrane-associated internalizing proteins using the second binding domain, the antigen binding molecule causes the EGFR protein to be internalized into the target cell with the membrane-associated internalizing protein. Once internalized, the EGFR protein will be sequestered and/or degraded (e.g., via lysosomal degradation) within the target cell.

Membrane-associated internalizing proteins, such as ITGB6, for use in methods and antigen binding molecules of the present disclosure include cell-surface proteins that internalize upon binding of an antigen binding molecule (e.g., an antibody) to the protein. In some embodiments, the second antigen binding domain of the present disclosure is a membrane associated internalizing protein. In some embodiments, the membrane associated internalizing protein is ITGB6.

Methods and antigen binding molecules of the present disclosure may utilize membrane-associated degrading proteins, such as ITGB6, to cause degradation of the EGFR protein. The present disclosure may use the membrane-associated degrading proteins to cause ubiquitination upon binding of an antigen binding molecule to the membrane-associated degrading protein. By also binding to EGFR at the first antigen binding domain and binding to a membrane-associated degrading protein, such as ITGB6, using the second antigen binding domain, the antigen binding molecule can cause the EGFR protein to be degraded with the membrane-associated degrading protein. In some embodiments, the second antigen binding domain of the present disclosure is a membrane-associated degrading protein. In some embodiments, membrane associated internalizing protein can be a membrane-associated degrading protein. In some embodiments, the membrane-associated degrading protein is ITGB6.

In some embodiments, the second antigen binding domain is derived from an antibody directed at a membrane associated internalizing protein or a degrading protein. Such antibodies are known to those skilled in the art and can be incorporated into methods and bispecific antigen binding molecules of the present disclosure. For example, in some embodiments, the complementarity-determining regions (“CDR”) of known antibodies directed at the membrane associated internalizing protein of interest or the membrane associated degrading protein of interest can be incorporated into multispecific antigen binding molecules and methods of the present disclosure using known techniques. Exemplary antibodies suitable for incorporation into the antigen binding molecules of the present disclosure include those described below.

For example, antibodies targeting ITGB6 are known in the art, including, for example the antibody SGN-B6A described in, for example, Patnaik, Amita, et al. “A phase 1 study of SGN-B6A, an antibody-drug conjugate targeting integrin beta-6, in patients with advanced solid tumors (SGN-B6A-001, Trial in Progress)” (2021). Another antibody suitable for incorporation into the present disclosure include the anti-ITGB6 antibodies TPS3144-TPS3144 described in Zheng, X₁aoxia, et al. “Silencing of ITGB6 inhibits the progression of cervical carcinoma via regulating JAK/STAT3 signaling pathway” Annals of Translational Medicine 9.9 (2021).

The second antigen binding domain can comprise an “arm” of an antibody (e.g., an antigen binding molecule disclosed herein). In some embodiments, the second antigen binding domain comprises an anti-ITGB6 arm of the antigen binding molecule disclosed herein. In some embodiments, the second antigen binding domain binds to a mutant ITGB6 protein. In some embodiments, the second antigen binding domain selectively binds to a mutant ITGB6 protein.

In some embodiments, the second antigen binding domain binds to ITGB6 in both an open conformation of ITGB6 and a closed conformation of ITGB6. In some embodiments, the second antigen binding domain binds an epitope that does not compete with LAP ligand.

In some embodiments, the second antigen binding domain binds an epitope that is distinct from ITGB6 antibodies STX-100 (US20210363259A1) and h2A2 (US20210198367A1).

In some embodiments, the antigen binding molecules disclosed herein targeting the internalizing receptor protein, such as ITGB6, may bind the same epitope as any one of the sequences listed in Table 1 (e.g., SEQ ID NOs: 7-12, and/or 55-96) and/or Table 2 (e.g., SEQ ID NOs: 97-102, and/or SEQ ID NOs: 112-113). The antigen binding molecules disclosed herein targeting the internalizing receptor protein, such as ITGB6, may bind to an epitope that comprises about 70% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules disclosed herein targeting the internalizing receptor protein, such as ITGB6, may bind to an epitope that comprises about 75% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules disclosed herein targeting the internalizing receptor protein, such as ITGB6, may bind to an epitope that comprises about 80% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules disclosed herein targeting the internalizing receptor protein, such as ITGB6, may bind to an epitope that comprises about 85% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules disclosed herein targeting the internalizing receptor protein, such as ITGB6, may bind to an epitope that comprises about 90% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules disclosed herein targeting the internalizing receptor protein, such as ITGB6, may bind to an epitope that comprises about 95% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules disclosed herein targeting the internalizing receptor protein, such as ITGB6, may bind to an epitope that comprises about 99% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules disclosed herein targeting the internalizing receptor protein, such as ITGB6, may bind to an epitope that comprises a different epitope than the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds.

In some embodiments, the antigen binding molecules targeting the degrader protein comprise sequences listed Table 1 (e.g., SEQ ID NOs: 7-12, and/or 55-96) and/or Table 2 (e.g., SEQ ID NOs: 97-102, and/or SEQ ID NOs: 112-113). In some embodiments, the antigen binding molecules targeting the degrader protein comprise at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to the sequences listed Table 1 (e.g., SEQ ID NOs: 7-12, and/or 55-96) and/or Table 2 (e.g., SEQ ID NOs: 97-102, and/or SEQ ID NOs: 112-113).

In some cases, the antigen binding molecules disclosed herein targeting the degrader protein, such as ITGB6, may bind the same epitope as any one of the sequences listed in Table 1 (e.g., SEQ ID NOs: 7-12, and/or 55-96) and/or Table 2 (e.g., SEQ ID NOs: 97-102, and/or SEQ ID NOs: 112-113). The antigen binding molecules disclosed herein targeting the degrader protein, such as ITGB6, may bind to an epitope that comprises about 70% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules disclosed herein targeting the degrader protein, such as ITGB6, may bind to an epitope that comprises about 75% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules disclosed herein targeting the degrader protein, such as ITGB6, may bind to an epitope that comprises about 80% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules disclosed herein targeting the degrader protein, such as ITGB6, may bind to an epitope that comprises about 85% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules disclosed herein targeting the degrader protein, such as ITGB6, may bind to an epitope that comprises about 90% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules disclosed herein targeting the degrader protein, such as ITGB6, may bind to an epitope that comprises about 95% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules disclosed herein targeting the degrader protein, such as ITGB6, may bind to an epitope that comprises about 99% sequence identity to the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds. The antigen binding molecules, disclosed herein, targeting the degrader protein, such as ITGB6, may bind to an epitope that comprises a different epitope than the epitope to which any one of the sequences listed in Table 1 (e.g., SEQ ID NOs: 7-12, and/or 55-96) and/or Table 2 (e.g., SEQ ID NOs: 97-102, and/or SEQ ID NOs: 112-113) binds.

In some embodiments, the antigen binding molecules disclosed herein targeting the internalizing receptor protein, such as ITGB6, may bind with a similar affinity as any one of the antibodies listed in Table 1 (e.g., SEQ ID NOs: 7-12, and/or 55-96) and/or Table 2 (e.g., SEQ ID NOs: 97-102, and/or SEQ ID NOs: 112-113). In certain embodiments, the antigen binding molecules have a Kd less than, more than, within 10%, within 20%, within 30%, within 40%, within 50%, withing 75%, or within 100% of the binding affinity of a monovalent antigen binding molecule. For example, the binding affinity of a monovalent antigen binding molecule may have a Kd of between 0.1 nM and 100 nM. When incorporated into the antigen binding molecule disclosed herein the Kd may be within the same range.

Alternatively, the binding affinity may be slightly greater than, but within two-fold of the monovalent binding affinity. The binding affinity may be within three-fold of the monovalent binding affinity.

In some embodiments, the bispecific antigen binding molecule disclosed herein selectively binds to integrin subunit beta 6 (ITGB6) without blocking latent-associated peptide (LAP) binding. The binding affinity of the bispecific antigen binding molecule disclosed herein can be influenced by the presence of LAP bound to its ITGB6 epitope. In certain embodiments, a LAP-bound antigen binding molecules have a Kd less than, more than, within 10%, within 20%, within 30%, within 40%, within 50%, withing 75%, or within 100% relative to the binding affinity of a corresponding antigen binding molecule without LAP bound. For example, LAP binding to an ITGB6 epitopes can induce conformational changes in ITGB6, altering its binding properties. As an additional example, LAP binding to an ITGB6 epitopes can result in competitive inhibition (e.g., if LAP binds a close-proximity epitope with the antigen binding molecules disclosed herein it may reduce the binding affinity), allosteric effects (e.g., if LAP binds to one epitope, inducing a conformational change in ITGB6, the binding affinity may increase), or may have little to no effect.

In some embodiments, the second antigen binding domain comprises a constant heavy chain (CH) sequence, a variable heavy (VH) sequence, a constant light chain (CL) sequence, and a variable light (VL) sequence. In some embodiments, the second antigen binding domain comprises a CH sequence and a VH sequence. The second antigen binding domain comprising a CH sequence, a VH sequence, a CL sequence, and a VL sequence may comprise one or more sequences listed in Table 1 (e.g., SEQ ID NOs: 7-12, and/or 55-96) and/or Table 2 (e.g., SEQ ID NOs: 97-102, and/or SEQ ID NOs: 112-113). The second antigen binding domain comprising a CH sequence, a VH sequence, a CL sequence, and a VL sequence may comprise at least 70% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 75% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 80% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 85% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 90% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 91% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 92% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 93% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 94% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 95% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 96% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 97% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 98% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 99% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 99.5% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113). In some cases, the second antigen binding domain comprising a CH sequence, a VH sequence, a CL sequence, and a VL sequence comprises at least 99.9% sequence identity to one or more sequences listed in Table 1 (e.g., SEQ ID NOs: 7-12, and/or 55-96) and/or Table 2 (e.g., SEQ ID NOs: 97-102, and/or SEQ ID NOs: 112-113).

In some embodiments, the antigen binding molecules targeting the internalizing receptor protein comprise sequences listed Table 1 (e.g., SEQ ID NOs: 7-12, and/or 55-96) and/or Table 2 (e.g., SEQ ID NOs: 97-102, and/or SEQ ID NOs: 112-113). In some embodiments, the antigen binding molecules targeting the internalizing receptor protein comprise at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to the sequences listed Table 1 (e.g., SEQ ID NOs: 7-12, and/or 55-96) and/or Table 2 (e.g., SEQ ID NOs: 97-102, and/or SEQ ID NOs: 112-113).

In some embodiments, the second antigen binding domain comprises a heavy chain variable region (VH) comprising: (a) a heavy chain complementarity determining region 1 (HCDR1) amino acid sequence of SEQ ID NO: 7; (b) a heavy chain complementarity determining region 2 (HCDR2) amino acid sequence of SEQ ID NO: 8; and (c) a heavy chain complementarity determining region 3 (HCDR3) amino acid sequence of SEQ ID NO: 9. In some embodiments, the second antigen binding domain comprises a light chain variable region (VL) comprising: (a) a light chain complementarity determining region 1 (LCDR1) amino acid sequence of SEQ ID NO: 10; (b) a light chain complementarity determining region 2 (LCDR2) amino acid sequence of SEQ ID NO: 11; and (c) a light chain complementarity determining region 3 (LCDR3) amino acid sequence of SEQ ID NO: 12.

In some embodiments, the second antigen binding domain comprises a heavy chain variable region (VH) comprising a heavy chain complementarity determining region 1 (HCDR1) amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to any one of SEQ ID NOs: 58 or 67. In some embodiments, the second antigen binding domain comprises a heavy chain variable region (VH) comprising a heavy chain complementarity determining region 2 (HCDR2) amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to any one of SEQ ID NOs: 59 or 68. In some embodiments, the second antigen binding domain comprises a heavy chain variable region (VH) comprising a heavy chain complementarity determining region 3 (HCDR3) amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to any one of SEQ ID NOs: 60 or 69.

In some embodiments, the second antigen binding domain comprises a heavy chain variable region (VH) comprising a heavy chain complementarity determining region 1 (HCDR1) amino acid sequence of any one of SEQ ID NOs: 58 or 67. In some embodiments, the second antigen binding domain comprises a heavy chain variable region (VH) comprising a heavy chain complementarity determining region 1 (HCDR1) amino acid sequence of SEQ ID NO: 58. In some embodiments, the second antigen binding domain comprises a heavy chain variable region (VH) comprising a heavy chain complementarity determining region 1 (HCDR1) amino acid sequence of SEQ ID NO: 67. In some embodiments, the second antigen binding domain comprises a heavy chain variable region (VH) comprising a heavy chain complementarity determining region 2 (HCDR2) amino acid sequence of any one of SEQ ID NOs: 59 or 68. In some embodiments, the second antigen binding domain comprises a heavy chain variable region (VH) comprising a heavy chain complementarity determining region 2 (HCDR2) amino acid sequence of SEQ ID NO: 59. In some embodiments, the second antigen binding domain comprises a heavy chain variable region (VH) comprising a heavy chain complementarity determining region 2 (HCDR2) amino acid sequence of SEQ ID NO: 68. In some embodiments, the second antigen binding domain comprises a heavy chain variable region (VH) comprising a heavy chain complementarity determining region 3 (HCDR3) amino acid sequence of any one of SEQ ID NOs: 60 or 69. In some embodiments, the second antigen binding domain comprises a heavy chain variable region (VH) comprising a heavy chain complementarity determining region 3 (HCDR3) amino acid sequence of SEQ ID NO: 60. In some embodiments, the second antigen binding domain comprises a heavy chain variable region (VH) comprising a heavy chain complementarity determining region 3 (HCDR3) amino acid sequence of SEQ ID NO: 69.

In some embodiments, the second antigen binding domain comprises a light chain variable region (VL) comprising a light chain complementarity determining region 1 (LCDR1) amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to any one of SEQ ID NOs: 73 or 79. In some embodiments, the second antigen binding domain comprises a light chain variable region (VL) comprising a light chain complementarity determining region 2 (LCDR2) amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to any one of SEQ ID NOs: 74 or 80. In some embodiments, the second antigen binding domain comprises a light chain variable region (VL) comprising a light chain complementarity determining region 3 (LCDR3) amino acid sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.9%, or at least 99.9% sequence identity to any one of SEQ ID NOs: 75 or 81.

In some embodiments, the second antigen binding domain comprises a light chain variable region (VL) comprising a light chain complementarity determining region 1 (LCDR1) amino acid sequence of any one of SEQ ID NOs: 73 or 79. In some embodiments, the second antigen binding domain comprises a light chain variable region (VL) comprising a light chain complementarity determining region 1 (LCDR1) amino acid sequence of SEQ ID NO: 73. In some embodiments, the second antigen binding domain comprises a light chain variable region (VL) comprising a light chain complementarity determining region 1 (LCDR1) amino acid sequence of SEQ ID NO: 79. In some embodiments, the second antigen binding domain comprises a light chain variable region (VL) comprising a light chain complementarity determining region 2 (LCDR2) amino acid sequence of any one of SEQ ID NOs: 74 or 80. In some embodiments, the second antigen binding domain comprises a light chain variable region (VL) comprising a light chain complementarity determining region 2 (LCDR2) amino acid sequence of SEQ ID NO: 74. In some embodiments, the second antigen binding domain comprises a light chain variable region (VL) comprising a light chain complementarity determining region 2 (LCDR2) amino acid sequence of SEQ ID NO: 80. In some embodiments, the second antigen binding domain comprises a light chain variable region (VL) comprising a light chain complementarity determining region 3 (LCDR3) amino acid sequence of any one of SEQ ID NOs: 75 or 81. In some embodiments, the second antigen binding domain comprises a light chain variable region (VL) comprising a light chain complementarity determining region 3 (LCDR3) amino acid sequence of SEQ ID NO: 75. In some embodiments, the second antigen binding domain comprises a light chain variable region (VL) comprising a light chain complementarity determining region 3 (LCDR3) amino acid sequence of SEQ ID NO: 81.

In some embodiments, the second antigen binding domain comprises a VL comprising a sequence having at least 75%, at least 80%, at least 85%, at least 90%, at least 95%, or at least 99% sequence identity to the sequence of SEQ ID NO: 91. In some embodiments, the second antigen binding domain comprises a VL comprising the sequence of SEQ ID NO: 91. In some embodiments, the second antigen binding domain comprises a VL comprising a sequence having at least 75%, at least 80%, at least 85%, at least 90%, at least 95%, or at least 99% sequence identity to the sequence of SEQ ID NO: 94. In some embodiments, the second antigen binding domain comprises a VL comprising the sequence of SEQ ID NO: 94.

In some embodiments, the second antigen binding domain comprises a VH comprising a sequence having at least 75%, at least 80%, at least 85%, at least 90%, at least 95%, or at least 99% sequence identity to the sequence of SEQ ID NO: 85. In some embodiments, the second antigen binding domain comprises a VH comprising the sequence of SEQ ID NO: 85. In some embodiments, the second antigen binding domain comprises a VH comprising a sequence having at least 75%, at least 80%, at least 85%, at least 90%, at least 95%, or at least 99% sequence identity to the sequence of SEQ ID NO: 88. In some embodiments, the second antigen binding domain comprises a VH comprising the sequence of SEQ ID NO: 88.

TABLE 2
A summary of exemplary amino acid sequences

| SEQ ID | Exemplary | Exemplary | |
|---|---|---|---|
| NO | Antigen | Domain | Exemplary Sequence |
| 97 | ITGB6 | HCDR2 | VINPGSGRTNY X₁X₂KFQG, |
| | | (Kabat) | wherein X₁ is N/A, and X₂ is E/Q |
| 98 | ITGB6 | HCDR2 | VISPGSGIINYX1X2KFQG, |
| | | (Kabat) | wherein X₁ is N/A, and X₂ is E/Q |
| 99 | ITGB6 | VH | QVQLX₁QSGAEX₂X₃X₄PGX₅SVKVSCKAS |
| | | | GYDFNNDLIEWVX₆QX₇PGQX₈LEWX₉AVI |
| | | | NPGSGRTNYX₁₀X₁₁KFX₁₂GX₁₃X₁₄TX₁₅TAD |
| | | | KSX₁₆STVYMX₁₇LSSLX₁₈SX₁₉DX₂₀AVYX₂₁ |
| | | | CAMIYYGPHSYAMDYWGQGTX₂₂VTVSS, |
| | | | wherein X₁ is V/Q, X₂ is V/L, X₃ is K/V, X₄ is |
| | | | K/R, X₅ is A/T, X₆ is R/K, X₇ is A/R, X₈ is |
| | | | C/G, X₉ is M/I, X₁₀ is A/N, X₁₁ is Q/E, X₁₂ is |
| | | | Q/K, X₁₃ is R/K, X₁₄ is V/A, X₁₅ is M/L, X₁₆ is |
| | | | T/S, X₁₇is E/Q, X₁₈ is R/T, X₁₉ is E/D, X₂₀ is |
| | | | T/S, X₂₁ is Y/F, X₂₂ is L/S |
| 100 | ITGB6 | Heavy Chain | QVQLX₁QSGAEX₂X₃X₄PGX₅SVKVSCKAS |
| | | (VH-CH) | GYDFNNDLIEWVX₆QX₇PGQX₈LEWX₉AVI |
| | | | NPGSGRTNYX₁₀X₁₁KFX₁₂GX₁₃X₁₄TX₁₅TAD |
| | | | KSX₁₆STVYMX₁₇LSSLX₁₈SX₁₉DX₂₀AVYX₂₁ |
| | | | CAMIYYGPHSYAMDYWGQGTX₂₂VTVSS |
| | | | ASTKGPSVFPLAPSSKSTSGGTAALGCLV |
| | | | KDYFPEPVTVSWNSGALTSGVHTFPAVL |
| | | | QSSGLYSLSSVVTVPSSSLGTQTYICNVN |
| | | | HKPSNTKVDKKVEPKSCDKTHTCPPCPAP |
| | | | ELLGGPSVFLFPPKPKDTLMISRTPEVTCV |
| | | | VVDVSHEDPEVKFNWYVDGVEVHNAKT |
| | | | KPREEQYNSTYRVVSVLTVLHQDWLNGK |
| | | | EYKCKVSNKALPAPIEKTISKAKGQPREP |
| | | | QVYTLPPSRDELTKNQVSLTCLVKGFYPS |
| | | | DIAVEWESNGQPENNYKTTPPVLDSDGSF |
| | | | FLYSKLTVDKSRWQQGNVFSCSVMHEAL |
| | | | HNHYTQKSLSLSPGK, |
| | | | wherein X₁ is V/Q, X₂is V/L, X₃ is K/V, X₄ is |
| | | | K/R, X₅ is A/T, X₆ is R/K, X₇ is A/R, X₈ is |
| | | | C/G, X₉ is M/I, X₁₀ is A/N, X₁₁ is Q/E, X₁₂ is |
| | | | Q/K, X₁₃ is R/K, X₁₄ is V/A, X₁₅ is M/L, X₁₆ is |
| | | | T/S, X₁₇ is E/Q, X₁₈ is R/T, X₁₉ is E/D, X₂₀ is |
| | | | T/S, X₂₁ is Y/F, X₂₂ is L/S |
| 101 | ITGB6 | VL | DIX₁MTQSX₂X₃X₄X₅SX₆X₇VGDRVX₈ITCK |
| | | | ASLDVRTAVAWYQQKPGX₉X₁₀PKLLIYS |
| | | | ASYRYTGVPX₁₁RFX₁₂GSGSGTDFTFX₁₃IX₁₄ |
| | | | SX₁₅QX₁₆EDX₁₇AX₁₈YYCQQHYGIPWTFG |
| | | | GGTKLEIK, |
| | | | wherein X₁ is V/Q, X₂ is P/H, X₃ is S/K, X₄ is |
| | | | S/F, X₅ is M/L, X₆ is A/T, X₇ is V/S, X₈ is T/S, |
| | | | X₉ is K/Q, X₁₀ is A/S, X₁₁ is S/D, X₁₂ is S/T, |
| | | | X₁₃ is T/N, X₁₄ is S/R, X₁₅ is V/L, X₁₆ is P/A, |
| | | | X₁₇ is I/L, X₁₈ is V/T |
| 102 | ITGB6 | Light Chain | DIX₁MTQSX₂X₃X₄X₅SX₆X₇VGDRVX₈ITCK |
| | | (VL-CL) | ASLDVRTAVAWYQQKPGX₉X₁₀PKLLIYS |
| | | | ASYRYTGVPX₁₁RFX₁₂GSGSGTDFTFX₁₃IX₁₄ |
| | | | SX₁₅QX₁₆EDX₁₇AX₁₈YYCQQHYGIPWTFG |
| | | | GGTKLEIKRTVAAPSVFIFPPSDEQLKSGT |
| | | | ASVVCLLNNFYPREAKVQWKVDNALQS |
| | | | GNSQESVTEQDSKDSTYSLSSTLTLSKAD |
| | | | YEKHKVYACEVTHQGLSSPVTKSFNRGE |
| | | | C, |
| | | | wherein X₁ is V/Q, X₂ is P/H, X₃ is S/K, X₄ is |
| | | | S/F, X₅ is M/L, X₆ is A/T, X₇ is V/S, X₈ is T/S, |
| | | | X₉ is K/Q, X₁₀ is A/S, X₁₁ is S/D, X₁₂ is S/T, |
| | | | X₁₃ is T/N, X₁₄ is S/R, X₁₅ is V/L, X₁₆ is P/A, |
| | | | X₁₇ is I/L, X₁₈ is V/T |
| | EGFR | HCDR1 | X₁ X₂ X₃MH, |
| | | (Kabat) | wherein X₁ is D/A, X₂ is Y/A, X₃ is G/A |
| 104 | EGFR | HCDR2 | AIX₁X₂X₃X₄X₅X₆X₇YADSVX₈G, |
| | | (Kabat) | wherein X₁ is D/S, X₂ is R/A, X₃ is G/S, X₄ is |
| | | | G/A, X₅ is S/A, X₆ is T/A, X₇ is D/G/A/Y, X₈ is |
| | | | K/E |
| | EGFR | HCDR3 | X₁X₂X₃X₄X₅X₆X₇X₈X₉DV, |
| | | (Kabat) | wherein X₁ is D/A, X₂ is L/A, X₃ is E/A, X₄ is |
| | | | S/A, X₅ is G/A, X₆ is Y/A, X₇ is Y/A, X₈ is |
| | | | G/A, X₉ is P/F |
| 106 | EGFR | LCDR2 | AVSX₁X₂X₃S, |
| | | (Kabat) | wherein X₁ is T/N/A, X₂ is L/A, X₃ is Q/A |
| 107 | EGFR | LCDR3 | QQX₁X₂X₃X₄X₅YT, |
| | | (Kabat) | wherein X₁ is Y/A, X₂ is S/A, X₃ is T/A, X₄ is |
| | | | S/A, X₅ is V/P/A |
| 108 | | CH1 | ASTKGPSVFPX₁APSSKSTSGGTAALX₂CL |
| | | (Kabat) | VKDYFPEPVTVSWNSGALTSGVX₃TFPAV |
| | | | LQSSGLYSLSSVVX₄VPSSSLGTQTYICNV |
| | | | NHKPSNTKVDKKVEPKSC, |
| | | | wherein X₁ is L/S, X₂ is G/L, X₃ is H/K/D, X4 |
| | | | is T/K/D |
| 109 | | CL | RTVAAPSVX₁IX₂PPSDEQLKSGTASVVCL |
| | | (Kabat) | LX₃X₄FYPREAKVQWKVDNALQSGNSQES |
| | | | VTEQDSKDSTYSLSSTLTLSKADYEKHKV |
| | | | YACEVTHQGLSSPVTKSFNRGEC, |
| | | | wherein X₁ is F/T, X₂ is F/M, X₃ is N/D/K, X₄ |
| | | | is N/D/K |
| 110 | EGFR | Heavy Chain | EVQLVESGGGLVQPGRSLRLSCAASGFTF |
| | | (VH-CH) | SDYGMHWVRQAPGKGLEWVSAIDAGGS |
| | | | TDYADSVEGRFTISRDNAKNSLYLQMNS |
| | | | LRAEDTAVYYCAKDLEAGYYAPDVWGK |
| | | | GTTVTVSSASTKGPSVFPX₁APSSKSTSGG |
| | | | TAALX₂CLVKDYFPEPVTVSWNSGALTSG |
| | | | VX₃TFPAVLQSSGLYSLSSVVX₄VPSSSLG |
| | | | TQTYICNVNHKPSNTKVDKKVEPKSCDK |
| | | | THTCPPCPAPELLGGPSVFLFPPKPKDTLM |
| | | | ISRTPEVTCVVVDVSHEDPEVKFNWYVD |
| | | | GVEVHNAKTKPREEQYNSTYRVVSVLTV |
| | | | LHQDWLNGKEYKCKVSNKALPAPIEKTIS |
| | | | KAKGQPREPQVYTLPPSRDELTKNQVSL |
| | | | WCLVKGFYPSDIAVEWESNGQPENNYKT |
| | | | TPPVLDSDGSFFLYSKLTVDKSRWQQGN |
| | | | VFSCSVMHEALHNHYTQKSLSLSPGX5, |
| | | | wherein X₁ is L/S, X₂ is G/L, X₃ is H/K/D, X₄ |
| | | | is T/K/D, X5 is K or absent |
| 111 | EGFR | Light Chain | DIQMTQSPSSLSASVGDRVTITCRASQDIG |
| | | (VL-CL) | RFLAWYQQKPGKAPKLLIYAVSNLQSGV |
| | | | PSRFSGSGSGTDFTLTISSLQPEDVATYYC |
| | | | QQYSTSVYTFGGGTKVEIKRTVAAPSVX₁I |
| | | | X₂PPSDEQLKSGTASVVCLLX₃X₄FYPREA |
| | | | KVQWKVDNALQSGNSQESVTEQDSKDST |
| | | | YSLSSTLTLSKADYEKHKVYACEVTHQG |
| | | | LSSPVTKSFNRGEC, |
| | | | wherein X₁ is F/T, X₂ is F/M, X₃ is N/D/K, X₄ |
| | | | is N/D/K |
| 112 | ITGB6 | Heavy Chain | QVQLX₁QSGAEX₂X₃X₄PGX₅SVKVSCKAS |
| | | (VH-CH) | GYDFNNDLIEWVX₆QX₇PGQX₈LEWX₉AVI |
| | | | NPGSGRTNYX₁₀X₁₁KFX₁₂GX₁₃X₁₄TX₁₅TAD |
| | | | KSX₁₆STVYMX₁₇LSSLX₁₈SX₁₉DX₂₀AVYX₂1 |
| | | | CAMIYYGPHSYAMDYWGQGTX₂₂VTVSS |
| | | | ASTKGPSVFPX₂₃APSSKSTSGGTAALX₂₄C |
| | | | LVKDYFPEPVTVSWNSGALTSGVX₂₅TFP |
| | | | AVLQSSGLYSLSSVVX₂₆VPSSSLGTQTYIC |
| | | | NVNHKPSNTKVDKKVEPKSCDKTHTCPP |
| | | | CPAPELLGGPSVFLFPPKPKDTLMISRTPE |
| | | | VTCVVVDVSHEDPEVKFNWYVDGVEVH |
| | | | NAKTKPREEQYNSTYRVVSVLTVLHQDW |
| | | | LNGKEYKCKVSNKALPAPIEKTISKAKGQ |
| | | | PREPQVYTLPPSRDELTKNQVSLTCLVKG |
| | | | FYPSDIAVEWESNGQPENNYKTTPPVLDS |
| | | | DGSFFLYSKLTVDKSRWQQGNVFSCSVM |
| | | | HEALHNHYTQKSLSLSPGK, |
| | | | wherein X₁ is V/Q, X₂ is V/L, X₃ is K/V, X₄ is |
| | | | K/R, X₅ is A/T, X₆ is R/K, X₇ is A/R, X₈ is |
| | | | C/G, X₉ is M/I, X₁₀ is A/N, X₁₁ is Q/E, X₁₂ is |
| | | | Q/K, X₁₃ is R/K, X₁₄ is V/A, X₁₅ is M/L, X₁₆ is |
| | | | T/S, X₁₇ is E/Q, X₁₈ is R/T, X₁₉ is E/D, X₂₀ is |
| | | | T/S, X₂₁ is Y/F, X₂₂ is L/S, X₂₃ is L/S, X₂₄ is |
| | | | G/L, X₂₅ is H/K/D, X₂₆ is T/K/D |
| 113 | ITGB6 | Light Chain | DIX₁MTQSX₂X₃X₄X₅SX₆X₇VGDRVX₈ITCK |
| | | (VL-CL) | ASLDVRTAVAWYQQKPGX₉X₁₀PKLLIYS |
| | | | ASYRYTGVPX₁₁RFX₁₂GSGSGTDFTFX₁₃IX₁₄ |
| | | | SX₁₅QX₁₆EDX₁₇AX₁₈YYCQQHYGIPWTFG |
| | | | GGTKLEIKRTVAAPSVX₁₉IX₂₀PPSDEQLKS |
| | | | GTASVVCLLX₂₁X₂₂FYPREAKVQWKVDN |
| | | | ALQSGNSQESVTEQDSKDSTYSLSSTLTLS |
| | | | KADYEKHKVYACEVTHQGLSSPVTKSFN |
| | | | RGEC, |
| | | | wherein X₁ is V/Q, X₂ is P/H, X₃ is S/K, X₄ is |
| | | | S/F, X₅ is M/L, X₆ is A/T, X₇ is V/S, X₈ is T/S, |
| | | | X₉ is K/Q, X₁₀ is A/S, X₁₁ is S/D, X₁₂ is S/T, |
| | | | X₁₃ is T/N, X₁₄ is S/R, X₁₅ is V/L, X₁₆ is P/A, |
| | | | X₁₇ is I/L, X₁₈ is V/T, X₁₉ is F/T, X₂₀ is F/M, |
| | | | X₂₁ is N/D/K, X₂₂ is N/D/K |
| 114 | EGFR | Heavy Chain | EVQLVESGGGLVQPGRSLRLSCAASGFTF |
| | | (VH-CH) | SDYGMHWVRQAPGKGLEWVSAIDAGGS |
| | | | TDYADSVEGRFTISRDNAKNSLYLQMNS |
| | | | LRAEDTAVYYCAKDLEAGYYAPDVWGK |
| | | | GTTVTVSSASTKGPSVFPSAPSSKSTSGGT |
| | | | AALLCLVKDYFPEPVTVSWNSGALTSGV |
| | | | KTFPAVLQSSGLYSLSSVVKVPSSSLGTQ |
| | | | TYICNVNHKPSNTKVDKKVEPKSCDKTH |
| | | | TCPPCPAPELLGGPSVFLFPPKPKDTLMIS |
| | | | RTPEVTCVVVDVSHEDPEVKFNWYVDG |
| | | | VEVHNAKTKPREEQYNSTYRVVSVLTVL |
| | | | HQDWLNGKEYKCKVSNKALPAPIEKTIS |
| | | | KAKGQPREPQVYTLPPSRDELTKNQVSL |
| | | | WCLVKGFYPSDIAVEWESNGQPENNYKT |
| | | | TPPVLDSDGSFFLYSKLTVDKSRWQQGN |
| | | | VFSCSVMHEALHNHYTQKSLSLSPG |
| 115 | EGFR | Light Chain | DIQMTQSPSSLSASVGDRVTITCRASQDIG |
| | | (VL-CL) | RFLAWYQQKPGKAPKLLIYAVSNLQSGV |
| | | | PSRFSGSGSGTDFTLTISSLQPEDVATYYC |
| | | | QQYSTSVYTFGGGTKVEIKRTVAAPSVTI |
| | | | MPPSDEQLKSGTASVVCLLDDFYPREAK |
| | | | VQWKVDNALQSGNSQESVTEQDSKDSTY |
| | | | SLSSTLTLSKADYEKHKVYACEVTHQGLS |
| | | | SPVTKSFNRGEC |
| 116 | ITGB6 | Heavy Chain | QVQLVQSGAEVKKPGASVKVSCKASGY |
| | | (VH-CH) | DFNNDLIEWVRQAPGQCLEWMAVINPGS |
| | | | GRTNYAQKFQGRVTMTADKSTSTVYME |
| | | | LSSLRSEDTAVYYCAMIYYGPHSYAMDY |
| | | | WGQGTLVTVSSASTKGPSVFPLAPSSKST |
| | | | SGGTAALGCLVKDYFPEPVTVSWNSGAL |
| | | | TSGVDTFPAVLQSSGLYSLSSVVDVPSSSL |
| | | | GTQTYICNVNHKPSNTKVDKKVEPKSAD |
| | | | KTHTCPPCPAPELLGGPSVFLFPPKPKDTL |
| | | | MISRTPEVTCVVVDVSHEDPEVKFNWYV |
| | | | DGVEVHNAKTKPREEQYNSTYRVVSVLT |
| | | | VLHQDWLNGKEYKCKVSNKALPAPIEKT |
| | | | ISKAKGQPREPQVYTLPPSRDELTKNQVS |
| | | | LSCAVKGFYPSDIAVEWESNGQPENNYK |
| | | | TTPPVLDSDGSFFLVSKLTVDKSRWQQG |
| | | | NVFSCSVMHEALHNHYTQKSLSLSPG |
| 117 | ITGB6 | Light Chain | DIQMTQSPSSLSASVGDRVTITCKASLDV |
| | | (VL-CL) | RTAVAWYQQKPGKAPKLLIYSASYRYTG |
| | | | VPSRFSGSGSGTDFTFTISSLQPEDIATYYC |
| | | | QQHYGIPWTFGCGTKVEIKRTVAAPSVFI |
| | | | FPPSDEQLKSGTASVVCLLKKFYPREAKV |
| | | | QWKVDNALQSGNSQESVTEQDSKDSTYS |
| | | | LSSTLTLSKADYEKHKVYACEVTHQGLSS |
| | | | PVTKSFNRGE |

In some embodiments, the sequences listed in Table 2 (SEQ ID NOs: 97-107) are amino acid molecules. In some embodiments, the sequences listed in Table 2 (SEQ ID NOs: 97-107) are amino acid molecules that are synthetic constructs. In some embodiments, the sequences listed in Table 2 (SEQ ID NOs: 97-107) for CH sequences (constant heavy chain), VH sequence (variable heavy chain sequence), CL sequences (constant light chain), VL sequence (variable light chain sequence) are amino acid molecules that are synthetic constructs.

### Antibody-Like Frameworks or Scaffolds

A wide variety of antibody/immunoglobulin frameworks or scaffolds can be employed in the anti-EGFR antibody, anti-ITGB6 antibody, and/or the bispecific anti-EGFR and anti-ITGB6 antigen binding molecules as described herein, or multifunctional formats thereof, so long as the resulting polypeptide includes at least one binding region which specifically binds to the target antigen, e.g., an EGFR, an ITGB6, a tumor antigen, among others. Such frameworks or scaffolds include the 5 main idiotypes of human immunoglobulins, or fragments thereof, and include immunoglobulins of other animal species, preferably having humanized aspects. Novel frameworks, scaffolds and fragments continue to be discovered and developed by those skilled in the art.

In some embodiments, the anti-EGFR antibody, anti-ITGB6 antibody, and/or the bispecific anti-EGFR and anti-ITGB6 antibody as described herein, or multifunctional formats thereof, include non-immunoglobulin based antibodies using non-immunoglobulin scaffolds onto which CDRs can be grafted. Any non-immunoglobulin frameworks and scaffolds may be employed, as long as they comprise a binding region specific for the target antigen (e.g., EGFR or ITGB6). Exemplary non-immunoglobulin frameworks or scaffolds include, but are not limited to, fibronectin (Compound Therapeutics, Inc., Waltham, MA), ankyrin (Molecular Partners AG, Zurich, Switzerland), domain antibodies (Domantis, Ltd., Cambridge, MA, and Ablynx nv, Zwijnaarde, Belgium), lipocalin (Pieris Proteolab AG, Freising, Germany), small modular immuno-pharmaceuticals (Trubion Pharmaceuticals Inc., Seattle, WA), maxybodies (Avidia, Inc., Mountain View, CA), Protein A (Affibody AG, Sweden), and affilin (gamma-crystallin or ubiquitin) (Scil Proteins GmbH, Halle, Germany).

Fibronectin scaffolds are typically based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The ankyrin technology is based on using proteins with ankyrin derived repeat modules as scaffolds for bearing variable regions which can be used for binding to different targets. The ankyrin repeat module typically is a about 33 amino acid polypeptide consisting of two anti-parallel α-helices and a R-tum. Binding of the variable regions can be optimized by using ribosome display.

Avimers are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are known commercially, e.g., Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

Domain antibodies (dAbs) can be used in the anti-EGFR antibody, anti-ITGB6 antibody, and/or the bispecific anti-EGFR and anti-ITGB6 antibody as described herein or multifunctional formats thereof. Domain antibodies (dAbs) can be small functional binding fragments of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; 6,172,197; 6,696,245; European Patents 0368684 & 0616640; WO05/035572, WO04/101790, WO04/081026, WO04/058821, WO04/003019 and WO03/002609. Nanobodies are derived from the heavy chains of an antibody.

A nanobody typically comprises a single variable domain and two constant domains (CH2 and CH3) and retains antigen-binding capacity of the original antibody. Nanobodies can be prepared by methods known in the art (See e.g., U.S. Pat. Nos. 6,765,087, 6,838,254, WO 06/079372). Unibodies consist of one light chain and one heavy chain of an IgG4 antibody. Unibodies may be made by the removal of the hinge region of IgG4 antibodies. Further details of unibodies and methods of preparing them may be found in WO2007/059782.

### Anti-EGFR and Anti-ITGB6 Antibody Effector Function and Fc Variants

In some embodiments, an anti-EGFR antibody, an anti-ITGB6 antibody, and/or a bispecific anti-EGFR and anti-ITGB6 antibody as described herein comprises an Fc region, e.g., as described herein. In some embodiments, the Fc region is a wildtype Fc region, e.g., a wildtype human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

The Fc region of an antibody interacts with a number of receptors or ligands including Fc Receptors (e.g., FcγRI, FcγRIIA, FcγRIIIA), the complement protein CIq, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, an anti-EGFR antibody, an anti-ITGB6 antibody, and/or a bispecific anti-EGFR and anti-ITGB6 antibody comprising a variant Fc region has reduced, e.g., ablated, affinity for an Fc receptor, e.g., an Fc receptor described herein. In some embodiments, the reduced affinity is compared to an otherwise similar antibody with a wildtype Fc region.

In some embodiments, an anti-EGFR antibody, an anti-ITGB6 antibody, and/or a bispecific anti-EGFR and anti-ITGB6 antibody comprising a variant Fc region has one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region.

In some embodiments, an anti-EGFR antibody, an anti-ITGB6 antibody, and/or a bispecific anti-EGFR and anti-ITGB6 antibody comprising a variant Fc region has reduced affinity to a human Fc receptor, e.g., FcγR I, FcγR II and/or FcγR III. In some embodiments, the anti-EGFR antibody, anti-ITGB6 antibody, and/or the bispecific anti-EGFR and anti-ITGB6 antibody comprising a variant Fc region comprises a human IgG1 region or a human IgG4 region.

Exemplary Fc region variants are disclosed in Saunders 0, (2019) Frontiers in Immunology; vol 10, article1296, the entire contents of which is hereby incorporated by reference. In some embodiments, an anti-EGFR antibody, an anti-ITGB6 antibody, and/or a bispecific anti-EGFR and anti-ITGB6 antibody comprises a pro-body. In some embodiments, the antigen binding molecules disclosed herein comprise a pro-body. A pro-body, such as a “masked” antibody or molecule, can refer to a modified form of an antibody or therapeutic protein that is designed to remain inactive until it encounters a specific target in the body. For example, in a pro-body, the active binding region of the molecule is concealed or masked by an additional component, such as a peptide or a chemical linker. When the pro-body encounters its specific target, such as a specific enzyme or marker expressed on cancer cells, the masking component is selectively cleaved or modified. Once the masking component is removed, the active binding region of the pro-body is exposed, allowing can bind specifically to its target.

### Multifunctional Antigen Binding Molecules

As used herein, a “multifunctional” or a “multispecific” antigen binding molecule refers to antigen binding molecules, e.g., a polypeptide, that has two or more functionalities, e.g., two or more binding specificities. In some embodiments, the functionalities can include one or more immune cell engagers, one or more tumor binding molecules, and other moieties described herein. In some embodiments, the multispecific antigen binding molecule is a multispecific antibody, e.g., a bispecific antibody. In some embodiments, the multispecific antigen binding molecule includes an anti-EGFR antibody, an anti-ITGB6 antibody, and/or a bispecific anti-EGFR and anti-ITGB6 antibody as described herein.

In some embodiments, the multifunctional antigen binding molecules further includes a tumor antigen moiety. In some embodiments, the tumor-targeting moiety is an antigen, e.g., a cancer antigen. In some embodiments, the cancer antigen is a tumor antigen.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

In some embodiments, the tumor-targeting moiety, e.g., cancer antigen, is EGFR and/or ITGB6. In some embodiments, the multifunctional or multispecific antigen binding molecule, e.g., the EGFR targeting moiety, binds to an EGFR antigen on the surface of a cell, e.g., a cancer. The EGFR antigen can be present on a primary tumor cell, or a metastatic lesion thereof. In some embodiments, the cancer is a lung cancer, such as non-small cell lung cancer (NSCLC). In some embodiments, the cancer is a gastrointestinal cancer, such as colorectal cancer (CRC). In some embodiments, the cancer is squamous cell carcinoma, such as head and neck squamous cell carcinoma (HNSCC). In some embodiments, the cancer is esophageal cancer. In some embodiments, the cancer is bladder cancer. In some embodiments, the EGFR-targeting moiety is an antibody (e.g., Fab or scFv) that binds to EGFR. In some embodiments, the antigen binding molecule to EGFR comprises one, two, or three CDRs from any of the heavy chain variable domain sequences (e.g., corresponding to EGFR exemplary antigen) of Table 1, Table 2, Table 5, or a closely related CDR, e.g., CDRs which have at least one amino acid alteration, but not more than two, three or four alterations (e.g., substitutions, deletions, or insertions, e.g., conservative substitutions). In some embodiments, the antigen binding molecule to EGFR comprises a heavy chain variable domain sequence chosen from any of the amino acid sequences (e.g., corresponding to EGFR exemplary antigen) of Table 1, Table 2, Table 5, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)). In some embodiments, the multifunctional or multispecific antigen binding molecule, e.g., the ITGB6 targeting moiety, binds to an ITGB6 antigen on the surface of a cell, e.g., a cancer. The ITGB6 antigen can be present on a primary tumor cell, or a metastatic lesion thereof. In some embodiments, the cancer is non-small cell lung cancer (NSCLC). In some embodiments, the cancer is colorectal cancer (CRC). In some embodiments, the cancer is squamous cell carcinoma, such as head and neck squamous cell carcinoma (HNSCC). In some embodiments, the cancer is esophageal cancer. In some embodiments, the cancer is bladder cancer. In some embodiments, the ITGB6-targeting moiety includes an antigen binding molecule (e.g., Fab or scFv) that binds to ITGB6. In some embodiments, the antigen binding molecule to ITGB6 comprises one, two, or three CDRs from any of the heavy chain variable domain sequences of (e.g., corresponding to ITGB6 exemplary antigen) of Table 1, Table 2, Table 5, or a closely related CDR, e.g., CDRs which have at least one amino acid alteration, but not more than two, three or four alterations (e.g., substitutions, deletions, or insertions, e.g., conservative substitutions). In some embodiments, the antigen binding molecule to ITGB6 comprises a heavy chain variable domain sequence chosen from any of the amino acid sequences of (e.g., corresponding to ITGB6 exemplary antigen) of Table 1, Table 2, Table 5, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)).

Alternatively, or in combination with the heavy chain to EGFR as described herein, the antigen binding molecule to EGFR comprises one, two, or three CDRs from any of the light chain variable domain sequences of (e.g., corresponding to EGFR exemplary antigen) of Table 1, Table 2, Table 5, or a closely related CDR, e.g., CDRs which have at least one amino acid alteration, but not more than two, three or four alterations (e.g., substitutions, deletions, or insertions, e.g., conservative substitutions) from any of the CDR sequences of (e.g., corresponding to EGFR exemplary antigen) of Table 1, Table 2, and/or Table 5. In some embodiments, the antigen binding molecule to EGFR comprises a light chain variable domain sequence chosen from any of the amino acid sequences of (e.g., corresponding to EGFR exemplary antigen) of Table 1, Table 2, Table 5, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)). Alternatively, or in combination with the heavy chain to ITGB6 as described herein, the antigen binding molecule to ITGB6 comprises one, two, or three CDRs from any of the light chain variable domain sequences of (e.g., corresponding to ITGB6 exemplary antigen) of Table 1, Table 2, Table 5, or a closely related CDR, e.g., CDRs which have at least one amino acid alteration, but not more than two, three or four alterations (e.g., substitutions, deletions, or insertions, e.g., conservative substitutions) from any of the CDR sequences of (e.g., corresponding to ITGB6 exemplary antigen) of Table 1, Table 2, and/or Table 5. In some embodiments, the antigen binding molecule to EGFR comprises a light chain variable domain sequence chosen from any of the amino acid sequences of (e.g., corresponding to ITGB6 exemplary antigen) of Table 1, Table 2, Table 5, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)).

In some embodiments, the multifunctional or multispecific (e.g., bi-, tri-, tetra-specific) antigen binding molecules as described herein further include, e.g., are engineered to further contain, one or more tumor specific targeting moieties that direct the antigen binding molecule to a tumor cell.

In certain embodiments, the multifunctional or multispecific antigen binding molecules as described herein further include a tumor-targeting moiety. The tumor targeting moiety can be chosen from an antigen binding molecule (e.g., an antigen binding domain as described herein), a receptor or a receptor fragment, or a ligand or a ligand fragment, or a combination thereof. In some embodiments, the tumor targeting moiety associates with, e.g., binds to, a tumor cell (e.g., a molecule, e.g., antigen, present on the surface of the tumor cell).

In certain embodiments, the tumor targeting moiety targets, e.g., directs the multifunctional or multispecific antigen binding molecules as described herein to a cancer (e.g., a cancer or tumor cells). In some embodiments, the cancer is chosen from a solid cancer, a metastatic cancer, or a combination thereof.

In some embodiments, the multifunctional or multispecific antigen binding molecule, e.g., the tumor-targeting moiety, binds to a solid tumor antigen or a stromal antigen. The solid tumor antigen can be present on a solid tumor, or a metastatic lesion thereof. In some embodiments, the solid tumor is a lung cancer, such as non-small cell lung cancer (NSCLC). In some embodiments, the solid tumor is a gastrointestinal cancer, such as colorectal cancer (CRC). In some embodiments, the solid tumor is a squamous cell carcinoma, such as head and neck squamous cell carcinoma (HNSCC). In some embodiments, the solid tumor is an esophageal cancer. In some embodiments, the solid tumor is a bladder cancer. For example, the solid tumor antigen can be present on a tumor, e.g., a tumor of a class typified by having one or more of limited tumor perfusion, or compressed blood vessels.

### Antibody

In some embodiments, the antigen binding molecule is an antibody, or functional fragment thereof. In some embodiments, the antigen binding molecule binds to a cancer antigen, e.g., a tumor antigen or a stromal antigen. In some embodiments, the cancer antigen is, e.g., a mammalian, e.g., a human, cancer antigen. For example, the antigen binding molecule binds specifically to an epitope, e.g., linear or conformational epitope, on the cancer antigen.

In some embodiments, an antigen binding molecule is a multispecific or multifunctional antibody, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a multispecific antigen binding molecule comprises a third, fourth or fifth immunoglobulin variable domain. In some embodiments, a multispecific antibody is a bispecific antibody, a trispecific antibody, or a tetraspecific antibody.

In some embodiments, a multispecific antibody is a bispecific antibody. A bispecific antibody has specificity for no more than two antigens. A bispecific antibody can be characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody comprises a half antibody having binding specificity for a first epitope and a half antibody having binding specificity for a second epitope. In some embodiments, a bispecific antibody comprises a half antibody, or fragment thereof, having binding specificity for a first epitope and a half antibody, or fragment thereof, having binding specificity for a second epitope. In some embodiments, a bispecific antibody comprises a scFv or a Fab, or fragment thereof, have binding specificity for a first epitope and a scFv or a Fab, or fragment thereof, have binding specificity for a second epitope.

In some embodiments, an antibody comprises a diabody, and a single-chain molecule, as well as an antigen-binding fragment of an antibody (e.g., Fab, F(ab′)2, and Fv). For example, an antibody can include a heavy (H) chain variable domain sequence (abbreviated herein as VH), and a light (L) chain variable domain sequence (abbreviated herein as VL). In some embodiments, an antibody comprises or consists of a heavy chain and a light chain (referred to herein as a half antibody. In another example, an antibody includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibodies can be monoclonal or polyclonal. An antibody can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody include: (i) a Fab fragment, a monovalent fragment consisting of the VL, VH, CL and C_{H}1 domains; (ii) a F(ab′)2 fragment, a bivalent fragment comprising two Fab fragments linked by a disulfide bridge at the hinge region; (iii) a Fd fragment consisting of the VH and C_{H}1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibodies include intact antigen binding molecules as well as functional fragments thereof. Constant regions of the antibody can be altered, e.g., mutated, to modify the properties of the antibody (e.g., to increase or decrease one or more of: Fc receptor binding, antibody glycosylation, the number of cysteine residues, effector cell function, or complement function).

Antibodies can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDRT), 50-65 (HCDR2), and 95-102 (HCDR3); and the CDR amino acid residues in the light chain variable domain (VL) are numbered 24-34 (LCDRT), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody can be a polyclonal or a monoclonal antibody.

The terms “monoclonal antibody” or “monoclonal antibody composition” as used herein refer to a preparation of antibodies of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods, or by yeast display.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

The yeast display method for generating or identifying antibodies is known in the art, e.g., as described in Chao et al. (2006) Nature Protocols 1(2):755-68, the entire contents of which is incorporated by reference herein.

In some embodiments, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In some embodiments, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody has a heavy chain constant region chosen from, e.g., the heavy chain constant regions of IgG1, IgG2, IgG3, IgG4, IgM, IgAQ1, IgA2, IgD, and IgE; particularly, chosen from, e.g., the (e.g., human) heavy chain constant regions of IgG1, IgG2, IgG3, and IgG4. In another embodiment, the antibody has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody can be derivatized or linked to another functional antigen binding molecule (e.g., another peptide or protein). As used herein, a “derivatized” antibody is one that has been modified. Methods of derivatization include but are not limited to the addition of a fluorescent moiety, a radionucleotide, a toxin, an enzyme or an affinity ligand such as biotin. Accordingly, the antibodies of the invention are intended to include derivatized and otherwise modified forms of the antibodies described herein, including immunoadhesion molecules. For example, an antibody can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### CDR-Grafted Scaffolds

In some embodiments, the antibody is a CDR-grafted scaffold domain. In some embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In some embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In some embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In some embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In some embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent antigen binding molecules with bivalent binding specificity for each antigen. Combinations of scFvs and scFabs with IgGs enable the production of antigen binding molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, U.S. Pat. No. 9,359,437, US20150018529, WO2016115274A1, WO2016087416A1, US20080069820A1, U.S. Pat. Nos. 9,145,588B, 7,919,257, and US20150232560A1. Exemplary multispecific antigen binding molecules utilizing a full antibody-Fab/scFab format include those described in the following, U.S. Pat. No. 9,382,323B2, US20140072581A1, US20140308285A1, US20130165638A1, US20130267686A1, US20140377269A1, U.S. Pat. No. 7,741,446B2, and WO1995009917A1. Exemplary multispecific antigen binding molecules utilizing a domain exchange format include those described in the following, US20150315296A1, WO2016087650A1, US20160075785A1, WO2016016299A1, US20160130347A1, US20150166670, U.S. Pat. No. 8,703,132B2, US20100316645, U.S. Pat. No. 8,227,577B2, US20130078249.

### Fc-Containing Multifunctional or Multispecific Antigen Binding Molecules

In some embodiments, the multifunctional or multispecific antigen binding molecules as described herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multifunctional or multispecific antigen binding molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional antigen binding molecule includes a half-life extender, e.g., a human serum albumin or an antibody to human serum albumin.

### Methods

### Degradation

The present disclosure provides methods of degrading a target protein on a surface of a target cell, the method comprising: contacting an endogenous internalizing receptor and the target protein on the surface of the target cell with the antigen binding molecule of the present disclosure, wherein the antigen binding molecule specifically binds to: (i) an endogenous internalizing receptor, wherein the endogenous internalizing receptor comprises ITGB6; and (ii) the target protein, wherein the target protein comprises EGFR

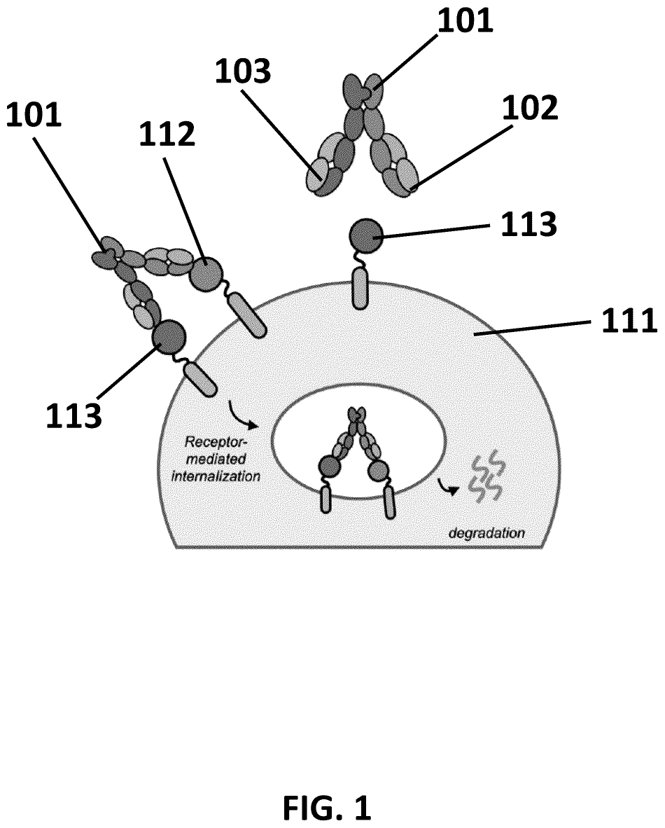

The present disclosure provides methods of degrading an EGFR protein on a target cell as shown in FIG. 1. The method utilizes an antigen binding molecule 101 that binds specifically to both (1) an extracellular epitope on the EGFR protein 112; and (2) an extracellular epitope on a membrane-associated internalizing protein 113, such as ITGB6, on a target cell 111. The antigen binding molecule 101 disclosed herein, comprises a first antigen binding domain 102 that selectively binds to the EGFR protein 112 and a second antigen binding domain 103 that selectively binds to membrane-associated internalizing protein 113, such as ITGB6. Simultaneous binding of the multispecific antigen binding molecule 101 to the EGFR protein 112 and the membrane-associated internalizing protein 113, such as ITGB6, leads to internalization of both the EGFR protein 112 and the membrane-associated internalizing protein 113, such as ITGB6, into the target cell 111. Following internalization, the EGFR protein 112 is degraded by the target cell 111 (e.g., via trafficking to the lysosome).

Provided herein are methods of engaging ITGB6 internalization for degradation. In some embodiments, methods disclosed herein include methods of engaging ITGB6 internalization for degradation of a cell surface target protein comprising contacting a cell with a ITGB6 antibody that also binds to the cell surface target protein that competes with and/or binds the same epitope as a reference antibody, wherein the reference antibody comprises: a heavy chain variable region (VH) comprising the amino acid sequence set forth in SEQ ID NO: 85; and a light chain variable region (VL) comprising the amino acid sequence set forth in SEQ ID NO: 91.

Provided herein are methods of degrading EGFR on the surface of a cancer cell. In some embodiments, methods disclosed herein include methods of degrading EGFR on the surface of a cancer cell comprising, contacting the cell with an EGFR×ITGB6 bispecific antibody that competes with and/or binds the same epitope as a reference antibody, wherein the reference antibody comprises: a heavy chain variable region (VH) comprising the amino acid sequence set forth in SEQ ID NO: 43; and a light chain variable region (VL) comprising the amino acid sequence set forth in SEQ ID NO: 49. In some embodiments, methods disclosed herein include methods of degrading EGFR on the surface of a cancer cell comprising, contacting the cell with an EGFR×ITGB6 bispecific antibody that competes with and/or binds the same epitope as a reference antibody, wherein the reference antibody comprises: a heavy chain variable region (VH) comprising the amino acid sequence set forth in SEQ ID NO: 85; and a light chain variable region (VL) comprising the amino acid sequence set forth in SEQ ID NO: 91.

Provided herein are methods of selectively killing an EFGR expressing cancer cell. In some embodiments, methods disclosed herein include methods of selectively killing an EFGR expressing cancer cell comprising, contacting the cell with an EGFR×ITGB6 bispecific antibody that competes with and/or binds the same epitope as a reference antibody, wherein the reference antibody comprises: a heavy chain variable region (VH) comprising the amino acid sequence set forth in SEQ ID NO: 43; and a light chain variable region (VL) comprising the amino acid sequence set forth in SEQ ID NO: 49. In some embodiments, methods disclosed herein include methods of selectively killing an EFGR expressing cancer cell comprising, contacting the cell with an EGFR×ITGB6 bispecific antibody that competes with and/or binds the same epitope as a reference antibody, wherein the reference antibody comprises: a heavy chain variable region (VH) comprising the amino acid sequence set forth in SEQ ID NO: 85; and a light chain variable region (VL) comprising the amino acid sequence set forth in SEQ ID NO: 91.

In some embodiments, the cancer cell is a non-small cell lung cancer (NSCLC) cell, a colorectal cancer (CRC) cell, or a squamous cell carcinoma (HNSCC) cell. In some embodiments, the cancer cell is a NSCLC cell.

### Binding On Target Cells

The binding activity of the antigen binding molecules of the present disclosure can be assayed by any suitable method known in the art. The binding activity of the antigen binding molecules of the present disclosure can be assayed by any suitable method known in the art for assaying antibodies. For example, the binding activity of antigen binding molecules of the present disclosure can be determined by, e.g., Scatchard analysis (Munsen et al., Analyt Biochem (1980) 107:220-39). Specific binding may be assessed using techniques known in the art including but not limited to competition ELISA, BIACORE® assays and/or KINEXA® assays. An antibody that preferentially or specifically binds (used interchangeably herein) to a target antigen or target epitope is a term well understood in the art, and methods to determine such specific or preferential binding are also known in the art. An antibody is said to exhibit specific or preferential binding if it reacts or associates more frequently, more rapidly, with greater duration and/or with greater affinity with a particular antigen or epitope than it does with alternative antigens or epitopes. An antibody specifically or preferentially binds to a target if it binds with greater affinity, avidity, more readily, and/or with greater duration than it binds to other substances. Also, an antibody specifically or preferentially binds to a target if it binds with greater affinity, avidity, more readily, and/or with greater duration to that target in a sample than it binds to other substances present in the sample. For example, an antibody that specifically or preferentially binds to an ITGB6 epitope is an antibody that binds this epitope with greater affinity, avidity, more readily, and/or with greater duration than it binds to other ITGB6 epitopes or non-ITGB6 epitopes. It is also understood by reading this definition, for example, that an antibody which specifically or preferentially binds to a first target antigen may or may not specifically or preferentially bind to a second target antigen. As such, specific binding and preferential binding do not necessarily require (although it can include) exclusive binding.

In some embodiments, the antigen binding molecules of the present disclosure decrease expression of EGFR on the cancer cell by at least 40%, at least 50%, at least 55%, at least 60%, at least 65%, at least 70%, at least 75%, or at least 80%. In some embodiments, the antigen binding molecules of the present disclosure decrease expression of EGFR on the cancer cell by about 40%-80%, about 50%-80%, about 60%-80%, about 70%-80%, about 40%-70%, about 50%-70%, about 60%-70%, about 40%-60%, or about 50%-60%. In some embodiments, expression of EGFR on a target cell is determined relative to expression of EGFR on a control cancer cell not contacted with the antigen binding molecule.

In some embodiments, the antigen binding molecules of the present disclosure increases surface removal of EGFR on a target cancer cell by at least 20%, at least 30%, at least 40%, at least 50%, at least 60%, at least 65%, at least 70%, at least 75%, at least 80%, at least 85%, or at least 90%. In some embodiments, the antigen binding molecules of the present disclosure increases cell surface removal of EGFR by about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30%. In some embodiments, cell surface removal of EGFR on a target cell is determined relative to cell surface removal of EGFR on a control cancer cell not contacted with the antigen binding molecule. In some embodiments, the antigen binding molecules of the present disclosure increases internalization of EGFR on a target cancer cell by at least 20%, at least 30%, at least 40%, at least 50%, at least 60%, at least 65%, at least 70%, at least 75%, at least 80%, at least 85%, or at least 90%. In some embodiments, the antigen binding molecules of the present disclosure increases internalization of EGFR by about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30%. In some embodiments, internalization of EGFR on a target cell is determined relative to internalization of EGFR on a control cancer cell not contacted with the antigen binding molecule.

In some embodiments, the antigen binding molecules of the present disclosure increases degradation of EGFR on a target cancer cell by at least 20%, at least 30%, at least 40%, at least 50%, at least 60%, at least 65%, at least 70%, at least 75%, at least 80%, at least 85%, or at least 90%. In some embodiments, the antigen binding molecules of the present disclosure increases degradation of EGFR by about 20-90%, about 30-90%, about 40-90%, about 50-90%, about 60-90%, about 70-90%, about 80-90%, about 20-80%, about 30-80%, about 40-80%, about 50-80%, about 60-80%, about 70-80%, about 20-70%, about 30-70%, about 40-70%, about 50-70%, about 60-70%, about 20-60%, about 30-60%, about 40-60%, about 50-60%, about 20-50%, about 30-50%, about 40-50%, about 20-40%, about 30-40%, or about 20-30%. In some embodiments, degradation of EGFR on a target cell is determined relative to degradation of EGFR on a control cancer cell not contacted with the antigen binding molecule.

In some embodiments, the antigen binding molecules of the present disclosure increases susceptibility of the cancer cell to cancer therapeutic agents. In some embodiments, the antigen binding molecules of the present disclosure increases susceptibility of the cancer cell to cytotoxic agents. In some embodiments, the antigen binding molecules of the present disclosure reduces proliferation of the target cancer cell. In some embodiments, the antigen binding molecules of the present disclosure increases death of the cancer cell. In some embodiments, the antigen binding molecules of the present disclosure contacts a target cancer cell in vivo.

### Multispecific and Multifunctional Antigen Binding Molecules

Exemplary structures of multispecific and multifunctional antigen binding molecules defined herein are described throughout. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

In some embodiments, multifunctional or multispecific antigen binding molecules can comprise more than one antigen-binding site, where different sites are specific for different antigens. In some embodiments, multifunctional or multispecific antigen binding molecules can bind more than one (e.g., two or more) epitopes on the same antigen. In some embodiments, multifunctional or multispecific antigen binding molecules comprise an antigen-binding site specific for a target cell (e.g., cancer cell) and a different antigen-binding site specific for an immune effector cell. In some embodiments, the multifunctional or multispecific antigen binding molecule is a bispecific antibody. Bispecific antibodies can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, kl-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in kl-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibodies. For example, monovalent IgG can be engineered to have bispecificity by appending an additional antigen-binding unit onto the monovalent IgG, e.g., at the N- or C- terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-1α and IL-10; and ABT-122 (AbbVie), which binds TNF and IL-17A.

Bispecific antibody fragments (BsAb) are a format of bispecific antibodies that lack some or all of the antibody constant domains. For example, some BsAb lack an Fc region. In some embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFvs bind to a surface antigen on cancer cells.

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In some embodiments, the dock-and-lock (DNL) method can be used to generate bispecific antibodies with higher valency. Also, fusions to albumin binding proteins or human serum albumin can be extend the serum half-life of antibody fragments. See Id.

In some embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

The antigen binding molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antigen binding molecules can be produced by separate expression of the components in different host cells and subsequent purification/assembly. Alternatively, the antigen binding molecules can be produced by expression of the components in a single host cell. Purification of bispecific antigen binding molecules can be performed by various methods such as affinity chromatography, e.g., using protein A and sequential pH elution. In other embodiments, affinity tags can be used for purification, e.g., histidine-containing tag, myc tag, or streptavidin tag.

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-in-Hole

Knob-in-Hole as described in U.S. Pat. Nos. 5,731,116, 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary Fc mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic- Met antibody inhibits glioblastoma growth in vivo. Clin Cancer Res 2006; 12:6144-52; PMID:17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. No. 9,309,311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1, U.S. Pat. No. 7,855,275B2, and U.S. Pat. No. 9,000,130B2.

### Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis J H et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID:20299542 and U.S. Pat. No. 8,871,912. The contents of each of which are incorporated by reference herein).

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Duobody

“Duobody” technology to produce bispecific antibodies with correct heavy chain pairing are known. The DuoBody technology involves three basic steps to generate stable bispecific human IgGlantibodies in a post-production exchange reaction. In a first step, two IgG1s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013; 110(13):5145-5150 and Labrijn et al. Nature Protocols 2014; 9(10):2450-63, the contents of each of which are incorporated by reference herein).

### Electrostatic Interactions

Methods of making multifunctional or multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multifunctional or multispecific antigen binding molecules using electrostatic interactions are described in the following references, the contents of each of which is incorporated by reference herein, include US20100015133, U.S. Pat. No. 8,592,562B2, U.S. Pat. No. 9,200,060B2, US20140154254A1, and U.S. Pat. No. 9,358,286A1.

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081A1, and EP3055329A1, the contents of each of which is incorporated by reference herein.

### CrossMab

Another option to reduce light chain mispairing is the CrossMab technology which avoids non-specific L chain mispairing by exchanging C_{H}1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (C_{H}1) and constant light (CL) domains of one antibody are exchanged (Antibody A), keeping the variable heavy (VH) and variable light (VL) domains consistent. The exchange of the C_{H}1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28);
doi:10.1038/scibx.2011.783, the contents of which are incorporated by reference herein).

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein. Expression of two different heavy and light chains in a single cell can create misassembled unwanted species. such as heavy-light chain mispairing. These impurities can be difficult to remove due to their similarity to the correct format. Enhancing the formation of a desired bispecific antibody from a mixture of monomers can also be achieved through correct heavy-light chain pairing. For example, bYlok® bispecific pairing technology can be used to engineer differential cysteine binding between the light and heavy chain to help with correct pairing (e.g., engineering a native disulfide bridge and relocating it from one of the constant domains, such as CH1/CL, to the variable domains, such as VH/VL). In some cases, one or more mutations can be incorporated into the antigen binding molecules disclosed herein. In some embodiments, the one or more mutations are configured to improve scFv stability and/or create diabodies. In some embodiments, the one or more mutations are configured to drive correct heavy-light chain pairing. In some embodiments, a native disulfide bridge between a C_{H}1 region and a CL region is relocated to be between a VH region and a VL region. In some embodiments, a native C_{H}1 region cysteine and a native CL region cysteine are relocated to a VH region and a VL region, respectively. In some embodiments, the light chain (LC) of the first antigen binding domain of the antigen binding molecules disclosed herein comprise a 100C and/or a C214del mutation, according to Kabat numbering. In some embodiments, the light chain (LC) of the second antigen binding domain of the antigen binding molecules disclosed herein comprise a 100C and/or a C214del mutation, according to Kabat numbering. In some embodiments, the heavy chain (HC) of a first arm of the antigen binding molecules disclosed herein comprise a 44C and/or a C233A (also referred to as C220A with Eu numbering) mutation, according to Kabat numbering. In some embodiments, the heavy chain (HC) of a second arm of the antigen binding molecules disclosed herein comprise a 44C and/or a C233A mutation, according to Kabat numbering. In some embodiments, the antigen binding molecules disclosed herein can comprise one or more mutations, according to Kabat numbering, selected from the following: HC 44-LC 100 (e.g., bYlok@), HC 44-LC 106, HC 44-LC 105, HC 45-LC 103, and HC 46-LC 103. In some embodiments, the one or more mutations configured to drive correct heavy-light chain pairing can be selected from one or more of the following: HC 44-LC 100 (e.g., bYlok®), HC 44-LC 106, HC 44-LC 105, HC 45-LC 103, and HC 46-LC 103, according to Kabat numbering. In some embodiments, the antigen binding molecules disclosed herein can comprise one or more mutations, according to Kabat numbering, selected from the following: HC G44 and LC G100 (e.g., bYlok®), HC G44 and LC 1106, HC G44 and LC E105, HC L45 and LC K103, and HC E46 and LC K103. In some embodiments, the one or more mutations configured to drive correct heavy-light chain pairing can be selected from one or more of the following: HC G44-LC G100 (e.g., bYlok®), HC G44-LC 1106, HC G44-LC E105, HC L45-LC K103, and HC E46-LC K103, according to Kabat numbering.

In some embodiments, the antigen binding molecule comprises: (a) a first C_{H}1 domain (C_{H}1) and a first C_{L} domain (C_{L}), the first C_{H}1 domain and the first C_{L} domain interacting together at a first C_{H}C_{L} interface to form a first C_{H}C_{L} domain (C_{H}C_{L}); (b) a second C_{H}1 domain (C_{H}1) and a second C_{L} domain (C_{L}), the second C_{H}1 domain and the second C_{L} domain interacting together at a second C_{H}C_{L} interface to form a second C_{H}C_{L} domain (C_{H}C_{L}). In some embodiments, the first C_{H}1 domain and/or the second C_{H}1 domain have at least one mutation relative to a human immunoglobulin C_{H}1 domain, referred to as the C_{H}1 mutant residue(s). In some embodiments, each C_{H}1 mutant residue is only present in one of first C_{H}1 domain or the second C_{H}1 domain. In some embodiments, the first C_{L} domain and/or the second C_{L} domain have at least one mutation relative to a human immunoglobulin C_{L} domain, referred to as the C_{L} mutant residue(s). In some embodiments, each C_{L} mutant residue is only present in one of first C_{L} domain or the second C_{L} domain. In some embodiments, the C_{H}1 mutant residue(s) and the C_{L} mutant residue(s) comprise charged amino acids such that a first C_{H}1 mutant residue and a first C_{L} mutant residue comprise a charge pair. In some embodiments, the C_{H}1 mutant residues and the C_{L} mutant residues comprise a steric pair such that (a) (i) the second C_{H}1 mutant residue has steric conflict with the first C_{L} domain or the second C_{L} domain or (ii) the second C_{L} mutant residue has steric conflict with the first CHT domain or the second CHT domain and (b) the second C_{H}1 mutant residue and the second C_{L} mutant residue do not have steric conflict. In some embodiments, (i) the first CHT mutant residue is located at H172 and/or T192 and the first C_{L} mutant residue is located at N137 and/or N138. In some embodiments, the C_{H}1 mutant residue(s) and the CL mutant residue(s) comprise a steric pair such that (a) (i) a first C_{H}1 mutant residue has steric conflict with the first C_{L} domain or the second C_{L} domain or (ii) a first C_{L} mutant residue has steric conflict with the first CHT domain or the second CHT domain and (b) the first C_{H}1 mutant residue and the first C_{L} mutant residue do not have steric conflict, and wherein (i) the first CHT mutant residue is located at L124 and/or G141 and the a first C_{L} mutant residue is located at F116 and/or F118. In some embodiments, the first CHT domain is attached to a first variable heavy domain (V_{H}), and the first C_{L} domain is attached to a first variable light domain (V_{L}), and the second C_{H}1 domain is attached to a second VH domain, and the second C_{L} domain is attached to a second V_{L} domain, such that when combined, the first VH domain, first V_{L} domain, first CH domain and first C_{L} domain together form a first Fab, and when combined, the second VH domain, second V_{L} domain, second C_{H}1 domain, and second C_{L} domain form a second Fab. In some embodiments, the first VH domain or the second VH domain has at least one mutation relative to a human immunoglobulin VH domain, referred to as the VH mutant residue(s); and the first V_{L} domain or the second V_{L} domain has at least one mutation relative to a human immunoglobulin V_{L} domain, referred to as the V_{L} mutant residue(s).

In some embodiments, the C_{H}1 mutant residue(s) and the C_{L} mutant residue(s) comprise charged amino acids such that the C_{H}1 mutant residue(s) and the C_{L} mutant residue(s) comprise a charge pair. In some embodiments, the C_{H}1 mutant residue(s) and the C_{L} mutant residue(s) comprise at least two charge pairs.

In some embodiments, the charge pair(s) comprise at least one charge pair located (i) at H172 and/or T192 in the first CHT domain and at N137 and/or N138 in the first C_{L} domain; and/or (ii) at H172 and/or T192 in the second CHT domain and at N137 and/or N138 in the second C_{L} domain. In some embodiments, the CHT mutant residue(s) comprise an arginine, a histidine, or a lysine, and wherein the C_{L} mutant residue(s) comprise an aspartic acid or a glutamic acid. In some embodiments, the CHT mutant residue(s) comprise an aspartic acid or a glutamic acid, and wherein the C_{L} mutant residue(s) comprise an arginine, a histidine, or a lysine. In some embodiments, the charge pair(s) comprise at least one charge pair comprising: (i) H172K and/or T192K in the first CHT domain and N137D and/or N138D in the first C_{L} domain; and/or (ii) H172K and/or T192K in the second CHT domain and N137D and/or N138D in the second C_{L} domain. In some embodiments, the charge pair(s) comprise at least one charge pair comprising: (i) H172D and/or T192D in the first C_{H}1 domain and N137K and/or N138K in the first C_{L} domain; and/or (ii) H172D and/or T192D in the second CHT domain and N137K and/or N138K in the second C_{L} domain. In some embodiments, the charge pair(s) comprise at least one charge pair comprising: (i) H172K and/or T192K in the first CHT domain and N137D and/or N138D in the first C_{L} domain; and (ii) H172D and/or T192D in the second CHT domain and N137K and/or N138K in the second C_{L} domain. In some embodiments, the antigen binding molecule disclosed herein comprises: (i) H172K and T192K in the first CHT domain and N137D and N138D in the first C_{L} domain; and (ii) H172D and T192D in the second C_{H}1 domain and N137K and N138K in the second C_{L} domain.

In some embodiments, the charge pairs comprise at least one charge pair on the first C_{H}C_{L} domain and at least one charge pair on the second C_{H}C_{L} domain located at the same positions. A charged pair interaction of amino acid residues can refer to the electrostatic attraction or repulsion between two amino acids that carry a net positive or negative charge on their side chains or functional groups. For example, a common charged pair interaction is between the amino acids lysine (Lys) and glutamic acid (Glu). Lysine has a positively charged side chain due to the presence of an amino group (NH3+), while glutamic acid has a negatively charged side chain due to the carboxyl group (COO-). These opposite charges create an attractive force between the two amino acids. The charged pair interaction can play a role in protein/protein interactions. The charged pair interaction can attract each other, forming a salt bridge or ionic bond. In protein-protein interactions, complementary charged amino acids can attract each other, facilitating the formation of protein complexes, such as the bispecific antigen binding molecules disclosed herein.

In some embodiments, the first C_{H}C_{L} domain and the C_{H}1 mutant residue(s) in the second C_{H}C_{L} domain are opposing charges. In some embodiments, the C_{H}1 mutant residue(s) and the C_{L} mutant residue(s) comprise a steric pair. A steric pair of amino acid residues can refer to two or more amino acids that have similar structures but differ in the position of a specific side chain or functional group. This difference in the side chain or functional group can significantly affect the chemical properties and interactions of the amino acids. For example, a steric pair could include the amino acids alanine (Ala) and valine (Val). Both alanine and valine are non-polar amino acids with similar structures consisting of a central carbon atom bonded to a hydrogen atom, a carboxyl group, an amino group, and a side chain. However, the side chain in alanine is a single methyl group (CH₃), while in valine, it is a branched isopropyl group (CH(CH3)2). The presence of the isopropyl group in valine introduces steric hindrance, making it bulkier compared to alanine. Valine's larger side chain can disrupt interactions between protein chains. Alanine's smaller side chain can allow for more compact packing between protein structures, particularly when interacting with an amino acid with a larger side chain.

In some embodiments, the C_{H}1 mutant residue(s) and the C_{L} mutant residue(s) comprise at least two steric pairs. In some embodiments, the steric pair(s) comprise at least one steric pair located (i) at L124 and/or G141 in the first C_{H}1 domain and at F116 and/or F118 in the first C_{L} domain; and/or (ii) at L124 and/or G141 in the second C_{H}1 domain and at F116 and/or F118 in the second C_{L} domain. In some embodiments, the steric pair(s) comprise at least one steric pair comprising: (i) L124S and/or G141L in the first C_{H}1 domain and F1.16T and/or F1.18M in the first C_{L} domain; and/or (ii) L124S and/or G141L in the second C_{H}1 domain and F116T and/or F118M in the second C_{L} domain. In some embodiments, the antigen binding molecule disclosed herein comprises: (i) F116T and F118M in the first C_{L} domain; and (ii) L124S and G141L in the second C_{H}1 domain. In some embodiments, the first C_{H}C_{L} domain comprises at least one charge pair and the second C_{H}C_{L} domain comprises at least one steric pair. In some embodiments, the first C_{H}1 domain comprises an amino acid sequence having at least 75%, at least 80%, at least 85%, at least 90%, at least 95%, at least 99%, or 100% sequence identity to any one of SEQ ID NO: 118, 120, 122, and 124. In some embodiments, the first C_{L} domain comprises an amino acid sequence having at least 75%, at least 80%, at least 85%, at least 90%, at least 95%, at least 99%, or 100% sequence identity to any one of SEQ ID NO: 119, 121, 123, and 125. In some embodiments, the second C_{H}1 domain comprises an amino acid sequence having at least 75%, at least 80%, at least 85%, at least 90%, at least 95%, at least 99%, or 100% sequence identity to any one of SEQ ID NO: 118, 120, 122, and 124. In some embodiments, the second C_{L} domain comprises an amino acid sequence having at least 75%, at least 80%, at least 85%, at least 90%, at least 95%, at least 99%, or 100% sequence identity to any one of SEQ ID NO: 119, 121, 123, and 125. In some embodiments, the antigen binding molecule comprises an amino acid sequence having at least 75%, at least 80%, at least 85%, at least 90%, at least 95%, at least 99%, or 100% sequence identity to any one of SEQ ID NO: 108-113. In some embodiments, the antigen binding molecule comprises an amino acid sequence having at least 75%, at least 80%, at least 85%, at least 90%, at least 95%, at least 99%, or 100% sequence identity to any one of SEQ ID NO: 114-117. In some embodiments, the antigen binding molecule comprises an amino acid sequence of SEQ ID NO: 114 and an amino acid sequence of SEQ ID NO: 115. In some embodiments, the antigen binding molecule comprises an amino acid sequence of SEQ ID NO: 116 and an amino acid sequence of SEQ ID NO: 117. In some embodiments, the antigen binding molecule comprises: (i) an amino acid sequence of SEQ ID NO: 110, (ii) an amino acid sequence of SEQ ID NO: 111, (iii) an amino acid sequence of SEQ ID NO: 112, and (iv) an amino acid sequence of SEQ ID NO: 113. In some embodiments, the antigen binding molecule comprises: (i) an amino acid sequence of SEQ ID NO: 114, (ii) an amino acid sequence of SEQ ID NO: 115, (iii) an amino acid sequence of SEQ ID NO: 116, and (iv) an amino acid sequence of SEQ ID NO: 117.

In some embodiments, the C_{H}1 mutant residue(s) and the C_{L} mutant residue(s) interact with each other in preference to corresponding non-mutated C_{H}1 residue(s) or corresponding non-mutated C_{L} residue(s). In some embodiments, the C_{H}1 mutant residue(s) repel a C_{L} domain comprising the corresponding non-mutated C_{L} residue(s) or the C_{L} mutant residue(s) repel a C_{H}1 domain comprising the corresponding non-mutated C_{H}1 residue(s). In some embodiments, the first V_{H} domain or the second V_{H} domain has at least one mutation relative to a human immunoglobulin V_{H} domain, referred to as the V_{H} mutant residue(s); and the first V_{L} domain or the second V_{L} domain has at least one mutation relative to a human immunoglobulin V_{L} domain, referred to as the V_{L} mutant residue(s). In some embodiments, the V_{H} mutant residue(s) and the V_{L} mutant residue(s) comprise a disulfide bridge pair. A disulfide bridge pair between amino acid residues can refer to the covalent bond formed between two cysteine amino acids through a redox reaction. Cysteine contains a unique sulfur-containing side chain called a thiol group (—SH). Under oxidizing conditions, the thiol groups of two cysteine residues within a protein can undergo oxidation, resulting in the formation of a disulfide bond (S-S) between the two cysteines. This bond creates a covalent link, often referred to as a disulfide bridge. These bridges can affect protein-protein interactions by forming specific intermolecular covalent links between proteins.

In some embodiments, the V_{H} mutant residue(s) and the V_{L} mutant residue(s) comprise at least two disulfide bridge pairs. In some embodiments, the disulfide bridge pair(s) comprise at least one disulfide bridge pair located (i) at G44 in the first V_{H} domain and at G100 in the first V_{L} domain; and/or (ii) at G44 in the second V_{H} domain and at G100 in the second V_{L} domain. In some embodiments, the disulfide bridge pair(s) comprise at least one disulfide bridge pair comprising: (i) G44C in the first V_{H} domain and G100C in the first VL domain; and/or (ii) G44C in the second V_{H} domain and G100C in the second V_{L} domain.

In some embodiments, the first C_{H}C_{L} domain comprises at least one charge pair or at least one steric pair, and where the second V_{H} domain and the second V_{L} domain comprise the VH mutant residue(s).

### Amino Acid Modifications

Alternative compositions and methods of producing multispecific antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US20140072581A1, US20160039947A1, and US20150368352.

### Lambda Kappa Formats

Multifunctional or multispecific antigen binding molecules (e.g., multifunctional or multispecific antibodies) that include the lambda light chain polypeptide and a kappa light chain polypeptides, can be used to allow for heterodimerization. Methods for generating bispecific antigen binding molecules comprising the lambda light chain polypeptide and a kappa light chain polypeptides are disclosed in PCT/US17/53053 filed on Sep. 22, 2017 and designated publication number WO 2018/057955, incorporated herein by reference in its entirety.

In some embodiments, the multifunctional or multispecific antigen binding molecule includes a multispecific antibody, e.g., an antibody comprising two binding specificities, such as a bispecific antibody. The multifunctional or multispecific antigen binding molecule can include:
a lambda light chain polypeptide 1 (LLCP1) specific for a first epitope;
a heavy chain polypeptide 1 (HCP1) specific for the first epitope;
a kappa light chain polypeptide 2 (KLCP2) specific for a second epitope; and
a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In some embodiments, it comprises all or a fragment of a CH1 region. In some embodiments, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In some embodiments, it comprises all or a fragment of a CH1 region. In some embodiments, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CH1region. In some embodiments, it comprises all or a fragment of a CH2 and/or CH3 region. In some embodiments, an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CH1region. In some embodiments, it comprises all or a fragment of a CH2 and/or CH3 region. In some embodiments, an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments, in the multifunctional polypeptide antigen binding molecule as described herein:
LLCP1 has a higher affinity for HCP1 than for HCP2; and/or
KLCP2 has a higher affinity for HCP2 than for HCP1.

In some embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75, 80, 90, 95, 98, 99, 99.5, or 99.9% of the multifunctional or multispecific antigen binding molecules have a LLCP1complexed, or interfaced with, a HCP1.

In some embodiments, in the multifunctional polypeptide antigen binding molecule as described herein:
the HCP1 has a greater affinity for HCP2, than for a second molecule of HCP1; and/or
the HCP2 has a greater affinity for HCP1, than for a second molecule of HCP2.

In some embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99.99.5 or 99.9% of the multifunctional or multispecific antigen binding molecules have a HCPlcomplexed, or interfaced with, a HCP2.

In another aspect, described herein is a method for making, or producing, a multifunctional or multispecific antigen binding molecule. The method includes:
(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));
(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));
(iii) providing a lambda chain polypeptide (e.g., a lambda light variable region (VLλ), a lambda light constant chain (VLλ), or both) that preferentially associates with the first heavy chain polypeptide (e.g., the first VH); and
(iv) providing a kappa chain polypeptide (e.g., a lambda light variable region (VLλ), a lambda light constant chain (VLλ), or both) that preferentially associates with the second heavy chain polypeptide (e.g., the second VH), under conditions where (i)-(iv) associate.

In some embodiments, the first and second heavy chain polypeptides form an Fc interface that enhances heterodimerization.

In some embodiments, (i)-(iv) (e.g., nucleic acid encoding (i)-(iv)) are introduced in a single cell, e.g., a single mammalian cell, e.g., a CHO cell. In some embodiments, (i)-(iv) are expressed in the cell. In some embodiments, (i)-(iv) (e.g., nucleic acid encoding (i)-(iv)) are introduced in different cells, e.g., different mammalian cells, e.g., two or more CHO cell. In some embodiments, (i)-(iv) are expressed in the cells.

In some embodiments, the method further comprises purifying a cell-expressed antigen binding molecule, e.g., using a lambda- and/or- kappa-specific purification, e.g., affinity chromatography.

In some embodiments, the method further comprises evaluating the cell-expressed multifunctional or multispecific antigen binding molecule. For example, the purified cell-expressed multifunctional or multispecific antigen binding molecule can be analyzed by techniques known in the art, include mass spectrometry. In some embodiments, the purified cell-expressed antigen binding molecule is cleaved, e.g., digested with papain to yield the Fab moieties and evaluated using mass spectrometry.

In some embodiments, the method produces correctly paired kappa/lambda multifunctional or multispecific, e.g., bispecific, antigen binding molecules in a high yield, e.g., at least 75%, 80, 90, 95, 98, 99 99.5 or 99.9%.

In other embodiments, the multifunctional or multispecific, e.g., a bispecific, antigen binding molecule that includes:
(i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;
(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;
(iii) a lambda light chain polypeptide (LLCP1) (e.g., a lambda light variable region (VLλ), a lambda light constant chain (VLλ), or both) that preferentially associates with the first heavy chain polypeptide (e.g., the first VH), e.g., wherein the LLCP1 binds to a first epitope; and (iv) a kappa light chain polypeptide (KLCP2) (e.g., a kappa light variable region (VLκ), a kappa light constant chain (VLκ), or both) that preferentially associates with the second heavy chain polypeptide (e.g., the second VH), e.g., wherein the KLCP2 binds to a second epitope.

In some embodiments, the first and second heavy chain polypeptides form an Fc interface that enhances heterodimerization. In some embodiments, the multifunctional or multispecific antigen binding molecule has a first binding specificity that includes a hybrid VLλ-CLλ heterodimerized to a first heavy chain variable region connected to the Fc constant, CH2-CH3 domain (having a knob modification) and a second binding specificity that includes a hybrid VLκ-CLκ heterodimerized to a second heavy chain variable region connected to the Fc constant, CH2-CH3 domain (having a hole modification).

### Nucleic Acid Molecules

Provided herein, are methods of making the antigen binding molecules of the present disclosure, including recombinant polynucleotide molecules, vectors comprising the recombinant polynucleotide molecules, and cells comprising the recombinant polynucleotide molecules. Antigen binding molecules of the present disclosure are synthesized using the techniques of recombinant DNA and protein expression. For example, for the synthesis of DNA encoding a dual IgG of the disclosure, suitable DNA sequences encoding the constant domains of the heavy and light chains are widely available.

In some embodiments, “nucleic acid” or “polynucleotide” can refer to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell.

### Probes 8:91-98 (1994)).

Described herein, in certain embodiments, is an isolated nucleic acid molecule comprising a nucleotide sequence having at least 75%, 80%, 85%, 90%, 95%, 99%, 99.5%, 99.9%, or 100% sequence identity to the nucleotide sequence encoding the multifunctional polypeptide molecule as described herein.

Nucleic acids encoding the aforementioned antigen binding molecules, e.g., anti-EGFR antigen binding molecules, anti-ITGB6 antigen binding molecules, bispecific anti-EGFR and anti-ITGB6 antigen binding molecules, multispecific or multifunctional antigen binding molecules are also disclosed.

In certain embodiments, the invention features nucleic acids comprising nucleotide sequences that encode heavy and light chain variable regions and CDRs or hypervariable loops of the antigen binding molecules, as described herein. For example, the invention features a first and second nucleic acid encoding heavy and light chain variable regions, respectively, of an antigen binding molecule chosen from one or more of the antigen binding molecules as described herein. The nucleic acid can encode an amino acid sequence as set forth in Table 1. The nucleic acid can encode an amino acid sequence as set forth in Table 2.

In certain embodiments, the nucleic acid can comprise a nucleotide sequence encoding at least one, two, or three CDRs or hypervariable loops from a heavy chain variable region having an amino acid sequence as set forth in Table 1, Table 2, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in Table 1, Table 2, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in Table 1, Table 2, or a sequence substantially homologous thereto (e.g., a sequence at least about 85%, 90%, 95%, 99% or more identical thereto, and/or having one or more substitutions, e.g., conserved substitutions).

Provided herein, are recombinant polynucleotide molecule comprising the polynucleotide sequences encoding the antigen binding molecule of the present disclosure. In some embodiments, encoding can refer to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. For example, a gene, cDNA, or RNA, encodes a protein if transcription and translation of mRNA corresponding to that gene produces the protein in a cell or other biological system. In some cases, the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. In some embodiments, a “nucleotide sequence encoding an amino acid sequence” includes all nucleotide sequences that are degenerate versions of each other and that encode the same amino acid sequence. The phrase nucleotide sequence that encodes a protein or RNA may also include introns to the extent that the nucleotide sequence encoding the protein may in some versions contain one or more introns.

In some embodiments, the recombinant polynucleotide molecule is an isolated recombinant polynucleotide molecule. Sequences encoding the selected variable domains are inserted by standard methods, and the resulting nucleic acids encoding full-length heavy and light chains are transduced into suitable host cells and expressed. Alternatively, the nucleic acids can be expressed in a cell-free expression system, which can provide more control over oxidation and reduction conditions, pH, folding, glycosylation, and the like.

### Vectors

Described herein, in certain embodiments, is a vector comprising one or more of the nucleic acid molecules as described herein.

Further provided herein are vectors comprising the nucleotide sequences encoding antigen binding molecules, e.g., anti-EGFR antigen binding molecules, anti-ITGB6 antigen binding molecules, bispecific anti-EGFR and anti-ITGB6 antigen binding molecules, or a multispecific or multifunctional antigen binding molecule described herein. In some embodiments, the vectors comprise nucleic acid sequences encoding antigen binding molecules, e.g., anti-EGFR antigen binding molecules, anti-ITGB6 antigen binding molecules, bispecific anti-EGFR and anti-ITGB6 antigen binding molecules, or multispecific or multifunctional antigen binding molecule described herein. In some embodiments, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antigen binding molecule produced are known to those skilled in the art, and may be varied or optimized depending upon the specific expression vector and mammalian host cell employed, based upon the present description.

Provided herein, are vectors comprising the recombinant polynucleotide molecule of the present disclosure. In one aspect, the antigen binding molecules of the present disclosure relate to nucleic acid molecules comprising nucleotide sequences encoding the antigen binding molecules of the disclosure, including expression cassettes, and expression vectors containing these nucleic acid molecules operably linked to heterologous nucleic acid sequences such as, for example, regulatory sequences which direct in vivo expression of the protein in a host cell.

In some embodiments, a vector, a plasmid, or a virus contains one or more of the nucleic acid molecules encoding any antigen binding molecule disclosed herein. In some embodiments, a vector, a plasmid, or a virus contains one or more of the nucleic acid molecules encoding four of the heavy/light chains of the antigen binding molecule disclosed herein (e.g., one vector may contain nucleic acid molecules encoding both a heavy chain and a light chain of the anti-EGFR arm of the antigen binding molecule disclosed herein as wells as a heavy chain and a light chain of the anti-ITGB6 arm of the antigen binding molecule disclosed herein). The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Methods disclosed herein may include collecting the recombinant polynucleotide molecule described herein. Methods disclosed herein may include collecting the recombinant polynucleotide described herein. Methods disclosed herein may include collecting the polypeptide (e.g., bispecific antigen binding molecule) described herein. In some embodiments, the collecting comprises lysing a cell. A cell may be lysed via high pressure, osmotic shock or pressure, low temperature, sonication, homogenization, or a combination thereof. The resulting solution, or lysate of the cell, may be collected and subjected to downstream analysis or processing such as purification. In some embodiments, the collecting the polypeptide comprises purifying the polypeptide. In some embodiments, purifying does not comprise lysing the cell. In some embodiments, the polypeptide may be secreted. The secretion of the polypeptide may allow the polypeptide to be collected in the media. The secretion of the polypeptide may allow the polypeptide to be collected without lysis of the cell.

The recombinant polynucleotide molecule may be purified, or otherwise concentrated or isolated such a solution contains predominantly the recombinant polynucleotide molecule. In some embodiments, the purification is performed on the soluble fraction of the cell lysate. In some embodiments, the soluble fraction of the cell lysate is obtained by the centrifugation of cell lysate and collecting the supernatant. In some embodiments, the purification is performed on the insoluble fraction of the cell lysate. In some embodiments, the insoluble fraction of the cell lysate is obtained by the centrifugation of cell lysate and collecting the pellet formed. In some embodiments, the insoluble fraction comprises inclusion bodies that include the recombinant polynucleotide molecule. In some embodiments, the purifying comprises isolating the polypeptide is purified from the inclusion bodies. In some embodiments, purifying comprises isolating from inclusion bodies. Isolating the polypeptide from the inclusion bodies may comprise the use of denaturants and chaotropes, such as guanidine or urea, to solubilize the inclusion bodies. In some embodiments, the purifying uses an agent that specifically binds the polypeptide. For example, the agent may be protein with a binding affinity to the polypeptide. In some embodiment, the agent that specifically binds the polypeptide is immobilized on a solid support such as a bead and is used to capture the polypeptide.

In some embodiments, purifying comprises using a chromatography. Chromatography may be performed by using the physical characteristics of the polypeptide, such as charge, shape, or polarity, to separate the polypeptide from other protein. In some embodiments, the chromatography comprises ion exchange chromatography, size exclusion chromatography, gel chromatography, reverse phase chromatography, affinity chromatography, or a combination thereof. Multiple forms of chromatography may be used sequentially to increase the purity of the polypeptide. Chromatography may be performed by introducing the cell lysate or polypeptide containing solution into a column containing resin or other solid supports. The resin may interact different with different proteins and polypeptides resulting in a separation of the proteins based on physical characteristics. For example, the resin may contain nickel and may interact with a His tag on the polypeptide. In some embodiments, the chromatography is selected from the group consisting of an ion exchange chromatography, a size exclusion chromatography, a reverse phase chromatography, and an affinity chromatography. In some embodiments, purifying comprises using an agent that specifically binds the polypeptide. In some cases, the tag or affinity tag of the polypeptide as described herein can be used for the purification of the polypeptide as described herein.

In some embodiments, purifying comprises using a dialysis. Dialysis can be used as a method for purification of the polypeptides disclosed herein. Dialysis can involve the separation of the polypeptides disclosed herein from smaller molecules using a semipermeable membrane. Dialysis takes advantage of the principle of diffusion, where molecules move from an area of higher concentration to an area of lower concentration. In some embodiments, the dialysis is performed using a dialysis membrane. In some embodiments, the dialysis is performed using a dialysis tubing. A dialysis membrane and/or a dialysis tubing can be a semipermeable membrane with specific molecular weight cutoff (MWCO). The MWCO can determine the size of molecules that can pass through the semipermeable membrane. For example, the MWCO can allow for sugar moieties to pass through the semipermeable membrane, but not the polypeptides disclosed herein.

The methods as disclosed elsewhere herein may yield a particular polypeptide yield. In some embodiments, the methods as disclosed elsewhere herein produce polypeptide at an amount of at least 0.1 mgs(milligrams) per liter(L) of media. In some embodiments, the methods produce polypeptide at an amount of at least 1 mg per liter of media. In some embodiments, the methods produce polypeptide at an amount of at least 5 mgs per liter of media. In some embodiments, the methods produce polypeptide at an amount of at least 10 mgs per liter of media. In some embodiments, the methods produce polypeptide at an amount of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 4.0, 5.0, 6.0,7.0,8.0, 9.0, 10, 15, 20, 30, 40, 50, 60, 70, 80, 90, 100, 200, 300, 400, 500, 600, 700, 800, 900, 1000, or more mgs per liter of media.

### Cells

Described herein, in certain embodiments, is a cell comprising the nucleic acid as described herein or the vector as described herein.

In another aspect, described herein are host cells and vectors containing the nucleic acids. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

In some embodiments, described herein are host cells comprising a nucleic acid encoding an antigen binding molecule as described herein.

In some embodiments, described herein are the host cells genetically engineered to comprise nucleic acids encoding the antigen binding molecule.

In some embodiments, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

In some embodiments, described herein are host cells comprising the vectors described herein. The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells.

Suitable insect cells include, but are not limited to, Sf9 cells.

In some embodiments, the methods described can be performed in a host cell, or in vitro, in cell-free synthetic systems. Host cells may be any that can be robustly recoded.

These can be bacterial cells that have well developed genetic systems, of which E. coli is exemplary. Other bacterial species can also be used. In some embodiments, cell-free systems for producing the proteins may be coupled transcription/translation systems or only translation systems. Notably, in some embodiments, biological syntheses are utilized rather than chemical synthesis.

In one aspect the present disclosure provides a cell comprising the recombinant polynucleotide molecule disclosed herein. Culturing of recoded cells with the constructed nucleic acid sequences may be by any means known in the art. In some embodiments, the culturing may be batch or continuous, in shaker flasks or in fermenters or immobilized on solid surfaces, such as small particles contained in larger vessels.

In some embodiments, the cell can be a plurality of cells. In some cases, the plurality of cells can be from about 1 cell to about 1 billion cells. In some cases, the plurality of cells can be about 0 cells, about 10 cells, about 100 cells, about 1,000 cells, about 10,000 cells, about 100,000 cells, about 1,000,000 cells, about 10,000,000 cells, about 100,000,000 cells, about 1,000,000,000 cells. In some cases, the plurality of cells can be at least about 0 cells, about at least 10 cells, about at least 100 cells, about at least 1,000 cells, about at least 10,000 cells, about at least 100,000 cells, about at least 1,000,000 cells, about at least 10,000,000 cells, about at least 100,000,000 cells, about at least 1,000,000,000 cells, or more.

In some embodiments, the cell can be a bacterial cell. In some cases, the bacterial cell can be a bio-contained strain. In some cases, the bacterial cell can be a multi-virus resistance bacterial cell. In some embodiments, the bacterial cell can be from a bacterial genus. Non-limiting examples of bacterial geneses include Staphylococcus, Streptococcus, Enterococcus, Moraxella, Neisseria, Corynebacterium, Bacillus, Lactobacillus, Listeria, Citrobacter, Enterobacter, Escherichia, Klebsiella, Proteus, Serratia, Hafnia, Morganella, Providencia, Salmonella, Shigella, Yersinia, Acinetobacter, Pseudomonas, Strenotrophomonas, Burkholderia, Haemophilus, Legionella, Achromobacter, Aeromonas, Alcaligenes, Campylobacter, Flavobacterium, Helicobacter, Pasteurella, Bacteroides, Clostridium, Propionibacterium, Prevotella, Mycobacterium, Mycoplasma, Actinomyces, Acetobacter, Bordetella, Vibrio, and Nocardia. In some embodiments, the bacterial cell can be a bacterial species. Non-limiting examples of bacterial species include Staphylococcus aureus, Staphylococcus epidermidis, Staphylococcus haemolyticus, Streptococcus pneumoniae, Streptococcus agalactiae, Streptococcus pyogenes, Enterococcus faecalis, Enterococcus faecium, Moraxella catharralis, Neisseria meningitidis, Listeria monocytogenes, Acetobacter aurantius, Acinetobacter baumannii, Actinomyces israelii, Bacillus brevis, Bacillus cereus, Bacillus fusiformis, Bacillus mycoides, Bacillus subtilis, Bacteroides fragilis, Bacteroides gingivalis, Bordetella bronchiseptica, Bordetella pertussis, Burkholderia mallei, Burkholderiahosphora, Campylobacter jejuni, Campylobacter pylori, Clostridium botulinum, Clostridium difficule, Corynebacterium diphtheria Corynebacterium fusiforme, Enterococcus avium, Enterococcus durans, Enterococcus gallinarum, Enterococcus maloratus, Haemophilus influenzae, Haemophilus pertussis, Haemophilus parainfluenzae, Helicobacter pylori, Klebsiella pneumoniae, Lactobacillus acidophilus, Lactobacillus bulgaricus, Lactobacillus casei, Lactobacillus lactis, Legionella pneumophila, Mycobacterium avium, Mycobacterium bovis, Mycoplasma hominis, Mycoplasma fermentans, Neisseria gonorrhoeae, Neisseria meningitidis, Pasteurella multocida, Pasteurella tulanensis, Prevotella melaninogenica, Pseudomonas aeruginosa, Salmonella enteritidis, Salmonella typhi, Vibrio comma, Vibrio vulnificus, and Yersinia enterocolitica.

In some embodiments, the cell can be a mammalian cell. In some embodiments, the cell can be a mammalian cell line. Non-limiting examples of mammalian cell lines include HeLa cells, HEK293 cells, CHO cells, NIH/3T3 cells, Jurkat cells, RAW 264.7 cells, SH-SY5Y cells, MCF-7 cells, A549 cells, and U87 cells.

### Methods of Making

Provided herein are method of making a bispecific antigen binding molecule disclosed herein. In some embodiments, the method of making a bispecific antigen binding molecule comprises: (i) providing a first C_{H}1 domain, a second C_{H}1 domain, a first C_{L} domain, and a second C_{L} domain. In some embodiments, the first C_{H}1 domain and/or the second C_{H}1 domain have at least one mutation relative to a human immunoglobulin C_{H}1 domain, referred to as the C_{H}1 mutant residue(s). In some embodiments, each C_{H}1 mutant residue is only present in one of first C_{H}1 domain or the second C_{H}1 domain. In some embodiments, the first CL domain and/or the second C_{L} domain have at least one mutation relative to a human immunoglobulin CL domain, referred to as the CL mutant residue(s). In some embodiments, each CL mutant residue is only present in one of first CL domain or the second C_{L} domain. In some embodiments, the method of making a bispecific antigen binding molecule further comprises: (ii) mixing the first C_{H}1 domain, the second C_{H}1 domain, the first C_{L} domain, and the second C_{L} domain, thereby generating a plurality of bispecific antigen binding molecules. In some embodiments, the plurality of bispecific antigen binding molecules comprises the desired heterodimer, and wherein the purity of the desired heterodimer relative to total heterodimers is at least 60%, at least 70%, at least 80%, at least 90%, at least 95%, at least 97%, at least 98%, or at least 99%.

### Pharmaceutical Compositions

Provided herein, are pharmaceutical composition comprising the antigen binding molecule of the present disclosure, the recombinant polynucleotide of the present disclosure, the vector of the present disclosure, or the cell of the present disclosure, and a pharmaceutically acceptable carrier, excipient, or diluent. In some embodiments, the antigen binding molecules, nucleic acids, and recombinant cells of the disclosure can be incorporated into compositions, including pharmaceutical compositions. Such compositions typically include the antigen binding molecules, and a pharmaceutically acceptable excipient, e.g., a carrier. Antigen binding molecules of the present disclosure can be administered using formulations used for administering antibodies and antibody-based therapeutics, or formulations based thereon.

Pharmaceutical compositions or formulations comprising the agent, e.g., the multifunctional or multispecific antigen binding molecules, of the described compositions and for use in any of the described methods can be prepared according to conventional techniques well known in the pharmaceutical industry and described in the published literature. In some embodiments, a pharmaceutical composition or formulation for treating a subject comprises an effective amount of any the multifunctional or multispecific antigen binding molecules or the compositions as described herein, or a pharmaceutically acceptable salt, solvate, hydrate or ester thereof. The pharmaceutical formulation comprising the multifunctional or multispecific antigen binding molecules as described herein may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some embodiments, a pharmaceutical formulation or composition as described herein includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In some embodiments, liposomes also include sterically stabilized liposomes, e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the present disclosure employs a penetration enhancer to effect the efficient delivery of the multifunctional or multispecific antigen binding molecules or the compositions as described herein, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple multifunctional or multispecific antigen binding molecules as described herein.

In some embodiments, the pharmaceutical composition is formulated for oral administration, intravenous injection, intradermal injection, subcutaneous injection, intrathecal administration, intracerebral administration, intracerebroventricular injection, topical administration, inhalation, or nasal administration. In some embodiments, the pharmaceutical composition is formulated for transdermal administration (which may include a penetration enhancement agent), buccal administration, or suppository administration. In some embodiments, the pharmaceutical composition of the present disclosure may be administered, either orally or parenterally, systemically or locally. For example, intravenous injection such as drip infusion, intramuscular injection, intrapleural injection, intraperitoneal injection, subcutaneous injection, and the like can be selected, and the method of administration may be chosen, as appropriate, depending on the age and the condition of the subject.

In some embodiments, the pharmaceutical composition is a liquid, a suspension, a solution, or an emulsion. The herein described pharmaceutical compositions may be formulated into different forms; for example, they may be formulated into a liquid form (e.g., suspension, solution, and syrup).

In some embodiments, the pharmaceutical composition is prepared by a method further comprising combining one or more additional therapeutics with one or more polypeptides. For example, in some embodiments, the additional therapeutic may be combined with the dispersible powder that contains one or more polypeptides. For another example, in some embodiments, the additional therapeutic may be combined with one or more polypeptides to produce the liquid composition. For yet another example, in some embodiments, the additional therapeutic may be added into the liquid composition that comprises one or more polypeptides. In some embodiments, the pharmaceutical composition is in a liquid form such as suspension, or solution.

### Methods of Treatment

The present disclosure discloses a method of treating a subject with a condition or a disease. In some embodiments, the method comprises administering to the subject a therapeutically effective amount of the pharmaceutical composition described herein.

Administration of any one or more of the therapeutic compositions described herein, e.g., antigen binding molecules and pharmaceutical compositions, can be used to treat individuals having a neoplastic disease, such as cancers.

In one aspect, provided herein are methods of treating a condition or disease in a subject in need thereof, comprising administering to the subject a therapeutically effective amount of the antigen binding molecule disclosed herein, the recombinant polynucleotide disclosed herein, the vector disclosed herein, the cell disclosed herein, the pharmaceutical composition disclosed herein, or any combination thereof, thereby treating the condition or disease in the subject. In some cases, the condition or disease is cancer. In some embodiments, the cancer is a lung cancer, such as non-small cell lung cancer (NSCLC). In some embodiments, the cancer is NSCLC. In some embodiments, the NSCLC is characterized as having an oncogenic EGFR alteration. In some embodiments, the cancer is characterized as having an oncogenic EGFR alteration (such as oncogenic alterations considered classical EGFR mutations). In some embodiments, the oncogenic EGFR alteration is an EGFR L858 mutation. In some embodiments, the EGFR L858 mutation is a L858R mutation. In some embodiments, the oncogenic EGFR alteration is an EGFR T790 mutation. In some embodiments, the EGFR T790 mutation is a T790M mutation. In some embodiments, the oncogenic EGFR alteration is an EGFR C797 mutation. In some embodiments, the EGFR C797 mutation is a C797S mutation. In some embodiments, the oncogenic EGFR alteration is an Exon 19 mutation. In some embodiments, the Exon 19 mutation is an Exon 19 deletion mutation. In some embodiments, the cancer is a gastrointestinal cancer, such as colorectal cancer (CRC). In some embodiments, the method of treating cancer comprises treatment of squamous cell carcinoma, such as head and neck squamous cell carcinoma (HNSCC). In some embodiments, the cancer is esophageal cancer. In some embodiments, the cancer is bladder cancer. In some embodiments, the subject has relapsed after prior therapy. In some embodiments, the subject has acquired resistance to prior therapy. In some embodiments, the prior therapy comprises treatment with an EGFR tyrosine kinase inhibitor. In some embodiments, the EGFR tyrosine kinase inhibitor is osimertinib.

In one aspect, provided herein are methods for treating a condition or disease in a subject, the method comprising: administering to a subject the antigen binding molecule disclosed herein, wherein the antigen binding molecule specifically binds to: (a) an endogenous internalizing receptor, wherein the endogenous internalizing receptor is expressed on a target cell, and wherein the endogenous internalizing receptor is ITGB6; and (b) the target protein, wherein the target protein comprises EGFR.

In one aspect, provided herein are methods of identifying a subject in need of treatment for a cancer, the method comprising: (a) determining whether the subject has cancer cells that express a higher level of EGFR, ITGB6, or any combination thereof as compared to a control sample; and (b) identifying the subject as a candidate for the treatment based on the determination that the subject has cancer cells that express a higher level of EGFR, ITGB6, or any combination thereof as compared to the control sample, wherein the treatment comprises the antigen binding molecule disclohosphrien. In some embodiments, the antigen binding molecule induces cancer cell death, cancer cell lysis, or both. In some embodiments, administering the antigen binding molecule induces more anti-tumor efficacy in the subject, relative to a subject not administered the antigen binding molecule. In some embodiments, administering the antigen binding molecule induces more cytotoxicity against tumor cells or cancer cells in the subject, relative to a subject not administered the antigen binding molecule. In some embodiments, the cancer is a solid tumor cancer, a metastatic cancer, a soft tissue tumor, or a combination thereof.

In one aspect, provided herein are methods of decreasing EGFR expression on the surface of a cancer cell, comprising: contacting EGFR with the antigen binding molecule of any one of claims 1-96, the recombinant polynucleotide of claim 97 or 98, the vector of claim 99, the cell of claim 100, the pharmaceutical composition of claim 101, or any combination thereof, wherein contacting EGFR with the antigen binding molecule results in decreased EGFR expression on the surface of the cancer cell by about 20%, 30%, 40%, 50%, 60%, 70% or more relative to EGFR expression on the surface of a corresponding cancer cell contacted with a control antigen binding molecule, thereby decreasing EGFR expression on the surface of a cancer cell. In another aspect, provided herein are methods of increasing EGFR degradation in a cancer cell, comprising: contacting EGFR with the antigen binding molecule of any one of claims 1-96, the recombinant polynucleotide of claim 97 or 98, the vector of claim 99, the cell of claim 100, the pharmaceutical composition of claim 101, or any combination thereof, wherein contacting EGFR with the antigen binding molecule results in increased EGFR degradation in the cancer cell by about 20%, 30%, 40%, 50%, 60%, 70% or more relative to EGFR degradation of a corresponding cancer cell contacted with a control antigen binding molecule, thereby increasing EGFR degradation in a cancer cell. Yet another aspect, provided herein are methods of increasing cell surface removal of EGFR on a cancer cell, comprising: contacting EGFR with the antigen binding molecule of any one of claims 1-96, the recombinant polynucleotide of claim 97 or 98, the vector of claim 99, the cell of claim 100, the pharmaceutical composition of claim 101, or any combination thereof, wherein contacting EGFR with the antigen binding molecule results in increased cell surface removal of EGFR on the cancer cell by about 20%, 30%, 40%, 50%, 60%, 70% or more relative to cell surface removal of EGFR on a corresponding cancer cell contacted with a control antigen binding molecule, thereby increasing cell surface removal of EGFR on a cancer cell.

In one aspect, provided herein are methods for inhibiting an activity of a target cell in an individual, the methods comprising the step of administering to the individual a first therapy including one or more of the antigen binding molecules and pharmaceutical compositions provided herein, wherein the first therapy inhibits an activity of the target cell by degrading a target surface protein. For example, an activity of the target cell may be inhibited if its proliferation is reduced, if its pathologic or pathogenic behavior is reduced, if it is destroyed or killed, or the like. Generally, the target cell of the disclosed methods can be any cancer cell.

In one aspect, provided herein are methods of decreasing tumor volume of a tumor, comprising: contacting the tumor with the antigen binding molecule of any one of claims 1-96, the recombinant polynucleotide of claim 97 or 98, the vector of claim 99, the cell of claim 100, the pharmaceutical composition of claim 101, or any combination thereof, wherein contacting the tumor with the antigen binding molecule results in decreased tumor volume of the tumor by about 20%, 30%, 40%, 50%, 60%, 70% or more relative to the tumor volume of a corresponding tumor contacted with a control antigen binding molecule, thereby decreasing tumor volume of a tumor.

In some embodiments, a method for treating cancer in a subject comprises administering to a subject an antigen binding molecule of the present disclosure, wherein the treatment results in a decrease in EGFR expression on the target cell of at least 20%, at least 30%, at least 40%, at least 50%, at least 60%, at least 70%, at least 80%, at least 90%, at least 95%, at least 99%, or more. In some embodiments, the compositions and methods disclosed herein can decrease expression of EGFR on the target cell. In some embodiments, the compositions and methods disclosed herein can decrease expression of EGFR on the target cell.

In some embodiments, a method for treating cancer in a subject comprises administering to a subject an antigen binding molecule of the present disclosure, wherein the method results in a decrease in tumor volume of at least at least 20%, at least 30%, at least 40%, at least 50%, at least 60%, at least 70%, at least 80%, at least 90%, at least 95%, or at least 99%, at least 100%, at least 1²⁵%, at least 150%, or more. In some embodiments, tumor volume of a tumor contacted with the antigen binding molecule is determined relative to the tumor volume of a tumor not contacted with the antigen binding molecule. In some embodiments, tumor volume of a tumor contacted with the antigen binding molecule is determined relative to the tumor volume of a tumor contacted with the Cetuximab.

In some embodiments, a method for treating cancer in a subject comprises administering to a subject an antigen binding molecule of the present disclosure, wherein the method results in a tumor volume of a tumor contacted with an antigen binding molecule that is less than the tumor volume of a tumor not contacted with the antigen binding molecule. In some embodiments, a method for treating cancer in a subject comprises administering to a subject an antigen binding molecule of the present disclosure, wherein the method results in a tumor volume of a tumor contacted with an antigen binding molecule that is less than the tumor volume of a tumor contacted with Cetuximab.

In some embodiments, the control antigen binding molecule is a single arm EGFR antigen binding molecule, such as a single arm EGFR antibody. In some embodiments, the control antigen binding molecule is Cetuximab.

In some embodiments, the half-life of the antigen binding molecule is at least 20%, at least 30%, at least 40%, at least 50%, at least 60%, at least 70%, at least 80%, at least 90%, at least 100%, at least 110%, or more, as long as the half-life of Cetuximab. In some embodiments, the clearance rate of the antigen binding molecule is within at least 20%, at least 30%, at least 40%, at least 50%, at least 60%, at least 70%, at least 80%, at least 90%, at least 100%, at least 110%, or more, as compared to the clearance rate of Cetuximab.

In some cases, the subjects may be at least about 2 years old, at least about 3 years old, at least about 4 years old, at least about 5 years old, at least about 6 years old, at least about 7 years old, at least about 8 years old, at least about 9 years old, at least about 10 years old, at least about 11 years old, at least about 12 years old, at least about 13 years old, at least about 14 years old, at least about 15 years old, at least about 16 years old, at least about 17 years old, at least about 18 years old, at least about 19 years old, at least about 20 years old, at least about 21 years old, at least about 22 years old, at least about 23 years old, at least about 24 years old, at least about 25 years old, at least about 26 years old, at least about 27 years old, at least about 28 years old, at least about 29 years old, at least about 30 years old, at least about 31 years old, at least about 32 years old, at least about 33 years old, at least about 34 years old, at least about 35 years old, at least about 36 years old, at least about 37 years old, at least about 38 years old, at least about 39 years old, at least about 40 years old, at least about 41 years old, at least about 42 years old, at least about 43 years old, at least about 44 years old, at least about 45 years old, at least about 46 years old, at least about 47 years old, at least about 48 years old, at least about 49 years old, at least about 50 years old, at least about 51 years old, at least about 52 years old, at least about 53 years old, at least about 54 years old, at least about 55 years old, at least about 56 years old, at least about 57 years old, at least about 58 years old, at least about 59 years old, at least about 60 years old, at least about 61 years old, at least about 62 years old, at least about 63 years old, at least about 64 years old, at least about 65 years old, at least about 66 years old, at least about 67 years old, at least about 68 years old, at least about 69 years old, at least about 70 years old, at least about 71 years old, at least about 72 years old, at least about 73 years old, at least about 74 years old, at least about 75 years old, at least about 76 years old, at least about 77 years old, at least about 78 years old, at least about 79 years old, at least about 80 years old, at least about 81 years old, at least about 82 years old, at least about 83 years old, at least about 84 years old, at least about 85 years old, at least about 86 years old, at least about 87 years old, at least about 88 years old, at least about 89 years old, at least about 90 years old, at least about 91 years old, at least about 92 years old, at least about 93 years old, at least about 94 years old, at least about 95 years old, at least about 96 years old, at least about 97 years old, at least about 98 years old, at least about 99 years old, or at least about 100 years old. In some cases, the subjects may be at most about 2 years old, at most about 3 years old, at most about 4 years old, at most about 5 years old, at most about 6 years old, at most about 7 years old, at most about 8 years old, at most about 9 years old, at most about 10 years old, at most about 11 years old, at most about 12 years old, at most about 13 years old, at most about 14 years old, at most about 15 years old, at most about 16 years old, at most about 17 years old, at most about 18 years old, at most about 19 years old, at most about 20 years old, at most about 21 years old, at most about 22 years old, at most about 23 years old, at most about 24 years old, at most about 25 years old, at most about 26 years old, at most about 27 years old, at most about 28 years old, at most about 29 years old, at most about 30 years old, at most about 31 years old, at most about 32 years old, at most about 33 years old, at most about 34 years old, at most about 35 years old, at most about 36 years old, at most about 37 years old, at most about 38 years old, at most about 39 years old, at most about 40 years old, at most about 41 years old, at most about 42 years old, at most about 43 years old, at most about 44 years old, at most about 45 years old, at most about 46 years old, at most about 47 years old, at most about 48 years old, at most about 49 years old, at most about 50 years old, at most about 51 years old, at most about 52 years old, at most about 53 years old, at most about 54 years old, at most about 55 years old, at most about 56 years old, at most about 57 years old, at most about 58 years old, at most about 59 years old, at most about 60 years old, at most about 61 years old, at most about 62 years old, at most about 63 years old, at most about 64 years old, at most about 65 years old, at most about 66 years old, at most about 67 years old, at most about 68 years old, at most about 69 years old, at most about 70 years old, at most about 71 years old, at most about 72 years old, at most about 73 years old, at most about 74 years old, at most about 75 years old, at most about 76 years old, at most about 77 years old, at most about 78 years old, at most about 79 years old, at most about 80 years old, at most about 81 years old, at most about 82 years old, at most about 83 years old, at most about 84 years old, at most about 85 years old, at most about 86 years old, at most about 87 years old, at most about 88 years old, at most about 89 years old, at most about 90 years old, at most about 91 years old, at most about 92 years old, at most about 93 years old, at most about 94 years old, at most about 95 years old, at most about 96 years old, at most about 97 years old, at most about 98 years old, at most about 99 years old, or at most about 100 years old.

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment.

For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the multifunctional or multispecific antigen binding molecules or the compositions as described herein to the fetus directly or indirectly (e.g., via the mother).

Suitable routes for administration of the multifunctional or multispecific antigen binding molecules or the compositions as described herein may vary depending on cell type to which delivery of the multifunctional or multispecific antigen binding molecules or the compositions is desired. The multifunctional or multispecific antigen binding molecules or the compositions as described herein may be administered to patients parenterally, for example, by intrathecal injection, intracerebroventricular injection, intraperitoneal injection, intramuscular injection, subcutaneous injection, or intravenous injection.

In some embodiments, the multifunctional or multispecific antigen binding molecules or the compositions as described herein are administered with one or more agents capable of promoting penetration of the subject the multifunctional or multispecific antigen binding molecules or the compositions as described herein across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the multifunctional or multispecific antigen binding molecules or the compositions as described herein are linked or conjugated with agents that provide desirable pharmaceutical or pharmacodynamic properties. In some embodiments, the multifunctional or multispecific antigen binding molecules or the compositions as described herein are coupled to a substance, known in the art to promote penetration or transport across the blood-brain barrier, e.g., an antibody to the transferrin receptor. In some embodiments, the multifunctional or multispecific antigen binding molecules or the compositions as described herein are linked with a viral vector.

In some embodiments, subjects treated using the methods and compositions are evaluated for improvement in condition using any methods known and described in the art.

The terms “treat,” “treating”, and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition.

In some embodiments, the terms “condition,” “disease,” or “disorder,” as used herein, are interchangeable.

By “treating or preventing a disease or a disorder” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique. A patient who is being treated for a disease or a disorder, is one who a medical practitioner has diagnosed as having such a condition. Diagnosis may be by any suitable means. Diagnosis and monitoring may involve, for example, detecting the presence of pathological cells in a biological sample (e.g., tissue biopsy, blood test, or urine test), detecting the level of a surrogate marker of the disorder in a biological sample, or detecting symptoms associated with the disorder. A patient in whom the development of a disorder is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

### Definitions

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. The use of the words “a” or “an” when used in conjunction with the term “comprising” herein may mean “one,” but it is also consistent with the meaning of “one or more,” “at least one,” and “one or more than one.”

It should also be noted that the term “or” is generally employed in its sense including “and/or” unless the content clearly dictates otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

“Antigen binding molecule” or “Antibody molecule” as used herein can refer to a protein, e.g., an immunoglobulin chain or fragment thereof, comprising at least one immunoglobulin variable domain structure and/or sequence. An antibody molecule or antigen binding molecule encompasses antibodies (e.g., full-length antibodies) and antibody fragments. In some embodiments, an antibody molecule or antigen binding molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule or antigen binding molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules or antigen binding molecules include full length antibodies and antibody fragments, e.g., dAb (domain antibody), single chain, Fab, Fab′, and F(ab′)2 fragments, and single chain variable fragments (scFvs). In some embodiments, the antibody molecule or antigen binding molecule is an antibody mimetic. In some embodiments, the antibody molecule or antigen binding molecule is, or comprises, an antibody-like framework or scaffold, such as, fibronectins, ankyrin repeats (e.g., designed ankyrin repeat proteins (DARPins)), avimers, affibody affinity ligands, anticalins, or affilin molecules.

The term “human-like antibody molecule” or “human-like antigen binding molecule” as used herein refers to a humanized antibody molecule/antigen binding molecule, human antibody molecule/antigen binding molecule or an antibody molecule/antigen binding molecule having at least 95% sequence identity with a non-murine germline framework region, e.g., FR1, FR2, FR3 and/or FR4. In some embodiments, the human-like antibody molecule/antigen binding molecule comprises a framework region having at least 95% sequence identity to a human germline framework region, e.g., a FR1, FR2, FR3 and/or FR4 of a human germline framework region. In some embodiments, the human-like antibody molecule/antigen binding molecule is a recombinant antibody. In some embodiments, the human-like antibody molecule/antigen binding molecule is a humanized antibody molecule/antigen binding molecule. In some embodiments, the human-like antibody molecule/antigen binding molecule is human antibody molecule/antigen binding molecule. In some embodiments, the human-like antibody molecule/antigen binding molecule is a phage display or a yeast display antibody molecule/antigen binding molecule. In some embodiments, the human-like antibody molecule/antigen binding molecule is a chimeric antibody molecule/antigen binding molecule. In some embodiments, the human-like antibody molecule/antigen binding molecule is a CDR grafted antibody molecule/antigen binding molecule.

The term “humanize” refers to replacement or substitution of certain amino acids in an antibody or nanobody derived from a non-human species, in particular in the framework regions and constant domains of the heavy and/or light chains, in order to avoid or minimize an immune response in humans.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule/antigen binding molecule is a bispecific antibody molecule/antigen binding molecule. “Bispecific antibody molecule/antigen binding molecule” as used herein refers to an antibody molecule/antigen binding molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4. The term “combined” when referring to the Chothia and Kabat numbering schemes for antibodies typically refers to a modified or enhanced numbering system that aims to incorporate the strengths of both approaches (see www.abysis.org/abysis/sequence_input/key_annotation/key_annotation.cgi).

A “nanobody” (Nb), as used herein, refers to the smallest antigen binding fragment or single variable domain (“VHH”) derived from naturally occurring heavy chain antibody and is known to the person skilled in the art. They are derived from heavy chain only antibodies, seen, for example, in camelid antibodies. The nanobodies hereof generally comprise a single amino acid chain that can be considered to comprise four “framework sequences” that make up the “scaffold” and three “complementarity-determining regions” or CDRs (as defined hereinbefore). It should be noted that the term “nanobody,” as used herein in its broadest sense, is not limited to a specific biological source or to a specific method of preparation.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

The term “functional fragment” refers to a polypeptide that has a partial amino acid sequence of a reference amino acid sequence, and is capable of having one or more activities of the reference amino acid sequence. In some embodiments, the functional fragment comprises at least about 30%, 35%, 40%, 45%, 50%, 55%, 60%, 65%, 70%, 75%, 80%, 85%, 90%, 91%, 92%, 93%, 94%, 95%, 96%, 97%, 98% 99%, 99.5%, or 99.9% amino acid sequence of a reference amino acid sequence. In some embodiments, the functional fragment comprises an amino acid sequence that has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid deletion from the reference amino acid sequence. In some embodiments, the functional fragment comprises an amino acid sequence that has at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, or 500 amino acids of the reference amino acid sequence.

Calculations of homology or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4. The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

It is understood that the molecules/antigen binding molecules of the present invention may have additional conservative or non-essential amino acid substitutions, which do not have a substantial effect on their functions.

As used herein, the term “molecule” or “antigen binding molecule” as used in, e.g., antigen binding molecule, receptor antigen binding molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule or antigen binding molecule remains.

The terms “administer”, “administered”, “administers” and “administering” are defined as providing a composition to a subject via a route known in the art, including but not limited to intravenous, intraarterial, intrathecal, oral, parenteral, perineural, buccal, topical, transdermal, rectal, intramuscular, subcutaneous, intraosseous, transmucosal, intraperitoneal, or nerve root sheath routes of administration. In certain embodiments, oral routes of administering a composition can be used. The terms “administer”, “administered”, “administers” and “administering” a therapeutic protein should be understood to mean providing a therapeutic protein of the present disclosure or a prodrug of a therapeutic protein of the present disclosure to the individual in need.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material.

Each carrier must be “acceptable” in the sense of being compatible with the other ingredients of the formulation and not injurious to the patient.

As used herein, the terms “polypeptide,” “protein,” and “peptide” are used interchangeably herein, and refer to a polymeric form of amino acids of any length, which can include coded and non-coded amino acids, chemically or biochemically modified or derivatized amino acids, and polypeptides having modified peptide backbones.

The terms “subject”, “individual,” and “patient” may be used interchangeably and refer to humans, as well as non-human mammals (e.g., non-human primates, canines, equines, felines, porcines, bovines, ungulates, lagomorphs, rodents, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a therapeutic protein described herein.

The term “specificity” as used herein, refers to the ability of a protein binding domain, in particular, an immunoglobulin or an immunoglobulin fragment, such as a nanobody, to bind preferentially to one antigen versus a different antigen, and does not necessarily imply high affinity.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a therapeutic protein or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

A “degrading protein” or “degrader protein,” as that term is used herein, may encompasses a range of moieties including, but not limited to membrane associated internalizing protein, an internalizing receptor, a membrane associated degrading receptor, a degrading receptor, a surface moiety configured to internalize an antigen binding molecule, a surface moiety configured to degrade an antigen binding molecule, combinations thereof, or variants thereof.

An “internalizing protein,” as that term is used here, may encompass a range of moieties including, but not limited to membrane associated internalizing protein, an internalizing receptor, a surface moiety configured to internalize an antigen binding molecule, combinations thereof, or variants thereof.

### Numbered Embodiments

Numbered embodiment 1. A binding agent, comprising: (i) a first antigen binding domain that binds to an epidermal growth factor receptor (EGFR); and (ii) a second antigen binding domain that binds to an integrin subunit beta 6 (ITGB6), wherein the first antigen binding domain comprises a heavy chain variable region (VH) comprising: (a) a heavy chain complementarity determining region 1 (HCDRT) amino acid sequence of SEQ ID NO: 1; (b) a heavy chain complementarity determining region 2 (HCDR2) amino acid sequence of SEQ ID NO: 2; and (c) a heavy chain complementarity determining region 3 (HCDR3) amino acid sequence of SEQ ID NO: 3.

Numbered embodiment 2. The binding agent of numbered embodiment 1, wherein the first antigen binding domain comprises a light chain variable region (VL) comprising: (a) a light chain complementarity determining region 1 (LCDR1) amino acid sequence of SEQ ID NO: 4; (b) a light chain complementarity determining region 2 (LCDR2) amino acid sequence of SEQ ID NO: 5; and (c) a light chain complementarity determining region 3 (LCDR3) amino acid sequence of SEQ ID NO: 6.

Numbered embodiment 3. A binding agent, comprising: (i) a first antigen binding domain that binds to an epidermal growth factor receptor (EGFR); and (ii) a second antigen binding domain that binds to an integrin subunit beta 6 (ITGB6), wherein the second antigen binding domain comprises a heavy chain variable region (VH) comprising: (a) a heavy chain complementarity determining region 1 (HCDR1) amino acid sequence of SEQ ID NO: 7; (b) a heavy chain complementarity determining region 2 (HCDR2) amino acid sequence of SEQ ID NO: 8; and (c) a heavy chain complementarity determining region 3 (HCDR3) amino acid sequence of SEQ ID NO: 9.

Numbered embodiment 4. The binding agent of any one of numbered embodiments 1-3, wherein the second antigen binding domain comprises a light chain variable region (VL) comprising: (a) a light chain complementarity determining region 1 (LCDR1) amino acid sequence of SEQ ID NO: 10; (b) a light chain complementarity determining region 2 (LCDR2) amino acid sequence of SEQ ID NO: 11; and (c) a light chain complementarity determining region 3 (LCDR3) amino acid sequence of SEQ ID NO: 12.

Numbered embodiment 5. The binding agent of any one of numbered embodiments 1-4, wherein the first antigen binding domain comprises a heavy chain variable region (VH) comprising: (a) a HCDR1 amino acid sequence of DYGMH (SEQ ID NO: 16), or NQGIS (SEQ ID NO: 25); (b) a HCDR2 amino acid sequence of AIDAGGSTDYADSVEG (SEQ ID NO: 17) or GFDPDAGETIYAQKFQG (SEQ ID NO: 26); (c) a HCDR3 amino acid sequence of DLEAGYYAPDV (SEQ ID NO: 18) or GFDPDAGETIYAQKFQG (SEQ ID NO: 26); or (d) any combination thereof.

Numbered embodiment 6. The binding agent of any one of numbered embodiments 1-5, wherein the first antigen binding domain comprises a light chain variable region (VL) comprising: (a) a LCDR1 amino acid sequence of RASQDIGRFLA (SEQ ID NO: 31), or RASQDIRHYLA (SEQ ID NO: 37); (b) a LCDR2 amino acid sequence of AVSNLQS (SEQ ID NO: 32) or DTFNRAT (SEQ ID NO: 38); (c) a LCDR3 amino acid sequence of QQYSTSVYT (SEQ ID NO: 33) or QQYHNLPYS (SEQ ID NO: 39); or (d) any combination thereof.

Numbered embodiment 7. The binding agent of any one of numbered embodiments 1-6, wherein the second antigen binding domain comprises a heavy chain variable region (VH) comprising: (a) a HCDR1 amino acid sequence of NDLIE (SEQ ID NO: 58), or NYLIE (SEQ ID NO: 67); (b) a HCDR2 amino acid sequence of VINPGSGRTNYAQKFQG (SEQ ID NO: 59) or VISPGSGIINYAQKFQG (SEQ ID NO: 68); (c) a HCDR3 amino acid sequence of IYYGPHSYAMDY (SEQ ID NO: 60) or IDYSGPYAVDD (SEQ ID NO: 69); or (d) any combination thereof.

Numbered embodiment 8. The binding agent of any one of numbered embodiments 1-7, wherein the second antigen binding domain comprises a light chain variable region (VL) comprising: (a) a LCDR1 amino acid sequence of KASLDVRTAVA (SEQ ID NO: 73), or KASQAVNTAVA (SEQ ID NO: 79); (b) a LCDR2 amino acid sequence of SASYRYT (SEQ ID NO: 74) or SASYGYT (SEQ ID NO: 80); (c) a LCDR3 amino acid sequence of QQHYGIPWT (SEQ ID NO: 75) or QHHYGVPWT (SEQ ID NO: 81); or (d) any combination thereof.

Numbered embodiment 9. The binding agent of any one of numbered embodiments 1-8, wherein the first antigen binding domain comprises a heavy chain variable region (VH) comprising: (a) a HCDR1 amino acid sequence of DYGMH (SEQ ID NO: 16; (b) a HCDR2 amino acid sequence of AIDAGGSTDYADSVEG (SEQ ID NO: 17); and (c) a HCDR3 amino acid sequence of DLEAGYYAPDV (SEQ ID NO: 18).

Numbered embodiment 10. The binding agent of any one of numbered embodiments 1-9, wherein the first antigen binding domain comprises a light chain variable region (VL) comprising: (a) a LCDR1 amino acid sequence of RASQDIGRFLA (SEQ ID NO: 31); (b) a LCDR2 amino acid sequence of AVSNLQS (SEQ ID NO: 32); and (c) a LCDR3 amino acid sequence of QQYSTSVYT (SEQ ID NO: 33).

Numbered embodiment 11. The binding agent of any one of numbered embodiments 1-10, wherein the first antigen binding domain comprises: (a) a VL comprising a LCDR1 amino acid sequence of RASQDIGRFLA (SEQ ID NO: 31), a LCDR2 amino acid sequence of AVSNLQS (SEQ ID NO: 32), and a LCDR3 amino acid sequence of QQYSTSVYT (SEQ ID NO: 33); and (b) a VH comprising a HCDR1 amino acid sequence of DYGMH (SEQ ID NO: 16), a HCDR2 amino acid sequence of AIDAGGSTDYADSVEG (SEQ ID NO: 17), and a HCDR3 amino acid sequence of DLEAGYYAPDV (SEQ ID NO: 18).

Numbered embodiment 12. The binding agent of any one of numbered embodiments 1-11, wherein the first antigen binding domain comprises a VL comprising a sequence having at least 75%, at least 80%, at least 85%, at least 90%, at least 95%, or at least 99% sequence identity to the sequence of SEQ ID NO: 49.

Numbered embodiment 13. The binding agent of any one of numbered embodiments 1-12, wherein the first antigen binding domain comprises a VL comprising the sequence of SEQ ID NO: 49.

Numbered embodiment 14. The binding agent of any one of numbered embodiments 1-13, wherein the first antigen binding domain comprises a VH comprising a sequence having at least 75%, at least 80%, at least 85%, at least 90%, at least 95%, or at least 99% sequence identity to the sequence of SEQ ID NO: 43.

Numbered embodiment 15. The binding agent of any one of numbered embodiments 1-14, wherein the first antigen binding domain comprises a VH comprising the sequence of SEQ ID NO: 43.

Numbered embodiment 16. The binding agent of any one of numbered embodiments 1-15, wherein the first antigen binding domain comprises a VL comprising a sequence having at least 75%, at least 80%, at least 85%, at least 90%, at least 95%, or at least 99% sequence identity to the sequence of SEQ ID NO: 49 and a VH comprising a sequence having at least 75%, at least 80%, at least 85%, at least 90%, at least 95%, or at least 99% sequence identity to the sequence of SEQ ID NO: 43.

Numbered embodiment 17. The binding agent of any one of numbered embodiments 1-16, wherein the first antigen binding domain comprises a VL comprising the sequence of SEQ ID NO: 49 and a VH comprising the sequence of SEQ ID NO: 43.

Numbered embodiment 18. The binding agent of any one of numbered embodiments 1-17, wherein the second antigen binding domain comprises a heavy chain variable region (VH) comprising: (a) a HCDR1 amino acid sequence of NDLIE (SEQ ID NO: 58); (b) a HCDR2 amino acid sequence of VINPGSGRTNYAQKFQG (SEQ ID NO: 59); and (c) a HCDR3 amino acid sequence of 1YYGPHSYAMDY (SEQ ID NO: 60).

Numbered embodiment 19. The binding agent of any one of numbered embodiments 1-18, wherein the second antigen binding domain comprises a light chain variable region (VL) comprising: (a) a LCDR1 amino acid sequence of KASLDVRTAVA (SEQ ID NO: 73); (b) a LCDR2 amino acid sequence of SASYRYT (SEQ ID NO: 74); and (c) a LCDR3 amino acid sequence of QQHYGIPWT (SEQ ID NO: 75).

Numbered embodiment 20. The binding agent of any one of numbered embodiments 1-19, wherein the second antigen binding domain comprises: (a) a VL comprising a LCDR1 amino acid sequence of KASLDVRTAVA (SEQ ID NO: 73), a LCDR2 amino acid sequence of SASYRYT (SEQ ID NO: 74), and a LCDR3 amino acid sequence of QQHYGIPWT (SEQ ID NO: 75); and (b) a VH comprising a HCDR1 amino acid sequence of NDLIE (SEQ ID NO: 58), a HCDR2 amino acid sequence of VINPGSGRTNYAQKFQG (SEQ ID NO: 59), and a HCDR3 amino acid sequence of IYYGPHSYAMDY (SEQ ID NO: 60).

Numbered embodiment 21. The binding agent of any one of numbered embodiments 1-20, wherein the second antigen binding domain comprises a VL comprising a sequence having at least 75%, at least 80%, at least 85%, at least 90%, at least 95%, or at least 99% sequence identity to the sequence of SEQ ID NO: 91.

Numbered embodiment 22. The binding agent of any one of numbered embodiments 1-21, wherein the second antigen binding domain comprises a VL comprising the sequence of SEQ ID NO: 91.

Numbered embodiment 23. The binding agent of any one of numbered embodiments 1-22, wherein the second antigen binding domain comprises a VH comprising a sequence having at least 75%, at least 80%, at least 85%, at least 90%, at least 95%, or at least 99% sequence identity to the sequence of SEQ ID NO: 85.

Numbered embodiment 24. The binding agent of any one of numbered embodiments 1-23, wherein the second antigen binding domain comprises a VH comprising the sequence of SEQ ID NO: 85.

Numbered embodiment 25. The binding agent of any one of numbered embodiments 1-24, wherein the second antigen binding domain comprises a VL comprising a sequence having at least 75%, at least 80%, at least 85%, at least 90%, at least 95%, or at least 99% sequence identity to the sequence of SEQ ID NO: 91 and a VH comprising a sequence having at least 75%, at least 80%, at least 85%, at least 90%, at least 95%, or at least 99% sequence identity to the sequence of SEQ ID NO: 85.

Numbered embodiment 26. The binding agent of any one of numbered embodiments 1-25, wherein the second antigen binding domain comprises a VL comprising the sequence of SEQ ID NO: 91 and a VH comprising the sequence of SEQ ID NO: 85.

Numbered embodiment 27. The binding agent of any one of numbered embodiments 1-26, wherein the second antigen binding domain comprises a heavy chain variable region (VH) comprising: (a) a HCDR1 amino acid sequence of NYLIE (SEQ ID NO: 67); (b) a HCDR2 amino acid sequence of VISPGSGIINYAQKFQG (SEQ ID NO: 68); and (c) a HCDR3 amino acid sequence of IDYSGPYAVDD (SEQ ID NO: 69).

Numbered embodiment 28. The binding agent of any one of numbered embodiments 1-27, wherein the second antigen binding domain comprises a light chain variable region (VL) comprising: (a) a LCDR1 amino acid sequence of KASQAVNTAVA (SEQ ID NO: 79); (b) a LCDR2 amino acid sequence of SASYGYT (SEQ ID NO: 80); and (c) a LCDR3 amino acid sequence of QHHYGVPWT (SEQ ID NO: 81).

Numbered embodiment 29. The binding agent of any one of numbered embodiments 1-28, wherein the second antigen binding domain comprises: (a) a VL comprising a LCDR1 amino acid sequence of KASQAVNTAVA (SEQ ID NO: 79), a LCDR2 amino acid sequence of SASYGYT (SEQ ID NO: 80), and a LCDR3 amino acid sequence of QHHYGVPWT (SEQ ID NO: 81); and (b) a VH comprising a HCDR1 amino acid sequence of NYLIE (SEQ ID NO: 67), a HCDR2 amino acid sequence of VISPGSGIINYAQKFQG (SEQ ID NO: 68), and a HCDR3 amino acid sequence of IDYSGPYAVDD (SEQ ID NO: 69).

Numbered embodiment 30. The binding agent of any one of numbered embodiments 1-29, wherein the second antigen binding domain comprises a VL comprising a sequence having at least 75%, at least 80%, at least 85%, at least 90%, at least 95%, or at least 99% sequence identity to the sequence of SEQ ID NO: 94.

Numbered embodiment 31. The binding agent of any one of numbered embodiments 1-30, wherein the second antigen binding domain comprises a VL comprising the sequence of SEQ ID NO: 94.

Numbered embodiment 32. The binding agent of any one of numbered embodiments 1-31, wherein the second antigen binding domain comprises a VH comprising a sequence having at least 75%, at least 80%, at least 85%, at least 90%, at least 95%, or at least 99% sequence identity to the sequence of SEQ ID NO: 88.

Numbered embodiment 33. The binding agent of any one of numbered embodiments 1-32, wherein the second antigen binding domain comprises a VH comprising the sequence of SEQ ID NO: 88.

Numbered embodiment 34. The binding agent of any one of numbered embodiments 1-33, wherein the second antigen binding domain comprises a VL comprising a sequence having at least 75%, at least 80%, at least 85%, at least 90%, at least 95%, or at least 99% sequence identity to the sequence of SEQ ID NO: 94 and a VH comprising a sequence having at least 75%, at least 80%, at least 85%, at least 90%, at least 95%, or at least 99% sequence identity to the sequence of SEQ ID NO: 88.

Numbered embodiment 35. The binding agent of any one of numbered embodiments 1-34, wherein the second antigen binding domain comprises a VL comprising the sequence of SEQ ID NO: 94 and a VH comprising the sequence of SEQ ID NO: 88.

Numbered embodiment 36. The binding agent of any one of numbered embodiments 1-8 and 18-35, wherein the first antigen binding domain comprises a heavy chain variable region (VH) comprising: (a) a HCDR1 amino acid sequence of NQGIS (SEQ ID NO: 25); (b) a HCDR2 amino acid sequence of GFDPDAGETIYAQKFQG (SEQ ID NO: 26); and (c) a HCDR3 amino acid sequence of GVDSYGYGRYNWFDP (SEQ ID NO: 27).

Numbered embodiment 37. The binding agent of any one of numbered embodiments 1-8 and 18-36, wherein the first antigen binding domain comprises a light chain variable region (VL) comprising: (a) a LCDR1 amino acid sequence of RASQDIRHYLA (SEQ ID NO: 37); (b) a LCDR2 amino acid sequence of DTFNRAT (SEQ ID NO: 38); and (c) a LCDR3 amino acid sequence of QQYHNLPYS (SEQ ID NO: 39).

Numbered embodiment 38. The binding agent of any one of numbered embodiments 1-8 and 18-37, wherein the first antigen binding domain comprises: (a) a VL comprising a LCDR1 amino acid sequence of RASQDIRHYLA (SEQ ID NO: 37), a LCDR2 amino acid sequence of DTFNRAT (SEQ ID NO: 38), and a LCDR3 amino acid sequence of QQYHNLPYS (SEQ ID NO: 39); and (b) a VH comprising a HCDR1 amino acid sequence of NQGIS (SEQ ID NO: 25), a HCDR2 amino acid sequence of GFDPDAGETIYAQKFQG (SEQ ID NO: 26), and a HCDR3 amino acid sequence of GVDSYGYGRYNWFDP (SEQ ID NO: 27).

Numbered embodiment 39. The binding agent of any one of numbered embodiments 1-8 and 18-38, wherein the first antigen binding domain comprises a VL comprising a sequence having at least 75%, at least 80%, at least 85%, at least 90%, at least 95%, or at least 99% sequence identity to the sequence of SEQ ID NO: 52.

Numbered embodiment 40. The binding agent of any one of numbered embodiments 1-8 and 18-39, wherein the first antigen binding domain comprises a VL comprising the sequence of SEQ ID NO: 52.

Numbered embodiment 41. The binding agent of any one of numbered embodiments 1-8 and 18-40, wherein the first antigen binding domain comprises a VH comprising a sequence having at least 75%, at least 80%, at least 85%, at least 90%, at least 95%, or at least 99% sequence identity to the sequence of SEQ ID NO: 46.

Numbered embodiment 42. The binding agent of any one of numbered embodiments 1-8 and 18-41, wherein the first antigen binding domain comprises a VH comprising the sequence of SEQ ID NO: 46.

Numbered embodiment 43. The binding agent of any one of numbered embodiments 1-8 and 18-42, wherein the first antigen binding domain comprises a VL comprising a sequence having at least 75%, at least 80%, at least 85%, at least 90%, at least 95%, or at least 99% sequence identity to the sequence of SEQ ID NO: 52 and a VH comprising a sequence having at least 75%, at least 80%, at least 85%, at least 90%, at least 95%, or at least 99% sequence identity to the sequence of SEQ ID NO: 46.

Numbered embodiment 44. The binding agent of any one of numbered embodiments 1-8 and 18-43, wherein the first antigen binding domain comprises a VL comprising the sequence of SEQ ID NO: 52 and a VH comprising the sequence of SEQ ID NO: 46.

Numbered embodiment 45. The binding agent of any one of numbered embodiments 1-44, wherein the binding agent comprises a first portion and a second portion.

Numbered embodiment 46. The binding agent of numbered embodiment 45, wherein the first portion comprises a first light chain constant region.

Numbered embodiment 47. The binding agent of numbered embodiment 45 or 46, wherein the second portion comprises a second light chain constant region.

Numbered embodiment 48. The binding agent of numbered embodiment 46 or 47, wherein the first light chain constant region or the second light chain constant region, or a combination thereof comprises a kappa light chain constant region or functional fragment thereof, a lambda light chain constant region or functional fragment thereof, or a combination thereof.

Numbered embodiment 49. The binding agent of any one of numbered embodiments 45-48, wherein the first portion comprises a Fab or a scFv.

Numbered embodiment 50. The binding agent of any one of numbered embodiments 45-49, wherein the second portion comprises a Fab or a scFv.

Numbered embodiment 51. The binding agent of any one of numbered embodiments 45-50, wherein the first portion comprises one or more heavy chain constant regions.

Numbered embodiment 52. The binding agent of any one of numbered embodiments 45-51, wherein the second portion comprises one or more heavy chain constant regions.

Numbered embodiment 53. The binding agent of numbered embodiments 51 or 52, wherein the one or more heavy chain constant regions selected from the group consisting of IgG1 heavy chain constant region or functional fragment thereof, IgG2 heavy chain constant region or functional fragment thereof, IgG3 heavy chain constant region or functional fragment thereof, IgGA1 heavy chain constant region or functional fragment thereof, IgGA2 heavy chain constant region or functional fragment thereof, IgG4 heavy chain constant region or functional fragment thereof, IgJ heavy chain constant region or functional fragment thereof, IgM heavy chain constant region or functional fragment thereof, IgD heavy chain constant region or functional fragment thereof, and IgE heavy chain constant region or functional fragment thereof.

Numbered embodiment 54. The binding agent of any one of numbered embodiments 45-53, wherein the first portion comprises a first immunoglobulin constant region (Fc region).

Numbered embodiment 55. The binding agent of any one of numbered embodiments 45-54, wherein the second portion comprises a second Fc region.

Numbered embodiment 56. The binding agent of numbered embodiment 54 or 55, wherein the first Fc region, the second Fc region, or a combination thereof is selected from the group consisting of an IgG1 Fc region or a functional fragment thereof, an IgG2 Fc region or a functional fragment thereof, an IgG3 Fc region or a functional fragment thereof, an IgGA1 Fc region or a functional fragment thereof, an IgGA2 Fc region or a functional fragment thereof, an IgG4 Fc region or a functional fragment thereof, an IgJ Fc region or a functional fragment thereof, an IgM Fc region or a functional fragment thereof, an IgD Fc region or a functional fragment thereof, and an IgE Fc region or a functional fragment thereof.

Numbered embodiment 57. The binding agent of any one of numbered embodiments 1-56, wherein the binding agent is a multispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab.

Numbered embodiment 58. The binding agent of any one of numbered embodiments 45-57, wherein the first portion further comprises: (a) the first antigen binding domain; (b) a first polypeptide; and (c) a second polypeptide, wherein the first polypeptide and the second polypeptide are non-contiguous.

Numbered embodiment 59. The binding agent of numbered embodiment 58, wherein: (a) the first polypeptide comprises a Light Chain Constant Region (CL); and (b) the second polypeptide comprises a Heavy Chain Constant Region (CH).

Numbered embodiment 60. The binding agent of any one of numbered embodiments 45-59, wherein the second portion further comprises: (a) the second antigen binding domain; (b) a third polypeptide; and (c) a fourth polypeptide, wherein the third polypeptide and the fourth polypeptide are non-contiguous.

Numbered embodiment 61. The binding agent of numbered embodiment 60, wherein: (a) the third polypeptide comprises a Light Chain Constant Region (CL); and (b) the fourth polypeptide comprises a Heavy Chain Constant Region (CH).

Numbered embodiment 62. The binding agent of numbered embodiment 61, wherein: (a) the VH of the first antigen binding domain comprises a dimerization domain; (b) the VL of the first antigen binding domain comprises a dimerization domain; (c) the VH of the second antigen binding domain comprises a dimerization domain; (d) the VL of the second antigen binding domain comprises a dimerization domain; (e) the CH of the second polypeptide comprises a dimerization domain; (f) the CL of the first polypeptide comprises a dimerization domain; (g) the CH of the fourth polypeptide comprises a dimerization domain; (h) the CL of the third polypeptide comprises a dimerization domain; or (i) a combination thereof.

Numbered embodiment 63. The binding agent of numbered embodiment 62, wherein: (d) the VH and VL of the first antigen binding domain are dimerized (e) the VH and VL of the second antigen binding domain are dimerized; (f) the CH of the fourth polypeptide and the CL of the third polypeptide are dimerized; (g) the CH of the second polypeptide and CL of the first polypeptide are dimerized; (h) the CH of the second polypeptide and the CH of the fourth polypeptide are dimerized; or (i) a combination thereof, wherein any one of (a)-(f) are linked through the dimerization domain.

Numbered embodiment 64. The binding agent of numbered embodiment 63, wherein the dimerization domain comprises a disulfide bond.

Numbered embodiment 65. The binding agent of any one of numbered embodiments 58-64, wherein the first polypeptide comprises a sequence having at least 75%, at least 80%, at least 85%, at least 90%, at least 95%, or at least 99% sequence identity to the sequence of any one of SEQ ID NO: 51 or 54.

Numbered embodiment 66. The binding agent of any one of numbered embodiments 58-65, wherein the first polypeptide comprises a sequence of any one of SEQ ID NO: 51 or 54.

Numbered embodiment 67. The binding agent of any one of numbered embodiments 58-66, wherein the second polypeptide comprises a sequence having at least 75%, at least 80%, at least 85%, at least 90%, at least 95%, or at least 99% sequence identity to the sequence of any one of SEQ ID NO: 45 or 48.

Numbered embodiment 68. The binding agent of any one of numbered embodiments 58-67, wherein the second polypeptide comprises a sequence of any one of SEQ ID NO: 45 or 48.

Numbered embodiment 69. The binding agent of any one of numbered embodiments 58-68, wherein the first polypeptide comprises a sequence having at least 75%, at least 80%, at least 85%, at least 90%, at least 95%, or at least 99% sequence identity to the sequence of any one of SEQ ID NOs: 51 or 54; and the second polypeptide comprises a sequence having at least 75%, at least 80%, at least 85%, at least 90%, at least 95%, or at least 99% sequence identity to the sequence of any one of SEQ ID NOs: 45 or 48.

Numbered embodiment 70. The binding agent of any one of numbered embodiments 58-69, wherein the first polypeptide comprises a sequence of any one of SEQ ID NOs: 51 or 54; and the second polypeptide comprises a sequence of any one of SEQ ID NOs: 45 or 48.

Numbered embodiment 71. The binding agent of any one of numbered embodiments 60-70, wherein the third polypeptide comprises a sequence having at least 75%, at least 80%, at least 85%, at least 90%, at least 95%, or at least 99% sequence identity to the sequence of any one of SEQ ID NOs: 93 or 96.

Numbered embodiment 72. The binding agent of any one of numbered embodiments 60-71, wherein the third polypeptide comprises a sequence of any one of SEQ ID NOs: 93 or 96.

Numbered embodiment 73. The binding agent of any one of numbered embodiments 60-72, wherein the fourth polypeptide comprises a sequence having at least 75%, at least 80%, at least 85%, at least 90%, at least 95%, or at least 99% sequence identity to the sequence of any one of SEQ ID NOs: 87 or 90.

Numbered embodiment 74. The binding agent of any one of numbered embodiments 60-73, wherein the fourth polypeptide comprises a sequence of any one of SEQ ID NOs: 87 or 90.

Numbered embodiment 75. The binding agent of any one of numbered embodiments 60-74, wherein the third polypeptide comprises a sequence having at least 75%, at least 80%, at least 85%, at least 90%, at least 95%, or at least 99% sequence identity to the sequence of any one of SEQ ID NOs: 93 or 96; and the fourth polypeptide comprises a sequence having at least 75%, at least 80%, at least 85%, at least 90%, at least 95%, or at least 99% sequence identity to the sequence of any one of SEQ ID NOs: 87 or 90.

Numbered embodiment 76. The binding agent of any one of numbered embodiments 60-75, wherein the third polypeptide comprises a sequence of any one of SEQ ID NOs: 93 or 96; and the fourth polypeptide comprises a sequence of any one of SEQ ID NOs: 87 or 90.

Numbered embodiment 77. The binding agent of any one of numbered embodiments 61-76, wherein the first portion comprises the CH of the second polypeptide linked to the VH of the first antigen binding domain, wherein the CH further comprises a CH1.

Numbered embodiment 78. The binding agent of any one of numbered embodiments 61-77, wherein the second portion comprises the CH of the fourth polypeptide linked to the VH of the second antigen binding domain, wherein the CH further comprises a CH1.

Numbered embodiment 79. The binding agent of numbered embodiment 77 or 78, wherein the CH1 is linked to the C-terminus of the VH.

Numbered embodiment 80. The binding agent of any one of numbered embodiments 61-79, wherein the first portion comprises a Heavy Chain (VH-CH) comprising a sequence having at least 75%, at least 80%, at least 85%, at least 90%, at least 95%, or at least 99% sequence identity to the sequence of any one of SEQ ID NOs: 44 or 47.

Numbered embodiment 81. The binding agent of numbered embodiment 80, wherein the first portion comprises a VH-CH comprising the sequence of any one of SEQ ID NOs: 44 or 47.

Numbered embodiment 82. The binding agent of any one of numbered embodiments 61-81, wherein the second portion comprises a Heavy Chain (VH-CH) comprising a sequence having at least 75%, at least 80%, at least 85%, at least 90%, at least 95%, or at least 99% sequence identity to the sequence of any one of SEQ ID NOs: 86 or 89.

Numbered embodiment 83. The binding agent of numbered embodiment 82, wherein the second portion comprises a VH-CH comprising the sequence of any one of SEQ ID NOs: 86 or 89.

Numbered embodiment 84. The binding agent of any one of numbered embodiments 61-83, wherein the first portion comprises the CL of the first polypeptide linked to the VL of the first antigen binding domain.

Numbered embodiment 85. The binding agent of any one of numbered embodiments 61-84, wherein the second portion comprises the CL of the third polypeptide linked to the VL of the second antigen binding domain.

Numbered embodiment 86. The binding agent of numbered embodiment 84 or 85, wherein the CL is linked to a C-terminus of the VL.

Numbered embodiment 87. The binding agent of any one of numbered embodiments 61-86, wherein the first portion comprises a Light Chain (VL-CL) comprising a sequence having at least 75%, at least 80%, at least 85%, at least 90%, at least 95%, or at least 99% sequence identity to the sequence of any one of SEQ ID NOs: 50 or 53.

Numbered embodiment 88. The binding agent of numbered embodiment 87, wherein the first portion comprises a VL-CL comprising the sequence of any one of SEQ ID NOs: 50 or 53.

Numbered embodiment 89. The binding agent of any one of numbered embodiments 61-88, wherein the second portion comprises a Light Chain (VL-CL) comprising a sequence having at least 75%, at least 80%, at least 85%, at least 90%, at least 95%, or at least 99% sequence identity to the sequence of any one of SEQ ID NOs: 92 or 95.

Numbered embodiment 90. The binding agent of numbered embodiment 89, wherein the second portion comprises a VL-CL comprising the sequence of any one of SEQ ID NOs: 92 or 95.

Numbered embodiment 91. The binding agent of any one of numbered embodiments 61-90, wherein the first portion comprises a VL-CL comprising a sequence having at least 75%, at least 80%, at least 85%, at least 90%, at least 95%, or at least 99% sequence identity to the sequence of any one of SEQ ID NOs: 50 or 53; and a VH-CH comprising a sequence having at least 75%, at least 80%, at least 85%, at least 90%, at least 95%, or at least 99% sequence identity to the sequence of any one of SEQ ID NOs: 44 or 47.

Numbered embodiment 92. The binding agent of any one of numbered embodiments 61-91, wherein the first portion comprises a VL-CL comprising the sequence of any one of SEQ ID NOs: 50 or 53; and a VH-CH comprising the sequence of any one of SEQ ID NOs: 44 or 47.

Numbered embodiment 93. The binding agent of any one of numbered embodiments 61-92, wherein the second portion comprises a VL-CL comprising a sequence having at least 75%, at least 80%, at least 85%, at least 90%, at least 95%, or at least 99% sequence identity to the sequence of any one of SEQ ID NOs: 92 or 95; and a VH-CH comprising a sequence having at least 75%, at least 80%, at least 85%, at least 90%, at least 95%, or at least 99% sequence identity to the sequence of any one of SEQ ID NOs: 86 or 89.

Numbered embodiment 94. The binding agent of any one of numbered embodiments 61-93, wherein the second portion comprises a VL-CL comprising the sequence of any one of SEQ ID NOs: 92 or 95; and a VH-CH comprising the sequence of any one of SEQ ID NOs: 86 or 89.

Numbered embodiment 95. The binding agent of any one of numbered embodiments 61-94, wherein: (a) the first portion comprises: (i) a VL-CL comprising a sequence having at least 75%, at least 80%, at least 85%, at least 90%, at least 95%, or at least 99% sequence identity to the sequence of any one of SEQ ID NOs: 50 or 53, and (ii) a VH-CH comprising a sequence having at least 75%, at least 80%, at least 85%, at least 90%, at least 95%, or at least 99% sequence identity to the sequence of any one of SEQ ID NOs: 44 or 47; and (b) the second portion comprises: (iii) a VL-CL comprising a sequence having at least 75%, at least 80%, at least 85%, at least 90%, at least 95%, or at least 99% sequence identity to the sequence of any one of SEQ ID NOs: 92 or 95, and (iv) a VH-CH comprising a sequence having at least 75%, at least 80%, at least 85%, at least 90%, at least 95%, or at least 99% sequence identity to the sequence of any one of SEQ ID NOs: 86 or 89.

Numbered embodiment 96. The binding agent of any one of numbered embodiments 61-95, wherein: (a) the first portion comprises: (v) a VL-CL comprising a sequence of any one of SEQ ID NOs: 50 or 53, and (vi) a VH-CH comprising a sequence of any one of SEQ ID NOs: 44 or 47; and (b) the second portion comprises: (vii) a VL-CL comprising a sequence of any one of SEQ ID NOs: 92 or 95, and (viii) a VH-CH comprising a sequence of any one of SEQ ID NOs: 86 or 89.

Numbered embodiment 97. A recombinant polynucleotide molecule comprising the polynucleotide sequences encoding the binding agent of any one of numbered embodiments 1-96.

Numbered embodiment 98. The recombinant polynucleotide molecule of numbered embodiment 97, wherein the recombinant polynucleotide molecule is an isolated recombinant polynucleotide molecule.

Numbered embodiment 99. A vector comprising the recombinant polynucleotide molecule of numbered embodiment 97 or 98.

Numbered embodiment 100. A cell comprising the recombinant polynucleotide molecule of numbered embodiment 97 or 98, or the vector of numbered embodiment 99.

Numbered embodiment 101. A pharmaceutical composition comprising the binding agent of any one of numbered embodiments 1-96, the recombinant polynucleotide of numbered embodiment 97 or 98, the vector of numbered embodiment 99, or the cell of numbered embodiment 100, and a pharmaceutically acceptable carrier, excipient, or diluent.

Numbered embodiment 102. A method of treating a condition or disease in a subject in need thereof, comprising administering to the subject a therapeutically effective amount of the binding agent of any one of numbered embodiments 1-96, the recombinant polynucleotide of numbered embodiment 97 or 98, the vector of numbered embodiment 99, the cell of numbered embodiment 100, the pharmaceutical composition of numbered embodiment 101, or any combination thereof, thereby treating the condition or disease in the subject.

Numbered embodiment 103. The method of numbered embodiment 102, wherein the condition or disease is cancer.

Numbered embodiment 104. A method of decreasing EGFR expression on the surface of a cancer cell, comprising: contacting EGFR with the binding agent of any one of numbered embodiments 1-96, the recombinant polynucleotide of numbered embodiment 97 or 98, the vector of numbered embodiment 99, the cell of numbered embodiment 100, the pharmaceutical composition of numbered embodiment 101, or any combination thereof, wherein contacting EGFR with the binding agent results in decreased EGFR expression on the surface of the cancer cell by about 20%, 30%, 40%, 50%, 60%, 70% or more relative to EGFR expression on the surface of a corresponding cancer cell contacted with a control binding agent, thereby decreasing EGFR expression on the surface of a cancer cell.

Numbered embodiment 105. A method of increasing EGFR degradation in a cancer cell, comprising: contacting EGFR with the binding agent of any one of numbered embodiments 1-96, the recombinant polynucleotide of numbered embodiment 97 or 98, the vector of numbered embodiment 99, the cell of numbered embodiment 100, the pharmaceutical composition of numbered embodiment 101, or any combination thereof, wherein contacting EGFR with the binding agent results in increased EGFR degradation in the cancer cell by about 20%, 30%, 40%, 50%, 60%, 70% or more relative to EGFR degradation of a corresponding cancer cell contacted with a control binding agent, thereby increasing EGFR degradation in a cancer cell.

Numbered embodiment 106. A method of increasing cell surface removal of EGFR on a cancer cell, comprising: contacting EGFR with the binding agent of any one of numbered embodiments 1-96, the recombinant polynucleotide of numbered embodiment 97 or 98, the vector of numbered embodiment 99, the cell of numbered embodiment 100, the pharmaceutical composition of numbered embodiment 101, or any combination thereof, wherein contacting EGFR with the binding agent results in increased cell surface removal of EGFR on the cancer cell by about 20%, 30%, 40%, 50%, 60%, 70% or more relative to cell surface removal of EGFR on a corresponding cancer cell contacted with a control binding agent, thereby increasing cell surface removal of EGFR on a cancer cell.

Numbered embodiment 107. The method of any one of numbered embodiments 103-106, wherein the cancer is a solid tumor cancer, a hematological cancer, a metastatic cancer, a soft tissue tumor, or a combination thereof.

Numbered embodiment 108. The method of any one of numbered embodiments 103-107, wherein the cancer is non-small cell lung cancer (NSCLC), colorectal cancer, or squamous cell carcinoma (HNSCC).

Numbered embodiment 109. The method of numbered embodiment 108, wherein the cancer is NSCLC.

Numbered embodiment 110. The method of numbered embodiment 109, wherein the NSCLC is characterized as having an oncogenic EGFR alteration.

Numbered embodiment 111. The method of numbered embodiment 110, wherein the oncogenic EGFR alteration is an EGFR L858 mutation, T790 mutation, C797 mutation, Exon19 mutation, or any combination thereof.

Numbered embodiment 112. The method of numbered embodiment 111, wherein the EGFR L858 mutation is a L858R mutation.

Numbered embodiment 113. The method of numbered embodiment 111, wherein the EGFR T790 mutation is a T790M mutation.

Numbered embodiment 114. The method of numbered embodiment 111, wherein the EGFR C797 mutation is a C797S mutation and/or the Exon19 mutation is an Exon19 deletion mutation.

Numbered embodiment 115. The method of any one of numbered embodiments 108-114, wherein the subject has relapsed after prior therapy.

Numbered embodiment 116. The method of any one of numbered embodiments 108-115, wherein the subject has acquired resistance to prior therapy.

Numbered embodiment 117. The method of numbered embodiment 116, wherein the prior therapy comprises treatment with an EGFR tyrosine kinase inhibitor.

Numbered embodiment 118. The method of numbered embodiment 117, wherein the EGFR tyrosine kinase inhibitor is osimertinib.

Numbered embodiment 119. The method of any one of numbered embodiments 103-118, wherein the method increases the susceptibility of the cancer cell to cancer therapeutic agents.

Numbered embodiment 120. The method of numbered embodiment 119, wherein the cancer therapeutic agent is a cytotoxic agent.

Numbered embodiment 121. The method of any one of numbered embodiments 103-120, wherein the method reduces proliferation of the cancer cell.

Numbered embodiment 122. The method of any one of numbered embodiments 103-121, wherein the method increases death of the cancer cell.

Numbered embodiment 123. A method of decreasing tumor volume of a tumor, comprising:
contacting the tumor with the binding agent of any one of numbered embodiments 1-96, the recombinant polynucleotide of numbered embodiment 97 or 98, the vector of numbered embodiment 99, the cell of numbered embodiment 100, the pharmaceutical composition of numbered embodiment 101, or any combination thereof,
wherein contacting the tumor with the binding agent results in decreased tumor volume of the tumor by about 20%, 30%, 40%, 50%, 60%, 70% or more relative to the tumor volume of a corresponding tumor not contacted with the binding agent, thereby decreasing tumor volume of a tumor.

Numbered embodiment 124. A method of decreasing tumor volume of a tumor, comprising:
contacting the tumor with the binding agent of any one of numbered embodiments 1-96, the recombinant polynucleotide of numbered embodiment 97 or 98, the vector of numbered embodiment 99, the cell of numbered embodiment 100, the pharmaceutical composition of numbered embodiment 101, or any combination thereof,
wherein contacting the tumor with the binding agent results in decreased tumor volume of the tumor by about 20%, 30%, 40%, 50%, 60%, 70% or more relative to the tumor volume of a corresponding tumor contacted with a control binding agent, thereby decreasing tumor volume of a tumor.

Numbered embodiment 125. The method of any one of numbered embodiments 104-124, wherein the contacting is performed in vivo.

Numbered embodiment 126. The method of any one of numbered embodiments 104-106, or 124, wherein the control binding agent is a single arm EGFR binding agent, such as a single arm EGFR antibody.

Numbered embodiment 127. The method of any one of numbered embodiments 104-106, or 124, wherein the control binding agent is Cetuximab.

Numbered embodiment 128. The method of any one of numbered embodiments 102-127, wherein the half-life of the binding agent is within 20% of the half-life of Cetuximab.

Numbered embodiment 129. The method of any one of numbered embodiments 102-128, wherein the clearance rate of the binding agent is within 20-95% of the clearance rate of Cetuximab.

Numbered embodiment 130. The method of any one of numbered embodiments 102-129, wherein the Kd of the binding agent is within two-fold of the binding affinity of Cetuximab to EGFR.

Numbered embodiment 131. The method of numbered embodiment 130, wherein the Kd of the binding agent is within five-fold of the binding affinity of Cetuximab to EGFR.

Numbered embodiment 132. The method of numbered embodiment 131, wherein the Kd of the binding agent is within ten-fold of the binding affinity of Cetuximab to EGFR.

Numbered embodiment 133. The method of any one of numbered embodiments 102-132, wherein the Kd of the binding affinity of the binding agent may be within an order of magnitude of the binding affinity of a monovalent binding agent.

Numbered embodiment 134. The method of any one of numbered embodiments 102-133, wherein the Kd of the binding agent is within +/−10% of the binding affinity of Cetuximab to EGFR.

Numbered embodiment 135. The method of numbered embodiment 134, wherein the Kd of the binding agent is within +/−20% of the binding affinity of Cetuximab to EGFR.

Numbered embodiment 136. The method of numbered embodiment 135, wherein the Kd of the binding agent is within +/−30% of the binding affinity of Cetuximab to EGFR.

Numbered embodiment 137. The method of any one of numbered embodiments 102-136, wherein the Kd of the binding agent is less than the binding affinity of Cetuximab to EGFR.

Numbered embodiment 138. The method of any one of numbered embodiments 102-137, wherein the Kd of the binding agent is more than the binding affinity of Cetuximab to EGFR.

Numbered embodiment 139. The method of any one of numbered embodiments 102-138, wherein binding of the binding agent to EGFR is configured to block the binding of epidermal growth factor (EGF).

Numbered embodiment 140. The method of any one of numbered embodiments 102-139, wherein the binding agent is configured to bind an epitope that overlaps with a cetuximab epitope.

Numbered embodiment 141. The method of any one of numbered embodiments 102-140, wherein the second antigen binding domain is configured to not bind to a non-ITGB6 epitope.

Numbered embodiment 142. The method of any one of numbered embodiments 102-141, wherein the second antigen binding domain is configured to bind to an epitope of ITGB6 on the target cell, wherein the epitope does not comprise an epitope to which latency-associated peptide (LAP) binds.

Numbered embodiment 143. The binding agent of any one of numbered embodiments 1-96, wherein the binding agent comprises: (a) a first CH1 domain (CH1) and a first CL domain (CL), the first CH1 domain and the first CL domain interacting together at a first CHCL interface to form a first CHCL domain (CH CL); (b) a second CH1 domain (CH1) and a second CL domain (CL), the second CH1 domain and the second CL domain interacting together at a second CHCL interface to form a second CHCL domain (CH CL); wherein the first CH1 domain and/or the second CH1 domain have at least one mutation relative to a human immunoglobulin CH1 domain, referred to as the CH1 mutant residue(s), and wherein each CH1 mutant residue is only present in one of first CH1 domain or the second CH1 domain; wherein the first CL domain and/or the second CL domain have at least one mutation relative to a human immunoglobulin CL domain, referred to as the CL mutant residue(s), and wherein each CL mutant residue is only present in one of first CL domain or the second CL domain; and wherein the first CH1 domain is attached to a first variable heavy domain (VH), and the first CL domain is attached to a first variable light domain (VL), and the second CH1 domain is attached to a second VH domain, and the second CL domain is attached to a second VL domain, such that when combined, the first VH domain, first VL domain, first CH domain and first CL domain together form a first Fab, and when combined, the second VH domain, second VL domain, second CH1 domain, and second CL domain form a second Fab.

Numbered embodiment 144. A binding agent, comprising: (a) a first CH1 domain (CH1) and a first CL domain (CL), the first CH1 and the first CL interacting together at a first CHCL interface to form a first CHCL domain (CH CL); (b) a second CH1 domain (CH1) and a second CL domain (CL), the second CH1 and the second CL interacting together at a second CHCL interface to form a second CHCL domain (CH CL); wherein the first CH1 domain and/or the second CH1 domain have at least one mutation relative to a human immunoglobulin CH1 domain, referred to as the CH1 mutant residue(s), and wherein each CH1 mutant residue is only present in one of first CH1 domain or the second CH1 domain; wherein the first CL domain and/or the second CL domain have at least one mutation relative to a human immunoglobulin CL domain, referred to as the CL mutant residue(s), and wherein each CL mutant residue is only present in one of first CL domain or the second CL domain; wherein the CH1 mutant residue(s) and the CL mutant residue(s) comprise charged amino acids such that a first CH1 mutant residue and a first CL mutant residue comprise a charge pair, and wherein the first CH1 mutant residue is located at H172 and/or T192 and the first CL mutant residue is located at N137 and/or N138; or the CH1 mutant residue(s) and the CL mutant residue(s) comprise a steric pair such that (a) (i) a first CH1 mutant residue has steric conflict with the first CL domain or the second CL domain or (ii) a first CL mutant residue has steric conflict with the first CH1 domain or the second CH1 domain and (b) the first CH1 mutant residue and the first CL mutant residue do not have steric conflict, and wherein the first CH1 mutant residue is located at L124 and/or G141 and the a first CL mutant residue is located at F116 and/or F118; and wherein the first CH1 domain is attached to a first variable heavy domain (VH), and the first CL domain is attached to a first variable light domain (VL), and the second CH1 domain is attached to a second VH domain, and the second CL domain is attached to a second VL domain, such that when combined, the first VH domain, first VL domain, first CH domain and first CL domain together form a first Fab, and when combined, the second VH domain, second VL domain, second CH1 domain, and second CL domain form a second Fab.

Numbered embodiment 145. A binding agent, comprising: (a) a first CH1 domain (CH1) and a first CL domain (CL), the first CH1 and the first CL interacting together at a first CHCL interface to form a first CHCL domain (CH CL); (b) a second CH1 domain (CH1) and a second CL domain (CL), the second CH1 and the second CL interacting together at a second CHCL interface to form a second CHCL domain (CH CL); wherein the first CH1 domain and/or the second CH1 domain have at least two mutations relative to a human immunoglobulin CH1 domain, referred to as the CH1 mutant residues, and wherein each CH1 mutant residue is only present in one of first CH1 domain or the second CH1 domain; wherein the first CL domain and/or the second CL domain have at least two mutations relative to a human immunoglobulin CL domain, referred to as the CL mutant residues, and wherein each CL mutant residue is only present in one of first CL domain or the second CL domain; wherein the CH1 mutant residues and the CL mutant residues comprise charged amino acids such that a first CH1 mutant residue and the a CL mutant residue comprise a charge pair; wherein the CH1 mutant residues and the CL mutant residues comprise a steric pair such that (a) (i) the second CH1 mutant residue has steric conflict with the first CL domain or the second CL domain or (ii) the second CL mutant residue has steric conflict with the first CH1 domain or the second CH1 domain and (b) the second CH1 mutant residue and the second CL mutant residue do not have steric conflict; and wherein the first CH1 domain is attached to a first variable heavy domain (VH) domain, and the first CL domain is attached to a first variable light domain (VL) domain, and the second CH1 domain is attached to a second VH domain, and the second CL domain is attached to a second VL domain, such that when combined, the first VH domain, first VL domain, first CH domain and first CL domain together form a first Fab, and when combined, the second VH domain, second VL domain, second CH1 domain, and second CL domain form a second Fab.

Numbered embodiment 146. A binding agent, comprising: (a) a first CH1 domain (CH1) and a first CL domain (CL), the first CH1 and the first CL interacting together at a first CHCL interface to form a first CHCL domain (CH CL); (b) a second CH1 domain (CH1) and a second CL domain (CL), the second CH1 and the second CL interacting together at a second CHCL interface to form a second CHCL domain (CH CL); wherein the first CH1 domain and/or the second CH1 domain has at least one mutation relative to a human immunoglobulin CH1 domain, referred to as the CH1 mutant residue(s), and wherein each CH1 mutant residue is only present in one of first CH1 domain or the second CH1 domain; wherein the first CL domain and/or the second CL domain has at least one mutation relative to a human immunoglobulin CL domain, referred to as the CL mutant residue(s), and wherein each CL mutant residue is only present in one of first CL domain or the second CL domain; wherein the CH1 mutant residue and the CL mutant residue comprise charged amino acids such that a first CH1 mutant residue and a first CL mutant residue comprise a charge pair; or the CH1 mutant residues and the CL mutant residues comprise a steric pair such that (a) (i) a first CH1 mutant residue has steric conflict with the first CL domain or the second CL domain or (ii) a first CL mutant residue has steric conflict with the first CH1 domain or the second CH1 domain and (b) the second CH1 mutant residue and the second CL mutant residue do not have steric conflict; wherein the first CH1 domain is attached to a first variable heavy domain (VH), and the first CL domain is attached to a first variable light domain (VL), and the second CH1 domain is attached to a second VH domain, and the second CL domain is attached to a second VL domain, wherein the first VH domain or the second VH domain has at least one mutation relative to a human immunoglobulin VH domain, referred to as the VH mutant residue(s); and the first VL domain or the second VL domain has at least one mutation relative to a human immunoglobulin VL domain, referred to as the VL mutant residue(s), and wherein the first VH domain, first VL domain, first CH domain and first CL domain together form a first Fab, and when combined, the second VH domain, second VL domain, second CH1 domain, and second CL domain form a second Fab.

Numbered embodiment 147. The binding agent of any one of numbered embodiments 143-146, wherein the CH1 mutant residue(s) and the CL mutant residue(s) comprise charged amino acids such that the CH1 mutant residue(s) and the CL mutant residue(s) comprise a charge pair.

Numbered embodiment 148. The binding agent of numbered embodiment 147, wherein the CH1 mutant residue(s) and the CL mutant residue(s) comprise at least two charge pairs.

Numbered embodiment 149. The binding agent of numbered embodiment 148, wherein the charge pair(s) comprise at least one charge pair located (i) at H172 and/or T192 in the first CH1 domain and at N137 and/or N138 in the first CL domain; and/or (ii) at H172 and/or T192 in the second CH1 domain and at N137 and/or N138 in the second CL domain.

Numbered embodiment 150. The binding agent of any one of numbered embodiments 147-149, wherein the CH1 mutant residue(s) comprise an arginine, a histidine, or a lysine, and wherein the CL mutant residue(s) comprise an aspartic acid or a glutamic acid.

Numbered embodiment 151. The binding agent of any one of numbered embodiments 143-150, wherein the CH1 mutant residue(s) comprise an aspartic acid or a glutamic acid, and wherein the CL mutant residue(s) comprise an arginine, a histidine, or a lysine.

Numbered embodiment 152. The binding agent of any one of numbered embodiments 151, wherein the charge pair(s) comprise at least one charge pair comprising (i) H172K and/or T192K in the first CH1 domain and N137D and/or N138D in the first CL domain; and/or (ii) H172D and/or T192D in the second CH1 domain and N137K and/or N138K in the second CL domain.

Numbered embodiment 153. The binding agent of any one of numbered embodiments 147-152, wherein the charge pairs comprise at least one charge pair on the first CHCL domain and at least one charge pair on the second CHCL domain located at the same positions.

Numbered embodiment 154. The binding agent of numbered embodiment 153, wherein the CH1 mutant residue(s) in the first CHCL domain and the CH1 mutant residue(s) in the second CHCL domain are opposing charges.

Numbered embodiment 155. The binding agent of any one of numbered embodiments 143-154, wherein the CH1 mutant residue(s) and the CL mutant residue(s) comprise a steric pair.

Numbered embodiment 156. The binding agent of numbered embodiment 155, wherein the CH1 mutant residue(s) and the CL mutant residue(s) comprise at least two steric pairs.

Numbered embodiment 157. The binding agent of numbered embodiment 156, wherein the steric pair(s) comprise at least one steric pair located (i) at L124 and/or G141 in the first CH1 domain and at F116 and/or F118 in the first CL domain; and/or (ii) at L124 and/or G141 in the second CH1 domain and at F116 and/or F118 in the second CL domain.

Numbered embodiment 158. The binding agent of any one of numbered embodiments 156-157, wherein the steric pair(s) comprise at least one steric pair comprising (i) L124S and/or G141L in the first CH1 domain and F116T and/or F118M in the first CL domain; and/or (ii) L124S and/or G141L in the second CH1 domain and F116T and/or F118M in the second CL domain.

Numbered embodiment 159. The binding agent of any one of numbered embodiments 157-158, wherein the first CHCL domain comprises at least one charge pair and the second CHCL domain comprises at least one steric pair.

Numbered embodiment 160. The binding agent of any one of numbered embodiments 143-159, wherein the first CH1 domain comprises an amino acid sequence having at least 75%, at least 80%, at least 85%, at least 90%, at least 95%, at least 99%, or 100% sequence identity to any one of SEQ ID NO: 118, 122, 126, 130, 134, and 138.

Numbered embodiment 161. The binding agent of any one of numbered embodiments 143-160, wherein the first CL domain comprises an amino acid sequence having at least 75%, at least 80%, at least 85%, at least 90%, at least 95%, at least 99%, or 100% sequence identity to any one of SEQ ID NO: 119, 123, 127, 131, 135, and 139.

Numbered embodiment 162. The binding agent of any one of numbered embodiments 143-161, wherein the second CH1 domain comprises an amino acid sequence having at least 75%, at least 80%, at least 85%, at least 90%, at least 95%, at least 99%, or 100% sequence identity to any one of SEQ ID NO: 120, 124, 128, 132, 136, and 140.

Numbered embodiment 163. The binding agent of any one of numbered embodiments 143-162, wherein the second CL domain comprises an amino acid sequence having at least 75%, at least 80%, at least 85%, at least 90%, at least 95%, at least 99%, or 100% sequence identity to any one of SEQ ID NO: 121, 125, 129, 133, 137, and 141.

Numbered embodiment 164. The binding agent of any one of numbered embodiments 1-163, wherein the binding agent comprises an amino acid sequence having at least 75%, at least 80%, at least 85%, at least 90%, at least 95%, at least 99%, or 100% sequence identity to any one of SEQ ID NO: 108-113.

Numbered embodiment 165. The binding agent of any one of numbered embodiments 1-164, wherein the binding agent comprises an amino acid sequence having at least 75%, at least 80%, at least 85%, at least 90%, at least 95%, at least 99%, or 100% sequence identity to any one of SEQ ID NO: 114-117.

Numbered embodiment 166. The binding agent of numbered embodiment 165, wherein the binding agent comprises an amino acid sequence of SEQ ID NO: 114 and an amino acid sequence of SEQ ID NO: 115.

Numbered embodiment 167. The binding agent of numbered embodiment 165 or numbered embodiment 166, wherein the binding agent comprises an amino acid sequence of SEQ ID NO: 116 and an amino acid sequence of SEQ ID NO: 117.

Numbered embodiment 168. The binding agent of numbered embodiment 167, wherein the binding agent comprises: (i) an amino acid sequence of SEQ ID NO: 114, (ii) an amino acid sequence of SEQ ID NO: 115, (iii) an amino acid sequence of SEQ ID NO: 116, and (iv) an amino acid sequence of SEQ ID NO: 117.

Numbered embodiment 169. The binding agent of any one of numbered embodiments 143-168, wherein the CH1 mutant residue(s) and the CL mutant residue(s) interact with each other in preference to corresponding non-mutated CH1 residue(s) or corresponding non-mutated CL residue(s).

Numbered embodiment 170. The binding agent of numbered embodiment 169, wherein the CH1 mutant residue(s) repel a CL domain comprising the corresponding non-mutated CL residue(s) or the CL mutant residue(s) repel a CH1 domain comprising the corresponding non-mutated CH1 residue(s).

Numbered embodiment 171. The binding agent of any one of numbered embodiments 143-170, wherein the first VH domain or the second VH domain has at least one mutation relative to a human immunoglobulin VH domain, referred to as the VH mutant residue(s); and the first VL domain or the second VL domain has at least one mutation relative to a human immunoglobulin VL domain, referred to as the VL mutant residue(s).

Numbered embodiment 172. The binding agent of numbered embodiment 171, wherein the VH mutant residue(s) and the VL mutant residue(s) comprise a disulfide bridge pair.

Numbered embodiment 173. The binding agent of numbered embodiment 172, wherein the VH mutant residue(s) and the VL mutant residue(s) comprise at least two disulfide bridge pairs.

Numbered embodiment 174. The binding agent of numbered embodiment 173, wherein the disulfide bridge pair(s) comprise at least one disulfide bridge pair located (i) at G44 in the first VH domain and at G100 in the first VL domain; and/or (ii) at G44 in the second VH domain and at G100 in the second VL domain.

Numbered embodiment 175. The binding agent of any one of numbered embodiments 172-174, wherein the disulfide bridge pair(s) comprise at least one disulfide bridge pair comprising (i) G44C in the first VH domain and G100C in the first VL domain; and/or (ii) G44C in the second VH domain and G100C in the second VL domain.

Numbered embodiment 176. The binding agent of any one of numbered embodiments 171-175, wherein the first CHCL domain comprises at least one charge pair or at least one steric pair, and where the second VH domain and the second VL domain comprise the VH mutant residue(s).

Numbered embodiment 177. A method of making a bispecific binding agent comprising: (i) providing a first CH1 domain, a second CH1 domain, a first CL domain, and a second CL domain, wherein the first CH1 domain and/or the second CH1 domain have at least one mutation relative to a human immunoglobulin CH1 domain, referred to as the CH1 mutant residue(s), and wherein each CH1 mutant residue is only present in one of first CH1 domain or the second CH1 domain; wherein the first CL domain and/or the second CL domain have at least one mutation relative to a human immunoglobulin CL domain, referred to as the CL mutant residue(s), and wherein each CL mutant residue is only present in one of first CL domain or the second CL domain; and (ii) mixing the first CH1 domain, the second CH1 domain, the first CL domain, and the second CL domain, thereby generating a plurality of bispecific binding agents.

Numbered embodiment 178. The method of numbered embodiment 177, wherein the plurality of bispecific binding agents comprises the desired heterodimer, and wherein the purity of the desired heterodimer relative to total heterodimers is at least 60%, at least 70%, at least 80%, at least 90%, at least 95%, at least 97%, at least 98%, or at least 99%.

### Examples

The application may be better understood by reference to the following non-limiting examples, which are provided as exemplary embodiments of the application. The following examples are included for illustrative purposes only and are not intended to limit the scope of the inventive concepts.

### Example 1—Bispecific Antibody Expression

Bispecifics are expressed as half IgGs and purified from mammalian cells (exemplary: Expi293F, ExpiCHO-S) using transient transfection following the manufacturer's protocol. At designated time point (exemplary: 4-14 days), media is harvested by centrifugation at 4,000×g for 20 minutes. Knob half IgGs and hole half IgGs are purified by Protein A affinity chromatography and buffer exchanged into PBS containing 20% glycerol, concentrated, and flash frozen for storage at −80° C. Knob and hole half IgGs are recombined under reducing conditions (exemplary: 10 mM Tris pH 7.5, 100 mM NaCl, 20% 800 mM L-Arg pH 8.5 plus 200-fold excess reduced glutathione), purified by cation exchange chromatography, buffer exchanged or SEC purified into 10 mM histidine, 10 mg/mL arginine, 5% trehalose, pH 6.0, concentrated, and then flash frozen for storage at −80° C. Purity and integrity of all proteins are assessed by SDS-PAGE and SEC.

### Example 2—EGFRxITGB6 Bispecific Antibodies Inhibit Tumor Growth in Mice

This example sought to determine whether bispecific antibodies that bind to EGFR and ITGB6 could pharmacologically inhibit tumor growth in mouse tumor models.

For this experiment, NCI-H1975 (non-small cell lung cancer carrying an EGFR mutation (T790M and L858R), and PIK3CA mutation typically found in NSCLC) cell lines were grown in tissue culture flasks containing RPMI-1640 medium supplemented with 10% fetal bovine serum (FBS) at 37° C. in an atmosphere of 5% CO2. Cells were harvested during exponential growth phase, and 5×10⁶ total cells were inoculated into the right front flank of BALB/c nude mice. Animals were monitored daily, and tumor volumes and body weights were measured twice per week according to IACUC guidelines. Tumors were grown until reaching approximately a volume of 200 mm3, at which point animals were randomized into groups (n=6/group) and given intraperitoneal doses of (1) bispecific antibodies that bind to EGFR and ITGB6, (2) bispecific antibodies that bind to EGFR and MUC1, (3) bispecific antibodies that bind to EGFR and RSV (single arm control), or (4) an isotype control. All antibodies that bound to EGFR had an ES11 EGFR binding arm. Bispecific antibodies were prepared in-house. Isotype control antibodies were purchased from BioXcell (BP0297). Dosing continued twice-per-week for 2 weeks and animals were monitored for up to 50 days from the initial dose. Tumor volume was calculated as V=(L×W×W)/2. Graphs and statistical analysis were done in Graphpad Prism using ordinary one-way ANOVA w/Tukey's multiple comparisons test, *p<0.05, **p<0.01, ***p<0.001.

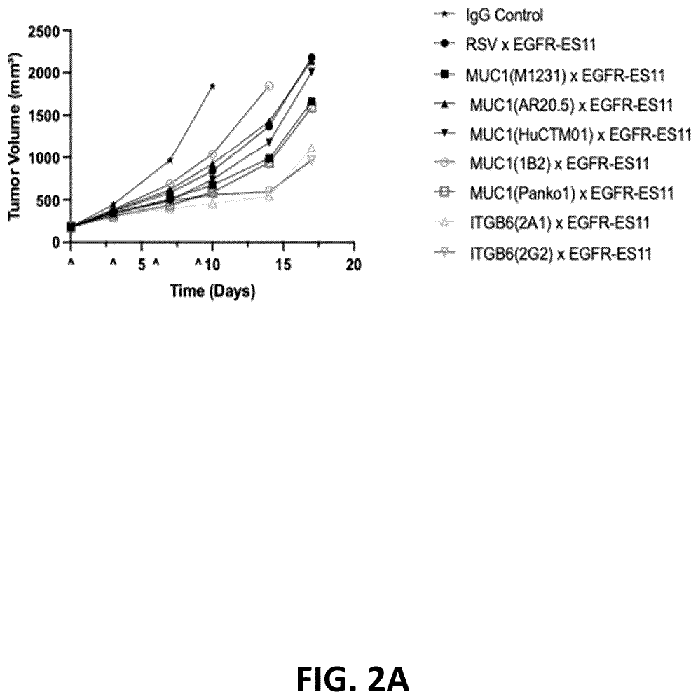

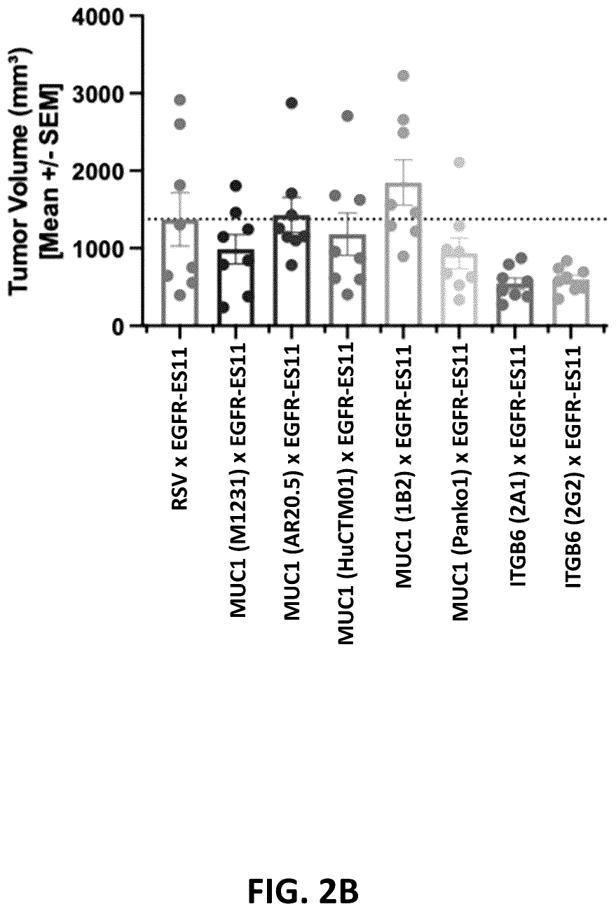

Tumor growth inhibition was compared between groups (FIGS. 2A and 2B; IgG Control, RSV×EGFR-ES11, MUC1 (M1231)×EGFR-ES11, MUC1 (AR20.5)×EGFR-ES11, MUC1 (HuCTMO1)×EGFR-ES 11, MUC1 (1B2)×EGFR-ES11, MUC1 (Pankol)×EGFR-ES11, ITGB6 (2A1)×EGFR-ES11, and ITGB6 (2G2)×EGFR-ES11). Bispecific antibodies that bind to EGFR and ITGB6 (ITGB6 (2A1)×EGFR-ES11, and ITGB6 (2G2)×EGFR-ES 11) and bispecific antibodies that bind to EGFR and MUC1 (MUC1 (M1231)×EGFR-ES11, MUC1 (AR20.5)×EGFR-ES11, MUC1 (HuCTMO1)×EGFR-ES11, MUC1 (1B2)×EGFR-ES11, MUC1 (Pankol)×EGFR-ES11) both demonstrated decreased tumor growth when compared to the isotype control or the single arm control. The strongest inhibition was seen with the bispecific antibodies that bind to EGFR and ITGB6 (ITGB6 (2A1)×EGFR-ES 11), and ITGB6 (2G2)×EGFR-ES 11).

### Example 3—Discovery of Human-Mouse Cross-Reactive Antibodies A2ainst EGFR

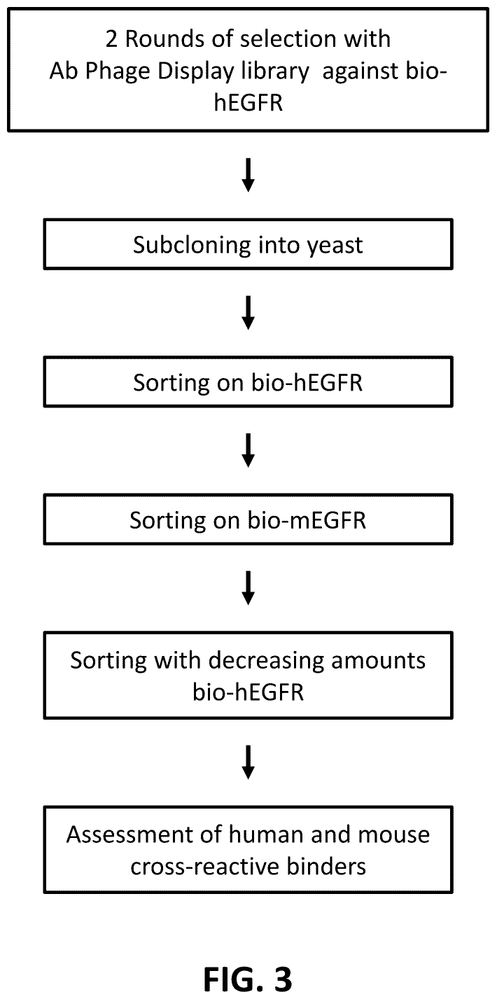

A panel of antibodies against EGFR was discovered from a scFv antibody library displayed on phage and yeast. The antibody library was built on well-behaved clinical antibody scaffolds and grafted with natural complementary-determining regions (CDRs) informatically purged of sequence liabilities from human antibodies, except HCDR3s which were amplified from B cells. The discovery was performed as follows (as shown in FIG. 3), the first 2 rounds of selection were performed with the library displayed on phage against biotinylated human EGFR, followed by the subcloning of the selected pool into yeast for further selection rounds. The subcloned library displayed on yeast was then sorted for binders on human and mouse biotinylated EGFR to select for cross-reactive human and mouse binders. Subsequent sorting was done with decreasing amounts of biotinylated human EGFR to drive the selection to high-affinity binders.

The human and mouse cross-reactive antibody panel was then cloned, expressed, purified, and quality controlled as IgG1. They were assessed for cell binding, polyreactivity, self-association, melting temperature, SEC RT, acid stability at pH 3.0, expression titer, and binding kinetics to human, mouse, and Cynomolgus monkey EGFR. This set of data allowed the selection of 4 clones (ES11, ES20, ES21, and ES30) for biological activity assessment.

### Example 4—Lead Optimization of Clone ES11

A mutational scan of clone ES11 was performed to remove sequence liabilities and modulate binding affinity to EGFR. A panel of alanine mutations and germline reversion mutations listed in Table 3 were generated and assessed for binding by SPR. Mutations M28T to remove an oxidation site and R53A and S98A to improve affinity were combined to make a high affinity clone ES11v23.

A second round of mutational analysis was performed to weaken the affinity of clone ESI1v23. An expanded panel of alanine mutations and germline reversion mutations listed in Table 4 were made with respect to ESI1v23 as the parental antibody. Binding kinetics to EGFR were measured by SPR. Clones ES11v37 and ES11v38 were selected as candidates to explore the impact of weakened EGFR affinity.

TABLE 3
ES11 Mutational Scan to Remove Sequence Liabilities
and Modulate Binding Affinity to EGFR

| | | huEGFR Kd |
|---|---|---|
| Clone | Mutation | (nM) |
| ES11 (SEQ ID NOs: 126, 181, 236, 291, | — | 1.3 |
| 346, 401, 456, 511, 566, 621) | | |
| ES11v2 (SEQ ID NOs: 127, 182, 237, 292, | HC: M285 | 1.1 |
| 347, 402, 457, 512, 567, 622) | | |
| ES11v3 (SEQ ID NOs: 128, 183, 238, 293, | HC: R53A | 0.73 |
| 348, 403, 458, 513, 568, 623) | | |
| ES11v4 (SEQ ID NOs: 129, 184, 239, 294, | HC: S56A | 1.1 |
| 349, 404, 459, 514, 569, 624) | | |
| ES11v5 (SEQ ID NOs: 130, 185, 240, 295, | HC: C58Y | 1.8 |
| 350, 405, 460, 515, 570, 625) | | |
| ES11v6 (SEQ ID NOs: 131, 186, 241, 296, | HC: E64K | 1.4 |
| 351, 406, 461, 516, 571, 626) | | |
| ES11v7 (SEQ ID NOs: 132, 187, 242, 297, | HC: E97A | 1.8 |
| 352, 407, 462, 517, 572, 627) | | |
| ES11v8 (SEQ ID NOs: 133, 188, 243, 298, | HC: S98A | 0.8 |
| 353, 408, 463, 518, 573, 628) | | |
| ES11v9 (SEQ ID NOs: 134, 189, 244, 299, | HC: Y100A | 5.2 |
| 354, 409, 464, 519, 574, 629) | | |
| ES11v10 (SEQ ID NOs: 135, 190, 245, 300, | HC: Y100aA | 1.5 |
| 355, 410, 465, 520, 575, 630) | | |
| ES11v11 (SEQ ID NOs: 136, 191, 246, 301, | HC: P100cF | 5.2 |
| 356, 411, 466, 521, 576, 631) | | |
| ES11v12 (SEQ ID NOs: 137, 192, 247, 302, | LC: R31N | 1.4 |
| 357, 412, 467, 522, 577, 632) | | |
| ES11v13 (SEQ ID NOs: 138, 193, 248, 303, | LC: F32A | 3.3 |
| 358, 413, 468, 523, 578, 633) | | |
| ES11v14 (SEQ ID NOs: 139, 194, 249, 304, | LC: V51A | 1.5 |
| 359, 414, 469, 524, 579, 634) | | |
| ES11v15 (SEQ ID NOs: 140, 195, 250, 305, | LC: N53T | 1.5 |
| 360, 415, 470, 525, 580, 635) | | |
| ES11v16 (SEQ ID NOs: 141, 196, 251, 306, | LC: Y91A | 1.3 |
| 361, 416, 471, 526, 581, 636) | | |
| ES11v17 (SEQ ID NOs: 142, 197, 252, 307, | LC: S92A | 1.4 |
| 362, 417, 472, 527, 582, 637) | | |
| ES11v18 (SEQ ID NOs: 143, 198, 253, 308, | LC: T93A | 1.1 |
| 363, 418, 473, 528, 583, 638) | | |
| ES11v19 (SEQ ID NOs: 144, 199, 254, 309, | LC: S94A | 1.6 |
| 364, 419, 474, 529, 584, 639) | | |
| ES11v20 (SEQ ID NOs: 145, 200, 255, 310, | LC: V95P | 1.8 |
| 365, 420, 475, 530, 585, 640) | | |
| ES11v21 (SEQ ID NOs: 146, 201, 256, 311, | HC: M28T + R53A | 0.17 |
| 366, 421, 476, 531, 586, 641) | | |
| ES11v22 (SEQ ID NOs: 147, 202, 257, 312, | HC: M28T + S98A | 0.2 |
| 367, 422, 477, 532, 587, 642) | | |
| ES11v23 (SEQ ID NOs: 148, 203, 258, 313, | HC: M28T + R53A + S98A | 0.13 |
| 368, 423, 478, 533, 588, 643) | | |
| ES11v24 (SEQ ID NOs: 149, 204, 259, 314, | HC: M28T + Y100A + LC: | Poor fit |
| 369, 424, 479, 534, 589, 644) | F32A | |
| ES11v25 (SEQ ID NOs: 150, 205, 260, 315, | HC: M28T + Y100A + | Poor fit |
| 370, 425, 480, 535, 590, 645) | P100cF + LC: F32A | |

TABLE 4
ES11v23 Mutational Scan to Weaken Affinity

| | | huEGFR Kd |
|---|---|---|
| Clone | Mutation | (nM) |
| ES11v23 (SEQ ID NOs: 148, 203, 258, 313, | — | 0.27 |
| 368, 423, 478, 533, 588, 643) | | |
| ES11v26 (SEQ ID NOs: 151, 206, 261, 316, | HC: D31A | 1.0 |
| 371, 426, 481, 536, 591, 646) | | |
| ES11v27 (SEQ ID NOs: 152, 207, 262, 317, | HC: Y32A | 1.4 |
| 372, 427, 482, 537, 592, 647) | | |
| ES11v28 (SEQ ID NOs: 153, 208, 263, 318, | HC: G33A | 3.2 |
| 373, 428, 483, 538, 593, 648) | | |
| ES11v29 (SEQ ID NOs: 154, 209, 264, 319, | HC: D52S | 0.35 |
| 374, 429, 484, 539, 594, 649) | | |
| ES11v30 (SEQ ID NOs: 155, 210, 265, 320, | HC: G54S | 0.33 |
| 375, 430, 485, 540, 595, 650) | | |
| ES11v31 (SEQ ID NOs: 156, 211, 266, 321, | HC: G55A | 0.28 |
| 376, 431, 486, 541, 596, 651) | | |
| ES11v32 (SEQ ID NOs: 157, 212, 267, 322, | HC: T57A | 0.26 |
| 377, 432, 487, 542, 597, 652) | | |
| ES11v33 (SEQ ID NOs: 158, 213, 268, 323, | HC: D58A | 0.44 |
| 378, 433, 488, 543, 598, 653) | | |
| ES11v34 (SEQ ID NOs: 159, 214, 269, 324, | HC: D58G | 0.55 |
| 379, 434, 489, 544, 599, 654) | | |
| ES11v35 (SEQ ID NOs: 160, 215, 270, 325, | HC: D95A | Poor fit |
| 380, 435, 490, 545, 600, 655) | | |
| ES11v36 (SEQ ID NOs: 161, 216, 271, 326, | HC: L96A | 0.25 |
| 381, 436, 491, 546, 601, 656) | | |
| ES11v37 (SEQ ID NOs: 162, 217, 272, 327, | HC: G99A | 55 |
| 382, 437, 492, 547, 602, 657) | | |
| ES11v38 (SEQ ID NOs: 163, 218, 273, 328, | HC: G100bA | 6.4 |
| 383, 438, 493, 548, 603, 658) | | |
| ES11v39 (SEQ ID NOs: 164, 219, 274, 329, | LC: D28G | 0.45 |
| 384, 439, 494, 549, 604, 659) | | |
| ES11v40 (SEQ ID NOs: 165, 220, 275, 330, | LC: G30S | 0.52 |
| 385, 440, 495, 550, 605, 660) | | |
| ES11v41 (SEQ ID NOs: 166, 221, 276, 331, | LC: R31S | 0.28 |
| 386, 441, 496, 551, 606, 661) | | |
| ES11v42 (SEQ ID NOs: 167, 222, 277, 332, | LC: F32Y | 0.26 |
| 387, 442, 497, 552, 607, 662) | | |
| ES11v43 (SEQ ID NOs: 168, 223, 278, 333, | LC: S52A | 0.24 |
| 388, 443, 498, 553, 608, 663) | | |
| ES11v44 (SEQ ID NOs: 169, 224, 279, 334, | LC: N53A | 0.26 |
| 389, 444, 499, 554, 609, 664) | | |
| ES11v45 (SEQ ID NOs: 170, 225, 280, 335, | LC: L54A | 0.28 |
| 390, 445, 500, 555, 610, 665) | | |
| ES11v46 (SEQ ID NOs: 171, 226, 281, 336, | LC: Q55A | 0.58 |
| 391, 446, 501, 556, 611, 666) | | |
| ES11v47 (SEQ ID NOs: 172, 227, 282, 337, | LC: S56A | 0.27 |
| 392, 447, 502, 557, 612, 667) | | |
| ES11v48 (SEQ ID NOs: 173, 228, 283, 338, | LC: V95A | 0.44 |
| 393, 448, 503, 558, 613, 668) | | |

TABLE 5
Amino Acid Sequences

| SEQ | | | |
|---|---|---|---|
| ID | | | |
| NO. | Antigen | Sequence | Domain |
| 126 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 127 | EGFR | EVQLVESGGGLVQPGRSLRLSCAASGFTFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 128 | EGFR | EVQLVESGGGLVQPGRSLRLSCAASGFMFSDYGMHWVRQAPGKGLEWVSA | Heavy |
| | | IDAGGSTDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 129 | EGFR | EVQLVESGGGLVQPGRSLRLSCAASGFMFSDYGMHWVRQAPGKGLEWVSA | Heavy |
| | | IDRGGATDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 130 | EGFR | EVQLVESGGGLVQPGRSLRLSCAASGFMFSDYGMHWVRQAPGKGLEWVSA | Heavy |
| | | IDRGGSTYYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 131 | EGFR | EVQLVESGGGLVQPGRSLRLSCAASGFMFSDYGMHWVRQAPGKGLEWVSA | Heavy |
| | | IDRGGSTDYADSVKGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 132 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLA | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 133 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 134 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGAYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 135 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGYAGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 136 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGYYGFDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 137 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 138 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 139 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 140 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 141 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 142 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 143 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 144 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 145 | EGFR | EVQLVESGGGLVQPGRSLRLSCAASGFMFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 146 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | SGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 147 | EGFR | EVQLVESGGGLVQPGRSLRLSCAASGFTFSDYGMHWVRQAPGKGLEWVSA | Heavy |
| | | IDRGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 148 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 149 | EGFR | EVQLVESGGGLVQPGRSLRLSCAASGFTFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGAYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 150 | EGFR | EVQLVESGGGLVQPGRSLRLSCAASGFTFSDYGMHWVRQAPGKGLEWVSA | Heavy |
| | | IDRGGSTDYADSVEGRFTISRDNAKNSLYLQMNSLRAEDTAVYYCAKDLE | Chain |
| | | SGAYGFDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 151 | EGFR | EVQLVESGGGLVQPGRSLRLSCAASGFTFSAYGMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 152 | EGFR | EVQLVESGGGLVQPGRSLRLSCAASGFTFSDAGMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 153 | EGFR | EVQLVESGGGLVQPGRSLRLSCAASGFTFSDYAMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 154 | EGFR | EVQLVESGGGLVQPGRSLRLSCAASGFTFSDYGMHWVRQAPGKGLEWVSA | Heavy |
| | | ISAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 155 | EGFR | EVQLVESGGGLVQPGRSLRLSCAASGFTFSDYGMHWVRQAPGKGLEWVSA | Heavy |
| | | IDASGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 156 | EGFR | EVQLVESGGGLVQPGRSLRLSCAASGFTFSDYGMHWVRQAPGKGLEWVSA | Heavy |
| | | IDAGASTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 157 | EGFR | EVQLVESGGGLVQPGRSLRLSCAASGFTFSDYGMHWVRQAPGKGLEWVSA | Heavy |
| | | IDAGGSADYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 158 | EGFR | EVQLVESGGGLVQPGRSLRLSCAASGFTFSDYGMHWVRQAPGKGLEWVSA | Heavy |
| | | IDAGGSTAYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 159 | EGFR | EVQLVESGGGLVQPGRSLRLSCAASGFTFSDYGMHWVRQAPGKGLEWVSA | Heavy |
| | | IDAGGSTGYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 160 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKALE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 161 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDAE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 162 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | AAYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 163 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | AGYYAPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 164 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 165 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 166 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 167 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 168 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 169 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 170 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 171 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 172 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 173 | EGFR | EVQLVESGGGLVQPGRSLRLSCAASGFTFSDYGMHWVRQAPGKGLEWVSA | Heavy |
| | | IDAGGSTDYADSVEGRFTISRDNAKNSLYLQMNSLRAEDTAVYYCAKDLE | Chain |
| | | AGYYGPDVWGKGTTVTVSSASTKGPSVFPLAPSSKSTSGGTAALGCLVKD | (VH-CH) |
| | | YFPEPVTVSWNSGALTSGVHTFPAVLQSSGLYSLSSVVTVPSSSLGTQTY | |
| | | ICNVNHKPSNTKVDKKVEPKSCDKTHTCPPCPAPELLGGPSVFLFPPKPK | |
| | | DTLMISRTPEVTCVVVDVSHEDPEVKFNWYVDGVEVHNAKTKPREEQYNS | |
| | | TYRVVSVLTVLHQDWLNGKEYKCKVSNKALPAPIEKTISKAKGQPREPQV | |
| | | YTLPPSRDELTKNQVSLTCLVKGFYPSDIAVEWESNGQPENNYKTTPPVL | |
| | | DSDGSFFLYSKLTVDKSRWQQGNVFSCSVMHEALHNHYTQKSLSLSPGK | |
| 174 | EGFR | QVQLVQSGAEVKKPGASVKVSCKVSGDTFSNQGISWVRQAPGKGLEWMGG | Heavy |
| | | FDPDAGETIYAQKFQGRVTMTEDTSTDTAYMELSSLKSEDTAVYYCARGV | Chain |
| | | DSYGYGRYNWFDPWGQGTLVTVSSASTKGPSVFPLAPSSKSTSGGTAALG | (VH-CH) |
| | | CLVKDYFPEPVTVSWNSGALTSGVHTFPAVLQSSGLYSLSSVVTVPSSSL | |
| | | GTQTYICNVNHKPSNTKVDKKVEPKSCDKTHTCPPCPAPELLGGPSVFLF | |
| | | PPKPKDTLMISRTPEVTCVVVDVSHEDPEVKFNWYVDGVEVHNAKTKPRE | |
| | | EQYNSTYRVVSVLTVLHQDWLNGKEYKCKVSNKALPAPIEKTISKAKGQP | |
| | | REPQVYTLPPSRDELTKNQVSLTCLVKGFYPSDIAVEWESNGQPENNYKT | |
| | | TPPVLDSDGSFFLYSKLTVDKSRWQQGNVFSCSVMHEALHNHYTQKSLSL | |
| | | SPGK | |
| 175 | EGFR | QVQLQESGPGLVKPSQTLSLTCTVSGASIRSGDYSWSWIRQPPGKGLEWI | Heavy |
| | | GYIEHSGRTDYNPSLKSRVTMSVDTSKNQFSLKVNSVTAADTAVYYCARA | Chain |
| | | SGWYGVYDAFDVWGKGTTVTVSSASTKGPSVFPLAPSSKSTSGGTAALGC | (VH-CH) |
| | | LVKDYFPEPVTVSWNSGALTSGVHTFPAVLQSSGLYSLSSVVTVPSSSLG | |
| | | TQTYICNVNHKPSNTKVDKKVEPKSCDKTHTCPPCPAPELLGGPSVFLFP | |
| | | PKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPR | |
| | | EPQVYTLPPSRDELTKNQVSLTCLVKGFYPSDIAVEWESNGQPENNYKTT | |
| | | PPVLDSDGSFFLYSKLTVDKSRWQQGNVFSCSVMHEALHNHYTQKSLSLS | |
| | | PGK | |
| 176 | EGFR | QVQLQESGPGLVKPSQTLSLTCTVSGASITSGSHYWSWIRQPPGKGLEWI | Heavy |
| | | GYMYHSGSADYNPSLKSRVTMSVDTSKNQFSLKVNSVTAADTAVYYCART | Chain |
| | | LGVYGDYDAFDIWGQGTMVTVSSASTKGPSVFPLAPSSKSTSGGTAALGC | (VH-CH) |
| | | LVKDYFPEPVTVSWNSGALTSGVHTFPAVLQSSGLYSLSSVVTVPSSSLG | |
| | | TQTYICNVNHKPSNTKVDKKVEPKSCDKTHTCPPCPAPELLGGPSVFLFP | |
| | | PKPKDTLMISRTPEVTCVVVDVSHEDPEVKFNWYVDGVEVHNAKTKPREE | |
| | | QYNSTYRVVSVLTVLHQDWLNGKEYKCKVSNKALPAPIEKTISKAKGQPR | |
| | | EPQVYTLPPSRDELTKNQVSLTCLVKGFYPSDIAVEWESNGQPENNYKTT | |
| | | PPVLDSDGSFFLYSKLTVDKSRWQQGNVFSCSVMHEALHNHYTQKSLSLS | |
| | | PGK | |
| 177 | ITGB6 | QVQLQQSGAELVRPGTSVKVSCKASGYDFNNDLIEWVKQRPGQGLEWIAV | Heavy |
| | | INPGSGRTNYNEKFKGKATLTADKSSSTVYMQLSSLTSDDSAVYFCAMIY | Chain |
| | | YGPHSYAMDYWGQGTSVTVSSASTKGPSVFPLAPSSKSTSGGTAALGCLV | (VH-CH) |
| | | KDYFPEPVTVSWNSGALTSGVHTFPAVLQSSGLYSLSSVVTVPSSSLGTQ | |
| | | TYICNVNHKPSNTKVDKKVEPKSCDKTHTCPPCPAPELLGGPSVFLFPPK | |
| | | PKDTLMISRTPEVTCVVVDVSHEDPEVKFNWYVDGVEVHNAKTKPREEQY | |
| | | NSTYRVVSVLTVLHQDWLNGKEYKCKVSNKALPAPIEKTISKAKGQPREP | |
| | | QVYTLPPSRDELTKNQVSLTCLVKGFYPSDIAVEWESNGQPENNYKTTPP | |
| | | VLDSDGSFFLYSKLTVDKSRWQQGNVFSCSVMHEALHNHYTQKSLSLSPG | |
| | | K | |
| 178 | ITGB6 | QVQLVQSGAEVKKPGASVKVSCKASGYDFNNDLIEWVRQAPGQGLEWMAV | Heavy |
| | | INPGSGRTNYAQKFQGRVTMTADKSTSTVYMELSSLRSEDTAVYYCAMIY | Chain |
| | | YGPHSYAMDYWGQGTLVTVSSASTKGPSVFPLAPSSKSTSGGTAALGCLV | (VH-CH) |
| | | KDYFPEPVTVSWNSGALTSGVHTFPAVLQSSGLYSLSSVVTVPSSSLGTQ | |
| | | TYICNVNHKPSNTKVDKKVEPKSCDKTHTCPPCPAPELLGGPSVFLFPPK | |
| | | PKDTLMISRTPEVTCVVVDVSHEDPEVKFNWYVDGVEVHNAKTKPREEQY | |
| | | NSTYRVVSVLTVLHQDWLNGKEYKCKVSNKALPAPIEKTISKAKGQPREP | |
| | | QVYTLPPSRDELTKNQVSLTCLVKGFYPSDIAVEWESNGQPENNYKTTPP | |
| | | VLDSDGSFFLYSKLTVDKSRWQQGNVFSCSVMHEALHNHYTQKSLSLSPG | |
| | | K | |
| 179 | ITGB6 | QVQLQQSGAELARPGTSVKVSCKASGYAFTNYLIEWVKQRPGQGLEWIGV | Heavy |
| | | ISPGSGIINYNEKFKGKATLTADKSSSTAYMQLSSLTSDDSAVYFCAAID | Chain |
| | | YSGPYAVDDWGQGTSVTVSSASTKGPSVFPLAPSSKSTSGGTAALGCLVK | (VH-CH) |
| | | DYFPEPVTVSWNSGALTSGVHTFPAVLQSSGLYSLSSVVTVPSSSLGTQT | |
| | | YICNVNHKPSNTKVDKKVEPKSCDKTHTCPPCPAPELLGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSRDELTKNQVSLTCLVKGFYPSDIAVEWESNGQPENNYKTTPPV | |
| | | LDSDGSFFLYSKLTVDKSRWQQGNVFSCSVMHEALHNHYTQKSLSLSPGK | |
| 180 | ITGB6 | QVQLVQSGAEVKKPGSSVKVSCKASGYAFTNYLIEWVRQAPGQGLEWMGV | Heavy |
| | | ISPGSGIINYAQKFQGRATLTADKSTSTAYMELSSLRSEDTAVYYCAAID | Chain |
| | | YSGPYAVDDWGQGTLVTVSSASTKGPSVFPLAPSSKSTSGGTAALGCLVK | (VH-CH) |
| | | DYFPEPVTVSWNSGALTSGVHTFPAVLQSSGLYSLSSVVTVPSSSLGTQT | |
| | | YICNVNHKPSNTKVDKKVEPKSCDKTHTCPPCPAPELLGGPSVFLFPPKP | |
| | | KDTLMISRTPEVTCVVVDVSHEDPEVKFNWYVDGVEVHNAKTKPREEQYN | |
| | | STYRVVSVLTVLHQDWLNGKEYKCKVSNKALPAPIEKTISKAKGQPREPQ | |
| | | VYTLPPSRDELTKNQVSLTCLVKGFYPSDIAVEWESNGQPENNYKTTPPV | |
| | | LDSDGSFFLYSKLTVDKSRWQQGNVFSCSVMHEALHNHYTQKSLSLSPGK | |
| 181 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | SGYYGPDVWGKGTTVTVSS | |
| 182 | EGFR | EVQLVESGGGLVQPGRSLRLSCAASGFTFSDYGMHWVRQAPGKGLEWVSA | VH |
| | | IDRGGSTDYADSVEGRFTISRDNAKNSLYLQMNSLRAEDTAVYYCAKDLE | |
| | | SGYYGPDVWGKGTTVTVSS | |
| 183 | EGFR | EVQLVESGGGLVQPGRSLRLSCAASGFMFSDYGMHWVRQAPGKGLEWVSA | VH |
| | | IDAGGSTDYADSVEGRFTISRDNAKNSLYLQMNSLRAEDTAVYYCAKDLE | |
| | | SGYYGPDVWGKGTTVTVSS | |
| 184 | EGFR | EVQLVESGGGLVQPGRSLRLSCAASGFMFSDYGMHWVRQAPGKGLEWVSA | VH |
| | | IDRGGATDYADSVEGRFTISRDNAKNSLYLQMNSLRAEDTAVYYCAKDLE | |
| | | SGYYGPDVWGKGTTVTVSS | |
| 185 | EGFR | EVQLVESGGGLVQPGRSLRLSCAASGFMFSDYGMHWVRQAPGKGLEWVSA | VH |
| | | IDRGGSTYYADSVEGRFTISRDNAKNSLYLQMNSLRAEDTAVYYCAKDLE | |
| | | SGYYGPDVWGKGTTVTVSS | |
| 186 | EGFR | EVQLVESGGGLVQPGRSLRLSCAASGFMFSDYGMHWVRQAPGKGLEWVSA | VH |
| | | IDRGGSTDYADSVKGRFTISRDNAKNSLYLQMNSLRAEDTAVYYCAKDLE | |
| | | SGYYGPDVWGKGTTVTVSS | |
| 187 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLA | |
| | | SGYYGPDVWGKGTTVTVSS | |
| 188 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | AGYYGPDVWGKGTTVTVSS | |
| 189 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | SGAYGPDVWGKGTTVTVSS | |
| 190 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | SGYAGPDVWGKGTTVTVSS | |
| 191 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | SGYYGFDVWGKGTTVTVSS | |
| 192 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | SGYYGPDVWGKGTTVTVSS | |
| 193 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | SGYYGPDVWGKGTTVTVSS | |
| 194 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | SGYYGPDVWGKGTTVTVSS | |
| 195 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | SGYYGPDVWGKGTTVTVSS | |
| 196 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | SGYYGPDVWGKGTTVTVSS | |
| 197 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | SGYYGPDVWGKGTTVTVSS | |
| 198 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | SGYYGPDVWGKGTTVTVSS | |
| 199 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | SGYYGPDVWGKGTTVTVSS | |
| 200 | EGFR | EVQLVESGGGLVQPGRSLRLSCAASGFMFSDYGMHWVRQAPGKGLEWVSA | VH |
| | | IDRGGSTDYADSVEGRFTISRDNAKNSLYLQMNSLRAEDTAVYYCAKDLE | |
| | | SGYYGPDVWGKGTTVTVSS | |
| 201 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | SGYYGPDVWGKGTTVTVSS | |
| 202 | EGFR | EVQLVESGGGLVQPGRSLRLSCAASGFTFSDYGMHWVRQAPGKGLEWVSA | VH |
| | | IDRGGSTDYADSVEGRFTISRDNAKNSLYLQMNSLRAEDTAVYYCAKDLE | |
| | | AGYYGPDVWGKGTTVTVSS | |
| 203 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 204 | EGFR | EVQLVESGGGLVQPGRSLRLSCAASGFTFSDYGMHWVRQAPGKGLEWVSA | VH |
| | | IDRGGSTDYADSVEGRFTISRDNAKNSLYLQMNSLRAEDTAVYYCAKDLE | |
| | | SGAYGPDVWGKGTTVTVSS | |
| 205 | EGFR | EVQLVESGGGLVQPGRSLRLSCAASGFTFSDYGMHWVRQAPGKGLEWVSA | VH |
| | | IDRGGSTDYADSVEGRFTISRDNAKNSLYLQMNSLRAEDTAVYYCAKDLE | |
| | | SGAYGFDVWGKGTTVTVSS | |
| 206 | EGFR | EVQLVESGGGLVQPGRSLRLSCAASGFTFSAYGMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 207 | EGFR | EVQLVESGGGLVQPGRSLRLSCAASGFTFSDAGMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 208 | EGFR | EVQLVESGGGLVQPGRSLRLSCAASGFTFSDYAMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 209 | EGFR | EVQLVESGGGLVQPGRSLRLSCAASGFTFSDYGMHWVRQAPGKGLEWVSA | VH |
| | | ISAGGSTDYADSVEGRFTISRDNAKNSLYLQMNSLRAEDTAVYYCAKDLE | |
| | | AGYYGPDVWGKGTTVTVSS | |
| 210 | EGFR | EVQLVESGGGLVQPGRSLRLSCAASGFTFSDYGMHWVRQAPGKGLEWVSA | VH |
| | | IDASGSTDYADSVEGRFTISRDNAKNSLYLQMNSLRAEDTAVYYCAKDLE | |
| | | AGYYGPDVWGKGTTVTVSS | |
| 211 | EGFR | EVQLVESGGGLVQPGRSLRLSCAASGFTFSDYGMHWVRQAPGKGLEWVSA | VH |
| | | IDAGASTDYADSVEGRFTISRDNAKNSLYLQMNSLRAEDTAVYYCAKDLE | |
| | | AGYYGPDVWGKGTTVTVSS | |
| 212 | EGFR | EVQLVESGGGLVQPGRSLRLSCAASGFTFSDYGMHWVRQAPGKGLEWVSA | VH |
| | | IDAGGSADYADSVEGRFTISRDNAKNSLYLQMNSLRAEDTAVYYCAKDLE | |
| | | AGYYGPDVWGKGTTVTVSS | |
| 213 | EGFR | EVQLVESGGGLVQPGRSLRLSCAASGFTFSDYGMHWVRQAPGKGLEWVSA | VH |
| | | IDAGGSTAYADSVEGRFTISRDNAKNSLYLQMNSLRAEDTAVYYCAKDLE | |
| | | AGYYGPDVWGKGTTVTVSS | |
| 214 | EGFR | EVQLVESGGGLVQPGRSLRLSCAASGFTFSDYGMHWVRQAPGKGLEWVSA | VH |
| | | IDAGGSTGYADSVEGRFTISRDNAKNSLYLQMNSLRAEDTAVYYCAKDLE | |
| | | AGYYGPDVWGKGTTVTVSS | |
| 215 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKALE | |
| | | AGYYGPDVWGKGTTVTVSS | |
| 216 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDAE | |
| | | AGYYGPDVWGKGTTVTVSS | |
| 217 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | AAYYGPDVWGKGTTVTVSS | |
| 218 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | AGYYAPDVWGKGTTVTVSS | |
| 219 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 220 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 221 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 222 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 223 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 224 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 225 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 226 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 227 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 228 | EGFR | EVQLVESGGGLVQPGRSLRLSCAASGFTFSDYGMHWVRQAPGKGLEWVSA | VH |
| | | IDAGGSTDYADSVEGRFTISRDNAKNSLYLQMNSLRAEDTAVYYCAKDLE | |
| | | AGYYGPDVWGKGTTVTVSS | |
| 229 | EGFR | QVQLVQSGAEVKKPGASVKVSCKVSGDTFSNQGISWVRQAPGKGLEWMGG | VH |
| | | FDPDAGETIYAQKFQGRVTMTEDTSTDTAYMELSSLKSEDTAVYYCARGV | |
| | | DSYGYGRYNWFDPWGQGTLVTVSS | |
| 230 | EGFR | QVQLQESGPGLVKPSQTLSLTCTVSGASIRSGDYSWSWIRQPPGKGLEWI | VH |
| | | GYIEHSGRTDYNPSLKSRVTMSVDTSKNQFSLKVNSVTAADTAVYYCARA | |
| | | SGWYGVYDAFDVWGKGTTVTVSS | |
| 231 | EGFR | QVQLQESGPGLVKPSQTLSLTCTVSGASITSGSHYWSWIRQPPGKGLEWI | VH |
| | | GYMYHSGSADYNPSLKSRVTMSVDTSKNQFSLKVNSVTAADTAVYYCART | |
| | | LGVYGDYDAFDIWGQGTMVTVSS | |
| 232 | ITGB6 | QVQLQQSGAELVRPGTSVKVSCKASGYDFNNDLIEWVKQRPGQGLEWIAV | VH |
| | | INPGSGRTNYNEKFKGKATLTADKSSSTVYMQLSSLTSDDSAVYFCAMIY | |
| | | YGPHSYAMDYWGQGTSVTVSS | |
| 233 | ITGB6 | QVQLVQSGAEVKKPGASVKVSCKASGYDFNNDLIEWVRQAPGQGLEWMAV | VH |
| | | INPGSGRTNYAQKFQGRVTMTADKSTSTVYMELSSLRSEDTAVYYCAMIY | |
| | | YGPHSYAMDYWGQGTLVTVSS | |
| 234 | ITGB6 | QVQLQQSGAELARPGTSVKVSCKASGYAFTNYLIEWVKQRPGQGLEWIGV | VH |
| | | ISPGSGIINYNEKFKGKATLTADKSSSTAYMQLSSLTSDDSAVYFCAAID | |
| | | YSGPYAVDDWGQGTSVTVSS | |
| 235 | ITGB6 | QVQLVQSGAEVKKPGSSVKVSCKASGYAFTNYLIEWVRQAPGQGLEWMGV | VH |
| | | ISPGSGIINYAQKFQGRATLTADKSTSTAYMELSSLRSEDTAVYYCAAID | |
| | | YSGPYAVDDWGQGTLVTVSS | |
| 236 | EGFR | DYGMH | HCDR1 |
| | | | (Kabat) |
| 237 | EGFR | DYGMH | HCDR1 |
| | | | (Kabat) |
| 238 | EGFR | DYGMH | HCDR1 |
| | | | (Kabat) |
| 239 | EGFR | DYGMH | HCDR1 |
| | | | (Kabat) |
| 240 | EGFR | DYGMH | HCDRI |
| | | | (Kabat) |
| 241 | EGFR | DYGMH | HCDR1 |
| | | | (Kabat) |
| 242 | EGFR | DYGMH | HCDR1 |
| | | | (Kabat) |
| 243 | EGFR | DYGMH | HCDR1 |
| | | | (Kabat) |
| 244 | EGFR | DYGMH | HCDR1 |
| | | | (Kabat) |
| 245 | EGFR | DYGMH | HCDR1 |
| | | | (Kabat) |
| 246 | EGFR | DYGMH | HCDRI |
| | | | (Kabat) |
| 247 | EGFR | DYGMH | HCDR1 |
| | | | (Kabat) |
| 248 | EGFR | DYGMH | HCDR1 |
| | | | (Kabat) |
| 249 | EGFR | DYGMH | HCDR1 |
| | | | (Kabat) |
| 250 | EGFR | DYGMH | HCDR1 |
| | | | (Kabat) |
| 251 | EGFR | DYGMH | HCDR1 |
| | | | (Kabat) |
| 252 | EGFR | DYGMH | HCDR1 |
| | | | (Kabat) |
| 253 | EGFR | DYGMH | HCDR1 |
| | | | (Kabat) |
| 254 | EGFR | DYGMH | HCDR1 |
| | | | (Kabat) |
| 255 | EGFR | DYGMH | HCDR1 |
| | | | (Kabat) |
| 256 | EGFR | DYGMH | HCDR1 |
| | | | (Kabat) |
| 257 | EGFR | DYGMH | HCDR1 |
| | | | (Kabat) |
| 258 | EGFR | DYGMH | HCDR1 |
| | | | (Kabat) |
| 259 | EGFR | DYGMH | HCDR1 |
| | | | (Kabat) |
| 260 | EGFR | DYGMH | HCDR1 |
| | | | (Kabat) |
| 261 | EGFR | AYGMH | HCDR1 |
| | | | (Kabat) |
| 262 | EGFR | DAGMH | HCDR1 |
| | | | (Kabat) |
| 263 | EGFR | DYAMH | HCDR1 |
| | | | (Kabat) |
| 264 | EGFR | DYGMH | HCDR1 |
| | | | (Kabat) |
| 265 | EGFR | DYGMH | HCDR1 |
| | | | (Kabat) |
| 266 | EGFR | DYGMH | HCDR1 |
| | | | (Kabat) |
| 267 | EGFR | DYGMH | HCDR1 |
| | | | (Kabat) |
| 268 | EGFR | DYGMH | HCDR1 |
| | | | (Kabat) |
| 269 | EGFR | DYGMH | HCDR1 |
| | | | (Kabat) |
| 270 | EGFR | DYGMH | HCDR1 |
| | | | (Kabat) |
| 271 | EGFR | DYGMH | HCDR1 |
| | | | (Kabat) |
| 272 | EGFR | DYGMH | HCDR1 |
| | | | (Kabat) |
| 273 | EGFR | DYGMH | HCDR1 |
| | | | (Kabat) |
| 274 | EGFR | DYGMH | HCDR1 |
| | | | (Kabat) |
| 275 | EGFR | DYGMH | HCDR1 |
| | | | (Kabat) |
| 276 | EGFR | DYGMH | HCDR1 |
| | | | (Kabat) |
| 277 | EGFR | DYGMH | HCDR1 |
| | | | (Kabat) |
| 278 | EGFR | DYGMH | HCDR1 |
| | | | (Kabat) |
| 279 | EGFR | DYGMH | HCDR1 |
| | | | (Kabat) |
| 280 | EGFR | DYGMH | HCDR1 |
| | | | (Kabat) |
| 281 | EGFR | DYGMH | HCDR1 |
| | | | (Kabat) |
| 282 | EGFR | DYGMH | HCDR1 |
| | | | (Kabat) |
| 283 | EGFR | DYGMH | HCDR1 |
| | | | (Kabat) |
| 284 | EGFR | NQGIS | HCDR1 |
| | | | (Kabat) |
| 285 | EGFR | SGDYSWS | HCDR1 |
| | | | (Kabat) |
| 286 | EGFR | SGSHYWS | HCDR1 |
| | | | (Kabat) |
| 287 | ITGB6 | NDLIE | HCDR1 |
| | | | (Kabat) |
| 288 | ITGB6 | NDLIE | HCDR1 |
| | | | (Kabat) |
| 289 | ITGB6 | NYLIE | HCDR1 |
| | | | (Kabat) |
| 290 | ITGB6 | NYLIE | HCDR1 |
| | | | (Kabat) |
| 291 | EGFR | AIDRGGSTDYADSVEG | HCDR2 |
| | | | (Kabat) |
| 292 | EGFR | AIDRGGSTDYADSVEG | HCDR2 |
| | | | (Kabat) |
| 293 | EGFR | AIDAGGSTDYADSVEG | HCDR2 |
| | | | (Kabat) |
| 294 | EGFR | AIDRGGATDYADSVEG | HCDR2 |
| | | | (Kabat) |
| 295 | EGFR | AIDRGGSTYYADSVEG | HCDR2 |
| | | | (Kabat) |
| 296 | EGFR | AIDRGGSTDYADSVKG | HCDR2 |
| | | | (Kabat) |
| 297 | EGFR | AIDRGGSTDYADSVEG | HCDR2 |
| | | | (Kabat) |
| 298 | EGFR | AIDRGGSTDYADSVEG | HCDR2 |
| | | | (Kabat) |
| 299 | EGFR | AIDRGGSTDYADSVEG | HCDR2 |
| | | | (Kabat) |
| 300 | EGFR | AIDRGGSTDYADSVEG | HCDR2 |
| | | | (Kabat) |
| 301 | EGFR | AIDRGGSTDYADSVEG | HCDR2 |
| | | | (Kabat) |
| 302 | EGFR | AIDRGGSTDYADSVEG | HCDR2 |
| | | | (Kabat) |
| 303 | EGFR | AIDRGGSTDYADSVEG | HCDR2 |
| | | | (Kabat) |
| 304 | EGFR | AIDRGGSTDYADSVEG | HCDR2 |
| | | | (Kabat) |
| 305 | EGFR | AIDRGGSTDYADSVEG | HCDR2 |
| | | | (Kabat) |
| 306 | EGFR | AIDRGGSTDYADSVEG | HCDR2 |
| | | | (Kabat) |
| 307 | EGFR | AIDRGGSTDYADSVEG | HCDR2 |
| | | | (Kabat) |
| 308 | EGFR | AIDRGGSTDYADSVEG | HCDR2 |
| | | | (Kabat) |
| 309 | EGFR | AIDRGGSTDYADSVEG | HCDR2 |
| | | | (Kabat) |
| 310 | EGFR | AIDRGGSTDYADSVEG | HCDR2 |
| | | | (Kabat) |
| 311 | EGFR | AIDAGGSTDYADSVEG | HCDR2 |
| | | | (Kabat) |
| 312 | EGFR | AIDRGGSTDYADSVEG | HCDR2 |
| | | | (Kabat) |
| 313 | EGFR | AIDAGGSTDYADSVEG | HCDR2 |
| | | | (Kabat) |
| 314 | EGFR | AIDRGGSTDYADSVEG | HCDR2 |
| | | | (Kabat) |
| 315 | EGFR | AIDRGGSTDYADSVEG | HCDR2 |
| | | | (Kabat) |
| 316 | EGFR | AIDAGGSTDYADSVEG | HCDR2 |
| | | | (Kabat) |
| 317 | EGFR | AIDAGGSTDYADSVEG | HCDR2 |
| | | | (Kabat) |
| 318 | EGFR | AIDAGGSTDYADSVEG | HCDR2 |
| | | | (Kabat) |
| 319 | EGFR | AISAGGSTDYADSVEG | HCDR2 |
| | | | (Kabat) |
| 320 | EGFR | AIDASGSTDYADSVEG | HCDR2 |
| | | | (Kabat) |
| 321 | EGFR | AIDAGASTDYADSVEG | HCDR2 |
| | | | (Kabat) |
| 322 | EGFR | AIDAGGSADYADSVEG | HCDR2 |
| | | | (Kabat) |
| 323 | EGFR | AIDAGGSTAYADSVEG | HCDR2 |
| | | | (Kabat) |
| 324 | EGFR | AIDAGGSTGYADSVEG | HCDR2 |
| | | | (Kabat) |
| 325 | EGFR | AIDAGGSTDYADSVEG | HCDR2 |
| | | | (Kabat) |
| 326 | EGFR | AIDAGGSTDYADSVEG | HCDR2 |
| | | | (Kabat) |
| 327 | EGFR | AIDAGGSTDYADSVEG | HCDR2 |
| | | | (Kabat) |
| 328 | EGFR | AIDAGGSTDYADSVEG | HCDR2 |
| | | | (Kabat) |
| 329 | EGFR | AIDAGGSTDYADSVEG | HCDR2 |
| | | | (Kabat) |
| 330 | EGFR | AIDAGGSTDYADSVEG | HCDR2 |
| | | | (Kabat) |
| 331 | EGFR | AIDAGGSTDYADSVEG | HCDR2 |
| | | | (Kabat) |
| 332 | EGFR | AIDAGGSTDYADSVEG | HCDR2 |
| | | | (Kabat) |
| 333 | EGFR | AIDAGGSTDYADSVEG | HCDR2 |
| | | | (Kabat) |
| 334 | EGFR | AIDAGGSTDYADSVEG | HCDR2 |
| | | | (Kabat) |
| 335 | EGFR | AIDAGGSTDYADSVEG | HCDR2 |
| | | | (Kabat) |
| 336 | EGFR | AIDAGGSTDYADSVEG | HCDR2 |
| | | | (Kabat) |
| 337 | EGFR | AIDAGGSTDYADSVEG | HCDR2 |
| | | | (Kabat) |
| 338 | EGFR | AIDAGGSTDYADSVEG | HCDR2 |
| | | | (Kabat) |
| 339 | EGFR | GFDPDAGETIYAQKFQG | HCDR2 |
| | | | (Kabat) |
| 340 | EGFR | YIEHSGRTDYNPSLKS | HCDR2 |
| | | | (Kabat) |
| 341 | EGFR | YMYHSGSADYNPSLKS | HCDR2 |
| | | | (Kabat) |
| 342 | ITGB6 | VINPGSGRTNYNEKFKG | HCDR2 |
| | | | (Kabat) |
| 343 | ITGB6 | VINPGSGRTNYAQKFQG | HCDR2 |
| | | | (Kabat) |
| 344 | ITGB6 | VISPGSGIINYNEKFKG | HCDR2 |
| | | | (Kabat) |
| 345 | ITGB6 | VISPGSGIINYAQKFQG | HCDR2 |
| | | | (Kabat) |
| 346 | EGFR | DLESGYYGPDV | HCDR3 |
| | | | (Kabat) |
| 347 | EGFR | DLESGYYGPDV | HCDR3 |
| | | | (Kabat) |
| 348 | EGFR | DLESGYYGPDV | HCDR3 |
| | | | (Kabat) |
| 349 | EGFR | DLESGYYGPDV | HCDR3 |
| | | | (Kabat) |
| 350 | EGFR | DLESGYYGPDV | HCDR3 |
| | | | (Kabat) |
| 351 | EGFR | DLESGYYGPDV | HCDR3 |
| | | | (Kabat) |
| 352 | EGFR | DLASGYYGPDV | HCDR3 |
| | | | (Kabat) |
| 353 | EGFR | DLEAGYYGPDV | HCDR3 |
| | | | (Kabat) |
| 354 | EGFR | DLESGAYGPDV | HCDR3 |
| | | | (Kabat) |
| 355 | EGFR | DLESGYAGPDV | HCDR3 |
| | | | (Kabat) |
| 356 | EGFR | DLESGYYGFDV | HCDR3 |
| | | | (Kabat) |
| 357 | EGFR | DLESGYYGPDV | HCDR3 |
| | | | (Kabat) |
| 358 | EGFR | DLESGYYGPDV | HCDR3 |
| | | | (Kabat) |
| 359 | EGFR | DLESGYYGPDV | HCDR3 |
| | | | (Kabat) |
| 360 | EGFR | DLESGYYGPDV | HCDR3 |
| | | | (Kabat) |
| 361 | EGFR | DLESGYYGPDV | HCDR3 |
| | | | (Kabat) |
| 362 | EGFR | DLESGYYGPDV | HCDR3 |
| | | | (Kabat) |
| 363 | EGFR | DLESGYYGPDV | HCDR3 |
| | | | (Kabat) |
| 364 | EGFR | DLESGYYGPDV | HCDR3 |
| | | | (Kabat) |
| 365 | EGFR | DLESGYYGPDV | HCDR3 |
| | | | (Kabat) |
| 366 | EGFR | DLESGYYGPDV | HCDR3 |
| | | | (Kabat) |
| 367 | EGFR | DLEAGYYGPDV | HCDR3 |
| | | | (Kabat) |
| 368 | EGFR | DLEAGYYGPDV | HCDR3 |
| | | | (Kabat) |
| 369 | EGFR | DLESGAYGPDV | HCDR3 |
| | | | (Kabat) |
| 370 | EGFR | DLESGAYGFDV | HCDR3 |
| | | | (Kabat) |
| 371 | EGFR | DLEAGYYGPDV | HCDR3 |
| | | | (Kabat) |
| 372 | EGFR | DLEAGYYGPDV | HCDR3 |
| | | | (Kabat) |
| 373 | EGFR | DLEAGYYGPDV | HCDR3 |
| | | | (Kabat) |
| 374 | EGFR | DLEAGYYGPDV | HCDR3 |
| | | | (Kabat) |
| 375 | EGFR | DLEAGYYGPDV | HCDR3 |
| | | | (Kabat) |
| 376 | EGFR | DLEAGYYGPDV | HCDR3 |
| | | | (Kabat) |
| 377 | EGFR | DLEAGYYGPDV | HCDR3 |
| | | | (Kabat) |
| 378 | EGFR | DLEAGYYGPDV | HCDR3 |
| | | | (Kabat) |
| 379 | EGFR | DLEAGYYGPDV | HCDR3 |
| | | | (Kabat) |
| 380 | EGFR | ALEAGYYGPDV | HCDR3 |
| | | | (Kabat) |
| 381 | EGFR | DAEAGYYGPDV | HCDR3 |
| | | | (Kabat) |
| 382 | EGFR | DLEAAYYGPDV | HCDR3 |
| | | | (Kabat) |
| 383 | EGFR | DLEAGYYAPDV | HCDR3 |
| | | | (Kabat) |
| 384 | EGFR | DLEAGYYGPDV | HCDR3 |
| | | | (Kabat) |
| 385 | EGFR | DLEAGYYGPDV | HCDR3 |
| | | | (Kabat) |
| 386 | EGFR | DLEAGYYGPDV | HCDR3 |
| | | | (Kabat) |
| 387 | EGFR | DLEAGYYGPDV | HCDR3 |
| | | | (Kabat) |
| 388 | EGFR | DLEAGYYGPDV | HCDR3 |
| | | | (Kabat) |
| 389 | EGFR | DLEAGYYGPDV | HCDR3 |
| | | | (Kabat) |
| 390 | EGFR | DLEAGYYGPDV | HCDR3 |
| | | | (Kabat) |
| 391 | EGFR | DLEAGYYGPDV | HCDR3 |
| | | | (Kabat) |
| 392 | EGFR | DLEAGYYGPDV | HCDR3 |
| | | | (Kabat) |
| 393 | EGFR | DLEAGYYGPDV | HCDR3 |
| | | | (Kabat) |
| 394 | EGFR | GVDSYGYGRYNWFDP | HCDR3 |
| | | | (Kabat) |
| 395 | EGFR | ASGWYGVYDAFDV | HCDR3 |
| | | | (Kabat) |
| 396 | EGFR | TLGVYGDYDAFDI | HCDR3 |
| | | | (Kabat) |
| 397 | ITGB6 | IYYGPHSYAMDY | HCDR3 |
| | | | (Kabat) |
| 398 | ITGB6 | IYYGPHSYAMDY | HCDR3 |
| | | | (Kabat) |
| 399 | ITGB6 | IDYSGPYAVDD | HCDR3 |
| | | | (Kabat) |
| 400 | ITGB6 | IDYSGPYAVDD | HCDR3 |
| | | | (Kabat) |
| 401 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWK | (VL-CL) |
| | | VDNALQSGNSQESVTEQDSKDSTYSLSSTLTLSKADYEKHKVYACEVTHQ | |
| | | GLSSPVTKSFNRGEC | |
| 402 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 403 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 404 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 405 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 406 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 407 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 408 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 409 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 410 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 411 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 412 | EGFR | DIQMTQSPSSLSASVGDRVTITCRASQDIGNFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 413 | EGFR | DIQMTQSPSSLSASVGDRVTITCRASQDIGRALAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 414 | EGFR | DIQMTQSPSSLSASVGDRVTITCRASQDIGRFLAWYQQKPGKAPKLLIYA | Light |
| | | ASNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 415 | EGFR | DIQMTQSPSSLSASVGDRVTITCRASQDIGRFLAWYQQKPGKAPKLLIYA | Light |
| | | VSTLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 416 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQASTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 417 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYATSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 418 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSASVYTFGG | Chain |
| | | GTKVEIKRTVAAPSVFIFPPSDEQLKSGTASVVCLLNNFYPREAKVQWK | (VL-CL) |
| | | VDNALQSGNSQESVTEQDSKDSTYSLSSTLTLSKADYEKHKVYACEVTHQ | |
| | | GLSSPVTKSFNRGEC | |
| 419 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTAVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 420 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSPYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 421 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 422 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 423 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 424 | EGFR | DIQMTQSPSSLSASVGDRVTITCRASQDIGRALAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 425 | EGFR | DIQMTQSPSSLSASVGDRVTITCRASQDIGRALAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 426 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 427 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 428 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 429 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 430 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 431 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 432 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 433 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 434 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 435 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 436 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 437 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 438 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 439 | EGFR | DIQMTQSPSSLSASVGDRVTITCRASQAIGRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 440 | EGFR | DIQMTQSPSSLSASVGDRVTITCRASQDISRFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 441 | EGFR | DIQMTQSPSSLSASVGDRVTITCRASQDIGSFLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 442 | EGFR | DIQMTQSPSSLSASVGDRVTITCRASQDIGRYLAWYQQKPGKAPKLLIYA | Light |
| | | VSNLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 443 | EGFR | DIQMTQSPSSLSASVGDRVTITCRASQDIGRFLAWYQQKPGKAPKLLIYA | Light |
| | | VANLQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 444 | EGFR | DIQMTQSPSSLSASVGDRVTITCRASQDIGRFLAWYQQKPGKAPKLLIYA | Light |
| | | VSALQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 445 | EGFR | DIQMTQSPSSLSASVGDRVTITCRASQDIGRFLAWYQQKPGKAPKLLIYA | Light |
| | | VSNAQSGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 446 | EGFR | DIQMTQSPSSLSASVGDRVTITCRASQDIGRFLAWYQQKPGKAPKLLIYA | Light |
| | | VSNLASGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 447 | EGFR | DIQMTQSPSSLSASVGDRVTITCRASQDIGRFLAWYQQKPGKAPKLLIYA | Light |
| | | VSNLQAGVPSRFSGSGSGTDFTLTISSLQPEDVATYYCQQYSTSVYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 448 | EGFR | DIQMTQSPSSLSASVGDRVTITCRASQDIGRFLAWYQQKPGKAPKLLIYA | Light |
| | | VSNLQSGVPSRFSGSGSGTDFTLTISSLQPEDVATYYCQQYSTSAYTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 449 | EGFR | DIQMTQSPSSVSASVGDRVTITCRASQDIRHYLAWYQQKPGKAPKLLIYD | Light |
| | | TFNRATGVPSRFSGSGSGTDFTLTISSLQPEDFANYYCQQYHNLPYSFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 450 | EGFR | EIVMTQSPATLSLSPGERATLSCRASQSVSHHLAWYQQKPGQAPRLLIYD | Light |
| | | TFNRAPGIPARFSGSGSGTDFTLTISSLEPEDFAVYYCQHYDTAPFSFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 451 | EGFR | EIVMTQSPATLSLSPGERATLSCRASQSVSHNLAWYQQKPGQAPRLLIYD | Light |
| | | TFNRAPGIPARFSGSGSGTDFTLTISSLEPEDFAVYYCQQYDTRPLTFGG | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 452 | ITGB6 | DIVMTQSHKFMSTVVGDRVSITCKASLDVRTAVAWYQQKPGQSPKLLIYS | Light |
| | | ASYRYTGVPDRFTGSGSGTDFTFNIRSVQAEDLAVYYCQQHYGIPWTFGG | Chain |
| | | GTKLEIKRTVAAPSVFIFPPSDEQLKSGTASVVCLLNNFYPREAKVQ | (VL-CL) |
| | | WKVDNALQSGNSQESVTEQDSKDSTYSLSSTLTLSKADYEKHKVYACEVT | |
| | | HQGLSSPVTKSFNRGEC | |
| 453 | ITGB6 | DIQMTQSPSSLSASVGDRVTITCKASLDVRTAVAWYQQKPGKAPKLLIYS | Light |
| | | ASYRYTGVPSRFSGSGSGTDFTFTISSLQPEDIATYYCQQHYGIPWTFGQ | Chain |
| | | GTKVEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 454 | ITGB6 | DIVMTQSHKFMSTSVGDRVSVTCKASQAVNTAVAWYQQKPGQSPKLLIYS | Light |
| | | ASYGYTGVPDRFTGSGSGTDFTLTISSVQAEDLAVYYCQHHYGVPWTFGG | Chain |
| | | GTKLEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 455 | ITGB6 | DIQMTQSPSSLSASVGDRVTITCKASQAVNTAVAWYQQKPGKAPKLLIYS | Light |
| | | ASYGYTGVPSRFSGSGSGTDFTLTISSLQPEDFATYYCQHHYGVPWTFGG | Chain |
| | | GTKLEIKRTVAAPSVFIFPPSDEQLKSGTASVVCLLNNFYPREAKVQWKV | (VL-CL) |
| | | DNALQSGNSQESVTEQDSKDSTYSLSSTLTLSKADYEKHKVYACEVTHQG | |
| | | LSSPVTKSFNRGEC | |
| 456 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 457 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 458 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 459 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 460 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 461 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 462 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 463 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 464 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 465 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 466 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 467 | EGFR | DIQMTQSPSSLSASVGDRVTITCRASQDIGNFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 468 | EGFR | DIQMTQSPSSLSASVGDRVTITCRASQDIGRALAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 469 | EGFR | DIQMTQSPSSLSASVGDRVTITCRASQDIGRFLAWYQQKPGKAPKLLIYA | VL |
| | | ASNLQSGVPSRFSGSGSGTDFTLTISSLQPEDVATYYCQQYSTSVYTFGG | |
| | | GTKVEIK | |
| 470 | EGFR | DIQMTQSPSSLSASVGDRVTITCRASQDIGRFLAWYQQKPGKAPKLLIYA | VL |
| | | VSTLQSGVPSRFSGSGSGTDFTLTISSLQPEDVATYYCQQYSTSVYTFGG | |
| | | GTKVEIK | |
| 471 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQASTSVYTFGG | |
| | | GTKVEIK | |
| 472 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYATSVYTFGG | |
| | | GTKVEIK | |
| 473 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSASVYTFGG | |
| | | GTKVEIK | |
| 474 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTAVYTFGG | |
| | | GTKVEIK | |
| 475 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSPYTFGG | |
| | | GTKVEIK | |
| 476 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 477 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 478 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 479 | EGFR | DIQMTQSPSSLSASVGDRVTITCRASQDIGRALAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 480 | EGFR | DIQMTQSPSSLSASVGDRVTITCRASQDIGRALAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 481 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 482 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 483 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 484 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 485 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 486 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 487 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 488 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 489 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 490 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 491 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 492 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 493 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 494 | EGFR | DIQMTQSPSSLSASVGDRVTITCRASQAIGRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 495 | EGFR | DIQMTQSPSSLSASVGDRVTITCRASQDISRFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 496 | EGFR | DIQMTQSPSSLSASVGDRVTITCRASQDIGSFLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 497 | EGFR | DIQMTQSPSSLSASVGDRVTITCRASQDIGRYLAWYQQKPGKAPKLLIYA | VL |
| | | VSNLQSGVPSRFSGSGSGTDFTLTISSLQPEDVATYYCQQYSTSVYTFGG | |
| | | GTKVEIK | |
| 498 | EGFR | DIQMTQSPSSLSASVGDRVTITCRASQDIGRFLAWYQQKPGKAPKLLIYA | VL |
| | | VANLQSGVPSRFSGSGSGTDFTLTISSLQPEDVATYYCQQYSTSVYTFGG | |
| | | GTKVEIK | |
| 499 | EGFR | DIQMTQSPSSLSASVGDRVTITCRASQDIGRFLAWYQQKPGKAPKLLIYA | VL |
| | | VSALQSGVPSRFSGSGSGTDFTLTISSLQPEDVATYYCQQYSTSVYTFGG | |
| | | GTKVEIK | |
| 500 | EGFR | DIQMTQSPSSLSASVGDRVTITCRASQDIGRFLAWYQQKPGKAPKLLIYA | VL |
| | | VSNAQSGVPSRFSGSGSGTDFTLTISSLQPEDVATYYCQQYSTSVYTFGG | |
| | | GTKVEIK | |
| 501 | EGFR | DIQMTQSPSSLSASVGDRVTITCRASQDIGRFLAWYQQKPGKAPKLLIYA | VL |
| | | VSNLASGVPSRFSGSGSGTDFTLTISSLQPEDVATYYCQQYSTSVYTFGG | |
| | | GTKVEIK | |
| 502 | EGFR | DIQMTQSPSSLSASVGDRVTITCRASQDIGRFLAWYQQKPGKAPKLLIYA | VL |
| | | VSNLQAGVPSRFSGSGSGTDFTLTISSLQPEDVATYYCQQYSTSVYTFGG | |
| | | GTKVEIK | |
| 503 | EGFR | DIQMTQSPSSLSASVGDRVTITCRASQDIGRFLAWYQQKPGKAPKLLIYA | VL |
| | | VSNLQSGVPSRFSGSGSGTDFTLTISSLQPEDVATYYCQQYSTSAYTFGG | |
| | | GTKVEIK | |
| 504 | EGFR | DIQMTQSPSSVSASVGDRVTITCRASQDIRHYLAWYQQKPGKAPKLLIYD | VL |
| | | TFNRATGVPSRFSGSGSGTDFTLTISSLQPEDFANYYCQQYHNLPYSFGG | |
| | | GTKVEIK | |
| 505 | EGFR | EIVMTQSPATLSLSPGERATLSCRASQSVSHHLAWYQQKPGQAPRLLIYD | VL |
| | | TFNRAPGIPARFSGSGSGTDFTLTISSLEPEDFAVYYCQHYDTAPFSFGG | |
| | | GTKVEIK | |
| 506 | EGFR | EIVMTQSPATLSLSPGERATLSCRASQSVSHNLAWYQQKPGQAPRLLIYD | VL |
| | | TFNRAPGIPARFSGSGSGTDFTLTISSLEPEDFAVYYCQQYDTRPLTFGG | |
| | | GTKVEIK | |
| 507 | ITGB6 | DIVMTQSHKFMSTVVGDRVSITCKASLDVRTAVAWYQQKPGQSPKLLIYS | VL |
| | | ASYRYTGVPDRFTGSGSGTDFTFNIRSVQAEDLAVYYCQQHYGIPWTFGG | |
| | | GTKLEIK | |
| 508 | ITGB6 | DIQMTQSPSSLSASVGDRVTITCKASLDVRTAVAWYQQKPGKAPKLLIYS | VL |
| | | ASYRYTGVPSRFSGSGSGTDFTFTISSLQPEDIATYYCQQHYGIPWTFGQ | |
| | | GTKVEIK | |
| 509 | ITGB6 | DIVMTQSHKFMSTSVGDRVSVTCKASQAVNTAVAWYQQKPGQSPKLLIYS | VL |
| | | ASYGYTGVPDRFTGSGSGTDFTLTISSVQAEDLAVYYCQHHYGVPWTFGG | |
| | | GTKLEIK | |
| 510 | ITGB6 | DIQMTQSPSSLSASVGDRVTITCKASQAVNTAVAWYQQKPGKAPKLLIYS | VL |
| | | ASYGYTGVPSRFSGSGSGTDFTLTISSLQPEDFATYYCQHHYGVPWTFGG | |
| | | GTKLEIK | |
| 511 | EGFR | RASQDIGRFLA | LCDR1 |
| | | | (Kabat) |
| 512 | EGFR | RASQDIGRFLA | LCDR1 |
| | | | (Kabat) |
| 513 | EGFR | RASQDIGRFLA | LCDR1 |
| | | | (Kabat) |
| 514 | EGFR | RASQDIGRFLA | LCDR1 |
| | | | (Kabat) |
| 515 | EGFR | RASQDIGRFLA | LCDR1 |
| | | | (Kabat) |
| 516 | EGFR | RASQDIGRFLA | LCDR1 |
| | | | (Kabat) |
| 517 | EGFR | RASQDIGRFLA | LCDR1 |
| | | | (Kabat) |
| 518 | EGFR | RASQDIGRFLA | LCDR1 |
| | | | (Kabat) |
| 519 | EGFR | RASQDIGRFLA | LCDR1 |
| | | | (Kabat) |
| 520 | EGFR | RASQDIGRFLA | LCDR1 |
| | | | (Kabat) |
| 521 | EGFR | RASQDIGRFLA | LCDR1 |
| | | | (Kabat) |
| 522 | EGFR | RASQDIGNFLA | LCDR1 |
| | | | (Kabat) |
| 523 | EGFR | RASQDIGRALA | LCDR1 |
| | | | (Kabat) |
| 524 | EGFR | RASQDIGRFLA | LCDR1 |
| | | | (Kabat) |
| 525 | EGFR | RASQDIGRFLA | LCDR1 |
| | | | (Kabat) |
| 526 | EGFR | RASQDIGRFLA | LCDR1 |
| | | | (Kabat) |
| 527 | EGFR | RASQDIGRFLA | LCDR1 |
| | | | (Kabat) |
| 528 | EGFR | RASQDIGRFLA | LCDR1 |
| | | | (Kabat) |
| 529 | EGFR | RASQDIGRFLA | LCDR1 |
| | | | (Kabat) |
| 530 | EGFR | RASQDIGRFLA | LCDR1 |
| | | | (Kabat) |
| 531 | EGFR | RASQDIGRFLA | LCDR1 |
| | | | (Kabat) |
| 532 | EGFR | RASQDIGRFLA | LCDR1 |
| | | | (Kabat) |
| 533 | EGFR | RASQDIGRFLA | LCDR1 |
| | | | (Kabat) |
| 534 | EGFR | RASQDIGRALA | LCDR1 |
| | | | (Kabat) |
| 535 | EGFR | RASQDIGRALA | LCDR1 |
| | | | (Kabat) |
| 536 | EGFR | RASQDIGRFLA | LCDR1 |
| | | | (Kabat) |
| 537 | EGFR | RASQDIGRFLA | LCDR1 |
| | | | (Kabat) |
| 538 | EGFR | RASQDIGRFLA | LCDR1 |
| | | | (Kabat) |
| 539 | EGFR | RASQDIGRFLA | LCDR1 |
| | | | (Kabat) |
| 540 | EGFR | RASQDIGRFLA | LCDR1 |
| | | | (Kabat) |
| 541 | EGFR | RASQDIGRFLA | LCDR1 |
| | | | (Kabat) |
| 542 | EGFR | RASQDIGRFLA | LCDR1 |
| | | | (Kabat) |
| 543 | EGFR | RASQDIGRFLA | LCDR1 |
| | | | (Kabat) |
| 544 | EGFR | RASQDIGRFLA | LCDR1 |
| | | | (Kabat) |
| 545 | EGFR | RASQDIGRFLA | LCDR1 |
| | | | (Kabat) |
| 546 | EGFR | RASQDIGRFLA | LCDR1 |
| | | | (Kabat) |
| 547 | EGFR | RASQDIGRFLA | LCDR1 |
| | | | (Kabat) |
| 548 | EGFR | RASQDIGRFLA | LCDR1 |
| | | | (Kabat) |
| 549 | EGFR | RASQAIGRFLA | LCDR1 |
| | | | (Kabat) |
| 550 | EGFR | RASQDISRFLA | LCDR1 |
| | | | (Kabat) |
| 551 | EGFR | RASQDIGSFLA | LCDR1 |
| | | | (Kabat) |
| 552 | EGFR | RASQDIGRYLA | LCDR1 |
| | | | (Kabat) |
| 553 | EGFR | RASQDIGRFLA | LCDR1 |
| | | | (Kabat) |
| 554 | EGFR | RASQDIGRFLA | LCDR1 |
| | | | (Kabat) |
| 555 | EGFR | RASQDIGRFLA | LCDR1 |
| | | | (Kabat) |
| 556 | EGFR | RASQDIGRFLA | LCDR1 |
| | | | (Kabat) |
| 557 | EGFR | RASQDIGRFLA | LCDR1 |
| | | | (Kabat) |
| 558 | EGFR | RASQDIGRFLA | LCDR1 |
| | | | (Kabat) |
| 559 | EGFR | RASQDIRHYLA | LCDR1 |
| | | | (Kabat) |
| 560 | EGFR | RASQSVSHHLA | LCDR1 |
| | | | (Kabat) |
| 561 | EGFR | RASQSVSHNLA | LCDR1 |
| | | | (Kabat) |
| 562 | ITGB6 | KASLDVRTAVA | LCDR1 |
| | | | (Kabat) |
| 563 | ITGB6 | KASLDVRTAVA | LCDR1 |
| | | | (Kabat) |
| 564 | ITGB6 | KASQAVNTAVA | LCDR1 |
| | | | (Kabat) |
| 565 | ITGB6 | KASQAVNTAVA | LCDR1 |
| | | | (Kabat) |
| 566 | EGFR | AVSNLQS | LCDR2 |
| | | | (Kabat) |
| 567 | EGFR | AVSNLQS | LCDR2 |
| | | | (Kabat) |
| 568 | EGFR | AVSNLQS | LCDR2 |
| | | | (Kabat) |
| 569 | EGFR | AVSNLQS | LCDR2 |
| | | | (Kabat) |
| 570 | EGFR | AVSNLQS | LCDR2 |
| | | | (Kabat) |
| 571 | EGFR | AVSNLQS | LCDR2 |
| | | | (Kabat) |
| 572 | EGFR | AVSNLQS | LCDR2 |
| | | | (Kabat) |
| 573 | EGFR | AVSNLQS | LCDR2 |
| | | | (Kabat) |
| 574 | EGFR | AVSNLQS | LCDR2 |
| | | | (Kabat) |
| 575 | EGFR | AVSNLQS | LCDR2 |
| | | | (Kabat) |
| 576 | EGFR | AVSNLQS | LCDR2 |
| | | | (Kabat) |
| 577 | EGFR | AVSNLQS | LCDR2 |
| | | | (Kabat) |
| 578 | EGFR | AVSNLQS | LCDR2 |
| | | | (Kabat) |
| 579 | EGFR | AASNLQS | LCDR2 |
| | | | (Kabat) |
| 580 | EGFR | AVSTLQS | LCDR2 |
| | | | (Kabat) |
| 581 | EGFR | AVSNLQS | LCDR2 |
| | | | (Kabat) |
| 582 | EGFR | AVSNLQS | LCDR2 |
| | | | (Kabat) |
| 583 | EGFR | AVSNLQS | LCDR2 |
| | | | (Kabat) |
| 584 | EGFR | AVSNLQS | LCDR2 |
| | | | (Kabat) |
| 585 | EGFR | AVSNLQS | LCDR2 |
| | | | (Kabat) |
| 586 | EGFR | AVSNLQS | LCDR2 |
| | | | (Kabat) |
| 587 | EGFR | AVSNLQS | LCDR2 |
| | | | (Kabat) |
| 588 | EGFR | AVSNLQS | LCDR2 |
| | | | (Kabat) |
| 589 | EGFR | AVSNLQS | LCDR2 |
| | | | (Kabat) |
| 590 | EGFR | AVSNLQS | LCDR2 |
| | | | (Kabat) |
| 591 | EGFR | AVSNLQS | LCDR2 |
| | | | (Kabat) |
| 592 | EGFR | AVSNLQS | LCDR2 |
| | | | (Kabat) |
| 593 | EGFR | AVSNLQS | LCDR2 |
| | | | (Kabat) |
| 594 | EGFR | AVSNLQS | LCDR2 |
| | | | (Kabat) |
| 595 | EGFR | AVSNLQS | LCDR2 |
| | | | (Kabat) |
| 596 | EGFR | AVSNLQS | LCDR2 |
| | | | (Kabat) |
| 597 | EGFR | AVSNLQS | LCDR2 |
| | | | (Kabat) |
| 598 | EGFR | AVSNLQS | LCDR2 |
| | | | (Kabat) |
| 599 | EGFR | AVSNLQS | LCDR2 |
| | | | (Kabat) |
| 600 | EGFR | AVSNLQS | LCDR2 |
| | | | (Kabat) |
| 601 | EGFR | AVSNLQS | LCDR2 |
| | | | (Kabat) |
| 602 | EGFR | AVSNLQS | LCDR2 |
| | | | (Kabat) |
| 603 | EGFR | AVSNLQS | LCDR2 |
| | | | (Kabat) |
| 604 | EGFR | AVSNLQS | LCDR2 |
| | | | (Kabat) |
| 605 | EGFR | AVSNLQS | LCDR2 |
| | | | (Kabat) |
| 606 | EGFR | AVSNLQS | LCDR2 |
| | | | (Kabat) |
| 607 | EGFR | AVSNLQS | LCDR2 |
| | | | (Kabat) |
| 608 | EGFR | AVANLQS | LCDR2 |
| | | | (Kabat) |
| 609 | EGFR | AVSALQS | LCDR2 |
| | | | (Kabat) |
| 610 | EGFR | AVSNAQS | LCDR2 |
| | | | (Kabat) |
| 611 | EGFR | AVSNLAS | LCDR2 |
| | | | (Kabat) |
| 612 | EGFR | AVSNLQA | LCDR2 |
| | | | (Kabat) |
| 613 | EGFR | AVSNLQS | LCDR2 |
| | | | (Kabat) |
| 614 | EGFR | DTFNRAT | LCDR2 |
| | | | (Kabat) |
| 615 | EGFR | DTFNRAP | LCDR2 |
| | | | (Kabat) |
| 616 | EGFR | DTFNRAP | LCDR2 |
| | | | (Kabat) |
| 617 | ITGB6 | SASYRYT | LCDR2 |
| | | | (Kabat) |
| 618 | ITGB6 | SASYRYT | LCDR2 |
| | | | (Kabat) |
| 619 | ITGB6 | SASYGYT | LCDR2 |
| | | | (Kabat) |
| 620 | ITGB6 | SASYGYT | LCDR2 |
| | | | (Kabat |
| 621 | EGFR | QQYSTSVYT | LCDR3 |
| | | | (Kabat) |
| 622 | EGFR | QQYSTSVYT | LCDR3 |
| | | | (Kabat) |
| 623 | EGFR | QQYSTSVYT | LCDR3 |
| | | | (Kabat) |
| 624 | EGFR | QQYSTSVYT | LCDR3 |
| | | | (Kabat) |
| 625 | EGFR | QQYSTSVYT | LCDR3 |
| | | | (Kabat) |
| 626 | EGFR | QQYSTSVYT | LCDR3 |
| | | | (Kabat) |
| 627 | EGFR | QQYSTSVYT | LCDR3 |
| | | | (Kabat) |
| 628 | EGFR | QQYSTSVYT | LCDR3 |
| | | | (Kabat) |
| 629 | EGFR | QQYSTSVYT | LCDR3 |
| | | | (Kabat) |
| 630 | EGFR | QQYSTSVYT | LCDR3 |
| | | | (Kabat) |
| 631 | EGFR | QQYSTSVYT | LCDR3 |
| | | | (Kabat) |
| 632 | EGFR | QQYSTSVYT | LCDR3 |
| | | | (Kabat) |
| 633 | EGFR | QQYSTSVYT | LCDR3 |
| | | | (Kabat) |
| 634 | EGFR | QQYSTSVYT | LCDR3 |
| | | | (Kabat) |
| 635 | EGFR | QQYSTSVYT | LCDR3 |
| | | | (Kabat) |
| 636 | EGFR | QQASTSVYT | LCDR3 |
| | | | (Kabat) |
| 637 | EGFR | QQYATSVYT | LCDR3 |
| | | | (Kabat) |
| 638 | EGFR | QQYSASVYT | LCDR3 |
| | | | (Kabat) |
| 639 | EGFR | QQYSTAVYT | LCDR3 |
| | | | (Kabat) |
| 640 | EGFR | QQYSTSPYT | LCDR3 |
| | | | (Kabat) |
| 641 | EGFR | QQYSTSVYT | LCDR3 |
| | | | (Kabat) |
| 642 | EGFR | QQYSTSVYT | LCDR3 |
| | | | (Kabat) |
| 643 | EGFR | QQYSTSVYT | LCDR3 |
| | | | (Kabat) |
| 644 | EGFR | QQYSTSVYT | LCDR3 |
| | | | (Kabat) |
| 645 | EGFR | QQYSTSVYT | LCDR3 |
| | | | (Kabat) |
| 646 | EGFR | QQYSTSVYT | LCDR3 |
| | | | (Kabat) |
| 647 | EGFR | QQYSTSVYT | LCDR3 |
| | | | (Kabat) |
| 648 | EGFR | QQYSTSVYT | LCDR3 |
| | | | (Kabat) |
| 649 | EGFR | QQYSTSVYT | LCDR3 |
| | | | (Kabat) |
| 650 | EGFR | QQYSTSVYT | LCDR3 |
| | | | (Kabat) |
| 651 | EGFR | QQYSTSVYT | LCDR3 |
| | | | (Kabat) |
| 652 | EGFR | QQYSTSVYT | LCDR3 |
| | | | (Kabat) |
| 653 | EGFR | QQYSTSVYT | LCDR3 |
| | | | (Kabat) |
| 654 | EGFR | QQYSTSVYT | LCDR3 |
| | | | (Kabat) |
| 655 | EGFR | QQYSTSVYT | LCDR3 |
| | | | (Kabat) |
| 656 | EGFR | QQYSTSVYT | LCDR3 |
| | | | (Kabat) |
| 657 | EGFR | QQYSTSVYT | LCDR3 |
| | | | (Kabat) |
| 658 | EGFR | QQYSTSVYT | LCDR3 |
| | | | (Kabat) |
| 659 | EGFR | QQYSTSVYT | LCDR3 |
| | | | (Kabat) |
| 660 | EGFR | QQYSTSVYT | LCDR3 |
| | | | (Kabat) |
| 661 | EGFR | QQYSTSVYT | LCDR3 |
| | | | (Kabat) |
| 662 | EGFR | QQYSTSVYT | LCDR3 |
| | | | (Kabat) |
| 663 | EGFR | QQYSTSVYT | LCDR3 |
| | | | (Kabat) |
| 664 | EGFR | QQYSTSVYT | LCDR3 |
| | | | (Kabat) |
| 665 | EGFR | QQYSTSVYT | LCDR3 |
| | | | (Kabat) |
| 666 | EGFR | QQYSTSVYT | LCDR3 |
| | | | (Kabat) |
| 667 | EGFR | QQYSTSVYT | LCDR3 |
| | | | (Kabat) |
| 668 | EGFR | QQYSTSAYT | LCDR3 |
| | | | (Kabat) |
| 669 | EGFR | QQYHNLPYS | LCDR3 |
| | | | (Kabat) |
| 670 | EGFR | QHYDTAPFS | LCDR3 |
| | | | (Kabat) |
| 671 | EGFR | QQYDTRPLT | LCDR3 |
| | | | (Kabat) |
| 672 | ITGB6 | QQHYGIPWT | LCDR3 |
| | | | (Kabat) |
| 673 | ITGB6 | QQHYGIPWT | LCDR3 |
| | | | (Kabat) |
| 674 | ITGB6 | QHHYGVPWT | LCDR3 |
| | | | (Kabat) |
| 675 | ITGB6 | QHHYGVPWT | LCDR3 |
| | | | (Kabat) |

### Example 5—Monovalent EGFR Cell Binding Correlates to Affinity/Off-Rate

To determine whether cell binding correlates to affinity/off-rate, the following experiment was conducted. Briefly, cells were seeded in 96-well plates in triplicate and incubated with monovalent antibodies (0.01 to 1000 nM) for 1 hour on ice. After incubation, cells were washed 3 times with ice cold staining buffer (PBS+2% FBS), and then incubated with a fluorescently labeled anti-human IgG-Fc antibody for 30 minutes on ice. Cells were washed 3 times and then acquired on a Cytek Northern Lights flow cytometer.

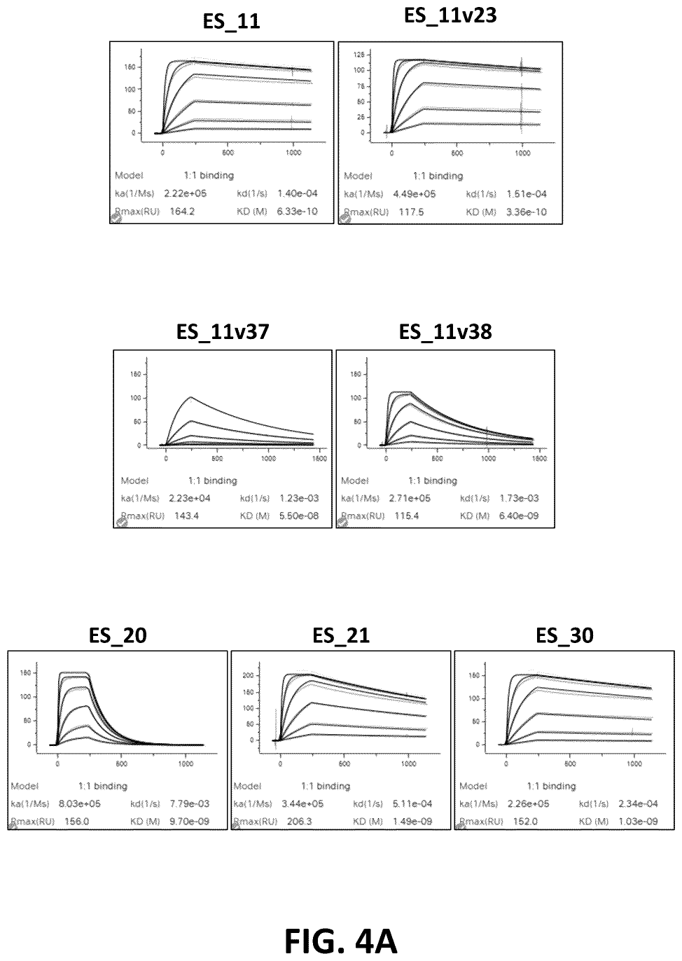

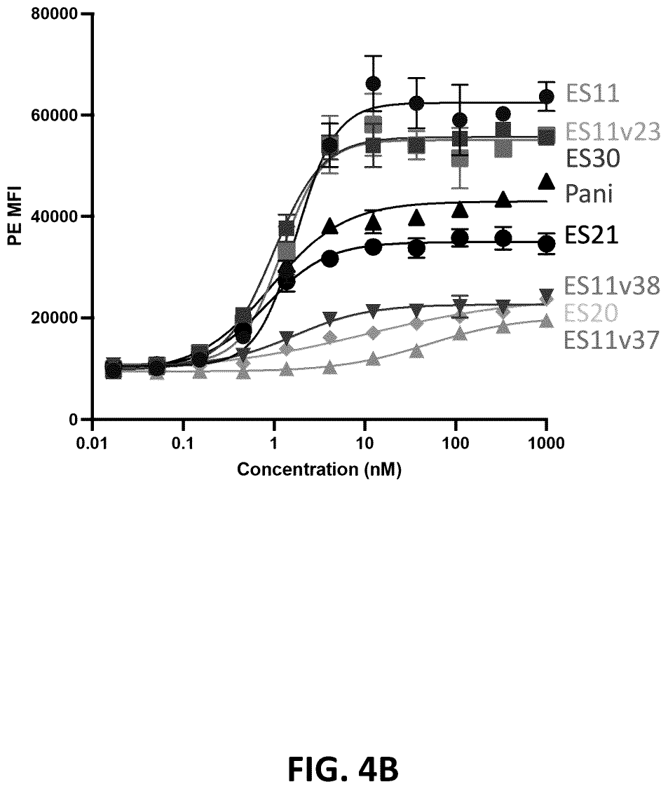

Measured EC50 binding values of monovalent EGFR antibodies on tumor cells closely mirrors the affinity (K_{d}) values determined by biophysical characterization (Octet and SPR), highlighting the fact that the antibodies display the expected behavior when binding EGFR in its native form on the surface of live cells (FIG. 4B and Table 7.

TABLE 7
Binding Properties of EGFR Binders

| | Octet Kd | Cell Binding | | SPR KD | T ½ |
|---|---|---|---|---|---|
| Binder | (nM) | EC50 (nM) | R² | (nM) | (min) |
| ES11v23 (SEQ ID NOs: 148, | 0.268 | 0.981 | 0.99 | .34 | 77 |
| 203, 258, 313, 368, 423, 478, | | | | | |
| 533, 588, 643) | | | | | |
| ES11 (SEQ ID NOs: 126, 181, | 0.544 | 1.768 | 0.97 | .63 | 82 |
| 236, 291, 346, 401, 456, 511, | | | | | |
| 566, 621) | | | | | |
| ES30 (SEQ ID NOs: 176, 231, | 0.889 | 1.216 | 0.97 | 1 | 49 |
| 286, 341, 396, 451, 506, 561, | | | | | |
| 616, 671) | | | | | |
| ES21 (SEQ ID NOs: 175, 230, | 1.56 | 0.826 | 0.98 | 1.5 | 23 |
| 285, 340, 395, 450, 505, 560, | | | | | |
| 615, 670) | | | | | |
| ES11v38 (SEQ ID NOs: 163, | 9.66 | 1.686 | 0.96 | 6.4 | 6.7 |
| 218, 273, 328, 383, 438, 493, | | | | | |
| 548, 603, 658) | | | | | |
| ES20 (SEQ ID NOs: 174, 229, | 12.5 | 10.61 | 0.96 | 9.7 | 1.5 |
| 284, 339, 394, 449, 504, 559, | | | | | |
| 614, 669) | | | | | |
| ES11v37 (SEQ ID NOs: 162, | 74.1 | 49.85 | 0.97 | 55 | 9.4 |
| 217, 272, 327, 382, 437, 492, | | | | | |
| 547, 602, 657) | | | | | |
| Pani | 0.582 | 0.931 | 0.98 | .7 | 33 |

### Example 6—EGFR Signal Blocking Potency Correlates with EGFR Affinity

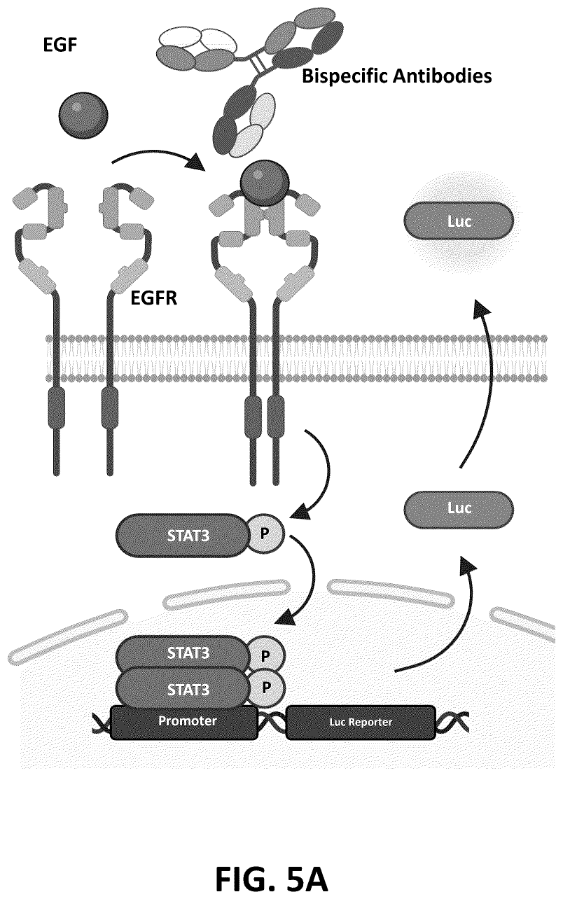

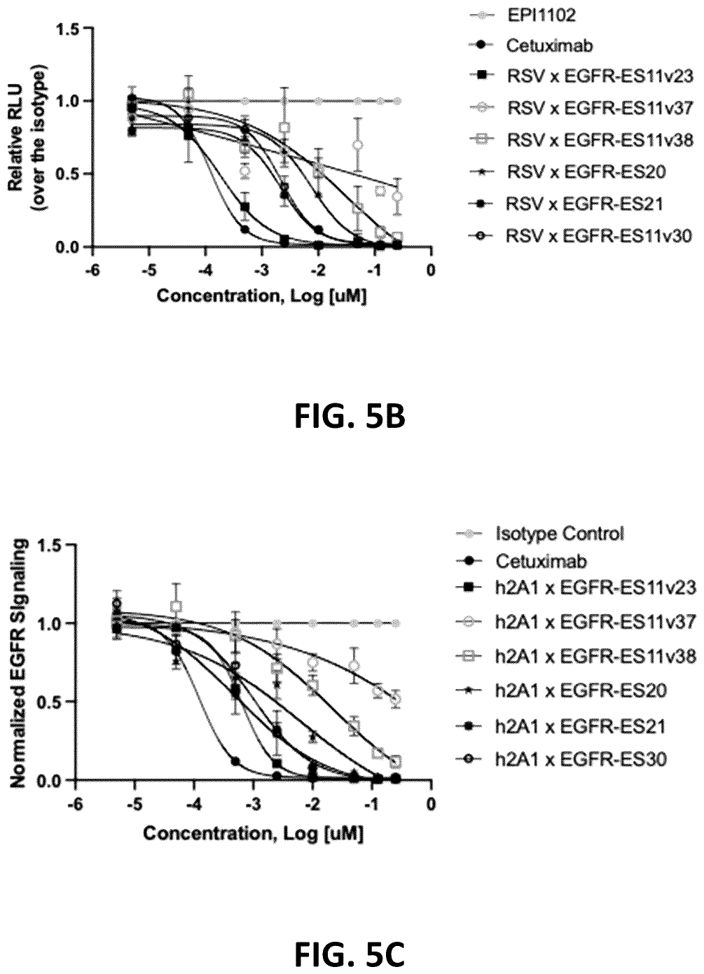

This example sought to determine whether antibodies that bind to EGFR could block EGF/EGFR signaling at different dose ranges and different binding affinities. Cetuximab, a known anti-EGFR monoclonal antibody, with strong binding and EGF/EGFR signaling inhibition ability was used as a positive control. The IgG1 isotype control, with no EGFR binding ability, was used as a negative control. Reporter cells that express EGFR but not ITG1B6 were thawed, resuspended in 6.4 mL of Cell Recovery Medium provided in the Indigo Biosciences EGFR1 Reporter Assay System kit (FIG. 5A). 200 μL/well of cell suspension was dispensed in the assay plate and the plate incubated at 37C for 4 hours. At the end of 4 hours, media was removed from the wells and 100 μL of a dose titration (250, 125, 50, 10, 25, 5, 0.5 and 0.05 nM) of single-arm anti-EGFR antibodies or EGFRxITGB6 antibodies (see FIGS. 5B and 5C), Cetuximab, or the isotype control. 100 μL of media containing 1.4 ng/mL EGF was added to each well. The assay plate was placed in an incubator for 22 hours at 37C. The luciferase detection reagent was generated by mixing the detection buffer and detection substrate. Media from the wells was removed and replaced with 100 μL of the detection reagent and luminescence was quantified. Normalized EGFR signaling was quantified as a ratio of relative light units of anti-EGFR antibody compared to the isotype control. Graphs and IC50s were generated using GraphPad Prism.

Cetuximab, single arm anti-EGFR, or EGFRxITGB6 bispecific antibodies inhibit EGF/EGFR signaling in a dose dependent manner, while an isotype control does not (FIG. 5B (treatment groups: EPI1102, Cetuximab, and RSV× any one of: EGFR-ES11v23, EGFR-ES11v37, EGFR-ES11v38, EGFR-ES20, EGFR-ES21 or ES11v30) and 5C (treatment groups: Isotype Control, Cetuximab, and h2A1× any one of: EGFR-ES11v23, EGFR-ES11v37, EGFR-ES11v38, EGFR-ES20, EGFR-ES21 or ES 1v30) The monovalent IC50 (IC50 of single arm anti-EGFR antibodies) and the EGFRxITGB6 IC50 (IC50 of the EGFR arm of EGFRxITGB6 bispecific antibody) were both determined (Table 8). Broadly, the IC50 of anti-EGFR antibodies corresponds to the EGFR binding affinities and monovalent IC50.

TABLE 8
Kd and IC50 of EGFR Binders

| | EGFR | | | Fold- |
|---|---|---|---|---|
| | Affinity | Single Arm | EGFR × ITGB6 | Change vs. |
| EGFR Binder | K_{D}, (nM) | IC50 (nM) | IC50 (nM) | Cetuximab |
| Cextuximab | 1.5 | 0.14 (Bivalent) | | — |
| ES11v23 (SEQ ID NOs: 148, | 0.34 | 0.18 | 0.63 | ~2 |
| 203, 258, 313, 368, 423, 478, | | | | |
| 533, 588, 643) | | | | |
| ES30 (SEQ ID NOs: 176, 231, | 1 | 2.13 | 0.98 | ~5 |
| 286, 341, 396, 451, 506, 561, | | | | |
| 616, 671) | | | | |
| ES21 (SEQ ID NOs: 175, 230, | 1.5 | 2.10 | 0.47 | ~5 |
| 285, 340, 395, 450, 505, 560, | | | | |
| 615, 670) | | | | |
| ES11v38 (SEQ ID NOs: 163, | 6.4 | 31.68 | 21.94 | ~150 |
| 218, 273, 328, 383, 438, 493, | | | | |
| 548, 603, 658) | | | | |
| ES20 (SEQ ID NOs: 174, 229, | 9.7 | 7.69 | 6.05 | ~40 |
| 284, 339, 394, 449, 504, 559, | | | | |
| 614, 669) | | | | |
| ES11v37 (SEQ ID NOs: 162, | 55 | >1000 | >1000 | ~10,000 |
| 217, 272, 327, 382, 437, 492, | | | | |
| 547, 602, 657) | | | | |

### Example 7—Low Affinity Anti-EGFR Antibodies Have Limited Activity on EGFR Signaling in Normal Skin Cells

This example sought to determine whether single-arm anti-EGFR antibodies with different binding affinities would affect EGFR signaling in normal skin cells. In this example, primary epidermal keratinocytes (HEKa) were seeded overnight at 50,000 cells/well of a 24-well plate. Media was removed and cells were treated with bivalent antibodies, Cetuximab, or an isotype control for 48 hours. Media was removed, and cells were treated with 100 ng/mL EGF for 15 minutes at 37° C. Cells were washed with PBS and protein lysates were prepared in RIPA lysis buffer. Protein quantification was measured by Pierce BCA Protein Assay Kit from ThermoFisher according to manufacturer's instructions. 15 μg of protein per sample was loaded and run on a NuPAGE, 4-12% Bio-Tris Midi Gel, followed by PVDF membrane transfer. Membranes were blocked in LICOR blocking buffer, followed by primary antibody, followed by 3 washes in TBST, then by secondary antibody in LICOR antibody diluent, 3 washes in TBST. Finally, the membrane was read on Licor Odyssey DLx. Actin was used as a housekeeping control.

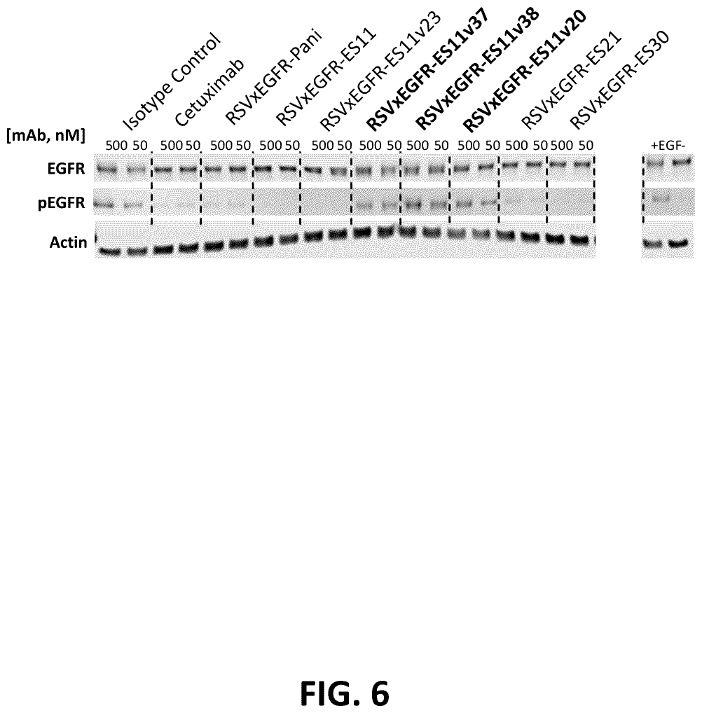

Treatment with all single-arm antibodies did not affect EGFR levels in HEKa cells (FIG. 6). However, only antibodies with lower affinity (ES11v37, ES11v38 and ES20) demonstrated limited impact on phospho-EGFR levels compared to high affinity antibodies. Low affinity EGFR antibodies have limited activity on EGFR signaling in normal skin cells.

### Example 8—PK Profiles of Anti-EGFR Antibodies Track with Mouse EGFR Affinity

This example sought to determine the pharmacokinetic properties of monovalent anti-EGFR antibodies in tumor-free mice. In this example, a cohort of 6-9-week-old female Athymic nude mice were randomized into groups (n=6) based on body weight, then injected with a single 5 μl/g volume dose of single arm anti-EGFR antibodies at either 3 mg/kg or 15 mg/kg or Cetuximab at 3 mg/kg, intravenously. The single arm anti-EGFR antibodies bind murine EGFR with similar or lower affinity than human, whereas Cetuximab does not bind murine EGFR. Single arm anti-EGFR antibodies (RSVxEGFR) were prepared in-house and cetuximab was purchased from MedChemExpress (HY-P9905). The initial dose was noted as time-point 0. Serum samples were collected and frozen from each animal. Two in-life (submandibular) cheek bleeds and one terminal (cardiac) bleed were performed on each mouse for sample collection, per IACUC guidelines, at 1, 24, 48, 96, 120 and 168 hours. Sub-groups (n=3/group) were utilized to stagger blood collection from individual animals. Animals were monitored daily and weighed multiple times per week, according to IACUC guidelines. Serum concentration (ng/ml) of each mAb was measured using the Human Therapeutic IgG1 ELISA kit (Cayman #500910) according to manufacturer's instructions. Concentrations of human IgG1 in serum was computed relative to a standard curve of positive control samples. Pharmacokinetic analysis was performed using WinNonlin Phoenix software (Certara, version 8.2). Graphs were created in GraphPad Prism on a log or linear scale. Dotted lines on the graph indicate 10,000 ng/mL for reference.

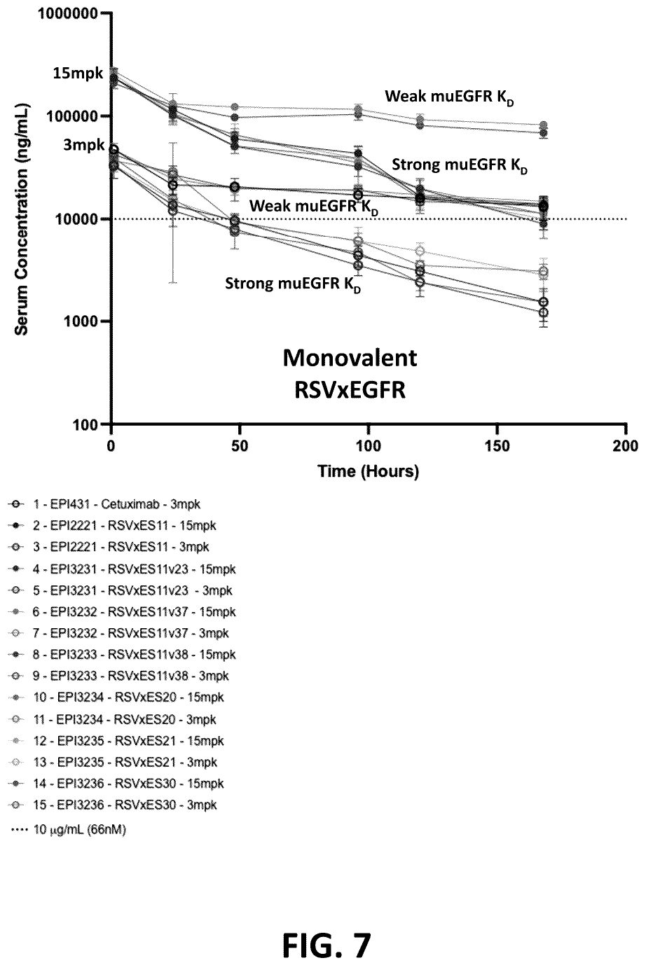

All treatment groups had measurable human IgG in serum at levels 10 μg/mL or greater at 24 hour time-point post-dose (FIG. 7). As shown in FIG. 7 and Table 9, mice treated with EST1v37 and ES11v38, with weak binding affinity to murine EGFR, demonstrated stabilized serum levels throughout the end of the study. The pharmacokinetic profiles and half-lives of ES 11v37 and ES 11v38 (weak affinity EGFR binders) as well as cetuximab were similar. Conversely, mice treated with (see FIG. 7) strong EGFR binders demonstrated lower half life and pharmacokinetics profiles compared to cetuximab. ES20, a strong murine EGFR binder with a faster target on/off rate, demonstrates a correlation between affinity and exposure suggesting target mediate drug disposition (TMDD).

TABLE 9
Pharmacokinetic Properties of EGFR Binders

| | | | | | | Clearance | Clearance |
|---|---|---|---|---|---|---|---|
| | | | | T½ (h) | T½ (h) | (mL/h/kg) | (mL/h/kg) |
| | huEGFR | muEGFR | | for 15 | for 3 | for 15 | for 3 |
| EGFR Binder | Kd (nM) | Kd (nM) | Valency | mg/kg | mg/kg | mg/kg | mg/kg |
| ES11v23 (SEQ | 0.34 | 1.7 | Mono | 39.6 | 47.4 | 1.59 | 2.39 |
| ID NOs: 158, | | | | | | | |
| 213, 268, 323, | | | | | | | |
| 378, 433, 488, | | | | | | | |
| 543, 598, 653) | | | | | | | |
| ES11 (SEQ ID | 0.63 | 3.1 | Mono | 41.4 | 47.8 | 1.3864 | 2.09 |
| NOs: 126, 181, | | | | | | | |
| 236, 291, 346, | | | | | | | |
| 401, 456, 511, | | | | | | | |
| 566, 621) | | | | | | | |
| ES30 (SEQ ID | 1 | 3.4 | Mono | 39.8 | 44.2 | 1.53 | 2.06 |
| NOs: 176, 231, | | | | | | | |
| 286, 341, 396, | | | | | | | |
| 451, 506, 561, | | | | | | | |
| 616, 671) | | | | | | | |
| ES21 (SEQ ID | 1.5 | 2.4 | Mono | 41.8 | 65.3 | 1.42 | 1.66 |
| NOs: 175, 230, | | | | | | | |
| 285, 340, 395, | | | | | | | |
| 450, 505, 560, | | | | | | | |
| 615, 670) | | | | | | | |
| ES11v38 (SEQ | 6.4 | 350 | Mono | 128.9 | 159.8 | 0.49 | 0.45 |
| ID NOs: 163, | | | | | | | |
| 218, 273, 328, | | | | | | | |
| 383, 438, 493, | | | | | | | |
| 548, 603, 658) | | | | | | | |
| ES20 (SEQ ID | 9.7 | 6 | Mono | 44.8 | 43.7 | 1.44 | 1.48 |
| NOs: | | | | | | | |
| 174, 229, 284, | | | | | | | |
| 339, 394, 449, | | | | | | | |
| 504, 559, 614, | | | | | | | |
| 669) | | | | | | | |
| ES11v37 (SEQ | 55 | ~2400 | Mono | 207.8 | 212.6 | 0.33 | 0.37 |
| ID NOs: 162, | | | | | | | |
| 217, 272, 327, | | | | | | | |
| 382, 437, 492, | | | | | | | |
| 547, 602, 657) | | | | | | | |
| Cetuximab | 1.5 | No | Bivalent | — | 188 | — | 0.43 |
| | | Binding | | | | | |

### Example 9—ITGB6 Binders Are Specific to ITGB6

To determine whether ITGB6 binders were specific to ITGB6, the following experiment was conducted. A375 cells which express alphaV integrin or A375 cells which were transiently overexpressed ITGB6 or ITGB8 were seeded at 4e5 cells in 6-well tissue culture plates. The following day cells were transiently transfected with FuGene HD and harvested for flow cytometry 48 hours post transfection. In the presence of Ca² (PBS+0.5% BSA), cells were incubated with 250 nM anti-ITGB6 antibodies for 20 minutes on ice. Subsequently cells were washed and stained with secondary PE anti-Human antibody for 20 minutes on ice, in the dark. Cells were analyzed on the Cytek Northern Lights flow cytometer.

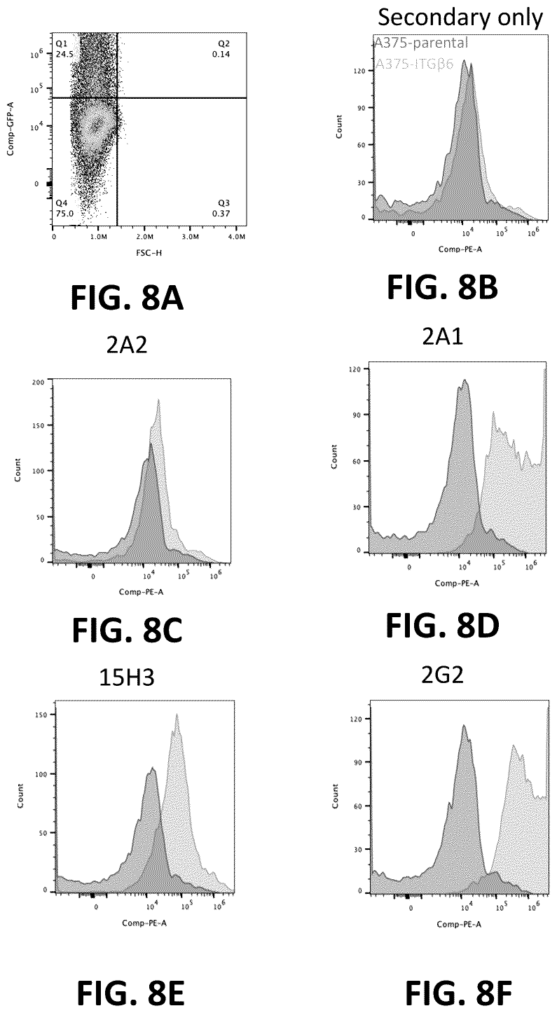

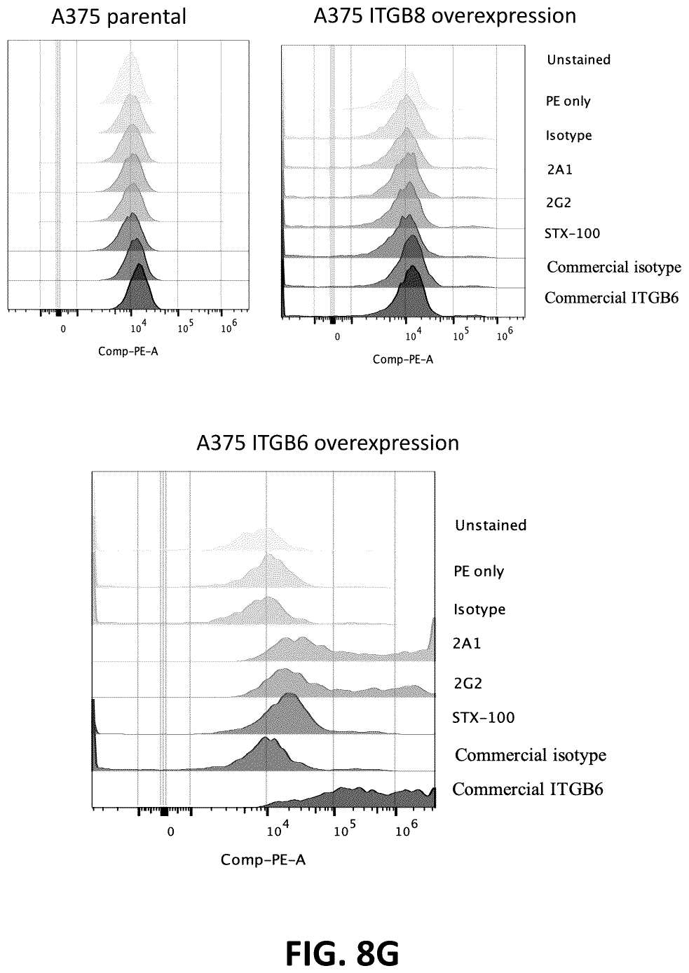

As shown in FIGS. 8A-8G, ITGB6 binders (2A2, 2A1, 15H3, 2G2, STX-100, Commercial ITGB6, commercial ITGB6, isotype) bound to cells overexpressing ITGB6 more than parental cells, indicating that all tested ITBG6 antibodies specifically bound to ITGB6 alone. No binding was observed in A375 parental lines that express integrins other than B6. Similarly, no binding was observed in A375 cells that overexpress ITGB8. However, expression was observed in A375 cells expressing ITGB6.

### Example 10—Bivalent ITGB6 Antibodies Have Extended Exposure

This example sought to determine the pharmacokinetic properties of bivalent antibodies in tumor-free mice. For this example, a cohort of 6-9-week-old female Athymic nude mice were randomized into groups (n=6) based on body weight, then injected with a single 5 μl/g volume dose of Cetuximab or bivalent anti-ITGB6 antibodies at either 3 mg/kg or 15 mg/kg, intravenously. Bivalent anti-ITGB6 antibodies were prepared in-house and cetuximab was purchased. The initial dose was noted as time-point 0. Serum samples were collected and frozen from each animal. Two in-life (submandibular) cheek bleeds and one terminal (cardiac) bleed were performed on each mouse for sample collection, per IACUC guidelines, at 1, 24, 48, 96, 120 and 168 hours. Sub-groups (n=3/group) were utilized to stagger blood collection from individual animals. Animals were monitored daily and weighed multiple times per week, according to IACUC guidelines. Serum concentration (ng/ml) of each antibody was measured using the Human Therapeutic IgG1 ELISA kit according to manufacturer's instructions. Concentrations of human IgG1 in serum was computed relative to a standard curve of positive control samples. Pharmacokinetic analysis was performed.

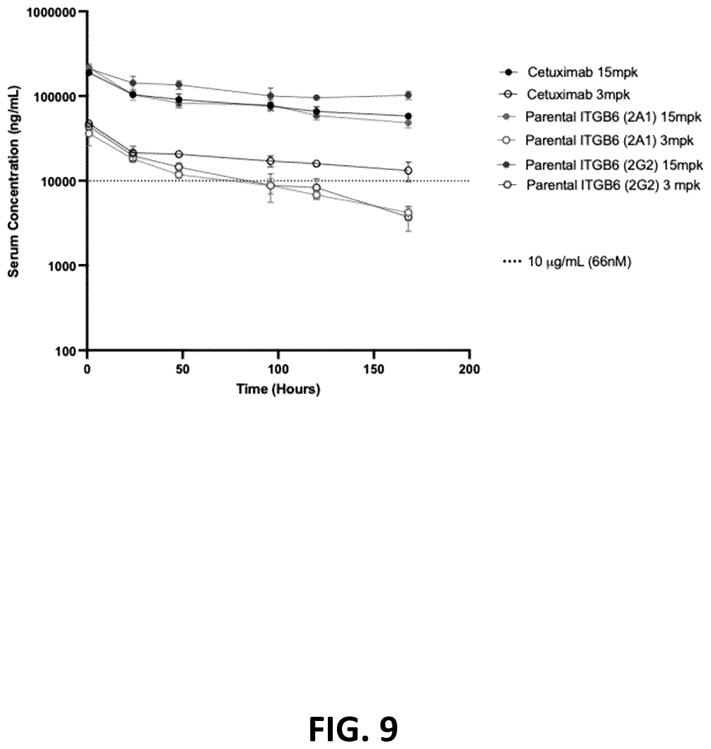

All treatment groups had measurable human IgG in serum at levels 10 μg/mL or greater at 48 hour time-point post-dose (FIG. 9; dotted lines on the graph indicate 10,000 ng/mL for reference). Cetuximab lacks binding to murine EGFR and hence showed stabilized IgG levels and PK profiles throughout the end of the study. Conversely, ITGB6 (2A1 and 2G2) demonstrate pharmacokinetic profiles in line with modest target mediated drug disposition (TMDD).

TABLE 10
Pharmacokinetic Properties of ITGB6 Binders

| | | | | | | Clearance | Clearance |
|---|---|---|---|---|---|---|---|
| | huTarget | muTarget | | T½ (h) | T½ (h) | (mL/h/kg) | (mL/h/kg) |
| Binder | Kd (nM) | Kd (nM) | Valency | 15 mg/kg | 3 mg/kg | 15 mg/kg | 3 mg/kg |
| Cetuximab | 1.5 | No | Bivalent | 170 | 188 | 0.5202 | 0.4361 |
| | | Binding | | | | | |
| ITGB6 (2A1) (SEQ | 8.9 | 4.2 | Bivalent | 137 | 67.5 | 0.629 | 1.2658 |
| ID NOs: 177, 232, | | | | | | | |
| 287, 342, 397, 452, | | | | | | | |
| 507, 562, 617, 672) | | | | | | | |
| ITGB6 (2G2) (SEQ | 23 | No data | Bivalent | 167 | 63.2 | 0.3323 | 1.164 |
| ID NOs: 179, 234, | | | | | | | |
| 289, 344, 399, 454, | | | | | | | |
| 509, 564, 619, 674) | | | | | | | |
| RNF43 (231) | 0.8 | No data | Bivalent | 148 | 128 | 0.5436 | 0.6123 |
| RSVxEGFR-ES11 | 0.63 | 3.1 | Mono | 41.4 | 47.8 | 1.3864 | 2.095 |

### Example 11—LAP Blocking

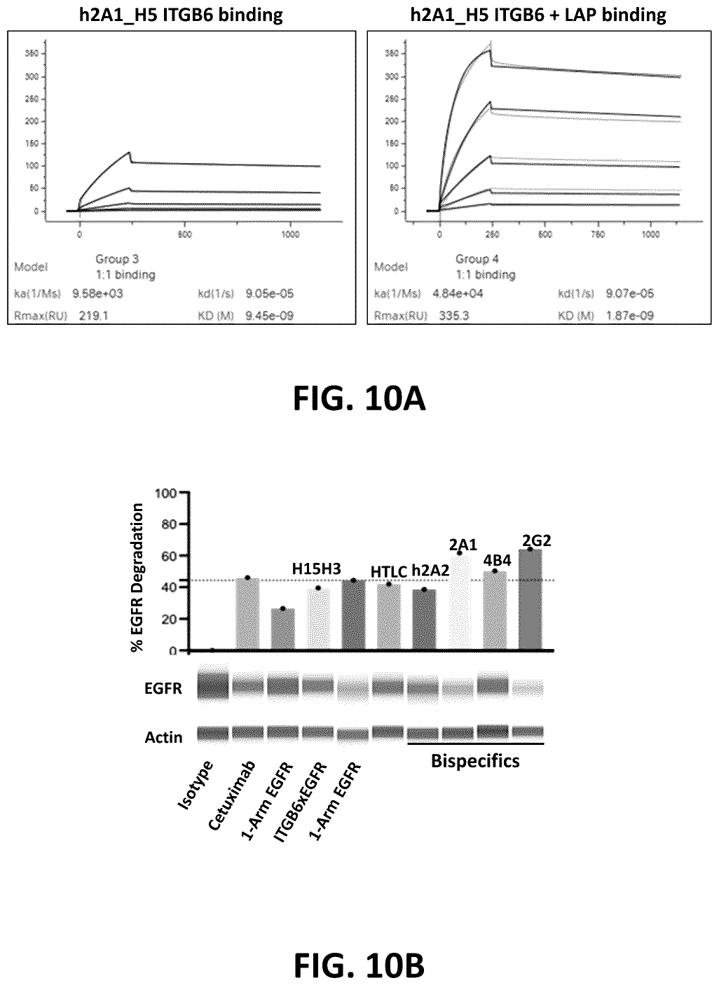

The ability of anti-ITGB6 antibodies to block latency associated peptide (LAP) was determined by biolayer interferometry (BLI) using a Sartorius Octet RH96 instrument. Experiments were performed at 25° C. in a HEPES buffered saline pH 7.4 solution containing 0.05% Tween-20, 1% BSA, and 0.3 mM calcium chloride. Biotinylated human aV06 ECD was captured on streptavidin sensors and saturated with an anti-ITGB6 IgG, then assessed for binding to recombinant LAP. Antibodies known to block LAP, clones h15H3-HTLC and STX-100, demonstrated the ability to block LAP from binding to aV06 ECD. Clones 2A1 and 2G2 were not able to block LAP binding to aV06 ECD (FIG. 10A).

### Example 12—EGFRxITGB6 Bispecific Antibodies that Do Not Block LAP Improve EGFR Degradation

To determine the effect of LAP blocking on EGFR degradation, the following experiment was performed. NCIH1975 cells were seeded at 4e5 cells in 6 well tissue culture plate. After approximately 16 hours of culture, a single concentration of antibodies was added to cells and treated for 48 hours. Media was removed and cells were lysed. Prepared samples were loaded onto a 4-12% BisTris gel and transferred to PVDF membrane. The membrane was probed with EGFR or p-EGFR and the housekeeping gene β-actin. Data was quantified using Empiria studio; percent degradation normalized to R-actin and compared to isotype control.

EGFR binders paired with non-LAP blocking ITGB6 binders (2A1 and 2G2) result in higher EGFR degradation than the isotype Cetuximab, 1-Arm EGFR, H15H3 (LAP blocking EGFRxITGB6 antibodies), h2A2 and 4B4 (bispecific). (FIG. 10B). 2A1 and 2G2 drive best degradation and as non-LAP blockers.

### Example 13—Humanization of ITGB6 Antibodies

Clone 2A1 was humanized by grafting CDR residues onto human frameworks VH1-46*02 and VK1-33*01. Various combinations of backmutations at Kabat positions 48, 49, 60, 61, 64, 67, 69, 71, 73, and 94 were introduced into the heavy chain and assessed for binding kinetics and biophysical properties. Clone h2A1_H5 was selected as it demonstrated similar binding kinetics and favorable biophysical properties.

Clone 2G2 was humanized by grafting CDR residues onto human frameworks VH1-69*02 and VK1-39*01. Various combinations of backmutations at Kabat positions 48, 60, 61, 64, 67, 69, and 94 were introduced into the heavy chain and assessed for binding kinetics and biophysical properties. Clone h2G2_H4 was selected as it demonstrated similar binding kinetics and favorable biophysical properties.

TABLE 11
Characterization of ITGB6 Binders

| | | | | | | LAP |
|---|---|---|---|---|---|---|
| | | | | | | Enhanced |
| | ITGB-Av | huITGB6 | cyITGB6 | muITGB6 | LAP | Binding |
| Clone | Binding Screen | KD (nM) | KD (nM) | KD (nM) | Blocking | (nM) |
| 2A1 (SEQ ID NOs: | ITGB6-specific | 8.9 | 5.3 | 4.2 | No | 1.8 |
| 177, 232, 287, 342, | | | | | | |
| 397, 452, 507, 562, | | | | | | |
| 617, 672) | | | | | | |
| H2A1_H5 (SEQ ID | Data Pending | 9.5 | 5.6 | 4.1 | No | 1.9 |
| NOs: 178, 233, 288, | | | | | | |
| 343, 398, 453, 508, | | | | | | |
| 563, 618, 673) | | | | | | |
| 2G2 (SEQ ID NOs: | ITGB6-specific | 23 | Data | Data | No | 4.4 |
| 179, 234, 289, 344, | | | Pending | Pending | | |
| 399, 454, 509, 564, | | | | | | |
| 619, 674) | | | | | | |
| H2G2_H4 (SEQ ID | Data Pending | 16 | 15 | 11 | No | 4.7 |
| NOs: 180, 235, 290, | | | | | | |
| 345, 400, 455, 510, | | | | | | |
| 565, 620, 675) | | | | | | |

### Example 14—Low Affinity EGFRxITGB6 Antibodies Have Limited Activity on EGFR Signaling in Normal Skin Cells

This example sought to determine whether EGFRxITGB6 antibodies with different binding affinities and high ITGB6 expression could affect EGFR signaling in normal skin cells. For this example, primary epidermal keratinocytes were seeded overnight at 50,000 cells/well of a 24-well plate. Media was removed and cells were treated with antibodies for 48 hours. Media was removed, and cells were treated with 100 ng/mL EGF for 15 minutes at 37° C. Cells were washed with PBS and protein lysates were prepared in lysis buffer. 15 μg of protein per sample was loaded and run on a Gel, followed by PVDF membrane transfer. Actin was used as a housekeeping control.

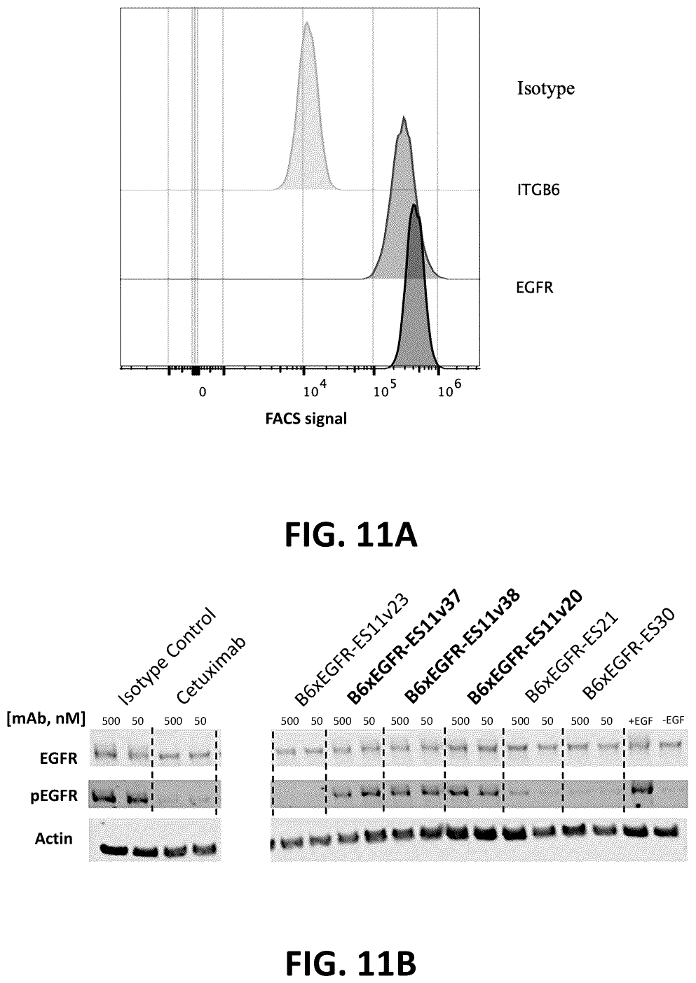

HEKa cells have expression of EGFR and ITGB6 on their cell surface (FIG. 11A). However, despite expression levels, B6xEGFR× any one of ESllv20, ES11v37 and ES11v38 (bispecific EGFRxITGB6 antibodies with low affinity EGFR binders) demonstrated limited impact on phospho-EGFR levels compared to EGFRxITGB6 antibodies with B6xEGFR×ES11v23, ES21, ES30 (high affinity EGFR binders). All EGFR×ITGB6 antibodies had little to no impact on total EGFR levels on HEKa cells. Despite expression levels of ITGB6 and EGFR, ITGB6 degraders paired with low affinity EGFR binders have limited activity on EGFR signaling.

### Example 15—EGFRxITGB6 Antibodies Cause Less Inflammation

For this example, skin tissues from abdominoplasty surgeries were obtained, adipose layer removed and 5 mm biopsy punches were made. Six 5 mm biopsies were added to each well of a 24-well plate and 600 μL of media treated with test articles was added to each well. Cetuximab and EGFRxITGB6 antibodies were treated at 500 μg/ml whereas Amivantamab was treated at 600 μg/ml. A cocktail of IL2, IL23, anti-CD3 and anti-CD28 was added as a positive control. 75 μl media was collected after 24 hours. 25 μl of media collected post treatment was run using a MILLIPLEX® Human Cytokine/Chemokine/Growth Factor Panel A 48 Plex Premixed Magnetic Bead Panel as per manufacturer's instructions. Data was analyzed using GraphPad Prism.

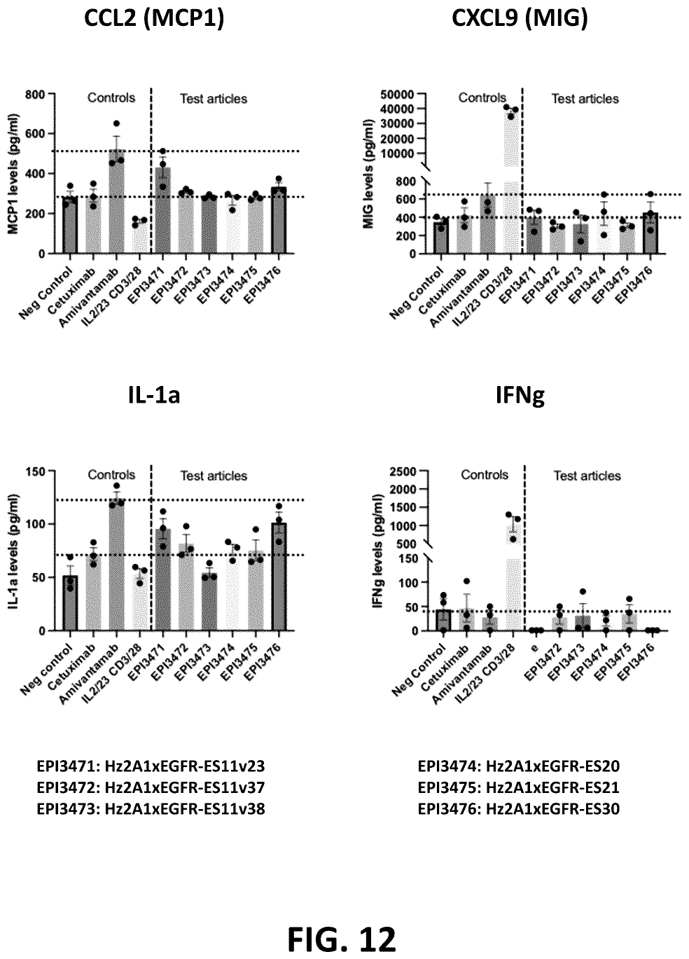

Compared to standard of care EGFR antagonists and positive controls, EGFRxITGB6 treatment (EPI3471-3476; EPI3471: Hz2A1xEGFR-ES11v23; EPI3472: Hz2A1xEGFR-ES11v37; EPI3473: Hz2A1xEGFR-ES11v38; EPI3474: Hz2A1xEGFR-ES20; EPI3475: Hz2A1xEGFR-ES21; EPI3476: Hz2A1xEGFR-ES30) did not result in upregulation of inflammatory cytokines (CCL2, CXCL9, IL-1a, IFN-g) in primary human skin tissue (FIG. 12).

### Example 16—Humanized 2A1/ES11 Variants Exhibit Reduction in EGFR Levels When Paired with Weaker Affinity EGFR Binders

To determine the effectiveness of EGFRxITGB6 antibodies, EGFR binders with different K_{d} were assayed for activity by flow cytometry to measure % EGFR cell surface removal and by western blot to measure % EGFR degradation. For EGFR cell surface removal, NCIH1975 cells were seeded at 1e4 cells in 96-well plates and incubated overnight at 37° C. and 5% CO2. Cells were then treated with a concentration of bispecific antibodies. After 72 hours of treatment, cells were harvested using a dissociation reagent, stained using a fluorescently labeled anti-EGFR antibody, and acquired on a Cytek Northern Lights flow cytometer. Percent EGFR cell surface removal was calculated using an untreated control sample after accounting for background with an isotype control. For Western Blot assay, NCIH1975 cells were seeded at 4e⁵ cells in 6-well tissue culture plate. After approximately 16 hours of culture, a single concentration of antibodies was added to cells in serum starved media and treated for 24-48 hours. Media was removed and stimulated with EGF in serum free media. Media was removed and cells were lysed. Prepared samples were loaded onto a 4-12% BisTris gel and transferred to PVDF membrane. Membrane was probed with EGFR or the housekeeping gene β-actin. Data quantified using Empiria studio; percent degradation normalized to R-actin and compared to PBS control.

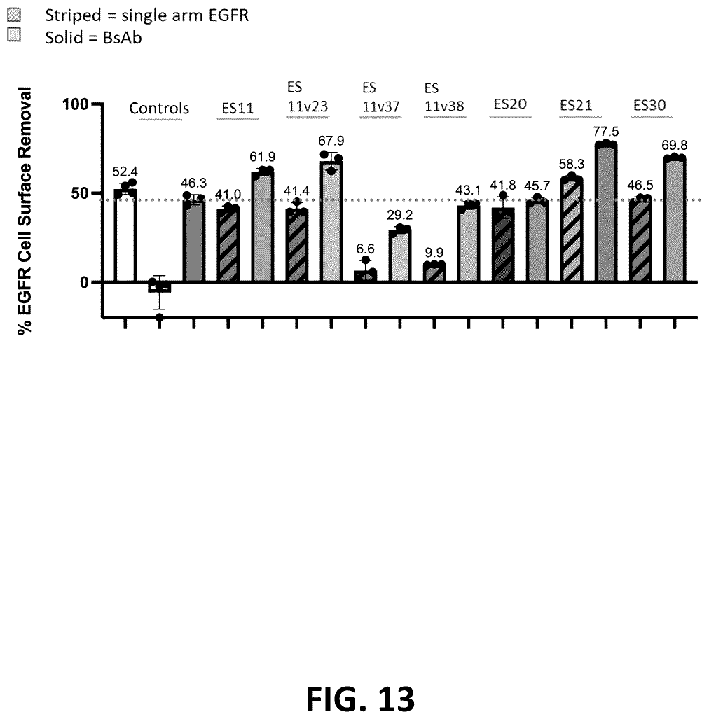

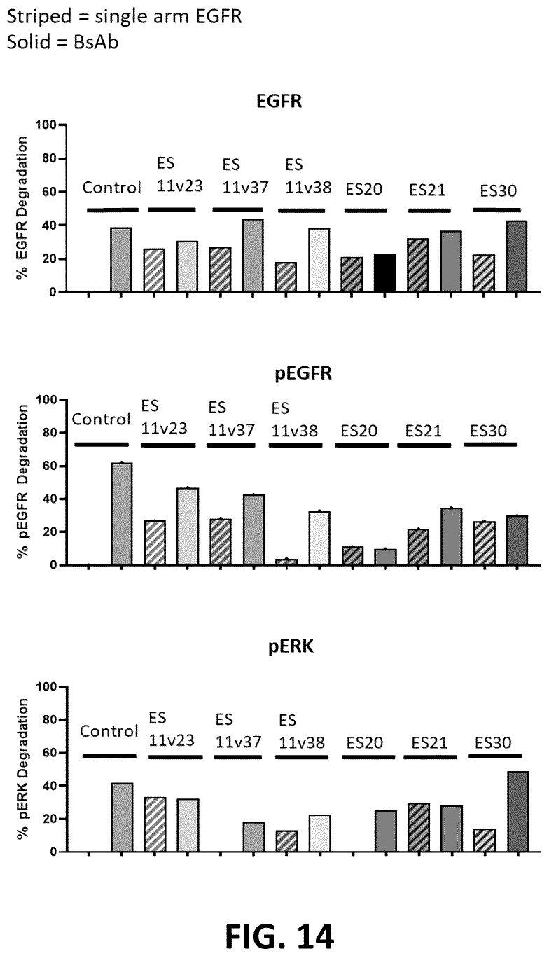

EGFRxITGB6 antibodies drove removal of EGFR from the surface and degraded EGFR (see FIG. 13 and FIG. 14). Removal and degradation were maintained for ESI 1v37 and ES11v38 (EGFRxITGB6 antibodies with lower affinity EGFR binders). Scaffolding signaling components (i.e. pERK) was also disrupted in EGFRxITGB6 antibodies with lower affinity EGFR binders.

### Example 17—Low affinity EGFRxITGB6 Antibodies Exhibit Tumor Cell Killing

This example sought to determine whether single arm EGFR binder with different binders can cause tumor cell killing and if the addition of an ITGB6 degrader arm (EGFRxITGB6) can potentially enhance tumor killing. For this example, NCI-H1975-GFP cells were seeded at a density of 3000 cells/well/100 μL of a clear bottom black plate. After resting overnight, media was removed, and cells were treated with single arm, bispecific antibodies and isotype as a negative control. To this, donor PBMCs pre-primed with 20 ng/mL IL2 (R&D systems) was added at an E:T ratio of 10:1. The plate was placed in an Incucyte® S3 Live-Cell Analysis System and readings taken every 3 hours for 96 hours. The Incucyte software was used to normalize the GFP signal from each time point to the 0 hour (0 h) reading. The relative GFP signal was plotted using GraphPad Prism.

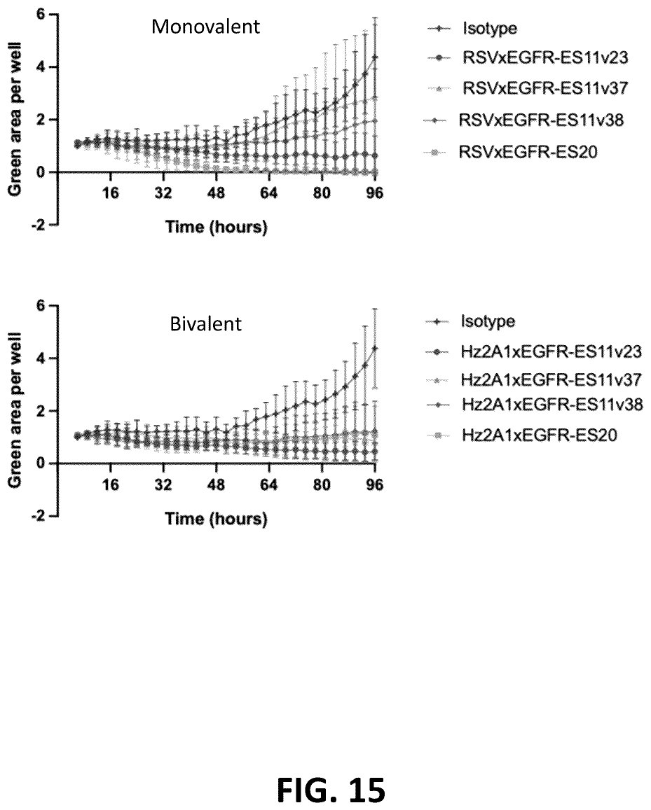

As shown in FIG. 15, treatment with ES11v23, with sub-nanomolar EGFR binding affinity, resulted in strong tumor cell killing, however, the ITGB6 arm did not have any positive effect on tumor killing. Conversely, the ITGB6 degrader arm on ES11v37 and ES11v38 enhanced tumor cell killing compared to their respective single arm EGFR binder.

### Example 18—EGFRxITGB6 Antibodies Suppress Tumor Growth

This example sought to determine whether I EGFRxITGB6 bispecific antibodies could phamacologically inhibit tumor growth in mouse tumor models. For this example, NCI-H1975 cells lines were grown in tissue culture flasks containing RPMI 1640 medium supplemented with 10% fetal bovine serum at 37° C. in an atmosphere of 5% CO₂. Cells were harvested during exponential growth phase, and 5×106 total cells were inoculated into the right front flank of BALB/c nude mice. Animals were monitored daily, and tumor volumes and body weights were measured twice per week according to IACUC guidelines. Tumors were grown until reaching approximately a volume of 200 mm³, at which point animals were randomized into groups (n=6/group) and given intraperitoneal doses of the indicated antibodies. EGFRxITGB6 antibodies and single arm EGFR antibodies were prepared in-house, cetuximab was purchased from MedChemExpress and isotype control antibodies were purchased from BioXcell. Dosing continued twice-per-week for 2 weeks and animals were monitored for up to 50 days from the initial dose. Tumor volume was calculated as V=(L×W×W)/2. Graphs and statistical analysis were done in Graphpad Prism using ordinary one-way ANOVA w/Tukey's multiple comparisons test, *p<0.05, **p<0.01, ***p<0.001.

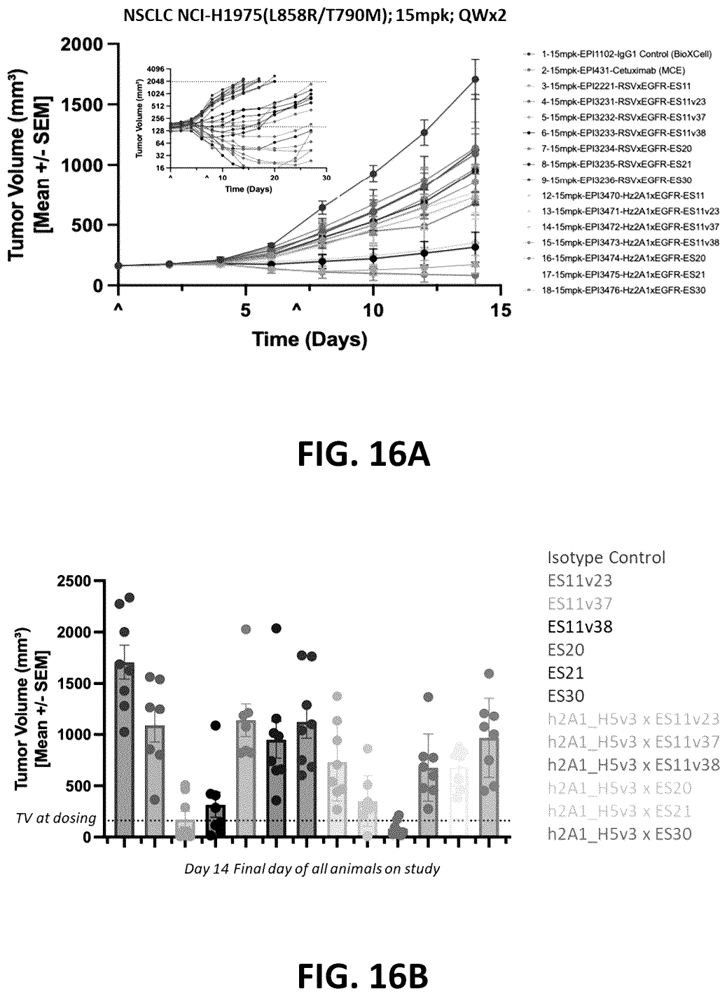

Tumor growth inhibition was compared between single arm EGFR antibodies and EGFRxITGB6 antibodies (see for example FIGS. 16A-B; Table 12). Significant tumor growth inhibition mediated by EGFRxITGB6 compared to single arm EGFR binder was observed in groups with ES11v23, ES 11v38 and ES20. While the delta between ES20 and its bispecific antibody was the highest, 2A1xEGFR-ES11v38 demonstrated the highest % TGI.

TABLE 12
Tumor Growth Inhibition of EGFRxITGB6 Antibodies

| Lead Op Candidate | Single arm | Bivalent | Delta |
|---|---|---|---|
| h2A1_H5v3 × | 36.1% | 57.0% | 20.9% |
| ES11v23 | | | |
| h2A1_H5v3 × | 89.8% | 79.3% | −10.5% |
| ES11v37 | | | |
| h2A1_H5v3 × | 81.5% | 95.2% | 13.7% |
| ES11v38 | | | |
| h2A1_H5v3 × ES20 | 33.2% | 60.3% | 27.2% |

### Example 19-bYlok® Placement on EGFR and ITGB6 Arms Effect on Antibody Function

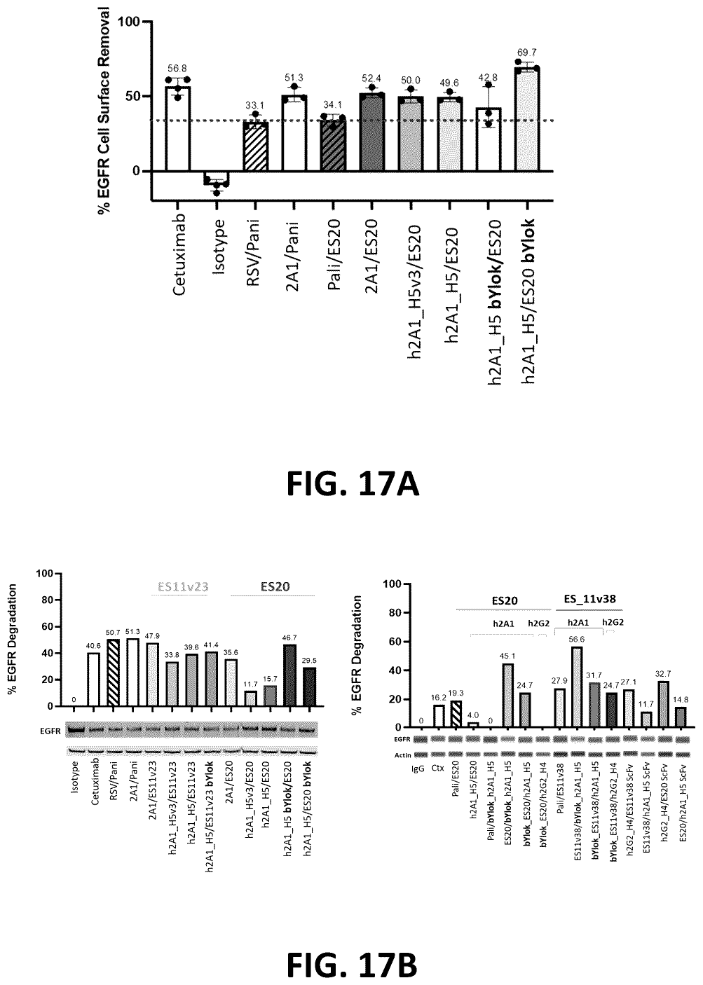

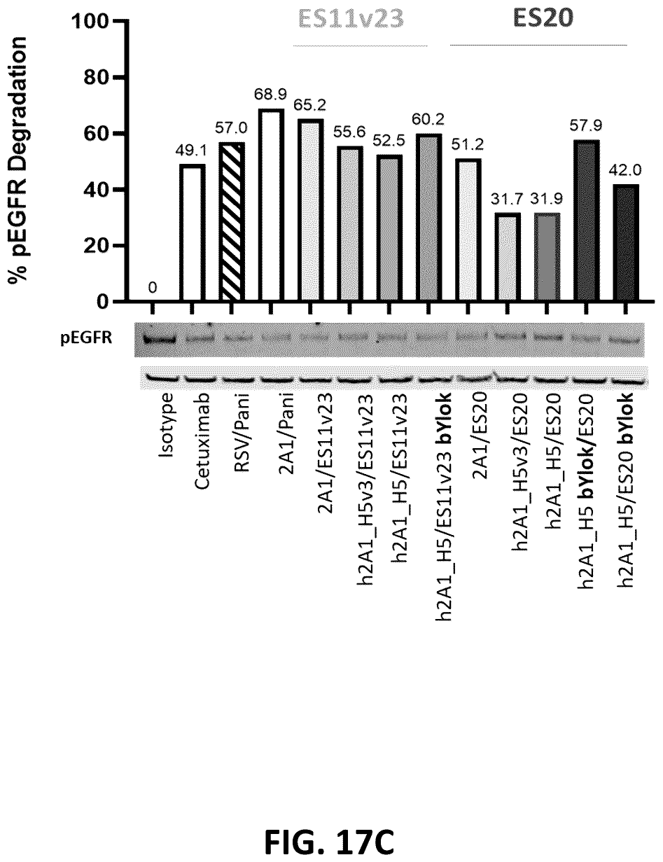

This example sought to determine whether bYlok® placement affects function of the bispecific antibodies (FIGS. 17A-17C). For cell surface removal assays, NCIH1975 cells were seeded at 1e4 cells in 96-well plates and incubated overnight at 37° C. and 5% CO₂. Cells were then treated with a concentration of bi-specific antibodies. After 72 hours of treatment, cells were harvested using a dissociation reagent, stained using a fluorescently labeled anti-EGFR antibody, and acquired on a Cytek Northern Lights flow cytometer. Percent EGFR cell surface removal was calculated using an untreated control sample after accounting for background with an isotype control. For Western Blot assay, NCIH1975 cells were seeded at 4e5 cells in 6-well tissue culture plate. After approximately 16 hours of culture, a single concentration of antibodies were added to cells and treated for 48 hours. Media was removed and cells were lysed. Prepared samples were loaded onto a 4-12% BisTris gel and transferred to PVDF membrane. Membrane was probed with EGFR or p-EGFR and the housekeeping gene β-actin. Data quantified using Empiria studio; percent degradation normalized to β-actin and compared to Isotype control.

Placement of bYlok® on EGFR knob does not affect functional activity levels of EGFR removal from cell surface and degradation (FIGS. 17A-17C). When implemented on ITGB6 arm, however, the bYlok® placement appears to induce greater levels of reduction in EGFR on cell surface, whole cell degradation, and downstream signaling.

### Example 20—CH1 domain and CL domain for Proper Bispecific Chain Pairing

When protein function is conserved throughout evolution, protein structure is also generally conserved and distant homologs with low sequence identity generally have highly similar structures. However, species-specific mutations can make interaction partners from different species incompatible, and the likelihood of incompatibility tends to increase with evolutionary distance between two species. We sought to exploit this feature of protein evolution to generate orthogonal pairs of heavy chains and light chains in antibodies where each set of heavy chains and light chains would efficiently pair with its cognate partner but not with a non-cognate domain, with the end goal of utilizing this technology to co-express all four chains of a bispecific antibody in a single cell and generate a single desired species with high fidelity. We hypothesized that antibodies from a species distantly related to humans would possess a similar tertiary and quaternary structure as human antibodies but mutations at select protein-protein interfaces, namely that of the CH1 domain and CL domain, would make it such that cross-species heavy chain-light chain pairing (e.g. human IgG1 heavy chain and shark kappa light chain) would be inefficient or impossible. Identifying the residues from the CH1 and CL domains of an alternative species responsible for this incompatibility and grafting them onto an otherwise human CH1 and CL pair could therefore generate orthogonal CH1-CL interfaces in which only cognate domains can efficiently pair, thus guiding the proper chain pairing.

To identify positions at the CH1-CL interface that vary across species, structural models of CH1 domains and CL domains from birds, reptiles, amphibians, fish, and sharks were generated. Sequences corresponding to these domains were identified by BLAST search and structural models generated using a crystal structure of a human Fab fragment as a template. Models of CH1 and CL domains from each species were aligned to the human CH1 or CL, respectively, in PyMOL to model the interface. Mutations were then identified through visual comparison of each interface residue (defined as all residues having an atom within 5 angstroms of the partner domain). Non-conservative substitutions and mutations in which there was a corresponding compensatory mutation on the partner domain were prioritized as these are the most likely to drive incompatibility with the native human interface.

From this analysis, two distinct clusters of mutations from a shark (Heterodontus francisci) CH1-CL interface were identified. The first, Cluster 1, comprises heavy chain residues 124 and 141 and light chain residues 116 and 118 (Kabat numbering for human IgG1 CH1 and human kappa CL, respectively) and contains a mixture of aliphatic and polar residues in direct contact. The non-conservative mutations HC L124S, HC G141L, LC F116T, and LC F118M are expected to generate steric conflicts with the WT human IgG1 CH1 and CL, disfavoring this interaction. The second cluster, comprising heavy chain residues 172 and 192 and light chains residues 137 and 138 (Kabat numbering for human IgG1 CH1 and human kappa CL, respectively), contains three polar residues mutated to charged residues and one polar residue mutated to a polar residue. Mutations H172K and T192K in the heavy chain place two lysines in close proximity to N137D and N138H in the light chain, allowing for a salt bridge and a potential hydrogen bonding interaction to occur. N138 was also mutated to Asp (N138D) to generate two salt bridges between the two lysines and two aspartic acids and further enhance the interaction. The mutations within Cluster 2 can also be reversed between the heavy chain and light chain to create a novel, orthogonal interface, generating an alternative CHT-CL pair where the heavy chain contains the H172D and T192D mutations and the light chain contains the N137K and N138K mutations. When a four chain bispecific antibody containing the full set of mutations (HCl: H172K, T192K; LC1 N137D, N138D; HC2: H172D, H192D; LC2: N137K, N138K) is expressed from a single cell, cognate pairing of HC1 and LCT or HC2 and LC2 generates two favorable salt bridges while mispairing creates electrostatic repulsion by bringing together either four lysines or four aspartic acids, depending on the mis-paired species. Mutations from the two clusters can also be combined to further increase the likelihood of correct pairing, with Cluster 1 mutations introduced onto a single arm of a bispecific already containing Cluster 2 mutations on one or both Fabs.

TABLE 13
Exemplary CH1 and CL Pairs

| CH1 Arm 1 | CL Arm 1 | CH1 Arm 2 | CL Arm 2 |
|---|---|---|---|
| ASTKGPSVFPSAPS | RTVAAPSVTIM | ASTKGPSVFPLA | RTVAAPSVFIFPPS |
| SKSTSGGTAALLC | PPSDEQLKSGT | PSSKSTSGGTAA | DEQLKSGTASVVCL |
| LVKDYFPEPVTVS | ASVVCLLDDFY | LGCLVKDYFPEP | LKKFYPREAKVQWK |
| WNSGALTSGVKT | PREAKVQWKVD | VTVSWNSGALTS | VDNALQSGNSQESV |
| FPAVLQSSGLYSLS | NALQSGNSQES | GVDTFPAVLQSS | TEQDSKDSTYSLSS |
| SVVKVPSSSLGTQ | VTEQDSKDSTY | GLYSLSSVVDVP | TLTLSKADYEKHKV |
| TYICNVNHKPSNT | SLSSTLTLSKA | SSSLGTQTYICN | YACEVTHQGLSSPV |
| KVDKKVEPKSC | DYEKHKVYACE | VNHKPSNTKVD | TKSFNRGEC (SEQ |
| (SEQ ID NO: 118) | VTHQGLSSPVT | KKVEPKSC (SEQ | ID NO: 121) |
| | KSFNRGEC (SEQ | ID NO: 120) | |
| | ID NO: 119) | | |
| ASTKGPSVFPLAPS | RTVAAPSVFIFP | ASTKGPSVFPSA | RTVAAPSVTIMPP |
| SKSTSGGTAALGC | PSDEQLKSGTAS | PSSKSTSGGTAA | SDEQLKSGTASVV |
| LVKDYFPEPVTVS | VVCLLKKFYPR | LLCLVKDYFPEP | CLLDDFYPREAKV |
| WNSGALTSGVDTF | EAKVQWKVDN | VTVSWNSGALTS | QWKVDNALQSGNS |
| PAVLQSSGLYSLSS | ALQSGNSQESV | GVKTFPAVLQSS | QESVTEQDSKDST |
| VVDVPSSSLGTQT | TEQDSKDSTYSL | GLYSLSSVVKVP | YSLSSTLTLSKAD |
| YICNVNHKPSNTK | SSTLTLSKADYE | SSSLGTQTYICN | YEKHKVYACEVTH |
| VDKKVEPKSC | KHKVYACEVTH | VNHKPSNTKVD | QGLSSPVTKSFNR |
| (SEQ ID NO: 120) | QGLSSPVTKSFN | KKVEPKSC (SEQ | GEC |
| | RGEC (SEQ ID | ID NO: 118) | (SEQ ID NO: 119) |
| | NO: 121) | | |
| ASTKGPSVFPSAPS | RTVAAPSVTIM | ASTKGPSVFPLA | RTVAAPSVFIFPPS |
| SKSTSGGTAALLC | PPSDEQLKSGTA | PSSKSTSGGTAA | DEQLKSGTASVVCL |
| LVKDYFPEPVTVS | SVVCLLKKFYP | LGCLVKDYFPEP | LDDFYPREAKVQWK |
| WNSGALTSGVDTF | REAKVQWKVD | VTVSWNSGALTS | VDNALQSGNSQESV |
| PAVLQSSGLYSLSS | NALQSGNSQES | GVKTFPAVLQSS | TEQDSKDSTYSLSS |
| VVDVPSSSLGTQT | VTEQDSKDSTY | GLYSLSSVVKVP | TLTLSKADYEKHKV |
| YICNVNHKPSNTK | SLSSTLTLSKAD | SSSLGTQTYICN | YACEVTHQGLSSPV |
| VDKKVEPKSC | YEKHKVYACEV | VNHKPSNTKVD | TKSFNRGEC (SEQ |
| (SEQ ID NO: 122) | THQGLSSPVTKS | KKVEPKSC | ID NO: 125) |
| | FNRGEC (SEQ | (SEQ ID NO: 124) | |
| | ID NO: 123) | | |
| ASTKGPSVFPLAPS | RTVAAPSVFIFP | ASTKGPSVFPSA | RTVAAPSVTIMPPS |
| SKSTSGGTAALGC | PSDEQLKSGTAS | PSSKSTSGGTAA | DEQLKSGTASVVC |
| LVKDYFPEPVTVS | VVCLLDDFYPR | LLCLVKDYFPEP | LLKKFYPREAKVQ |
| WNSGALTSGVKTF | EAKVQWKVDN | VTVSWNSGALTS | WKVDNALQSGNSQ |
| PAVLQSSGLYSLS | ALQSGNSQESV | GVDTFPAVLQSS | ESVTEQDSKDSTYS |
| SVVKVPSSSLGTQ | TEQDSKDSTYSL | GLYSLSSVVDVP | LSSTLTLSKADYEK |
| TYICNVNHKPSNT | SSTLTLSKADYE | SSSLGTQTYICN | HKVYACEVTHQGL |
| KVDKKVEPKSC | KHKVYACEVTH | VNHKPSNTKVDK | SSPVTKSFNRGEC |
| (SEQ ID NO: 124) | QGLSSPVTKSFN | KVEPKSC (SEQ | (SEQ ID NO: 123) |
| | RGEC (SEQ ID | ID NO: 122) | |
| | NO: 125) | | |
| ASTKGPSVFPLAP | RTVAAPSVFIFP | ASTKGPSVFPLA | RTVAAPSVFIFPPS |
| SSKSTSGGTAALG | PSDEQLKSGTAS | PSSKSTSGGTAA | DEQLKSGTASVVCL |
| CLVKDYFPEPVTV | VVCLLDDFYPR | LGCLVKDYFPEP | LKKFYPREAKVQWK |
| SWNSGALTSGVKT | EAKVQWKVDNA | VTVSWNSGALTS | VDNALQSGNSQESV |
| FPAVLQSSGLYSL | LQSGNSQESVT | GVDTFPAVLQSS | TEQDSKDSTYSLSS |
| SSVVKVPSSSLGT | EQDSKDSTYSL | GLYSLSSVVDVP | TLTLSKADYEKHKV |
| QTYICNVNHKPSN | SSTLTLSKADY | SSSLGTQTYICN | YACEVTHQGLSSPV |
| TKVDKKVEPKSC | EKHKVYACEVT | VNHKPSNTKVD | TKSFNRGEC |
| (SEQ ID NO: 124) | HQGLSSPVTKS | KKVEPKSC (SEQ | (SEQ ID NO: 121) |
| | FNRGEC | ID NO: 120) | |
| | (SEQ ID | | |
| | NO: 125) | | |
| ASTKGPSVFPLAPS | RTVAAPSVFIFP | ASTKGPSVFPLA | RTVAAPSVFIFPPS |
| SKSTSGGTAALGC | PSDEQLKSGTAS | PSSKSTSGGTAA | DEQLKSGTASVVCL |
| LVKDYFPEPVTVS | VVCLLKKFYPR | LGCLVKDYFPEP | LDDFYPREAKVQWK |
| WNSGALTSGVDTF | EAKVQWKVDN | VTVSWNSGALTS | VDNALQSGNSQESV |
| PAVLQSSGLYSLS | ALQSGNSQESV | GVKTFPAVLQSS | TEQDSKDSTYSLSS |
| SVVDVPSSSLGTQ | TEQDSKDSTYSL | GLYSLSSVVKVP | TLTLSKADYEKHKV |
| TYICNVNHKPSNT | SSTLTLSKADYE | SSSLGTQTYICN | YACEVTHQGLSSPV |
| KVDKKVEPKSC | KHKVYACEVTH | VNHKPSNTKVD | TKSFNRGEC |
| (SEQ ID NO: 120) | QGLSSPVTKSFN | KKVEPKSC (SEQ | (SEQ ID NO: 125) |
| | RGEC (SEQ ID | ID NO: 124) | |
| | NO: 121) | | |

### Methods

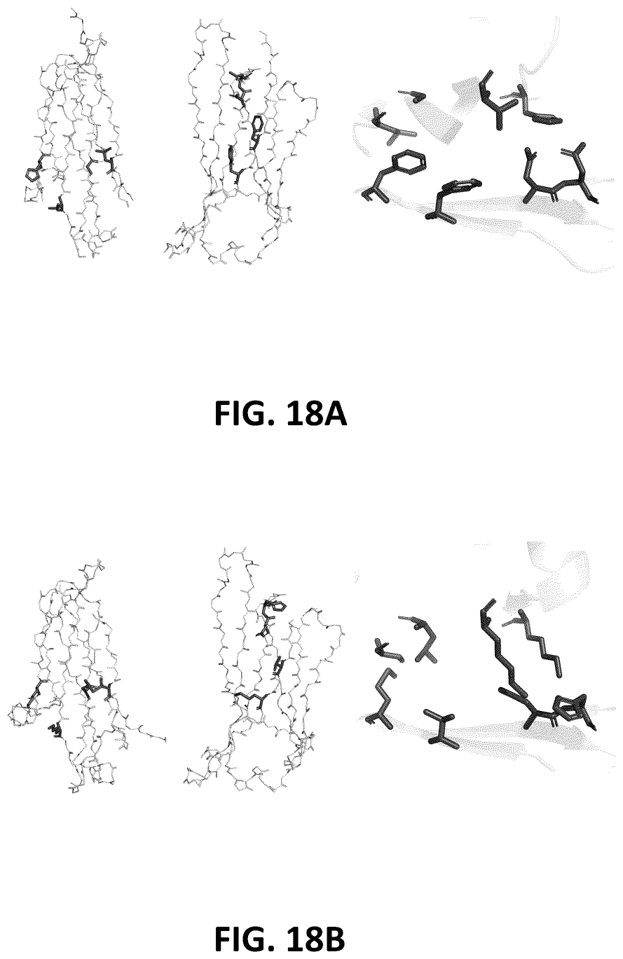

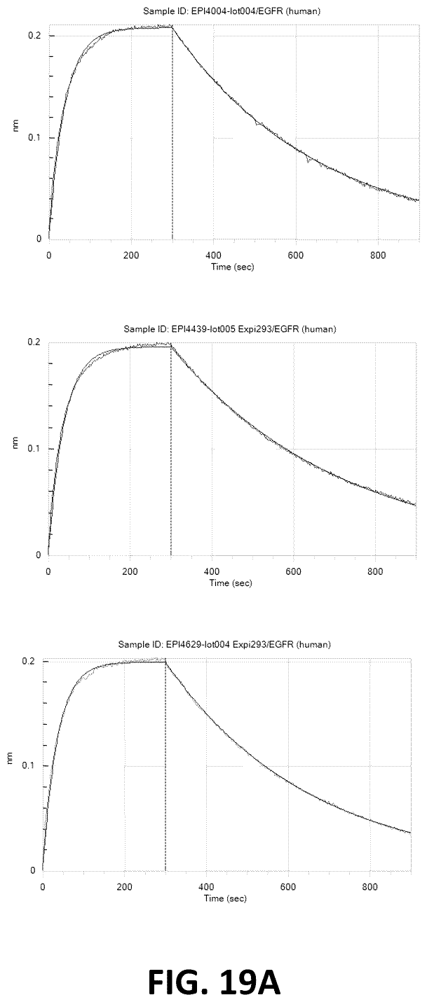

Structural models of the shark CH1 and CL were generated (FIG. 18B left and middle, respectively) using a crystal structure of a human Fab as a template (FIG. 18A). After generating models of each domain individually, models were aligned to the crystal structure in PyMOL to model the interface. All analysis was performed in PyMOL.

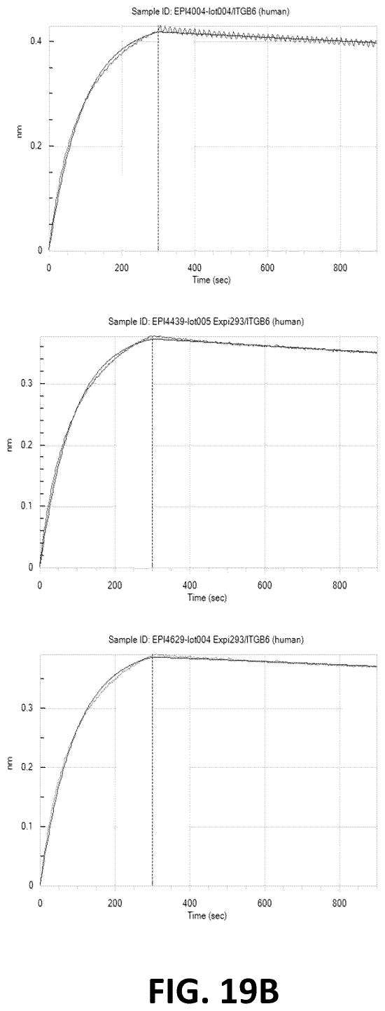

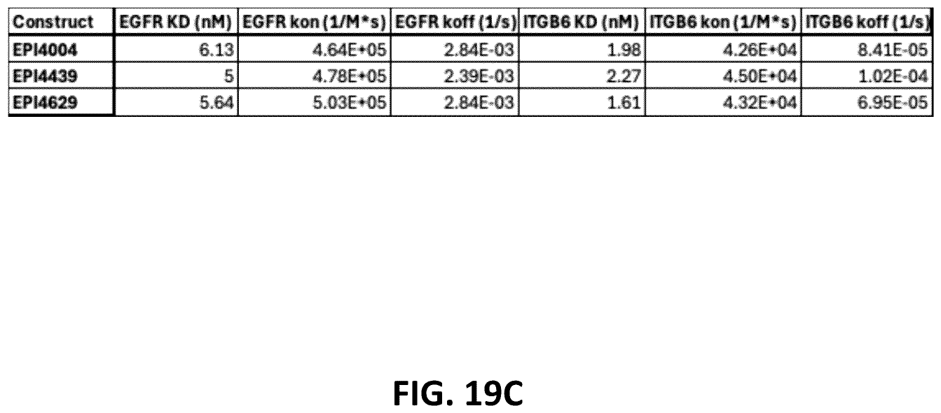

Binding assays were performed as follows. AHC2 sensors were prepared by equilibrating sensors in PBS+0.1% Casein buffer for 10 minutes and then regenerating sensors by exposing them to 10 mM glycine, pH 1.6 for 5 seconds and then neutralizing in 150 mM sodium phosphate, pH 7.0, for 4 cycles. This process was repeated a total of three times prior to measurement. Antibodies for binding assays were prepared by diluting to a final concentration of 10 nM in PBS+0.1% casein buffer. Antigens for binding assays were prepared by diluting to a final concentration of 50 nM (EGFR) or 250 nM (ITGBG) in PBS+01% casein buffer. For the binding experiment, sensors were first moved to a well containing buffer only for 60 seconds to establish a baseline. Sensors were then moved to wells containing 10 nM antibody to immobilize target antibodies for 120 seconds. Sensors were then moved to wells containing buffer for 180 seconds to reestablish a baseline. Sensors were then moved to wells containing target antigen for 300 seconds to measure association. Sensors were the moved back to wells containing buffer only to measure dissociation. Two empty sensors with no immobilized antibody were included to measure background binding of antigens to sensors. Data was analyzed using Octet BLI Analysis Version 12 software with the average signal from the background sensors being subtracted from the signal for all test articles (see FIGS. 19A-19C).

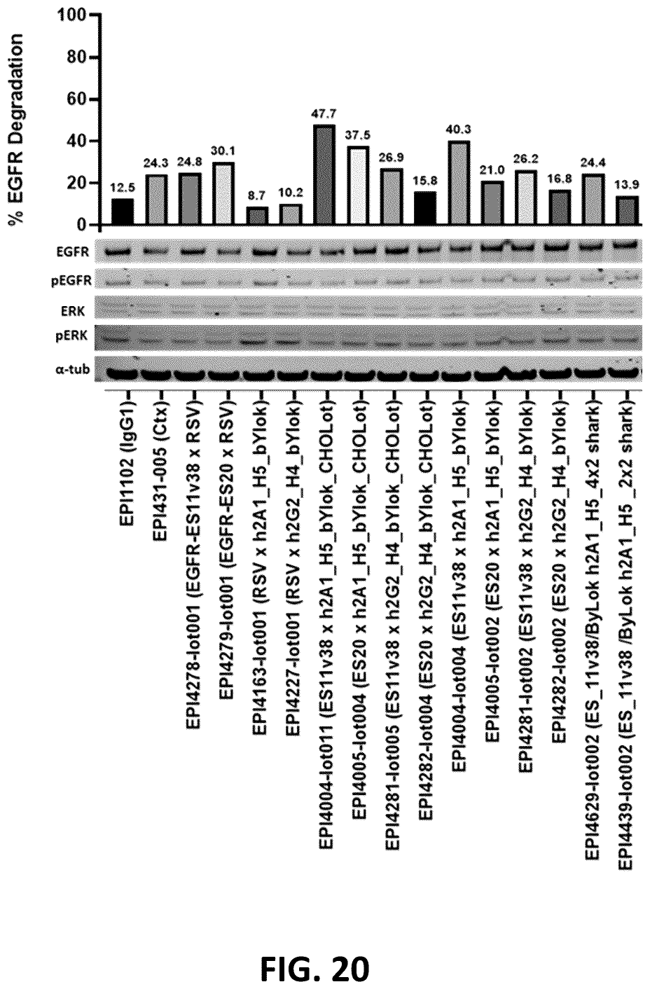

Western blot experiments for NCI-H1975 cells were performed as follows. NCI-H1975 cells were seeded overnight at 100,000 cells/well of a 12-well plate. Media was removed and cells were treated with EpiTACs for 48 hours. Media was removed, and cells were washed with PBS and protein lysates were prepared in RIPA lysis buffer. Prepared samples were loaded onto a 4-12% BisTris gel and transferred to PVDF membrane. Membrane was read on Licor Odyssey DLx. Data quantified using Empiria studio; percent degradation normalized to tubulin and compared to IgG control (see FIG. 20).

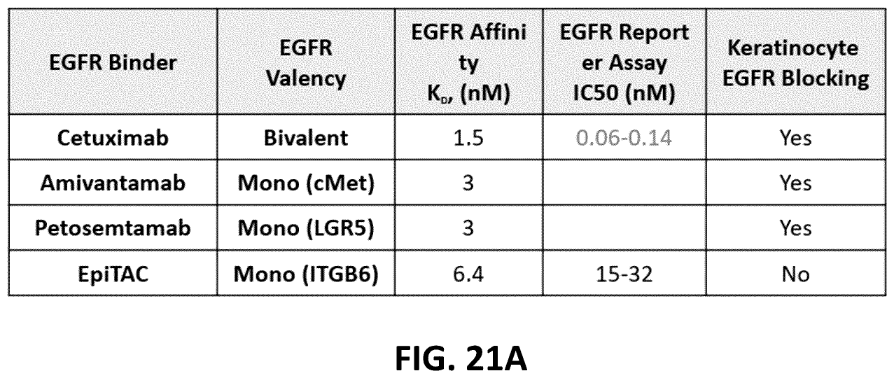

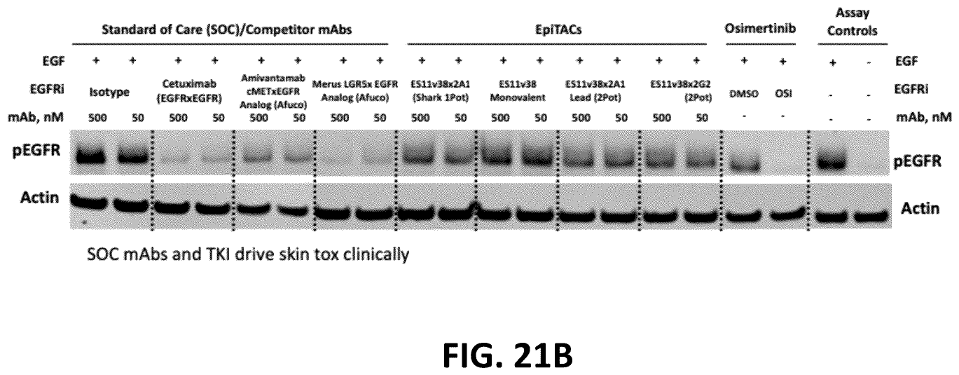

Western blot experiments for primary epidermal keratinocytes were performed as follows. Primary epidermal keratinocytes (HEKa) were seeded overnight at 50,000 cells/well of a 24-well plate. Media was removed and cells were treated with EpiTACs for 48 hours. Media was removed, and cells were treated with 100 ng/ml EGF for 15 minutes at 37° C. Cells were washed with PBS and protein lysates were prepared in RIPA lysis buffer. Protein quantification was measured by Pierce BCA Protein Assay Kit from ThermoFisher according to manufacturer's instructions. Protein from each sample (15 μg of protein per sample) was loaded and run on a NuPAGE, 4-12% Bio-Tris Midi Gel, followed by PVDF membrane transfer. Membranes were blocked in LICOR blocking buffer, followed by primary antibody, followed by 3 washes in TBST, then by secondary antibody in LICOR antibody diluent, 3 washes in TBST. Finally, the membrane was read on Licor Odyssey DLx. Actin was used as a housekeeping control (see FIG. 21B).

### Results

Visual identification of non-conservative mutations at the CH1-CL interface in the shark Fab compared with the human Fab identifies two clusters of residues that could be grafted into a human framework to generate an orthogonal interface to drive proper chain pairing in a bispecific antibody (see FIGS. 18A-18B).

Binding assays were performed to determine whether the modifications made to the CH1 domains of the charge-pair variants had an no effect on antigen recognition. Both charge-pair variants show equilibrium binding constants and kinetic rates that are indistinguishable from the parental molecule EPI4004, indicating that the charge-pair variants maintain WT binding (see FIGS. 19A-19C).

Western blot experiments for NCI-H1975 cells were performed to determine whether the charge complementarity mutations introduced into the EpiTACs, affected the ability to degrade EGFR or affect downstream signaling (see FIG. 20). EpiTAcs with incorporated mutations performed similarly to EpiTACs made as knobs and holes or knobs and holes with bYlok.

Western blot experiments for primary epidermal keratinocytes were performed to determine whether charge-complementary mutations in EpiTACs, compared to those produced as knobs and holes, affect EGFR signaling in normal skin cells. Compared to standard of care molecules (such as cetuximab, amivantamab, petosemtamab and Osimertinib) EpiTACs with or without the charge-complementary mutations did not affect EGFR signaling (pEGFR levels) in primary skin cells (see FIGS. 21A-21B). For FIG. 21B one pot bispecific antibodies (disclosed herein) were produced by expressing all 4 chains in a single cell line and then purifying the target molecules; while for two pot bispecific antibodies (disclosed herein), each half IgG (knob vs hole) was purified individually and then combined to make bispecific antibodies (as disclosed herein).

### Example 21—EGFR Levels in Cancer Cell Lines

The below is an example of determining the effectiveness of bispecific antibodies comprising an EGFR arm and an ITGB6 arm (EPI4004) in reducing EGFR levels (e.g., reduction in EGFR by internalization and/or degradation).

### Western Blot Methods

A431 cells (a human cell line derived from a skin biopsy of a patient with carcinoma where wild-type EGFR and ITGB6 are overexpressed), NCI-H1975 cells (a human non-small cell lung cancer (NSCLC) cell line derived from a patient with lung adenocarcinoma), and PC9 cells (a human-derived non-small cell lung cancer (NSCLC) cell line, which has a deletion mutation in exon 19 of the EGFR gene) were seeded overnight 100,000-500,000 cells/well of a 12-well plate. Media was removed and cells were treated with monovalent (single-arm EGFR antibody) and a bispecific antibody comprising an EGFR arm and an ITGB6 arm (EPI4004; SEQ ID NOs: 13-21, 31-36, 43-45, 49-51, 55-63, 73-78, 85-87, and 91-93) for 48 hours. A431 cells and PC9 cells were treated with EGF (epidermal growth factor) for 15 minutes. After stimulation with EGF, media was removed, and cells were washed with PBS and protein lysates were prepared in RIPA lysis buffer. Prepared samples were loaded onto a 4-12% BisTris gel and transferred to PVDF membrane. Membrane was read on Licor Odyssey DLx. Data quantified using Empiria studio; percent degradation of total EGFR was normalized to tubulin and compared to IgG control.

Xenograft Tumor Models

HCC 827 tumor cells with exon 19 deletion were grown in tissue culture flasks containing RPMI1640 medium supplemented with 10% fetal bovine serum at 37° C. in an atmosphere of 5% CO2. Cells were harvested during exponential growth phase, and 5×10⁶ total cells were inoculated into the right front flank of BALB/c nude mice. Animals were monitored daily and tumor volumes and body weights were measured twice per week according to IACUC guidelines. Tumors were grown until group mean tumor volumes reached 150 mm³, at which point animals were randomized into groups (n=8/grp) and given intravenous doses of the indicated antibodies at 10 mg/kg on study Day 0, 6, 12, 18 and 24. Osimertinib was purchased from MCE (HY-15772) and dosed orally daily from Day 0-24 at 1 mg/kg. EPI4004 was prepared in-house and isotype control mAbs were purchased from BioXcell (BP0297).

### Results

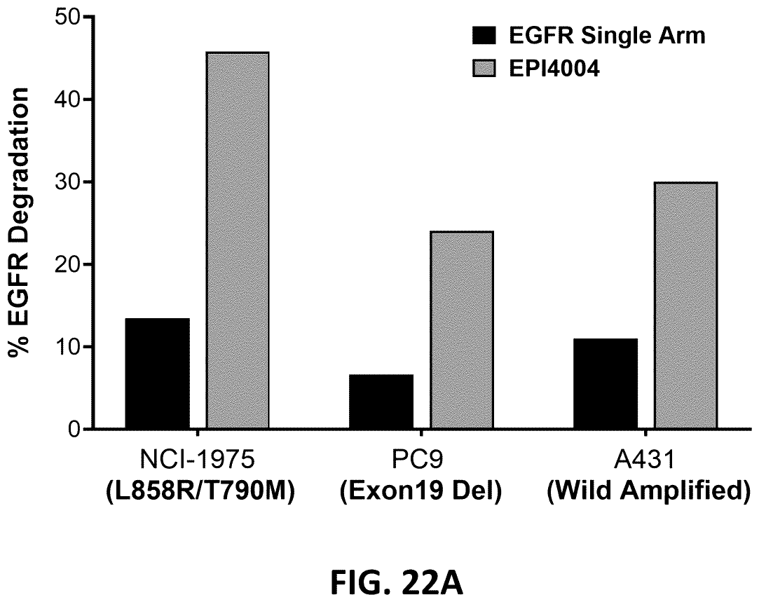

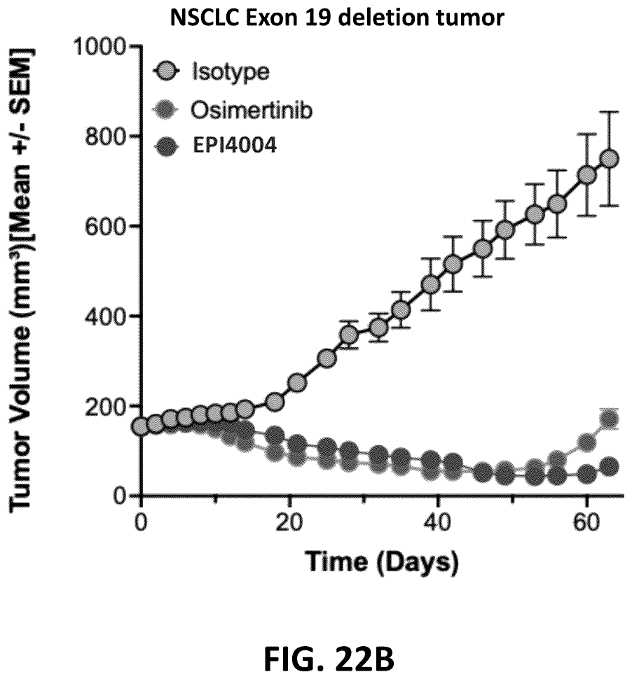

This example sought to determine whether the EGFR mutation status affected the ability of bispecific antibodies comprising an EGFR arm and an ITGB6 arm (EPI4004) to degrade EGFR. Here we demonstrated that EPI4004 was able to degrade EGFR in NSCLC and HNSCC that contain wildtype, EGFR amplified or various mutated forms of EGFR (NCI-1975 (L858R/T790M), PC9 (Exon19 Del), and A432 (Wild Amplified). These results demonstrate that the bispecific antibody (EPI4004) is mutation agnostic (see FIG. 22A). Results for the in vivo model (HCC827 with EGFR exon19 deletion), demonstrate in vivo activity of the bispecific antibody (EPI4004), including in the presence of an EGFR exon19 deletion (see FIG. 22B).

### Example 22—EGFR Degradation and Signaling in NSCLC Cells

The below is an example of determining the effectiveness of bispecific antibodies comprising an EGFR arm and an ITGB6 arm (EPI4004) in reducing EGFR levels (e.g., reduction in EGFR by degradation) and maintaining EGFR pathway signaling (e.g., EGFR, ERK, and/or HER3 phosphorylation).

### Western Blot Methods

H1975 cells (NCI-H1975 cells) were seeded overnight at 100,000 cells/well of a 12-well plate. Media was removed and cells were treated with monovalent (single-arm EGFR antibody), Cetuximab, and a bispecific antibody comprising an EGFR arm and an ITGB6 arm described herein (EPI4004) for 48 hours. Media was removed, and cells were washed with PBS and protein lysates were prepared in RIPA lysis buffer. Prepared samples were loaded onto a 4-12% BisTris gel and transferred to PVDF membrane. Membrane was read on Licor Odyssey DLx. Data quantified using Empiria studio; percent degradation of total EGFR was normalized to tubulin and compared to IgG control.

### Xenograft Tumor Models

NCI-H1975 cells lines were grown in tissue culture flasks containing RPMI 1640 medium supplemented with 10% fetal bovine serum at 37° C. in an atmosphere of 5% CO2. Cells were harvested during exponential growth phase, and 5×10⁶ total cells were inoculated into the right front flank of BALB/c nude mice. Animals were monitored daily, and tumor volumes and body weights were measured twice per week according to IACUC guidelines. Tumors were grown until reaching approximately a volume of 150 mm³, at which point animals were randomized into groups (n=6/grp) and given intravenous doses of the indicated antibodies at 15 mg/kg and taken down for tumor analysis at 24 and 72 hours. EPI4004 was prepared in-house and isotype control mAbs were purchased from BioXcell (BP0297).

Tumor IHC detection of EGFR and pEGFR. Tumors were collected from animals and formalin fixed, followed by paraffin embedding (FFPE). Cut sections were then stained with EGFR (non-competing with the treatment mAb) and pEGFR (Tyr1068) detection mAbs. Anti-recombinant EGFR rabbit monoclonal antibody (Abcam ab227642, Clone SP84) was used at 1:100 with citrate-based pH 6.2 Heat-Induced Epitope Retrieval. Followed by staining on the Biocare intelliPATH automated staining platform using the manufacturers recommendations.

### Results

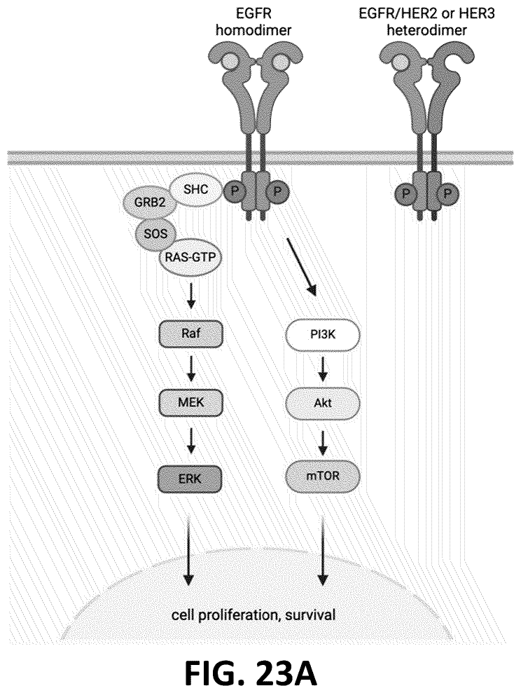

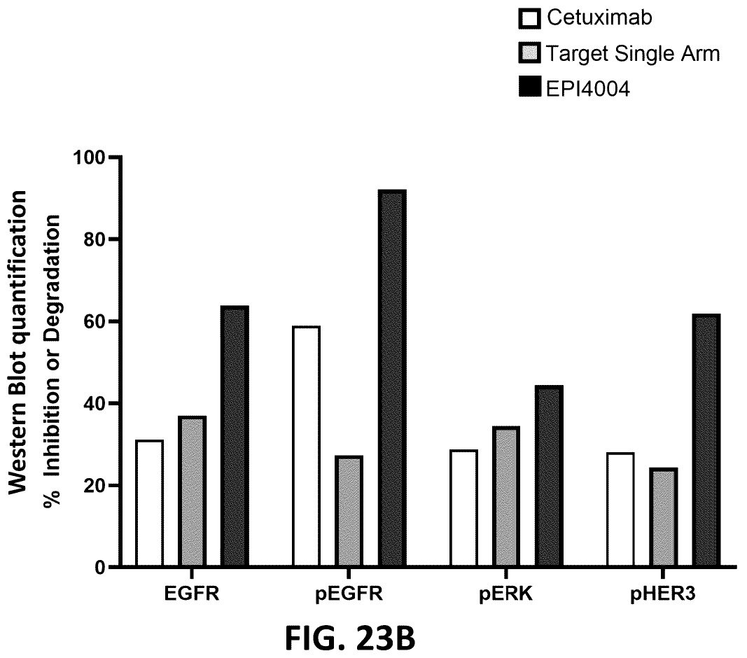

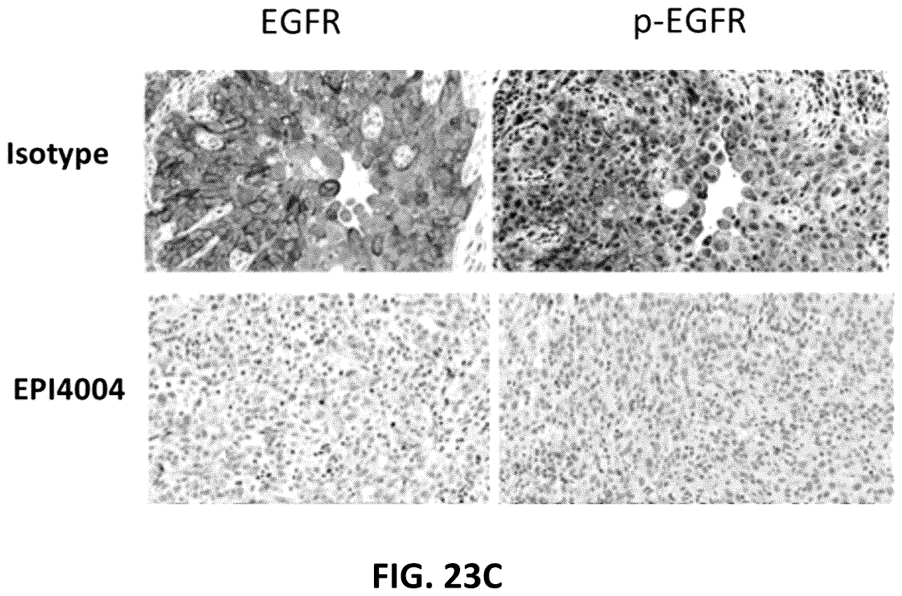

This example sought to determine the ability of bispecific antibodies comprising an EGFR arm and an ITGB6 arm described herein (EPI4004) to not only degrade EGFR but affect additional downstream signaling pathways (see FIG. 23A). Data suggests that a bispecific antibody comprising an EGFR arm and an ITGB6 arm (EP14004) was able to degrade EGFR and affect downstream pEGFR, pERK and pHER3 (e.g., inhibit p-EGFR levels) (see FIGS. 23B-C) compared to a control (single-arm EGFR antibody or Cetuximab).

### Example 23—Tumor Growth in NSCLC with L858R and T790M mutations in EGFR

The below is an example of determining the effectiveness of bispecific antibodies comprising an EGFR arm and an ITGB6 arm (EP14004, EP13473) in reducing tumor growth (e.g., suppressing tumor growth in a human non-small cell lung cancer (NSCLC) xenograft tumor model with L858R and T790M mutations in EGFR).

### Methods

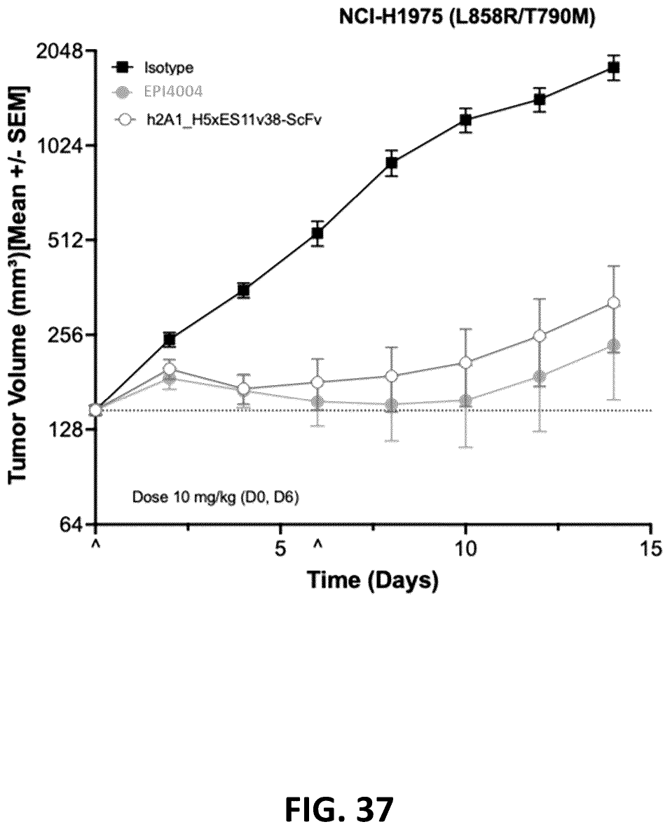

Xenograft tumor models. NCI-H1975 cell lines (human non-small cell lung cancer (NSCLC) cell line derived from a patient with lung adenocarcinoma) with L858R and T790M mutations in EGFR were grown in tissue culture flasks containing RPMI1640 medium supplemented with 10% fetal bovine serum at 37° C. in an atmosphere of 5% CO2. Cells were harvested during exponential growth phase, and 5×10⁶ total cells were inoculated into the right front flank of BALB/c nude mice. Animals were monitored daily, and tumor volumes and body weights were measured twice per week according to IACUC guidelines. Tumors were grown until reaching approximately a volume of 150 mm³, at which point animals were randomized into groups (n=8/grp) and given intravenous doses of the indicated antibodies (EPI4004, and EPI3473) at 10 mg/kg, and then dosed again on day 7. Separately, mice were also dosed at 10 mg/kg with an scFVxFab bispecific antibody (scFV: EGFR; Fab: ITGB6) (see FIG. 37). Bispecific antibodies comprising an EGFR arm and an ITGB6 arm (EPI4004, and EPI3473) were prepared in-house, Cetuximab was purchased from MedChemExpress (HY-P9905), and isotype control mAbs (single-arm EGFR antibody, single-arm degrader antibody) were purchased from BioXcell (BP0297).

Tumor western blot for protein degradation. Tumors were collected and immediately frozen in liquid nitrogen. Protein lysates were prepared by mechanical homogenization using a tissue homogenizer in RIPA lysis buffer. Protein quantification was measured by Pierce BCA Protein Assay Kit of ThermoFisher according to manufacturer's instructions. Equal amounts of protein per animal were loaded and run on NuPAGE, 4-12% Bio-Tris Midi Gels, followed by PVDF membrane transfer. Membranes were blocked in TBST+5% milk, followed by primary antibody, followed by 3 washes in TBST, then by secondary antibody in TBST+5% milk, 3 washes in TBST, then detection by Odyssey Infrared Imager. Separate gels were run and measured for EGFR, p-EGFR and GAPDH for each animal. Quantification of relative protein levels were performed by dividing the signal intensity in the EGFR lanes by the signal in the paired sample GAPDH lanes. A cross-gel normalization factor equating to an arbitrary value of 1 was defined using the mean value of the isotype control group animals which were included on each gel.

### Results

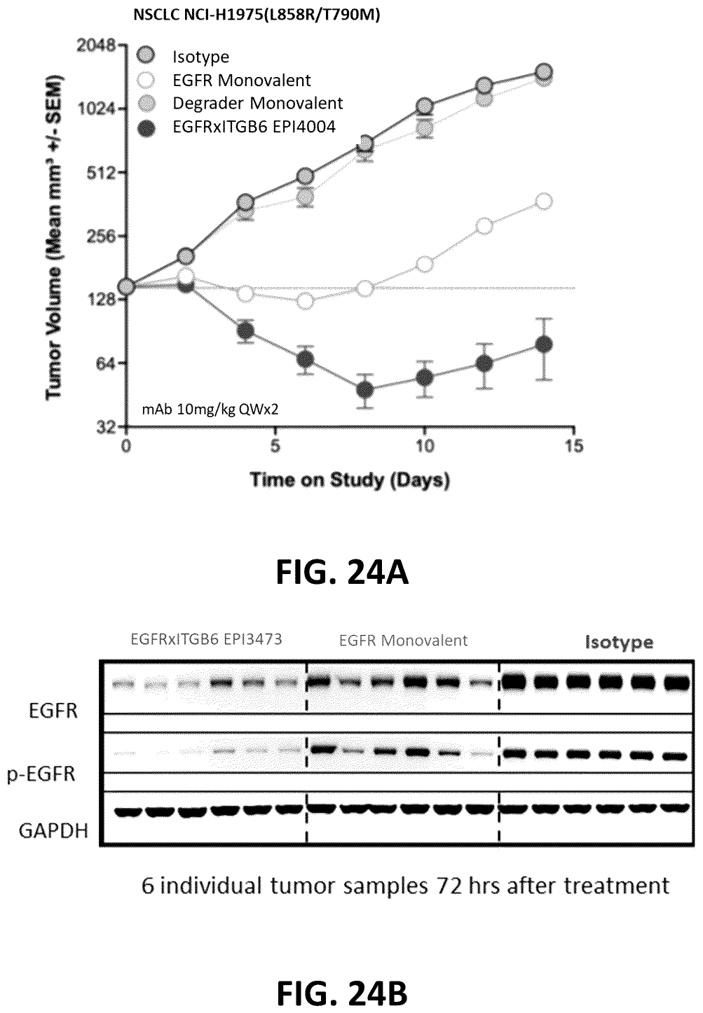

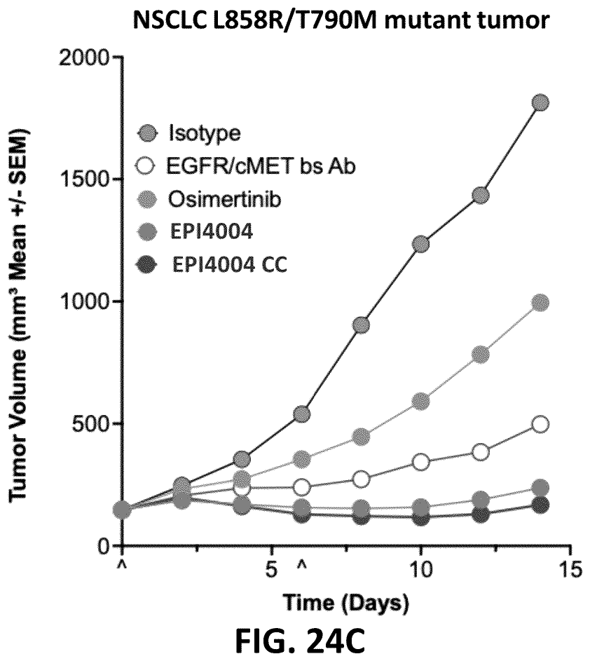

This example sought to determine the ability of bispecific antibodies comprising an EGFR arm and an ITGB6 arm described herein (EPI4004, and EPI3473) to suppress tumor growth in vivo, and to determine whether monovalent arms of bispecific antibodies (single-arm EGFR antibody, and single-arm ITGB6 antibody) functioned synergistically when combined into bispecific antibodies. Data suggests that a bispecific antibody comprising an EGFR arm and an ITGB6 arm (EPI4004, and EPI3473) synergistically suppressed tumor growth to levels greater than that of the monovalent control arms alone (single-arm EGFR antibody, and single-arm ITGB6 antibody), including tumor regressions (see FIGS. 24A-C). This example also sought to determine the ability of the bispecific antibody comprising an EGFR arm and an ITGB6 arm (EPI4004, and EPI3473) to cause synergistic EGFR degradation in the tumor, which was greater than the monovalent EGFR control mAb (single-arm EGFR antibody). Data for the scFVxFab bispecific antibody (scFV: EGFR; Fab: ITGB6) shows that the scFVxFab bispecific antibody is also capable of suppressing tumor growth to levels greater than that of the isotype control (see FIG. 37).

### Example 24—Tumor Growth in NSCLC with L858R, T790M, exon 19 deletion, and/or C797S EGFR Mutations

The below is an example of determining the effectiveness of bispecific antibodies comprising an EGFR arm and an ITGB6 arm (EPI4004) in reducing tumor growth (e.g., suppressing tumor growth in a human non-small cell lung cancer (NSCLC) xenograft tumor model with L858R, T790M, and/or C797S mutations in EGFR).

### Methods for Xenograft Tumor Models

Osimertinib-responsive model. For the Osimertinib-responsive model, wild type NCI-H1975 lung tumor cells were grown in tissue culture flasks containing RPMI1640 medium supplemented with 10% fetal bovine serum at 37° C. in an atmosphere of 5% CO2. Cells were harvested during exponential growth phase, and 5×10⁶ total cells were inoculated into the right front flank of BALB/c nude mice. Animals were monitored daily, and tumor volumes and body weights were measured twice per week according to IACUC guidelines. Animals came off study, and therefore counted against survival percentage, once an IACUC limit was reached (either TV>2000 mm³ or significant tumor ulceration). Two separate studies were combined to graph the survival kinetics for EpiTAC and Osimertinib. In the EPI4004 survival study, tumors were grown until reaching approximately a volume of 150 mm³, at which point animals were randomized into groups (n=8/grp), and given intravenous doses of the indicated antibodies at 10 mg/kg, and then dosed again on day 7. EPI4004 was prepared in-house. In the Osimertinib responsive study, tumors were grown until reaching approximately a volume of 200 mm³, at which point animals were randomized into groups (n=8/grp) and given 10 mg/kg daily oral Osimertinib from Day 0-7 (MedChemExpress; HY-15772).

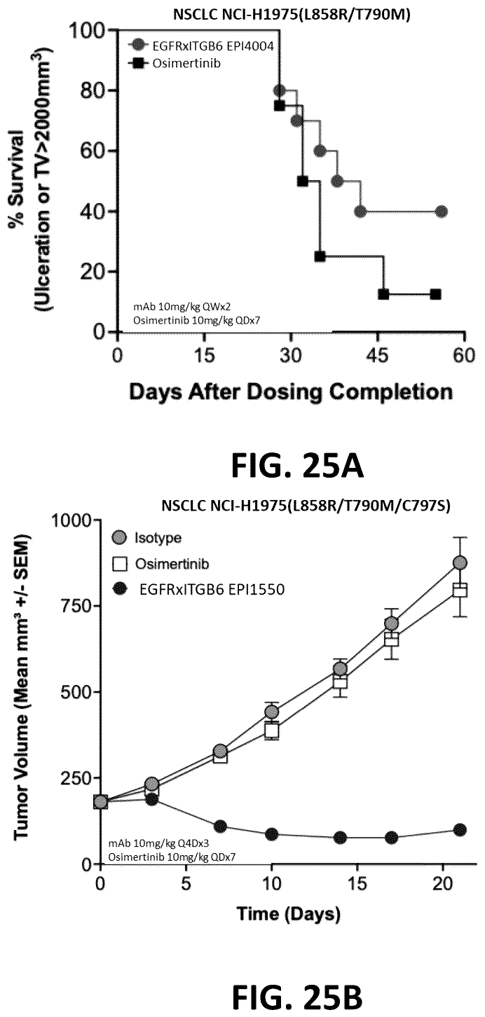

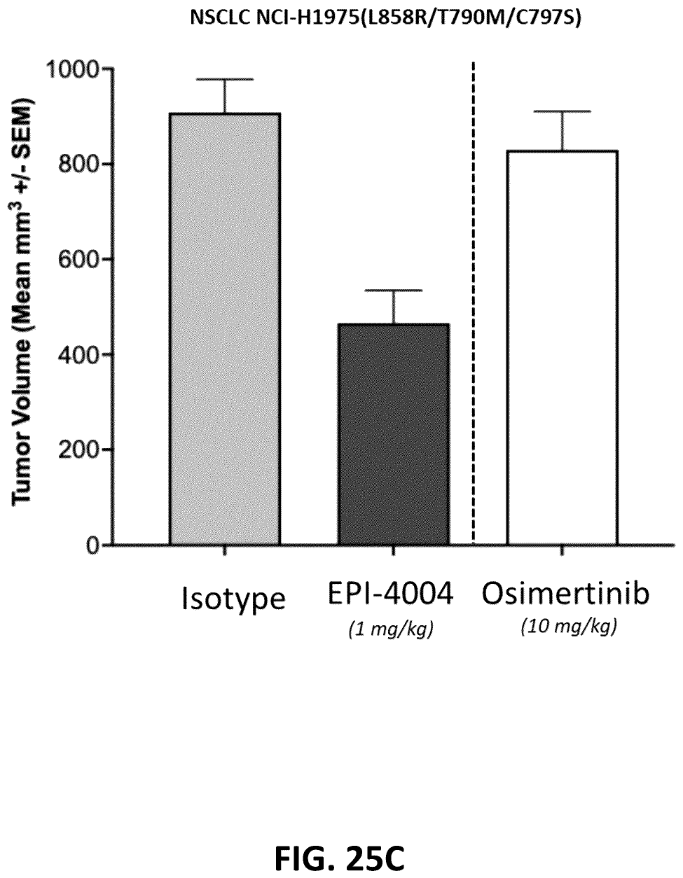

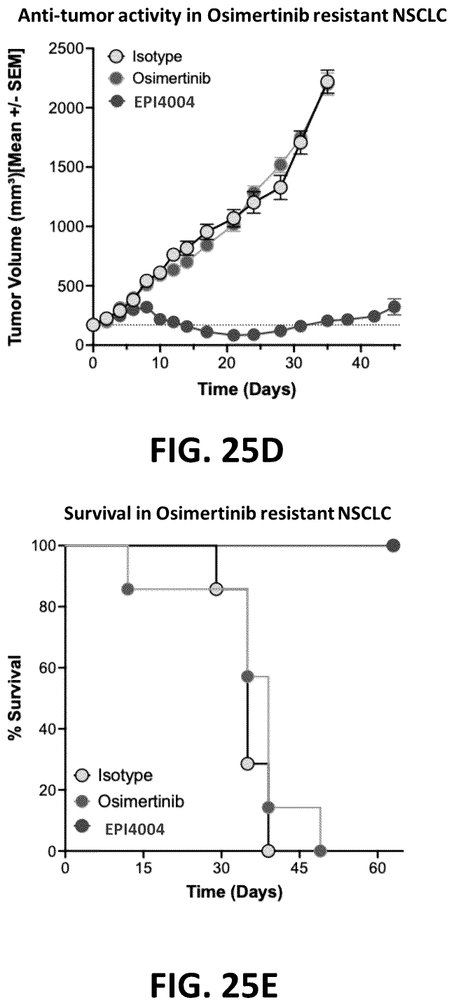

Osimertinib-resistant model. For the Osimertinib-resistant model, NCI-H1975 lung tumor cells were engineered to carry a resistance mutation (NCIH 1975-C797S; EGFR L858R/T790M mutations), and in vivo tumor xenograft models were run in a similar manner to those listed for the non-engineered Osimertinib-responsive models. In brief, NOD/SCID mice were implanted with tumors, and group dosing began (n=6-8/grp) when tumor volumes reached ˜170-180 mm³. In addition (see FIGS. 25D-E), animals were randomized into groups (n=7-8/grp) and given intravenous doses of the indicated antibodies at 10 mg/kg on study Day 0, 6 and 12. Osimertinib was purchased from MCE (HY-15772) and dosed orally daily from Day 0-12 at 1 mg/kg. EP14004 was prepared in-house and isotype control mAbs were purchased from BioXcell (BP0297).

### Results

This example sought to determine the ability of bispecific antibodies comprising an EGFR arm and an ITGB6 arm described herein (EPI4004) to suppress tumor growth in vivo in both drug-responsive EGFR-mutant NSCLC (Osimertinib-responsive L858R/T790M EGFR-mutant NSCLC; see FIG. 25A) and drug-resistant EGFR-mutant NSCLC (Osimertinib-resistant L858R/T790M/C797S EGFR-mutant NSCLC; see FIGS. 25B-E). Data suggests that a bispecific antibody comprising an EGFR arm and an ITGB6 arm (EPI4004) drives strong anti-tumor activity in both Osimertinib responsive and resistant tumor models (see FIGS. 25A-E).

### Example 25—RNA Expression of a Degrader Protein on Various Tissue Types

The below is an example of determining the RNA expression of a degrader protein (ITGB6) on various tissue types (e.g., normal skin, normal colon, NSCLC, HNSCC, esophageal tumor, bladder tumor, colorectal tumor), including tumors expressing distinct oncogenic forms of EGFR.

### Methods

Immunohistochemistry. Immunohistochemistry detection of EGFR: Anti-recombinant EGFR rabbit monoclonal antibody (Abcam, Cat. #ab227642, Clone: SP84) was used at 1:100 with citrate-based pH 6.2 Heat-Induced Epitope Retrieval; an isotype control (rabbit IgG) was used under the same conditions. FFPE sections (4 um) were stained on the Biocare intelliPATH automated staining platform using the manufacturer's recommended settings. The sections were incubated with Biocare Peroxidase Blocker (Biocare, Cat. #PX968) and Background Punisher (Biocare, Cat. #BP974M) to block non-specific background. For the detection of rabbit primary antibodies, MACH4 HRP-polymer Detection System (Biocare, Cat. #MRH534) was used. The chromogenic detection and counterstaining kits IntelliPATH FLX DAB chromogen (Biocare, Cat. #IPK5010) and IntelliPATH Hematoxylin (Biocare Medical, Cat. #XMF963) were used. Data Retrieval, Processing and Normalization. The Cancer Genome Atlas (TCGA, https://www.cancer.gov/tcga) and The Genotype-Tissue Expression (GTEx, https://gtexportal.org/) project sequencing data were retrieved from the recount3 project (https://ma.recount.bio) database using the recount3R package. Data was filtered to protein-coding genes only and between-sample (per-sample) TMM normalization was performed using the edgeR R package. Raw sequencing counts were standardized based on TMM-normalized library sizes to obtain log 2-transformed CPM (counts per million) values including a small pseudocount to deal with zero read counts. To allow comparisons between different genes, the CPM values were further normalized values by gene length in kilobases, yielding normalized fragments per kilobase of transcript per million mapped reads values (nFPKM). Batch effect removal was done using linear models with the removeBatchEffect function in the limma R package on log 2-transformed CPM values by assigning the sample source (GTEx and TCGA) as batch factor and grouping baseline and cancer normal samples in the same group (i.e. using the same factor level). Normal skin and normal colon data are RNA data from the GTex project. Cancer data are from TCGA project.

### Results

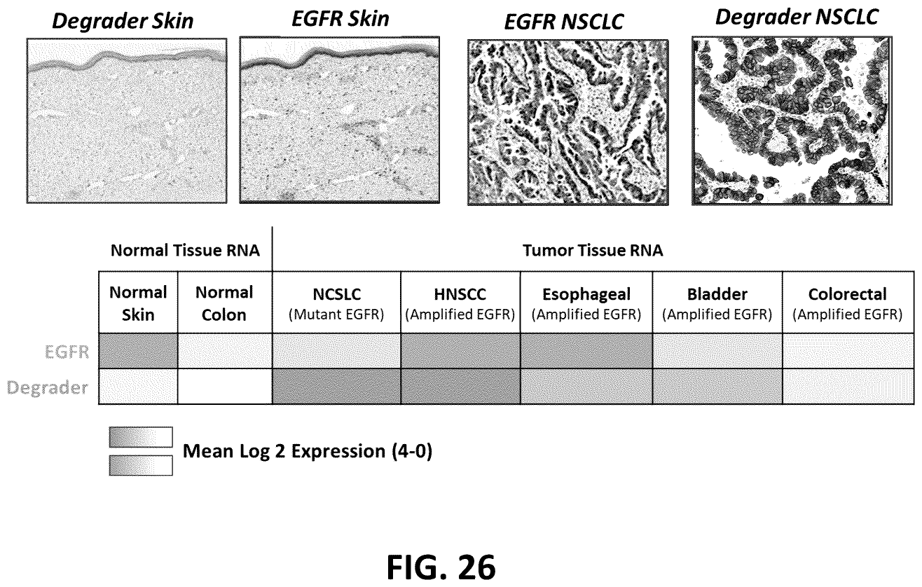

Immunohistochemistry and RNA expression analysis indicates that degrader receptor expression (ITGB6) may localize activity and drive degradation to tumors expressing distinct oncogenic forms of EGFR (see FIG. 26).

### Example 26—PK Profiles of Exemplary EGFRxITGB6 Bispecific Antibodies

This example sought to determine the pharmacokinetic properties of exemplary bispecific EGFRxITGB antibodies in tumor-free mice.

### Methods

In this example, a cohort of 6-9-week-old female Athymic nude mice were randomized into groups (n=6) based on body weight, then injected with a single 5 μl/g volume dose of single arm antibodies at either 3 mg/kg or 10 mg/kg, intravenously. Antibodies were prepared in-house. The initial dose was noted as time-point 0. Serum samples were collected and frozen from each animal. Two in-life (submandibular) cheek bleeds and one terminal (cardiac) bleed were performed on each mouse for sample collection, per IACUC guidelines, at 1, 24, 48, 96, 120 and 168 hours. Sub-groups (n=3/group) were utilized to stagger blood collection from individual animals. Animals were monitored daily and weighed multiple times per week, according to IACUC guidelines. Serum concentration (ng/ml) of each mAb was measured using the Human Therapeutic IgG1 ELISA kit (Cayman #500910) according to manufacturer's instructions. Concentrations of human IgG1 in serum was computed relative to a standard curve of positive control samples. Pharmacokinetic analysis was performed using WinNonlin Phoenix software (Certara, version 8.2). Graphs were created in GraphPad Prism on a log or linear scale. Dotted lines on the graph indicate 10,000 ng/ml for reference.

In addition, Non-GLP compliance exploratory single dose intravenous administration PK studies were performed in non-human primate (NHP, cynomolgus macaques) and rodent (rat, Sprague-Dawley) animals under veterinary observation at an accredited research facility. In brief, animals were dosed at a single dose level (NHP n=2/grp,1 male/female) at a dose volume of 1OmL/kg. After 1-2 weeks of observation and collection, the study proceeded to the next dose level. Blood was collected from a peripheral vein at pre-determined timepoints for serum chemistry, hematology and PK analysis. Serum was prepared using serum separator tubes at room temperature. PK analysis was performed using an ELISA based assay, and pharmacokinetic parameters were estimated using Phoenix@WinNonlin® version 8.4 (Certara) (see Table 14 and Table 15).

### Results

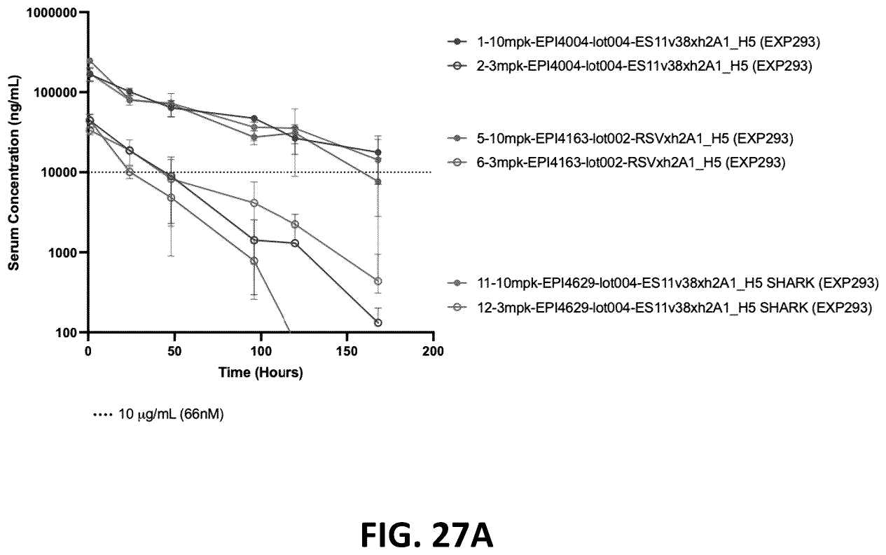

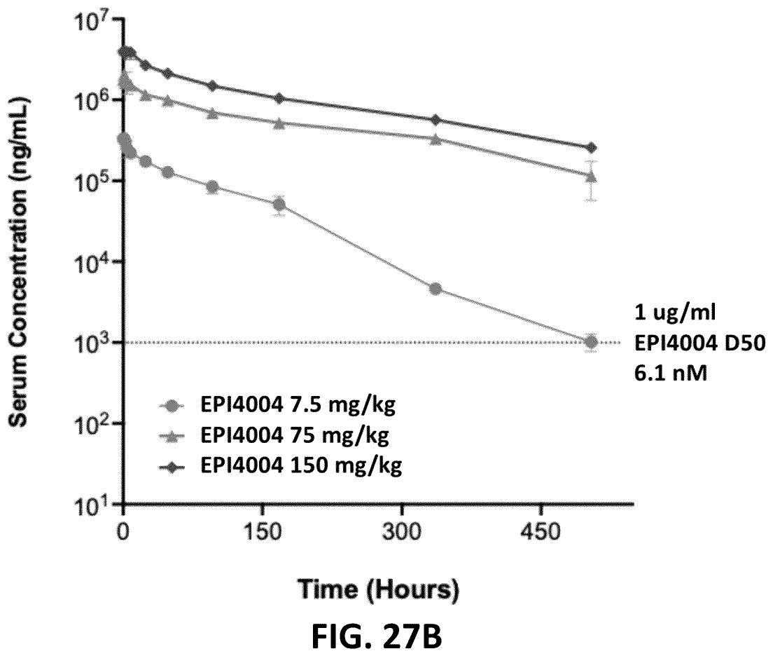

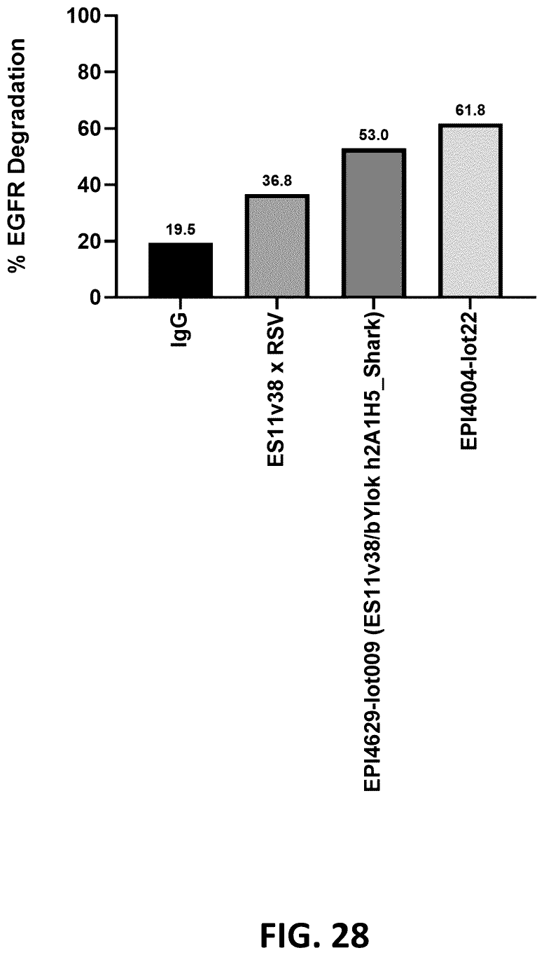

All treatment groups had measurable serum human IgG at all timepoints. The pharmacokinetic profiles and half-lives of EP14004 and EP14629 were extended compared to monovalent ITGB6 binders from those constructs, particularly at the lower 3 mg/kg dose level (see for example FIG. 27). This example also sought to determine tolerability and PK properties of a single ascending dose of EP14004 (EGFRxITGB6 bispecific antibody; SEQ ID NOs: 13-21, 31-36, 43-45, 49-51, 55-63, 73-78, 85-87, and 91-93) in primates and tolerability in rodents. Data suggests that single intravenous administration of bispecific antibodies comprising an EGFR arm and an ITGB6 arm described herein (EPI4004) was well tolerated up to 150 mg/kg in primates and up to 200 mg/kg in rats. PK analysis for bispecific antibodies comprising an EGFR arm and an ITGB6 arm described herein (EPI4004) in primates suggested exposure by mean Cmax and AUC increased with dose and the increases were dose proportional (see FIG. 27B, Table 14, and Table 15).

TABLE 14
Exemplary data from EPI4004 single dose intravenous
administration tolerability and PK study design at
multiple dose levels in non-human primate (NHP, cynomolgus
macaques) and rodent (rat, Sprague-Dawley).

| | Dose Level NHP | Dose Level Rat |
|---|---|---|
| Test Article | mg/kg | mg/kg |
| EPI4004 | 7.5 | 50 |
| EPI4004 | 75 | 100 |
| EPI4004 | 150 | 200 |

TABLE 15
Exemplary PK properties for EPI4004 after single IV dose in non-
human primate (NHP, cynomolgus macaques) at 3 dose levels.

| | EPI4004 | EPI4004 | EPI4004 |
|---|---|---|---|
| Parameter | 7.5 mg/kg | 75 mg/kg | 150 mg/kg |
| C₀ (μg/mL) | 330 | 1780 | 4060 |
| Cₘₐₓ (μg/mL) | 333 | 2060 | 4290 |
| AUC₀₋₅₀₄ (h* μg/mL) | 24100 | 255000 | 523000 |
| T_{1/2} (h) | 62.7 | 164 | 164 |
| CL (mL/h/kg) | 0.313 | 0.265 | 0.257 |
| V_{z} (mL/kg) | 28.3 | 62.0 | 60.9 |

### Example 27—Inhibited Tumor Growth and Induced Targeted Tumor EGFR Degradation in EGFR Mutant Xenograft Tumor Model

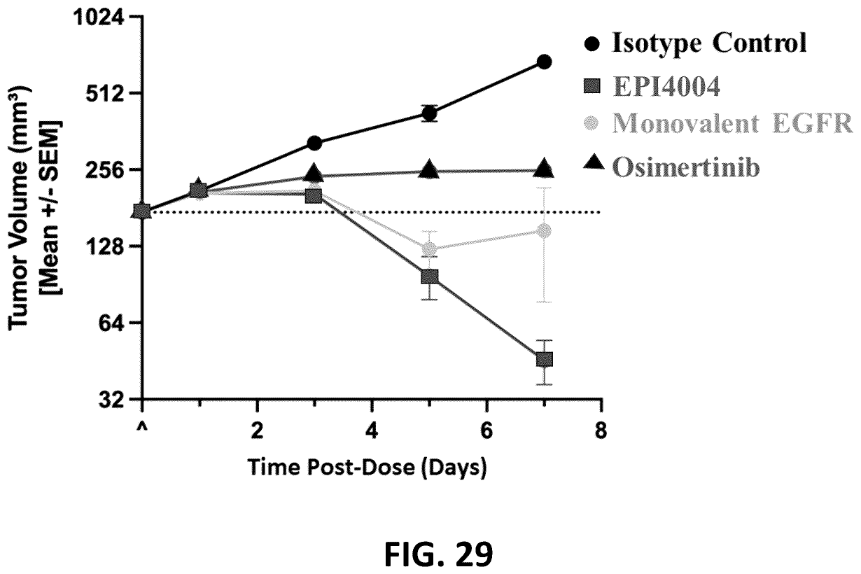

This example sought to determine the anti-tumor activity of a bispecific antibody comprising an EGFR arm and an ITGB6 arm (EPI4004) in an EGFR mutant NSCLC mouse xenograft tumor model and the underlying mechanism of its activity via targeted tumor EGFR degradation (see FIGS. 29, 30, and 31).

### Methods

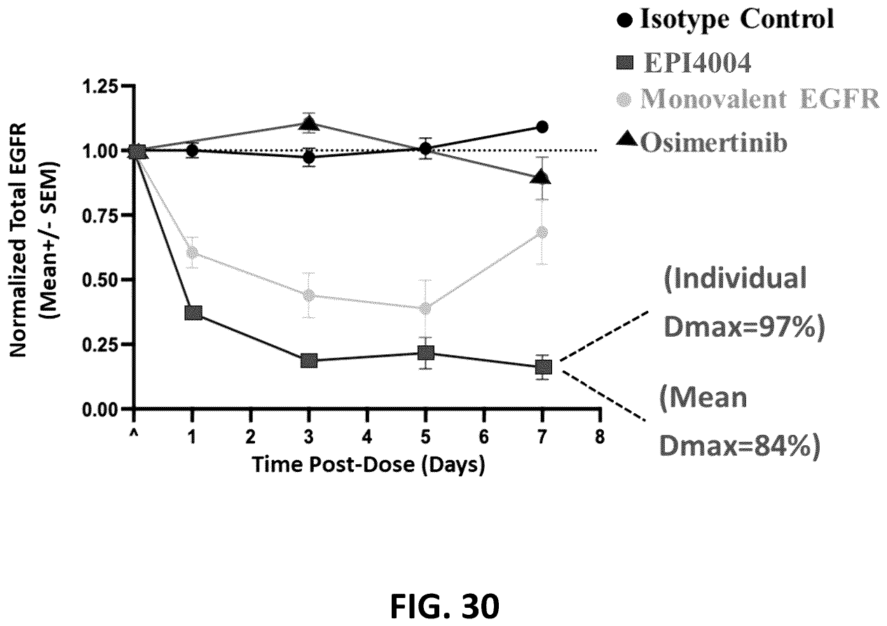

Xenograft tumor models. NCI-H1975 cells were grown in tissue culture flasks containing RPMI1640 medium supplemented with 10% fetal bovine serum at 37° C. in an atmosphere of 5% CO2. Cells were harvested during exponential growth phase, and 5×10⁶ total cells were inoculated into the right front flank of BALB/c nude mice. Animals were monitored daily and tumor volumes and body weights were measured twice per week according to IACUC guidelines. Tumors were grown until reaching approximately a volume of 200 mm³, at which point animals were randomized into groups (n=6/group/timepoint) and given a single intravenous dose of EPI4004 at 15 mg/kg or daily oral Osimertinib (MedChemExpress HY-15772) at 1 mg/kg. Tumors were collected at indicated timepoints. Tumor volume was calculated as V=(L×W×W)/2. Graphs of tumor growth kinetics were generated in Graphpad Prism (see FIGS. 29 and 30). Animals were included in the growth kinetic graphs until they were removed from the study.

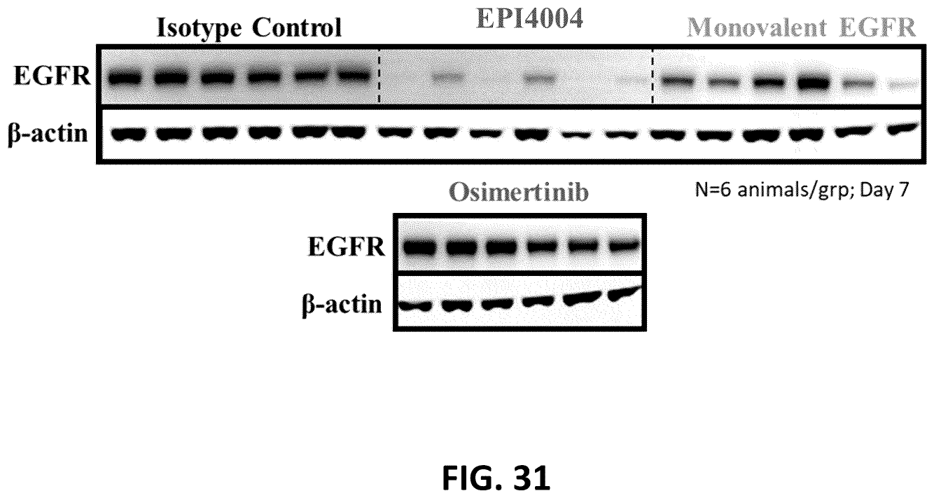

Western blot. Tumors were collected and immediately frozen in liquid nitrogen. Protein lysates were prepared by mechanical homogenization using a tissue homogenizer in RIPA lysis buffer. Protein quantification was measured by Pierce BCA Protein Assay Kit of ThermoFisher according to manufacturers instructions. Equal amounts of protein per animal were loaded and run on NuPAGE, 4-12% Bio-Tris Midi Gels, followed by PVDF membrane transfer. Membranes were blocked in TBST+5% milk, followed by primary antibody, followed by 3 washes in TBST, then by secondary antibody in TBST+5% milk, 3 washes in TBST, then detection by Odyssey Infrared Imager. Separate gels were run for each target and housekeeping gene for each animal tumor sample. Quantification of protein were performed by dividing the signal intensity in the EGFR lanes by the paired sample housekeeping gene. A normalization factor equating to an arbitrary value of 1 was defined using group 1 tumor samples, and each gel included the same set of samples, to normalize across all samples across gels (FIG. 31).

### Results

Significant tumor growth inhibition was observed at 15 mg/kg dose levels, greater than monovalent EGFR mAbs and Osimertinib (FIG. 29). Cohorts of animals were removed from the study and tumors were collected to measure EGFR degradation at pre-determined timepoints (Day 1, 3, 5 and 7). Robust tumoral EGFR degradation was apparent as early as Day 1, and continued to progress over the course of 1 week. Monovalent EGFR binder control initially demonstrated modest EGFR degradation, which reverted back to isotype control/pre-treated levels by Day 7. Osimertinib had no impact on total EGFR levels (FIG. 30). Group mean maximal tumoral EGFR degradation (Dmax) reached 84% and individual animal tumor EGFR Dmax reached 94%. These data suggest that the EPI4004 mediated anti-tumor activity in this model was likely due to targeted EGFR protein degradation.

### Example 28—Inhibited Tumor Growth and Induced Complete Regression Following an Extended Dosing Schedule in an EGFR Mutant Xenograft Tumor Model

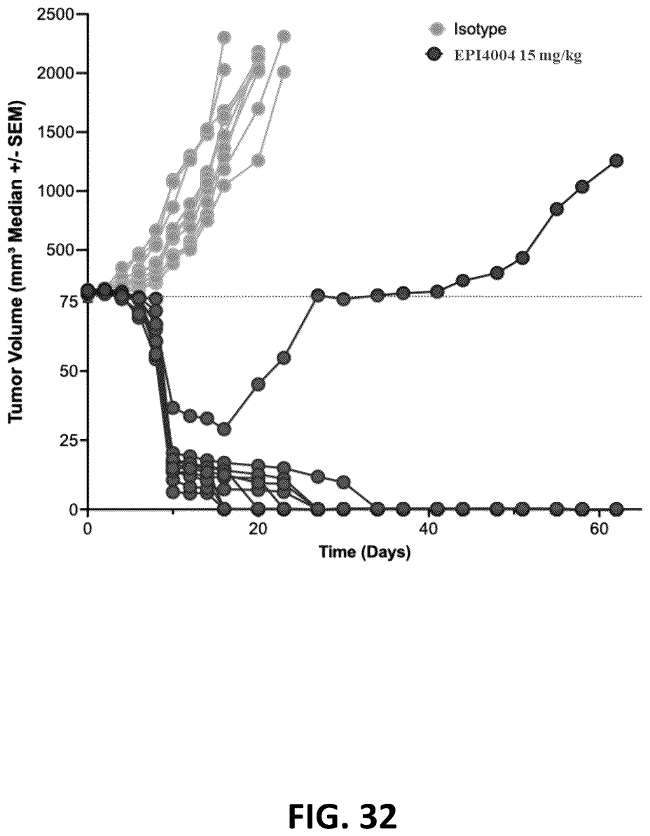

This example sought to determine whether extended dosing with a bispecific antibody comprising an EGFR arm and an ITGB6 arm (EPI4004) would induce complete responses in tumor bearing animals (see FIG. 32).

### Methods

Xenograft tumor models. NCI-H1975 cells were grown in tissue culture flasks containing RPM11640 medium supplemented with 10% fetal bovine serum at 37° C. in an atmosphere of 5% CO2. Cells were harvested during exponential growth phase, and 5×10⁶ total cells were inoculated into the right front flank of BALB/c nude mice. Animals were monitored daily and tumor volumes and body weights were measured twice per week according to IACUC guidelines. Tumors were grown until reaching approximately a volume of 150 mm³, at which point animals were randomized into groups (n=10/group) and given once-weekly intravenous doses of EP14004 at 15 mg/kg for 2 to 8 total doses. Tumor volume was calculated as V=(L×W×W)/2. Graphs were generated in Graphpad Prism.

### Results

Extended weekly dosing led to robust tumor growth inhibition, and 9 out of 10 animals lost measurable tumors (complete regressions) (see FIG. 32).

### Example 29—Inhibited Cell Line and Patient-derived HNSCC Xenograft Tumor Models

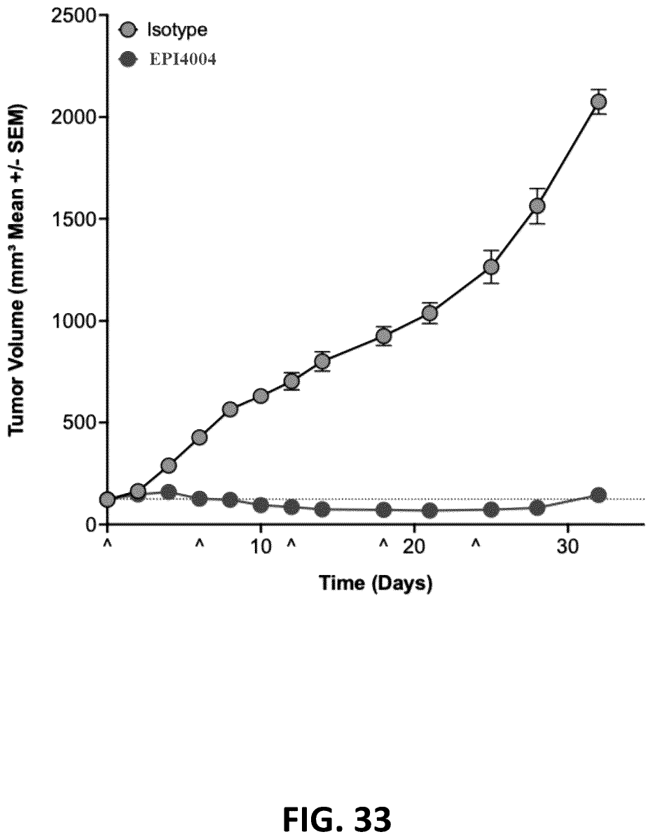

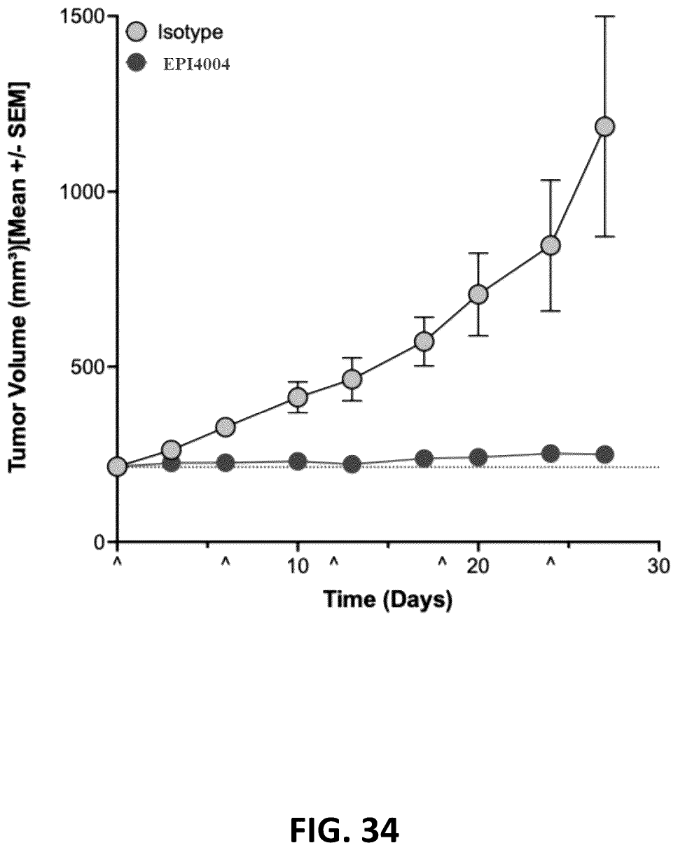

This example sought to determine the anti-tumor activity of a bispecific antibody comprising an EGFR arm and an ITGB6 arm (EPI4004) in HNSCC tumor models carrying wild type EGFR (see FIGS. 33 and 34).

### Methods

Cell line xenograft tumor models. Detroit562 head and neck squamous cell carcinoma tumor (HNSCC) cells were grown in tissue culture flasks containing MEM medium supplemented with 10% fetal bovine serum and 0.01 mM NEAA at 37° C. in an atmosphere of 5% CO2. Cells were harvested during exponential growth phase, and 5×10⁶ total cells were inoculated into the right front flank of female NPG mice. Animals were monitored daily and tumor volumes and body weights were measured twice per week according to IACUC guidelines. Tumors were grown until reaching approximately a volume of 120 mm³, at which point animals were randomized into groups (n=7/group) and given once-weekly 15 mg/kg intravenous doses of EPI4004 for 5 weeks. Tumor volume was calculated as V=(L×W×W)/2. Graphs were generated in Graphpad Prism.

Patient-derived xenograft tumor models. CTG-0149 tumors, from a human patient-derived head and neck squamous cell carcinoma tumor, were passaged as tumor fragments in immunocompromised mice. Fragments were implanted in female Athymic Nude NU(Ncr)-Foxn1nu 6-8 week old study mice. Animals were monitored daily and tumor volumes and body weights were measured twice per week according to IACUC guidelines. Pre-study tumor volumes were recorded until they reached 150-300 mm³, at which point animals were randomized into groups (n=7/group) and given once-weekly 15 mg/kg intravenous doses of EPI4004 weekly for 5 weeks. Tumor volume was calculated as TV=width×length×0.52. Graphs were generated in Graphpad Prism.

### Results

Significant tumor growth inhibition was observed at 15 mg/kg in both xenograft models (see FIGS. 33 and 34).

### Example 30—Inhibited Patient-derived CRC Xenograft Tumor Models

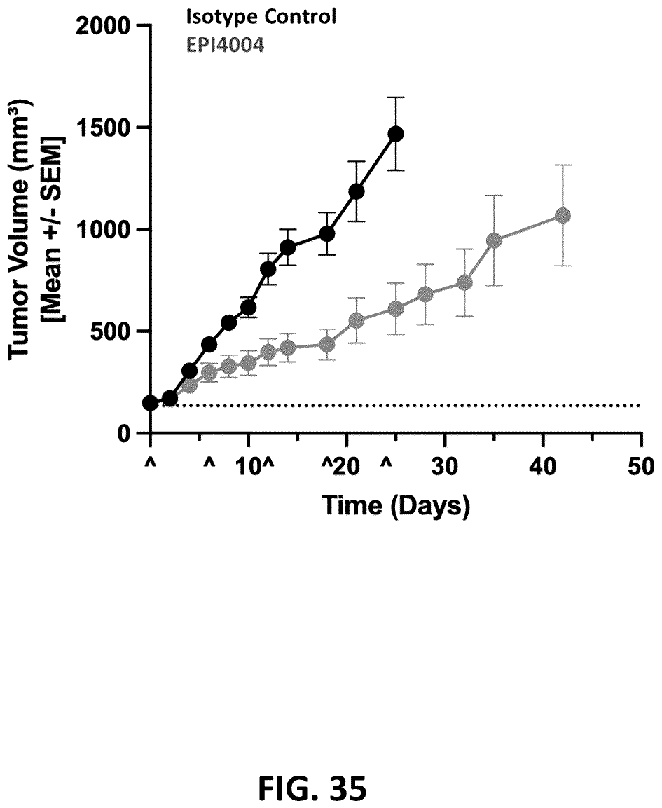

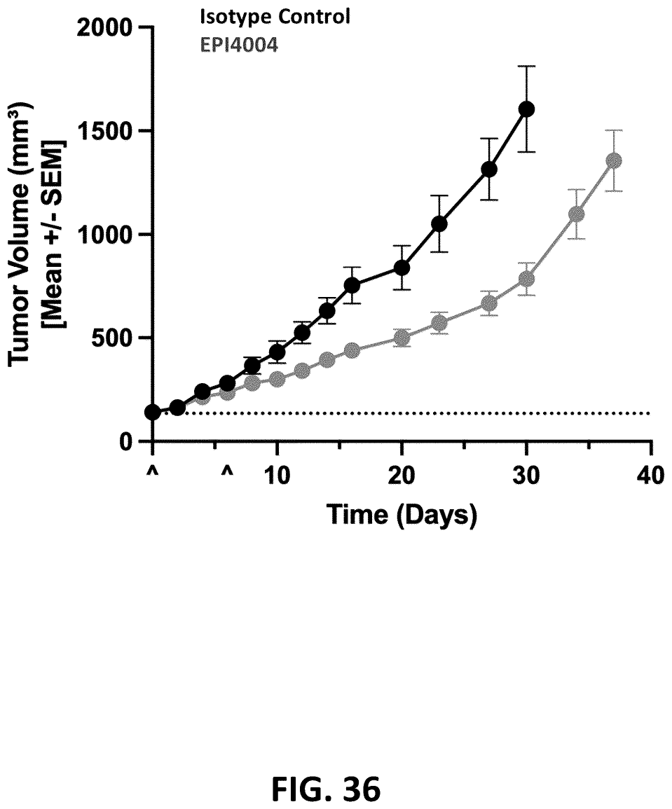

This example sought to determine the anti-tumor activity of a bispecific antibody comprising an EGFR arm and an ITGB6 arm (EPI4004) in colorectal cancer (CRC) tumor models carrying wild type EGFR, but other oncogenic mutations such as TP53, APC and NRAS (see FIGS. 35 and 36).

### Methods

Patient-derived xenograft tumor models. CR5030 and CR9510 tumors, from human patient-derived colorectal carcinoma tumors, were passaged as tumor fragments in immunocompromised mice. Fragments were implanted in Balb/c Nude 6-8 week old study mice. Animals were monitored daily and tumor volumes and body weights were measured twice per week according to IACUC guidelines. Tumors were grown until reaching approximately a volume of 150 mm³, at which point animals were randomized into groups (n=6/group) and given once-weekly 15 mg/kg intravenous doses of EPI4004 for 5 weeks. Tumor volume was calculated as V=(L×W×W)/2. Graphs were generated in Graphpad Prism. Genomic oncogenic mutation data was provided by the vendor.

### Results

CR5030 CRC PDX tumors (NRAS, TP53 mutant) and CR9510 CRC PDX tumors (APC, TP53 mutant) were treated weekly with EPI4004 and the effects on tumor growth kinetics were monitored. Treatment with EPI4004 resulted in tumor growth inhibition at 15 mg/kg in both models (see FIGS. 35 and 36).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An antigen binding molecule, comprising:
(i) a first antigen binding domain that binds to an epidermal growth factor receptor (EGFR); and
(ii) a second antigen binding domain that binds to an integrin subunit beta 6 (ITGB6),
wherein the first antigen binding domain comprises a heavy chain variable region (VH) and a light chain variable region (VL), wherein
A. the first antigen binding domain comprises
(a) a VH comprising a heavy chain complementarity determining region 1 (HCDR1) amino acid sequence of DYGMH (SEQ ID NO: 16), a heavy chain complementarity determining region 2 (HCDR2) amino acid sequence of AIDAGGSTDYADSVEG (SEQ ID NO: 17), or a heavy chain complementarity determining region 3 (HCDR3) amino acid sequence of DLEAGYYAPDV (SEQ ID NO: 18); or
(b) a VL comprising a light chain complementarity determining region 1 (LCDR1) amino acid sequence of RASQDIGRFLA (SEQ ID NO: 31), a light chain complementarity determining region 2 (LCDR2) amino acid sequence of AVSNLQS (SEQ ID NO: 32), or a light chain complementarity determining region 3 (LCDR3) amino acid sequence of QQYSTSVYT (SEQ ID NO: 33); or
B. the second antigen binding domain comprises a HCDR2 amino acid sequence of VINPGSGRTNYAQKFQG (SEQ ID NO: 59).

2. The antigen binding molecule of claim 1, wherein the first antigen binding domain comprises a heavy chain variable region (VH) comprising:
(a) a HCDR1 amino acid sequence of DYGMH (SEQ ID NO: 16);
(b) a HCDR2 amino acid sequence of AIDAGGSTDYADSVEG (SEQ ID NO: 17); or
(c) a HCDR3 amino acid sequence of DLEAGYYAPDV (SEQ ID NO: 18).

3. The antigen binding molecule of claim 1, wherein the first antigen binding domain comprises a light chain variable region (VL) comprising:
(a) a LCDR1 amino acid sequence of RASQDIGRFLA (SEQ ID NO: 31);
(b) a LCDR2 amino acid sequence of AVSNLQS (SEQ ID NO: 32); or
(c) a LCDR3 amino acid sequence of QQYSTSVYT (SEQ ID NO: 33).

4. The antigen binding molecule of claim 1, wherein the second antigen binding domain comprises a heavy chain variable region (VH) comprising a HCDR2 amino acid sequence of VINPGSGRTNYAQKFQG (SEQ ID NO: 59).

5. The antigen binding molecule of claim 1, wherein the first antigen binding domain comprises a heavy chain variable region (VH) comprising:
(a) a HCDR1 amino acid sequence of DYGMH (SEQ ID NO: 16);
(b) a HCDR2 amino acid sequence of AIDAGGSTDYADSVEG (SEQ ID NO: 17); and
(c) a HCDR3 amino acid sequence of DLEAGYYAPDV (SEQ ID NO: 18).

6. The antigen binding molecule of claim 1, wherein the first antigen binding domain comprises a light chain variable region (VL) comprising:
(a) a LCDR1 amino acid sequence of RASQDIGRFLA (SEQ ID NO: 31);
(b) a LCDR2 amino acid sequence of AVSNLQS (SEQ ID NO: 32); and
(c) a LCDR3 amino acid sequence of QQYSTSVYT (SEQ ID NO: 33).

7. The antigen binding molecule of claim 1, wherein the first antigen binding domain comprises:
(a) a VL comprising a LCDR1 amino acid sequence of RASQDIGRFLA (SEQ ID NO: 31), a LCDR2 amino acid sequence of AVSNLQS (SEQ ID NO: 32), and a LCDR3 amino acid sequence of QQYSTSVYT (SEQ ID NO: 33); and
(b) a VH comprising a HCDR1 amino acid sequence of DYGMH (SEQ ID NO: 16), a HCDR2 amino acid sequence of AIDAGGSTDYADSVEG (SEQ ID NO: 17), and a HCDR3 amino acid sequence of DLEAGYYAPDV (SEQ ID NO: 18).

8. The antigen binding molecule of claim 1, wherein the second antigen binding domain comprises a heavy chain variable region (VH) comprising:
(a) a HCDR1 amino acid sequence of NDLIE (SEQ ID NO: 58);
(b) a HCDR2 amino acid sequence of VINPGSGRTNYAQKFQG (SEQ ID NO: 59); and
(c) a HCDR3 amino acid sequence of IYYGPHSYAMDY (SEQ ID NO: 60).

9. The antigen binding molecule of claim 1, wherein the second antigen binding domain comprises:
(a) a VL comprising a LCDR1 amino acid sequence of KASLDVRTAVA (SEQ ID NO: 73), a LCDR2 amino acid sequence of SASYRYT (SEQ ID NO: 74), and a LCDR3 amino acid sequence of QQHYGIPWT (SEQ ID NO: 75); and
(b) a VH comprising a HCDR1 amino acid sequence of NDLIE (SEQ ID NO: 58), a HCDR2 amino acid sequence of VINPGSGRTNYAQKFQG (SEQ ID NO: 59), and a HCDR3 amino acid sequence of IYYGPHSYAMDY (SEQ ID NO: 60).

10. The antigen binding molecule of claim 7, wherein the second antigen binding domain comprises:
(a) a VL comprising a LCDR1 amino acid sequence of KASLDVRTAVA (SEQ ID NO: 73), a LCDR2 amino acid sequence of SASYRYT (SEQ ID NO: 74), and a LCDR3 amino acid sequence of QQHYGIPWT (SEQ ID NO: 75); and
(b) a VH comprising a HCDR1 amino acid sequence of NDLIE (SEQ ID NO: 58), a HCDR2 amino acid sequence of VINPGSGRTNYAQKFQG (SEQ ID NO: 59), and a HCDR3 amino acid sequence of IYYGPHSYAMDY (SEQ ID NO: 60).

11. The antigen binding molecule of claim 1, wherein
(a) the first antigen binding domain comprises (i) a VL that has at least 95% sequence identity to the sequence of SEQ ID NO: 49 or a VH that has at least 95% sequence identity to the sequence of SEQ ID NO: 43; or
(b) the second antigen binding domain comprises (i) a VL that has at least 95% sequence identity to the sequence of SEQ ID NO: 91 or a VH that has at least 95% sequence identity to the sequence of SEQ ID NO: 85.

12. The antigen binding molecule of claim 1, wherein the first antigen binding domain comprises
(a) a VL amino acid sequence of SEQ ID NO: 49; and
(b) a VH amino acid sequence of SEQ ID NO: 43.

13. The antigen binding molecule of claim 1, wherein the second antigen binding domain comprises
(a) a VL amino acid sequence of SEQ ID NO: 91; and
(b) a VH amino acid sequence of SEQ ID NO: 85.

14. The antigen binding molecule of claim 1, wherein the antigen binding molecule comprises
A. a first polypeptide and a second polypeptide, wherein the first polypeptide and the second polypeptide are non-contiguous, wherein:
(a) the first polypeptide comprises the VL of the first antigen binding domain and a first Light Chain Constant Region (CL), wherein the first CL is linked to the VL of the first antigen binding domain; and
(b) the second polypeptide comprises the VH of the first antigen binding domain and a first heavy chain constant region (CH), wherein the first CH is linked to the VH of the first antigen binding domain; and
B. a third polypeptide and a fourth polypeptide, wherein the third polypeptide and the fourth polypeptide are non-contiguous, wherein:
(a) the third polypeptide comprises the VL of the second antigen binding domain and a second CL, wherein the second CL is linked to the VL of the second antigen binding domain; and
(b) the fourth polypeptide comprises the VH of the second antigen binding domain and a second CH, wherein the second CH is linked to the VH of the second antigen binding domain.

15. The antigen binding molecule of claim 14, wherein the first CL comprises a sequence of SEQ ID NO: 51; or the first CH comprises a sequence of SEQ ID NO: 45.

16. The antigen binding molecule of claim 14, wherein the first CL comprises a sequence of SEQ ID NO: 51; and the first CH comprises a sequence of SEQ ID NO: 45.

17. The antigen binding molecule of claim 14, wherein the second CL comprises a sequence of SEQ ID NO: 93; or the second CH comprises a sequence of SEQ ID NO: 87.

18. The antigen binding molecule of claim 14, wherein the second CL comprises a sequence of SEQ ID NO: 93; and the second CH comprises a sequence of SEQ ID NO: 87.

19. The antigen binding molecule of claim 14, wherein the antigen binding molecule comprises:
(a) the first polypeptide, wherein the first polypeptide comprises the sequence of SEQ ID NO: 50;
(b) the second polypeptide, wherein the second polypeptide comprises the sequence of SEQ ID NO: 44;
(c) the third polypeptide, wherein the third polypeptide comprises the sequence of any one of SEQ ID NO: 92; or
(d) the fourth polypeptide, wherein the fourth polypeptide comprises the sequence of SEQ ID NO: 86.

20. The antigen binding molecule of claim 14, wherein the first polypeptide comprises the sequence of SEQ ID NO: 50; and the second polypeptide comprises the sequence of SEQ ID NO: 44.

21. The antigen binding molecule of claim 14, wherein the third polypeptide comprises the sequence of SEQ ID NO: 92; and the fourth polypeptide comprises the sequence of SEQ ID NO: 86.

22. The antigen binding molecule of claim 14, wherein the antigen binding molecule comprises:
(a) the first polypeptide, wherein the first polypeptide comprises the sequence of SEQ ID NO: 50;
(b) the second polypeptide, wherein the second polypeptide comprises the sequence of SEQ ID NO: 44;
(c) the third polypeptide, wherein the third polypeptide comprises the sequence of any one of SEQ ID NO: 92; and
(d) the fourth polypeptide, wherein the fourth polypeptide comprises the sequence of SEQ ID NO: 86.

23. The antigen binding molecule of claim 22, wherein the antigen binding molecule comprises:
(a) the first polypeptide, wherein the first polypeptide comprises the sequence of SEQ ID NO: 50;
(b) the second polypeptide, wherein the second polypeptide comprises the sequence of SEQ ID NO: 676;
(c) the third polypeptide, wherein the third polypeptide comprises the sequence of any one of SEQ ID NO: 92; and
(d) the fourth polypeptide, wherein the fourth polypeptide comprises the sequence of SEQ ID NO: 677.

24. The antigen binding molecule of claim 1, wherein the antigen binding molecule is a multispecific antibody, a bispecific antibody, a bispecific diabody, a bispecific Fab2, bispecific camelid antibody, a bispecific peptibody scFv-Fc, a bispecific IgG, a knob and hole bispecific IgG, a Fc-Fab, or a knob and hole bispecific Fc-Fab.

25. The antigen binding molecule of claim 1, wherein the antigen binding molecule or fragment thereof is conjugated or linked to a cytotoxic agent or a small molecule.

26. The antigen binding molecule of claim 1, wherein (i) binding of the antigen binding molecule to EGFR is configured to block or weakly block the binding of epidermal growth factor (EGF) to EGFR or (ii) binding of the antigen binding molecule to ITGB6 is configured to not block the binding of latent-associated peptide (LAP) to ITGB6.

27. The antigen binding molecule of claim 1, wherein the antibody or an antigen-binding portion thereof competes with and/or binds the same epitope as a reference antibody,
(a) wherein the reference antibody comprises (i) a heavy chain variable region (VH) comprising the amino acid sequence set forth in SEQ ID NO: 43 and (ii) a light chain variable region (VL) comprising the amino acid sequence set forth in SEQ ID NO: 49, and wherein the Kd of the antibody or an antigen-binding portion thereof to EGFR is within +/−10%, +/−20%, or +/−30% of the binding affinity of the reference antibody to EGFR; or
(b) wherein the reference antibody comprises (i) a heavy chain variable region (VH) comprising the amino acid sequence set forth in SEQ ID NO: 85 and (ii) a light chain variable region (VL) comprising the amino acid sequence set forth in SEQ ID NO: 91, and wherein the Kd of the antibody or an antigen-binding portion thereof to ITGB6 is within +/−10%, +/−20%, or +/−30% of the binding affinity of the reference antibody to ITGB6.

28. A recombinant polynucleotide molecule comprising the polynucleotide sequences encoding the antigen binding molecule of claim 1.

29. A pharmaceutical composition comprising the antigen binding molecule of claim 1 and a pharmaceutically acceptable carrier, excipient, or diluent.

30. A method of degrading EGFR on the surface of a cancer cell comprising, contacting the cell with the antigen binding molecule of claim 1.


SEQUENCE LISTING (680 sequences)

SEQ ID NO: 1 - skipped

SEQ ID NO: 2 - protein, 17 aa, synthetic construct
  VARIANT 1: note = A or G
  VARIANT 2: note = F or I
  VARIANT 4: note = P or absent
  VARIANT 5: note = A or D
  VARIANT 6: note = G or A
  VARIANT 8: note = S or E
  VARIANT 10: note = D or I
  VARIANT 13: note = D or Q
  VARIANT 14: note = S or K
  VARIANT 15: note = V or F
  VARIANT 16: note = E or Q
XXDXXXGXTXYAXXXXG

SEQ ID NO: 3 - skipped

SEQ ID NO: 4 - protein, 11 aa, synthetic construct
  VARIANT 7: note = G or R
  VARIANT 8: note = R or H
  VARIANT 9: note = F or Y
RASQDIXXXLA

SEQ ID NO: 5 - skipped

SEQ ID NO: 6 - protein, 9 aa, synthetic construct
  VARIANT 4: note = S or H
  VARIANT 5: note = T or N
  VARIANT 6: note = S or L
  VARIANT 7: note = V or P
  VARIANT 9: note = T or S
QQYXXXXYX

SEQ ID NO: 7 - protein, 5 aa, synthetic construct
  VARIANT 2: note = D or Y
NXLIE

SEQ ID NO: 8 - protein, 17 aa, synthetic construct
  VARIANT 3: note = N or S
  VARIANT 8: note = R or I
  VARIANT 9: note = T or I
VIXPGSGXXNYAQKFQG

SEQ ID NO: 9 - skipped

SEQ ID NO: 10 - protein, 11 aa, synthetic construct
  VARIANT 4: note = L or Q
  VARIANT 5: note = D or A
  VARIANT 7: note = R or N
KASXXVXTAVA

SEQ ID NO: 11 - protein, 7 aa, synthetic construct
  VARIANT 5: note = R or G
SASYXYT

SEQ ID NO: 12 - protein, 9 aa, synthetic construct
  VARIANT 2: note = Q or H
  VARIANT 6: note = I or V
QXHYGXPWT

SEQ ID NO: 13 - protein, 10 aa, synthetic construct
GFTFSDYGMH

SEQ ID NO: 14 - protein, 16 aa, synthetic construct
AIDAGGSTDYADSVEG

SEQ ID NO: 15 - protein, 11 aa, synthetic construct
DLEAGYYAPDV

SEQ ID NO: 16 - protein, 5 aa, synthetic construct
DYGMH

SEQ ID NO: 17 - protein, 16 aa, synthetic construct
AIDAGGSTDYADSVEG

SEQ ID NO: 18 - protein, 11 aa, synthetic construct
DLEAGYYAPDV

SEQ ID NO: 19 - protein, 7 aa, synthetic construct
GFTFSDY

SEQ ID NO: 20 - protein, 5 aa, synthetic construct
DAGGS

SEQ ID NO: 21 - protein, 11 aa, synthetic construct
DLEAGYYAPDV

SEQ ID NO: 22 - protein, 10 aa, synthetic construct
GDTFSNQGIS

SEQ ID NO: 23 - protein, 17 aa, synthetic construct
GFDPDAGETIYAQKFQG

SEQ ID NO: 24 - protein, 15 aa, synthetic construct
GVDSYGYGRYNWFDP

SEQ ID NO: 25 - protein, 5 aa, synthetic construct
NQGIS

SEQ ID NO: 26 - protein, 17 aa, synthetic construct
GFDPDAGETIYAQKFQG

SEQ ID NO: 27 - protein, 15 aa, synthetic construct
GVDSYGYGRYNWFDP

SEQ ID NO: 28 - protein, 7 aa, synthetic construct
GDTFSNQ

SEQ ID NO: 29 - protein, 6 aa, synthetic construct
DPDAGE

SEQ ID NO: 30 - protein, 15 aa, synthetic construct
GVDSYGYGRYNWFDP

SEQ ID NO: 31 - protein, 11 aa, synthetic construct
RASQDIGRFLA

SEQ ID NO: 32 - protein, 7 aa, synthetic construct
AVSNLQS

SEQ ID NO: 33 - protein, 9 aa, synthetic construct
QQYSTSVYT

SEQ ID NO: 34 - protein, 11 aa, synthetic construct
RASQDIGRFLA

SEQ ID NO: 35 - protein, 7 aa, synthetic construct
AVSNLQS

SEQ ID NO: 36 - protein, 9 aa, synthetic construct
QQYSTSVYT

SEQ ID NO: 37 - protein, 11 aa, synthetic construct
RASQDIRHYLA

SEQ ID NO: 38 - protein, 7 aa, synthetic construct
DTFNRAT

SEQ ID NO: 39 - protein, 9 aa, synthetic construct
QQYHNLPYS

SEQ ID NO: 40 - protein, 11 aa, synthetic construct
RASQDIRHYLA

SEQ ID NO: 41 - protein, 7 aa, synthetic construct
DTFNRAT

SEQ ID NO: 42 - protein, 9 aa, synthetic construct
QQYHNLPYS

SEQ ID NO: 43 - protein, 119 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDLEAGYYAPDVWGKGTTVTVSS

SEQ ID NO: 44 - protein, 448 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDLEAGYYAPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 124 aa, synthetic construct
QVQLVQSGAEVKKPGASVKVSCKVSGDTFSNQGISWVRQAPGKGLEWMGGFDPDAGETIYAQKFQGRVTMTEDTSTDTAYMELSSLKSEDTAVYYCARGVDSYGYGRYNWFDPWGQGTLVTVSS

SEQ ID NO: 47 - protein, 453 aa, synthetic construct
QVQLVQSGAEVKKPGASVKVSCKVSGDTFSNQGISWVRQAPGKGLEWMGGFDPDAGETIYAQKFQGRVTMTEDTSTDTAYMELSSLKSEDTAVYYCARGVDSYGYGRYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 329 aa, synthetic construct; identical to SEQ ID NO: 45

SEQ ID NO: 49 - protein, 107 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQYSTSVYTFGGGTKVEIK

SEQ ID NO: 50 - protein, 214 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 107 aa, synthetic construct [sequence omitted]

SEQ ID NO: 52 - protein, 107 aa, synthetic construct
DIQMTQSPSSVSASVGDRVTITCRASQDIRHYLAWYQQKPGKAPKLLIYDTFNRATGVPSRFSGSGSGTDFTLTISSLQPEDFANYYCQQYHNLPYSFGGGTKVEIK

SEQ ID NO: 53 - protein, 214 aa, synthetic construct
DIQMTQSPSSVSASVGDRVTITCRASQDIRHYLAWYQQKPGKAPKLLIYDTFNRATGVPSRFSGSGSGTDFTLTISSLQPEDFANYYCQQYHNLPYSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 107 aa, synthetic construct [sequence omitted]

SEQ ID NO: 55 - protein, 10 aa, synthetic construct
GYDFNNDLIE

SEQ ID NO: 56 - protein, 17 aa, synthetic construct
VINPGSGRTNYAQKFQG

SEQ ID NO: 57 - protein, 12 aa, synthetic construct
IYYGPHSYAMDY

SEQ ID NO: 58 - protein, 5 aa, synthetic construct
NDLIE

SEQ ID NO: 59 - protein, 17 aa, synthetic construct
VINPGSGRTNYAQKFQG

SEQ ID NO: 60 - protein, 12 aa, synthetic construct
IYYGPHSYAMDY

SEQ ID NO: 61 - protein, 7 aa, synthetic construct
GYDFNND

SEQ ID NO: 62 - protein, 6 aa, synthetic construct
NPGSGR

SEQ ID NO: 63 - protein, 12 aa, synthetic construct
IYYGPHSYAMDY

SEQ ID NO: 64 - protein, 10 aa, synthetic construct
GYAFTNYLIE

SEQ ID NO: 65 - protein, 17 aa, synthetic construct
VISPGSGIINYAQKFQG

SEQ ID NO: 66 - protein, 11 aa, synthetic construct
IDYSGPYAVDD

SEQ ID NO: 67 - protein, 5 aa, synthetic construct
NYLIE

SEQ ID NO: 68 - protein, 17 aa, synthetic construct
VISPGSGIINYAQKFQG

SEQ ID NO: 69 - protein, 11 aa, synthetic construct
IDYSGPYAVDD

SEQ ID NO: 70 - protein, 7 aa, synthetic construct
GYAFTNY

SEQ ID NO: 71 - protein, 6 aa, synthetic construct
SPGSGI

SEQ ID NO: 72 - protein, 11 aa, synthetic construct
IDYSGPYAVDD

SEQ ID NO: 73 - protein, 11 aa, synthetic construct
KASLDVRTAVA

SEQ ID NO: 74 - protein, 7 aa, synthetic construct
SASYRYT

SEQ ID NO: 75 - protein, 9 aa, synthetic construct
QQHYGIPWT

SEQ ID NO: 76 - protein, 11 aa, synthetic construct
KASLDVRTAVA

SEQ ID NO: 77 - protein, 7 aa, synthetic construct
SASYRYT

SEQ ID NO: 78 - protein, 9 aa, synthetic construct
QQHYGIPWT

SEQ ID NO: 79 - protein, 11 aa, synthetic construct
KASQAVNTAVA

SEQ ID NO: 80 - protein, 7 aa, synthetic construct
SASYGYT

SEQ ID NO: 81 - protein, 9 aa, synthetic construct
QHHYGVPWT

SEQ ID NO: 82 - protein, 11 aa, synthetic construct
KASQAVNTAVA

SEQ ID NO: 83 - protein, 7 aa, synthetic construct
SASYGYT

SEQ ID NO: 84 - protein, 9 aa, synthetic construct
QHHYGVPWT

SEQ ID NO: 85 - protein, 121 aa, synthetic construct
QVQLVQSGAEVKKPGASVKVSCKASGYDFNNDLIEWVRQAPGQCLEWMAVINPGSGRTNYAQKFQGRVTMTADKSTSTVYMELSSLRSEDTAVYYCAMIYYGPHSYAMDYWGQGTLVTVSS

SEQ ID NO: 86 - protein, 450 aa, synthetic construct
QVQLVQSGAEVKKPGASVKVSCKASGYDFNNDLIEWVRQAPGQCLEWMAVINPGSGRTNYAQKFQGRVTMTADKSTSTVYMELSSLRSEDTAVYYCAMIYYGPHSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 87 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 88 - protein, 120 aa, synthetic construct
QVQLVQSGAEVKKPGSSVKVSCKASGYAFTNYLIEWVRQAPGQCLEWMGVISPGSGIINYAQKFQGRATLTADKSTSTAYMELSSLRSEDTAVYYCAAIDYSGPYAVDDWGQGTLVTVSS

SEQ ID NO: 89 - protein, 449 aa, synthetic construct
QVQLVQSGAEVKKPGSSVKVSCKASGYAFTNYLIEWVRQAPGQCLEWMGVISPGSGIINYAQKFQGRATLTADKSTSTAYMELSSLRSEDTAVYYCAAIDYSGPYAVDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 90 - protein, 329 aa, synthetic construct; identical to SEQ ID NO: 87

SEQ ID NO: 91 - protein, 107 aa, synthetic construct
DIQMTQSPSSLSASVGDRVTITCKASLDVRTAVAWYQQKPGKAPKLLIYSASYRYTGVPSRFSGSGSGTDFTFTISSLQPEDIATYYCQQHYGIPWTFGCGTKVEIK

SEQ ID NO: 92 - protein, 213 aa, synthetic construct
DIQMTQSPSSLSASVGDRVTITCKASLDVRTAVAWYQQKPGKAPKLLIYSASYRYTGVPSRFSGSGSGTDFTFTISSLQPEDIATYYCQQHYGIPWTFGCGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 93 - protein, 106 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 94 - protein, 107 aa, synthetic construct
DIQMTQSPSSLSASVGDRVTITCKASQAVNTAVAWYQQKPGKAPKLLIYSASYGYTGVPSRFSGSGSGTDFTLTISSLQPEDFATYYCQHHYGVPWTFGCGTKLEIK

SEQ ID NO: 95 - protein, 213 aa, synthetic construct
DIQMTQSPSSLSASVGDRVTITCKASQAVNTAVAWYQQKPGKAPKLLIYSASYGYTGVPSRFSGSGSGTDFTLTISSLQPEDFATYYCQHHYGVPWTFGCGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 96 - protein, 106 aa, synthetic construct; identical to SEQ ID NO: 93

SEQ ID NO: 97 - protein, 17 aa, synthetic construct
  VARIANT 12: note = N or A
  VARIANT 13: note = E or Q
VINPGSGRTNYXXKFQG

SEQ ID NO: 98 - protein, 17 aa, synthetic construct
  VARIANT 12: note = N or A
  VARIANT 13: note = E or Q
VISPGSGIINYXXKFQG

SEQ ID NO: 99 - protein, 121 aa, synthetic construct
  VARIANT 5: note = V or Q
  VARIANT 11: note = V or L
  VARIANT 12: note = K or V
  VARIANT 13: note = K or R
  VARIANT 16: note = A or T
  VARIANT 38: note = R or K
  VARIANT 40: note = A or R
  VARIANT 44: note = C or G
  VARIANT 48: note = M or I
  VARIANT 61: note = A or N
  VARIANT 62: note = Q or E
  VARIANT 65: note = Q or K
  VARIANT 67: note = R or K
  VARIANT 68: note = V or A
  VARIANT 70: note = M or L
  VARIANT 76: note = T or S
  VARIANT 82: note = E or Q
  VARIANT 87: note = R or T
  VARIANT 89: note = E or D
  VARIANT 91: note = T or S
  VARIANT 95: note = Y or F
  VARIANT 116: note = L or S
QVQLXQSGAEXXXPGXSVKVSCKASGYDFNNDLIEWVXQXPGQXLEWXAVINPGSGRTNYXXKFXGXXTXTADKSXSTVYMXLSSLXSXDXAVYXCAMIYYGPHSYAMDYWGQGTXVTVSS

SEQ ID NO: 100 - protein, 451 aa, synthetic construct
  VARIANT 5: note = V or Q
  VARIANT 11: note = V or L
  VARIANT 12: note = K or V
  VARIANT 13: note = K or R
  VARIANT 16: note = A or T
  VARIANT 38: note = R or K
  VARIANT 40: note = A or R
  VARIANT 44: note = C or G
  VARIANT 48: note = M or I
  VARIANT 61: note = A or N
  VARIANT 62: note = Q or E
  VARIANT 65: note = Q or K
  VARIANT 67: note = R or K
  VARIANT 68: note = V or A
  VARIANT 70: note = M or L
  VARIANT 76: note = T or S
  VARIANT 82: note = E or Q
  VARIANT 87: note = R or T
  VARIANT 89: note = E or D
  VARIANT 91: note = T or S
  VARIANT 95: note = Y or F
  VARIANT 116: note = L or S
QVQLXQSGAEXXXPGXSVKVSCKASGYDFNNDLIEWVXQXPGQXLEWXAVINPGSGRTNYXXKFXGXXTXTADKSXSTVYMXLSSLXSXDXAVYXCAMIYYGPHSYAMDYWGQGTXVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 107 aa, synthetic construct
  VARIANT 3: note = V or Q
  VARIANT 8: note = P or H
  VARIANT 9: note = S or K
  VARIANT 10: note = S or F
  VARIANT 11: note = M or L
  VARIANT 13: note = A or T
  VARIANT 14: note = V or S
  VARIANT 20: note = T or S
  VARIANT 42: note = K or Q
  VARIANT 43: note = A or S
  VARIANT 60: note = S or D
  VARIANT 63: note = S or T
  VARIANT 74: note = T or N
  VARIANT 76: note = S or R
  VARIANT 78: note = V or L
  VARIANT 80: note = P or A
  VARIANT 83: note = I or L
  VARIANT 85: note = V or T
DIXMTQSXXXXSXXVGDRVXITCKASLDVRTAVAWYQQKPGXXPKLLIYSASYRYTGVPXRFXGSGSGTDFTFXIXSXQXEDXAXYYCQQHYGIPWTFGGGTKLEIK

SEQ ID NO: 102 - protein, 214 aa, synthetic construct
  VARIANT 3: note = V or Q
  VARIANT 8: note = P or H
  VARIANT 9: note = S or K
  VARIANT 10: note = S or F
  VARIANT 11: note = M or L
  VARIANT 13: note = A or T
  VARIANT 14: note = V or S
  VARIANT 20: note = T or S
  VARIANT 42: note = K or Q
  VARIANT 43: note = A or S
  VARIANT 60: note = S or D
  VARIANT 63: note = S or T
  VARIANT 74: note = T or N
  VARIANT 76: note = S or R
  VARIANT 78: note = V or L
  VARIANT 80: note = P or A
  VARIANT 83: note = I or L
  VARIANT 85: note = V or T
DIXMTQSXXXXSXXVGDRVXITCKASLDVRTAVAWYQQKPGXXPKLLIYSASYRYTGVPXRFXGSGSGTDFTFXIXSXQXEDXAXYYCQQHYGIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - skipped

SEQ ID NO: 104 - protein, 16 aa, synthetic construct
  VARIANT 3: note = D or S
  VARIANT 4: note = R or A
  VARIANT 5: note = G or S
  VARIANT 6: note = G or A
  VARIANT 7: note = S or A
  VARIANT 8: note = T or A
  VARIANT 9: note = D, G, A, or Y
  VARIANT 15: note = K or E
AIXXXXXXXYADSVXG

SEQ ID NO: 105 - skipped

SEQ ID NO: 106 - protein, 7 aa, synthetic construct
  VARIANT 4: note = T, N, or A
  VARIANT 5: note = L or A
  VARIANT 6: note = Q or A
AVSXXXS

SEQ ID NO: 107 - protein, 9 aa, synthetic construct
  VARIANT 3: note = Y or A
  VARIANT 4: note = S or A
  VARIANT 5: note = T or A
  VARIANT 6: note = S or A
  VARIANT 7: note = V, P, or A
QQXXXXXYT

SEQ ID NO: 108 - protein, 103 aa, synthetic construct
  VARIANT 11: note = L or S
  VARIANT 26: note = G or L
  VARIANT 51: note = H, K, or D
  VARIANT 70: note = T, K, or D
ASTKGPSVFPXAPSSKSTSGGTAALXCLVKDYFPEPVTVSWNSGALTSGVXTFPAVLQSSGLYSLSSVVXVPSSSLGTQTYICNVNHKPSNTKVDKKVEPKSC

SEQ ID NO: 109 - protein, 107 aa, synthetic construct
  VARIANT 9: note = F or T
  VARIANT 11: note = F or M
  VARIANT 30: note = N, D, or K
  VARIANT 31: note = N, D, or K
RTVAAPSVXIXPPSDEQLKSGTASVVCLLXXFYPREAKVQWKVDNALQSGNSQESVTEQDSKDSTYSLSSTLTLSKADYEKHKVYACEVTHQGLSSPVTKSFNRGEC

SEQ ID NO: 110 - protein, 449 aa, synthetic construct
  VARIANT 130: note = L or S
  VARIANT 145: note = G or L
  VARIANT 170: note = H, K, or D
  VARIANT 189: note = T, K, or D
  VARIANT 449: note = K or absent
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDLEAGYYAPDVWGKGTTVTVSSASTKGPSVFPXAPSSKSTSGGTAALXCLVKDYFPEPVTVSWNSGALTSGVXTFPAVLQSSGLYSLSSVVXVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 111 - protein, 214 aa, synthetic construct
  VARIANT 116: note = F or T
  VARIANT 118: note = F or M
  VARIANT 137: note = N, D, or K
  VARIANT 138: note = N, D, or K
DIQMTQSPSSLSASVGDRVTITCRASQDIGRFLAWYQQKPGKAPKLLIYAVSNLQSGVPSRFSGSGSGTDFTLTISSLQPEDVATYYCQQYSTSVYTFGGGTKVEIKRTVAAPSVXIXPPSDEQLKSGTASVVCLLXXFYPREAKVQWKVDNALQSGNSQESVTEQDSKDSTYSLSSTLTLSKADYEKHKVYACEVTHQGLSSPVTKSFNRGEC

SEQ ID NO: 112 - protein, 451 aa, synthetic construct
  VARIANT 5: note = V or Q
  VARIANT 11: note = V or L
  VARIANT 12: note = K or V
  VARIANT 13: note = K or R
  VARIANT 16: note = A or T
  VARIANT 38: note = R or K
  VARIANT 40: note = A or R
  VARIANT 44: note = C or G
  VARIANT 48: note = M or I
  VARIANT 61: note = A or N
  VARIANT 62: note = Q or E
  VARIANT 65: note = Q or K
  VARIANT 67: note = R or K
  VARIANT 68: note = V or A
  VARIANT 70: note = M or L
  VARIANT 76: note = T or S
  VARIANT 82: note = E or Q
  VARIANT 87: note = R or T
  VARIANT 89: note = E or D
  VARIANT 91: note = T or S
  VARIANT 95: note = Y or F
  VARIANT 116: note = L or S
  VARIANT 132: note = L or S
  VARIANT 147: note = G or L
  VARIANT 172: note = H, K, or D
  VARIANT 191: note = T, K, or D
QVQLXQSGAEXXXPGXSVKVSCKASGYDFNNDLIEWVXQXPGQXLEWXAVINPGSGRTNYXXKFXGXXTXTADKSXSTVYMXLSSLXSXDXAVYXCAMIYYGPHSYAMDYWGQGTXVTVSSASTKGPSVFPXAPSSKSTSGGTAALXCLVKDYFPEPVTVSWNSGALTSGVXTFPAVLQSSGLYSLSSVVXVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 214 aa, synthetic construct
  VARIANT 3: note = V or Q
  VARIANT 8: note = P or H
  VARIANT 9: note = S or K
  VARIANT 10: note = S or F
  VARIANT 11: note = M or L
  VARIANT 13: note = A or T
  VARIANT 14: note = V or S
  VARIANT 20: note = T or S
  VARIANT 42: note = K or Q
  VARIANT 43: note = A or S
  VARIANT 60: note = S or D
  VARIANT 63: note = S or T
  VARIANT 74: note = T or N
  VARIANT 76: note = S or R
  VARIANT 78: note = V or L
  VARIANT 80: note = P or A
  VARIANT 83: note = I or L
  VARIANT 85: note = V or T
  VARIANT 116: note = F or T
  VARIANT 118: note = F or M
  VARIANT 137: note = N, D, or K
  VARIANT 138: note = N, D, or K
DIXMTQSXXXXSXXVGDRVXITCKASLDVRTAVAWYQQKPGXXPKLLIYSASYRYTGVPXRFXGSGSGTDFTFXIXSXQXEDXAXYYCQQHYGIPWTFGGGTKLEIKRTVAAPSVXIXPPSDEQLKSGTASVVCLLXXFYPREAKVQWKVDNALQSGNSQESVTEQDSKDSTYSLSSTLTLSKADYEKHKVYACEVTHQGLSSPVTKSFNRGEC

SEQ ID NO: 114 - protein, 448 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDLEAGYYAPDVWGKGTTVTVSSASTKGPSVFPSAPSSKSTSGGTAALLCLVKDYFPEPVTVSWNSGALTSGVKTFPAVLQSSGLYSLSSVVKVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 214 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQYSTSVYTFGGGTKVEIKRTVAAPSVTIMPPSDEQLKSGTASVVCLLDDFYPREAKVQWKVDNALQSGNSQESVTEQDSKDSTYSLSSTLTLSKADYEKHKVYACEVTHQGLSSPVTKSFNRGEC

SEQ ID NO: 116 - protein, 450 aa, synthetic construct
QVQLVQSGAEVKKPGASVKVSCKASGYDFNNDLIEWVRQAPGQCLEWMAVINPGSGRTNYAQKFQGRVTMTADKSTSTVYMELSSLRSEDTAVYYCAMIYYGPHSYAMDYWGQGTLVTVSSASTKGPSVFPLAPSSKSTSGGTAALGCLVKDYFPEPVTVSWNSGALTSGVDTFPAVLQSSGLYSLSSVVDVPSSSLGTQTYICNVNHKPSNTKVDKKVEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 117 - protein, 213 aa, synthetic construct
DIQMTQSPSSLSASVGDRVTITCKASLDVRTAVAWYQQKPGKAPKLLIYSASYRYTGVPSRFSGSGSGTDFTFTISSLQPEDIATYYCQQHYGIPWTFGCGTKVEIKRTVAAPSVFIFPPSDEQLKSGTASVVCLLKKFYPREAKVQWKVDNALQSGNSQESVTEQDSKDSTYSLSSTLTLSKADYEKHKVYACEVTHQGLSSPVTKSFNRGE

SEQ ID NO: 118 - protein, 103 aa, synthetic construct
ASTKGPSVFPSAPSSKSTSGGTAALLCLVKDYFPEPVTVSWNSGALTSGVKTFPAVLQSSGLYSLSSVVKVPSSSLGTQTYICNVNHKPSNTKVDKKVEPKSC

SEQ ID NO: 119 - protein, 107 aa, synthetic construct
RTVAAPSVTIMPPSDEQLKSGTASVVCLLDDFYPREAKVQWKVDNALQSGNSQESVTEQDSKDSTYSLSSTLTLSKADYEKHKVYACEVTHQGLSSPVTKSFNRGEC

SEQ ID NO: 120 - protein, 103 aa, synthetic construct
ASTKGPSVFPLAPSSKSTSGGTAALGCLVKDYFPEPVTVSWNSGALTSGVDTFPAVLQSSGLYSLSSVVDVPSSSLGTQTYICNVNHKPSNTKVDKKVEPKSC

SEQ ID NO: 121 - protein, 107 aa, synthetic construct
RTVAAPSVFIFPPSDEQLKSGTASVVCLLKKFYPREAKVQWKVDNALQSGNSQESVTEQDSKDSTYSLSSTLTLSKADYEKHKVYACEVTHQGLSSPVTKSFNRGEC

SEQ ID NO: 122 - protein, 103 aa, synthetic construct
ASTKGPSVFPSAPSSKSTSGGTAALLCLVKDYFPEPVTVSWNSGALTSGVDTFPAVLQSSGLYSLSSVVDVPSSSLGTQTYICNVNHKPSNTKVDKKVEPKSC

SEQ ID NO: 123 - protein, 107 aa, synthetic construct
RTVAAPSVTIMPPSDEQLKSGTASVVCLLKKFYPREAKVQWKVDNALQSGNSQESVTEQDSKDSTYSLSSTLTLSKADYEKHKVYACEVTHQGLSSPVTKSFNRGEC

SEQ ID NO: 124 - protein, 103 aa, synthetic construct
ASTKGPSVFPLAPSSKSTSGGTAALGCLVKDYFPEPVTVSWNSGALTSGVKTFPAVLQSSGLYSLSSVVKVPSSSLGTQTYICNVNHKPSNTKVDKKVEPKSC

SEQ ID NO: 125 - protein, 107 aa, synthetic construct
RTVAAPSVFIFPPSDEQLKSGTASVVCLLDDFYPREAKVQWKVDNALQSGNSQESVTEQDSKDSTYSLSSTLTLSKADYEKHKVYACEVTHQGLSSPVTKSFNRGEC

SEQ ID NO: 126 - protein, 449 aa, synthetic construct
EVQLVESGGGLVQPGRSLRLSCAASGFMFSDYGMHWVRQAPGKGLEWVSAIDRGGSTDYADSVEGRFTISRDNAKNSLYLQMNSLRAEDTAVYYCAKDLESGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 449 aa, synthetic construct
EVQLVESGGGLVQPGRSLRLSCAASGFTFSDYGMHWVRQAPGKGLEWVSAIDRGGSTDYADSVEGRFTISRDNAKNSLYLQMNSLRAEDTAVYYCAKDLESGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 449 aa, synthetic construct
EVQLVESGGGLVQPGRSLRLSCAASGFMFSDYGMHWVRQAPGKGLEWVSAIDAGGSTDYADSVEGRFTISRDNAKNSLYLQMNSLRAEDTAVYYCAKDLESGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 449 aa, synthetic construct
EVQLVESGGGLVQPGRSLRLSCAASGFMFSDYGMHWVRQAPGKGLEWVSAIDRGGATDYADSVEGRFTISRDNAKNSLYLQMNSLRAEDTAVYYCAKDLESGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 449 aa, synthetic construct
EVQLVESGGGLVQPGRSLRLSCAASGFMFSDYGMHWVRQAPGKGLEWVSAIDRGGSTYYADSVEGRFTISRDNAKNSLYLQMNSLRAEDTAVYYCAKDLESGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 449 aa, synthetic construct
EVQLVESGGGLVQPGRSLRLSCAASGFMFSDYGMHWVRQAPGKGLEWVSAIDRGGSTDYADSVKGRFTISRDNAKNSLYLQMNSLRAEDTAVYYCAKDLESGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 449 aa, synthetic construct
EVQLVESGGGLVQPGRSLRLSCAASGFMFSDYGMHWVRQAPGKGLEWVSAIDRGGSTDYADSVEGRFTISRDNAKNSLYLQMNSLRAEDTAVYYCAKDLASGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 449 aa, synthetic construct
EVQLVESGGGLVQPGRSLRLSCAASGFMFSDYGMHWVRQAPGKGLEWVSAIDRGGSTDYADSVEGRFTISRDNAKNSLYLQMNSLRAEDTAVYYCAKDLEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 449 aa, synthetic construct
EVQLVESGGGLVQPGRSLRLSCAASGFMFSDYGMHWVRQAPGKGLEWVSAIDRGGSTDYADSVEGRFTISRDNAKNSLYLQMNSLRAEDTAVYYCAKDLESGAYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 449 aa, synthetic construct
EVQLVESGGGLVQPGRSLRLSCAASGFMFSDYGMHWVRQAPGKGLEWVSAIDRGGSTDYADSVEGRFTISRDNAKNSLYLQMNSLRAEDTAVYYCAKDLESGYAGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 449 aa, synthetic construct
EVQLVESGGGLVQPGRSLRLSCAASGFMFSDYGMHWVRQAPGKGLEWVSAIDRGGSTDYADSVEGRFTISRDNAKNSLYLQMNSLRAEDTAVYYCAKDLESGYYGFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 449 aa, synthetic construct; identical to SEQ ID NO: 126

SEQ ID NO: 138 - protein, 449 aa, synthetic construct; identical to SEQ ID NO: 126

SEQ ID NO: 139 - protein, 449 aa, synthetic construct; identical to SEQ ID NO: 126

SEQ ID NO: 140 - protein, 449 aa, synthetic construct; identical to SEQ ID NO: 126

SEQ ID NO: 141 - protein, 449 aa, synthetic construct; identical to SEQ ID NO: 126

SEQ ID NO: 142 - protein, 449 aa, synthetic construct; identical to SEQ ID NO: 126

SEQ ID NO: 143 - protein, 449 aa, synthetic construct; identical to SEQ ID NO: 126

SEQ ID NO: 144 - protein, 449 aa, synthetic construct; identical to SEQ ID NO: 126

SEQ ID NO: 145 - protein, 449 aa, synthetic construct; identical to SEQ ID NO: 126

SEQ ID NO: 146 - protein, 449 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDLESGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 449 aa, synthetic construct
EVQLVESGGGLVQPGRSLRLSCAASGFTFSDYGMHWVRQAPGKGLEWVSAIDRGGSTDYADSVEGRFTISRDNAKNSLYLQMNSLRAEDTAVYYCAKDLEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 449 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDLEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 449 aa, synthetic construct
EVQLVESGGGLVQPGRSLRLSCAASGFTFSDYGMHWVRQAPGKGLEWVSAIDRGGSTDYADSVEGRFTISRDNAKNSLYLQMNSLRAEDTAVYYCAKDLESGAYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 449 aa, synthetic construct
EVQLVESGGGLVQPGRSLRLSCAASGFTFSDYGMHWVRQAPGKGLEWVSAIDRGGSTDYADSVEGRFTISRDNAKNSLYLQMNSLRAEDTAVYYCAKDLESGAYGFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 449 aa, synthetic construct
EVQLVESGGGLVQPGRSLRLSCAASGFTFSAYGMHWVRQAPGKGLEWVSAIDAGGSTDYADSVEGRFTISRDNAKNSLYLQMNSLRAEDTAVYYCAKDLEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 449 aa, synthetic construct
EVQLVESGGGLVQPGRSLRLSCAASGFTFSDAGMHWVRQAPGKGLEWVSAIDAGGSTDYADSVEGRFTISRDNAKNSLYLQMNSLRAEDTAVYYCAKDLEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 449 aa, synthetic construct
EVQLVESGGGLVQPGRSLRLSCAASGFTFSDYAMHWVRQAPGKGLEWVSAIDAGGSTDYADSVEGRFTISRDNAKNSLYLQMNSLRAEDTAVYYCAKDLEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 449 aa, synthetic construct
EVQLVESGGGLVQPGRSLRLSCAASGFTFSDYGMHWVRQAPGKGLEWVSAISAGGSTDYADSVEGRFTISRDNAKNSLYLQMNSLRAEDTAVYYCAKDLEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 449 aa, synthetic construct
EVQLVESGGGLVQPGRSLRLSCAASGFTFSDYGMHWVRQAPGKGLEWVSAIDASGSTDYADSVEGRFTISRDNAKNSLYLQMNSLRAEDTAVYYCAKDLEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 449 aa, synthetic construct
EVQLVESGGGLVQPGRSLRLSCAASGFTFSDYGMHWVRQAPGKGLEWVSAIDAGASTDYADSVEGRFTISRDNAKNSLYLQMNSLRAEDTAVYYCAKDLEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 449 aa, synthetic construct
EVQLVESGGGLVQPGRSLRLSCAASGFTFSDYGMHWVRQAPGKGLEWVSAIDAGGSADYADSVEGRFTISRDNAKNSLYLQMNSLRAEDTAVYYCAKDLEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 449 aa, synthetic construct
EVQLVESGGGLVQPGRSLRLSCAASGFTFSDYGMHWVRQAPGKGLEWVSAIDAGGSTAYADSVEGRFTISRDNAKNSLYLQMNSLRAEDTAVYYCAKDLEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 449 aa, synthetic construct
EVQLVESGGGLVQPGRSLRLSCAASGFTFSDYGMHWVRQAPGKGLEWVSAIDAGGSTGYADSVEGRFTISRDNAKNSLYLQMNSLRAEDTAVYYCAKDLEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 449 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKALEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 449 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDAEAGYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 449 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDLEAAYYGPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 449 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDLEAGYYAPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 449 aa, synthetic construct; identical to SEQ ID NO: 148

SEQ ID NO: 165 - protein, 449 aa, synthetic construct; identical to SEQ ID NO: 148

SEQ ID NO: 166 - protein, 449 aa, synthetic construct; identical to SEQ ID NO: 148

SEQ ID NO: 167 - protein, 449 aa, synthetic construct; identical to SEQ ID NO: 148

SEQ ID NO: 168 - protein, 449 aa, synthetic construct; identical to SEQ ID NO: 148

SEQ ID NO: 169 - protein, 449 aa, synthetic construct; identical to SEQ ID NO: 148

SEQ ID NO: 170 - protein, 449 aa, synthetic construct; identical to SEQ ID NO: 148

SEQ ID NO: 171 - protein, 449 aa, synthetic construct; identical to SEQ ID NO: 148

SEQ ID NO: 172 - protein, 449 aa, synthetic construct; identical to SEQ ID NO: 148

SEQ ID NO: 173 - protein, 449 aa, synthetic construct; identical to SEQ ID NO: 148

SEQ ID NO: 174 - protein, 454 aa, synthetic construct
QVQLVQSGAEVKKPGASVKVSCKVSGDTFSNQGISWVRQAPGKGLEWMGGFDPDAGETIYAQKFQGRVTMTEDTSTDTAYMELSSLKSEDTAVYYCARGVDSYGYGRYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 453 aa, synthetic construct
QVQLQESGPGLVKPSQTLSLTCTVSGASIRSGDYSWSWIRQPPGKGLEWIGYIEHSGRTDYNPSLKSRVTMSVDTSKNQFSLKVNSVTAADTAVYYCARASGWYGVYDAFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 453 aa, synthetic construct
QVQLQESGPGLVKPSQTLSLTCTVSGASITSGSHYWSWIRQPPGKGLEWIGYMYHSGSADYNPSLKSRVTMSVDTSKNQFSLKVNSVTAADTAVYYCARTLGVYGDYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 451 aa, synthetic construct
QVQLQQSGAELVRPGTSVKVSCKASGYDFNNDLIEWVKQRPGQGLEWIAVINPGSGRTNYNEKFKGKATLTADKSSSTVYMQLSSLTSDDSAVYFCAMIYYGPHSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 451 aa, synthetic construct
QVQLVQSGAEVKKPGASVKVSCKASGYDFNNDLIEWVRQAPGQGLEWMAVINPGSGRTNYAQKFQGRVTMTADKSTSTVYMELSSLRSEDTAVYYCAMIYYGPHSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 450 aa, synthetic construct
QVQLQQSGAELARPGTSVKVSCKASGYAFTNYLIEWVKQRPGQGLEWIGVISPGSGIINYNEKFKGKATLTADKSSSTAYMQLSSLTSDDSAVYFCAAIDYSGPYAVDDWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 450 aa, synthetic construct
QVQLVQSGAEVKKPGSSVKVSCKASGYAFTNYLIEWVRQAPGQGLEWMGVISPGSGIINYAQKFQGRATLTADKSTSTAYMELSSLRSEDTAVYYCAAIDYSGPYAVDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 119 aa, synthetic construct
EVQLVESGGGLVQPGRSLRLSCAASGFMFSDYGMHWVRQAPGKGLEWVSAIDRGGSTDYADSVEGRFTISRDNAKNSLYLQMNSLRAEDTAVYYCAKDLESGYYGPDVWGKGTTVTVSS

SEQ ID NO: 182 - protein, 119 aa, synthetic construct
EVQLVESGGGLVQPGRSLRLSCAASGFTFSDYGMHWVRQAPGKGLEWVSAIDRGGSTDYADSVEGRFTISRDNAKNSLYLQMNSLRAEDTAVYYCAKDLESGYYGPDVWGKGTTVTVSS

SEQ ID NO: 183 - protein, 119 aa, synthetic construct
EVQLVESGGGLVQPGRSLRLSCAASGFMFSDYGMHWVRQAPGKGLEWVSAIDAGGSTDYADSVEGRFTISRDNAKNSLYLQMNSLRAEDTAVYYCAKDLESGYYGPDVWGKGTTVTVSS

SEQ ID NO: 184 - protein, 119 aa, synthetic construct
EVQLVESGGGLVQPGRSLRLSCAASGFMFSDYGMHWVRQAPGKGLEWVSAIDRGGATDYADSVEGRFTISRDNAKNSLYLQMNSLRAEDTAVYYCAKDLESGYYGPDVWGKGTTVTVSS

SEQ ID NO: 185 - protein, 119 aa, synthetic construct
EVQLVESGGGLVQPGRSLRLSCAASGFMFSDYGMHWVRQAPGKGLEWVSAIDRGGSTYYADSVEGRFTISRDNAKNSLYLQMNSLRAEDTAVYYCAKDLESGYYGPDVWGKGTTVTVSS

SEQ ID NO: 186 - protein, 119 aa, synthetic construct
EVQLVESGGGLVQPGRSLRLSCAASGFMFSDYGMHWVRQAPGKGLEWVSAIDRGGSTDYADSVKGRFTISRDNAKNSLYLQMNSLRAEDTAVYYCAKDLESGYYGPDVWGKGTTVTVSS

SEQ ID NO: 187 - protein, 119 aa, synthetic construct
EVQLVESGGGLVQPGRSLRLSCAASGFMFSDYGMHWVRQAPGKGLEWVSAIDRGGSTDYADSVEGRFTISRDNAKNSLYLQMNSLRAEDTAVYYCAKDLASGYYGPDVWGKGTTVTVSS

SEQ ID NO: 188 - protein, 119 aa, synthetic construct
EVQLVESGGGLVQPGRSLRLSCAASGFMFSDYGMHWVRQAPGKGLEWVSAIDRGGSTDYADSVEGRFTISRDNAKNSLYLQMNSLRAEDTAVYYCAKDLEAGYYGPDVWGKGTTVTVSS

SEQ ID NO: 189 - protein, 119 aa, synthetic construct
EVQLVESGGGLVQPGRSLRLSCAASGFMFSDYGMHWVRQAPGKGLEWVSAIDRGGSTDYADSVEGRFTISRDNAKNSLYLQMNSLRAEDTAVYYCAKDLESGAYGPDVWGKGTTVTVSS

SEQ ID NO: 190 - protein, 119 aa, synthetic construct
EVQLVESGGGLVQPGRSLRLSCAASGFMFSDYGMHWVRQAPGKGLEWVSAIDRGGSTDYADSVEGRFTISRDNAKNSLYLQMNSLRAEDTAVYYCAKDLESGYAGPDVWGKGTTVTVSS

SEQ ID NO: 191 - protein, 119 aa, synthetic construct
EVQLVESGGGLVQPGRSLRLSCAASGFMFSDYGMHWVRQAPGKGLEWVSAIDRGGSTDYADSVEGRFTISRDNAKNSLYLQMNSLRAEDTAVYYCAKDLESGYYGFDVWGKGTTVTVSS

SEQ ID NO: 192 - protein, 119 aa, synthetic construct; identical to SEQ ID NO: 181

SEQ ID NO: 193 - protein, 119 aa, synthetic construct; identical to SEQ ID NO: 181

SEQ ID NO: 194 - protein, 119 aa, synthetic construct; identical to SEQ ID NO: 181

SEQ ID NO: 195 - protein, 119 aa, synthetic construct; identical to SEQ ID NO: 181

SEQ ID NO: 196 - protein, 119 aa, synthetic construct; identical to SEQ ID NO: 181

SEQ ID NO: 197 - protein, 119 aa, synthetic construct; identical to SEQ ID NO: 181

SEQ ID NO: 198 - protein, 119 aa, synthetic construct; identical to SEQ ID NO: 181

SEQ ID NO: 199 - protein, 119 aa, synthetic construct; identical to SEQ ID NO: 181

SEQ ID NO: 200 - protein, 119 aa, synthetic construct; identical to SEQ ID NO: 181

SEQ ID NO: 201 - protein, 119 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDLESGYYGPDVWGKGTTVTVSS

SEQ ID NO: 202 - protein, 119 aa, synthetic construct
EVQLVESGGGLVQPGRSLRLSCAASGFTFSDYGMHWVRQAPGKGLEWVSAIDRGGSTDYADSVEGRFTISRDNAKNSLYLQMNSLRAEDTAVYYCAKDLEAGYYGPDVWGKGTTVTVSS

SEQ ID NO: 203 - protein, 119 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDLEAGYYGPDVWGKGTTVTVSS

SEQ ID NO: 204 - protein, 119 aa, synthetic construct
EVQLVESGGGLVQPGRSLRLSCAASGFTFSDYGMHWVRQAPGKGLEWVSAIDRGGSTDYADSVEGRFTISRDNAKNSLYLQMNSLRAEDTAVYYCAKDLESGAYGPDVWGKGTTVTVSS

SEQ ID NO: 205 - protein, 119 aa, synthetic construct
EVQLVESGGGLVQPGRSLRLSCAASGFTFSDYGMHWVRQAPGKGLEWVSAIDRGGSTDYADSVEGRFTISRDNAKNSLYLQMNSLRAEDTAVYYCAKDLESGAYGFDVWGKGTTVTVSS

SEQ ID NO: 206 - protein, 119 aa, synthetic construct
EVQLVESGGGLVQPGRSLRLSCAASGFTFSAYGMHWVRQAPGKGLEWVSAIDAGGSTDYADSVEGRFTISRDNAKNSLYLQMNSLRAEDTAVYYCAKDLEAGYYGPDVWGKGTTVTVSS

SEQ ID NO: 207 - protein, 119 aa, synthetic construct
EVQLVESGGGLVQPGRSLRLSCAASGFTFSDAGMHWVRQAPGKGLEWVSAIDAGGSTDYADSVEGRFTISRDNAKNSLYLQMNSLRAEDTAVYYCAKDLEAGYYGPDVWGKGTTVTVSS

SEQ ID NO: 208 - protein, 119 aa, synthetic construct
EVQLVESGGGLVQPGRSLRLSCAASGFTFSDYAMHWVRQAPGKGLEWVSAIDAGGSTDYADSVEGRFTISRDNAKNSLYLQMNSLRAEDTAVYYCAKDLEAGYYGPDVWGKGTTVTVSS

SEQ ID NO: 209 - protein, 119 aa, synthetic construct
EVQLVESGGGLVQPGRSLRLSCAASGFTFSDYGMHWVRQAPGKGLEWVSAISAGGSTDYADSVEGRFTISRDNAKNSLYLQMNSLRAEDTAVYYCAKDLEAGYYGPDVWGKGTTVTVSS

SEQ ID NO: 210 - protein, 119 aa, synthetic construct
EVQLVESGGGLVQPGRSLRLSCAASGFTFSDYGMHWVRQAPGKGLEWVSAIDASGSTDYADSVEGRFTISRDNAKNSLYLQMNSLRAEDTAVYYCAKDLEAGYYGPDVWGKGTTVTVSS

SEQ ID NO: 211 - protein, 119 aa, synthetic construct
EVQLVESGGGLVQPGRSLRLSCAASGFTFSDYGMHWVRQAPGKGLEWVSAIDAGASTDYADSVEGRFTISRDNAKNSLYLQMNSLRAEDTAVYYCAKDLEAGYYGPDVWGKGTTVTVSS

SEQ ID NO: 212 - protein, 119 aa, synthetic construct
EVQLVESGGGLVQPGRSLRLSCAASGFTFSDYGMHWVRQAPGKGLEWVSAIDAGGSADYADSVEGRFTISRDNAKNSLYLQMNSLRAEDTAVYYCAKDLEAGYYGPDVWGKGTTVTVSS

SEQ ID NO: 213 - protein, 119 aa, synthetic construct
EVQLVESGGGLVQPGRSLRLSCAASGFTFSDYGMHWVRQAPGKGLEWVSAIDAGGSTAYADSVEGRFTISRDNAKNSLYLQMNSLRAEDTAVYYCAKDLEAGYYGPDVWGKGTTVTVSS

SEQ ID NO: 214 - protein, 119 aa, synthetic construct
EVQLVESGGGLVQPGRSLRLSCAASGFTFSDYGMHWVRQAPGKGLEWVSAIDAGGSTGYADSVEGRFTISRDNAKNSLYLQMNSLRAEDTAVYYCAKDLEAGYYGPDVWGKGTTVTVSS

SEQ ID NO: 215 - protein, 119 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKALEAGYYGPDVWGKGTTVTVSS

SEQ ID NO: 216 - protein, 119 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDAEAGYYGPDVWGKGTTVTVSS

SEQ ID NO: 217 - protein, 119 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDLEAAYYGPDVWGKGTTVTVSS

SEQ ID NO: 218 - protein, 119 aa, synthetic construct; identical to SEQ ID NO: 43

SEQ ID NO: 219 - protein, 119 aa, synthetic construct; identical to SEQ ID NO: 203

SEQ ID NO: 220 - protein, 119 aa, synthetic construct; identical to SEQ ID NO: 203

SEQ ID NO: 221 - protein, 119 aa, synthetic construct; identical to SEQ ID NO: 203

SEQ ID NO: 222 - protein, 119 aa, synthetic construct; identical to SEQ ID NO: 203

SEQ ID NO: 223 - protein, 119 aa, synthetic construct; identical to SEQ ID NO: 203

SEQ ID NO: 224 - protein, 119 aa, synthetic construct; identical to SEQ ID NO: 203

SEQ ID NO: 225 - protein, 119 aa, synthetic construct; identical to SEQ ID NO: 203

SEQ ID NO: 226 - protein, 119 aa, synthetic construct; identical to SEQ ID NO: 203

SEQ ID NO: 227 - protein, 119 aa, synthetic construct; identical to SEQ ID NO: 203

SEQ ID NO: 228 - protein, 119 aa, synthetic construct; identical to SEQ ID NO: 203

SEQ ID NO: 229 - protein, 124 aa, synthetic construct; identical to SEQ ID NO: 46

SEQ ID NO: 230 - protein, 123 aa, synthetic construct
QVQLQESGPGLVKPSQTLSLTCTVSGASIRSGDYSWSWIRQPPGKGLEWIGYIEHSGRTDYNPSLKSRVTMSVDTSKNQFSLKVNSVTAADTAVYYCARASGWYGVYDAFDVWGKGTTVTVSS

SEQ ID NO: 231 - protein, 123 aa, synthetic construct
QVQLQESGPGLVKPSQTLSLTCTVSGASITSGSHYWSWIRQPPGKGLEWIGYMYHSGSADYNPSLKSRVTMSVDTSKNQFSLKVNSVTAADTAVYYCARTLGVYGDYDAFDIWGQGTMVTVSS

SEQ ID NO: 232 - protein, 121 aa, synthetic construct
QVQLQQSGAELVRPGTSVKVSCKASGYDFNNDLIEWVKQRPGQGLEWIAVINPGSGRTNYNEKFKGKATLTADKSSSTVYMQLSSLTSDDSAVYFCAMIYYGPHSYAMDYWGQGTSVTVSS

SEQ ID NO: 233 - protein, 121 aa, synthetic construct
QVQLVQSGAEVKKPGASVKVSCKASGYDFNNDLIEWVRQAPGQGLEWMAVINPGSGRTNYAQKFQGRVTMTADKSTSTVYMELSSLRSEDTAVYYCAMIYYGPHSYAMDYWGQGTLVTVSS

SEQ ID NO: 234 - protein, 120 aa, synthetic construct
QVQLQQSGAELARPGTSVKVSCKASGYAFTNYLIEWVKQRPGQGLEWIGVISPGSGIINYNEKFKGKATLTADKSSSTAYMQLSSLTSDDSAVYFCAAIDYSGPYAVDDWGQGTSVTVSS

SEQ ID NO: 235 - protein, 120 aa, synthetic construct
QVQLVQSGAEVKKPGSSVKVSCKASGYAFTNYLIEWVRQAPGQGLEWMGVISPGSGIINYAQKFQGRATLTADKSTSTAYMELSSLRSEDTAVYYCAAIDYSGPYAVDDWGQGTLVTVSS

SEQ ID NO: 236 - protein, 5 aa, synthetic construct
DYGMH

SEQ ID NO: 237 - protein, 5 aa, synthetic construct
DYGMH

SEQ ID NO: 238 - protein, 5 aa, synthetic construct
DYGMH

SEQ ID NO: 239 - protein, 5 aa, synthetic construct
DYGMH

SEQ ID NO: 240 - protein, 5 aa, synthetic construct
DYGMH

SEQ ID NO: 241 - protein, 5 aa, synthetic construct
DYGMH

SEQ ID NO: 242 - protein, 5 aa, synthetic construct
DYGMH

SEQ ID NO: 243 - protein, 5 aa, synthetic construct
DYGMH

SEQ ID NO: 244 - protein, 5 aa, synthetic construct
DYGMH

SEQ ID NO: 245 - protein, 5 aa, synthetic construct
DYGMH

SEQ ID NO: 246 - protein, 5 aa, synthetic construct
DYGMH

SEQ ID NO: 247 - protein, 5 aa, synthetic construct
DYGMH

SEQ ID NO: 248 - protein, 5 aa, synthetic construct
DYGMH

SEQ ID NO: 249 - protein, 5 aa, synthetic construct
DYGMH

SEQ ID NO: 250 - protein, 5 aa, synthetic construct
DYGMH

SEQ ID NO: 251 - protein, 5 aa, synthetic construct
DYGMH

SEQ ID NO: 252 - protein, 5 aa, synthetic construct
DYGMH

SEQ ID NO: 253 - protein, 5 aa, synthetic construct
DYGMH

SEQ ID NO: 254 - protein, 5 aa, synthetic construct
DYGMH

SEQ ID NO: 255 - protein, 5 aa, synthetic construct
DYGMH

SEQ ID NO: 256 - protein, 5 aa, synthetic construct
DYGMH

SEQ ID NO: 257 - protein, 5 aa, synthetic construct
DYGMH

SEQ ID NO: 258 - protein, 5 aa, synthetic construct
DYGMH

SEQ ID NO: 259 - protein, 5 aa, synthetic construct
DYGMH

SEQ ID NO: 260 - protein, 5 aa, synthetic construct
DYGMH

SEQ ID NO: 261 - protein, 5 aa, synthetic construct
AYGMH

SEQ ID NO: 262 - protein, 5 aa, synthetic construct
DAGMH

SEQ ID NO: 263 - protein, 5 aa, synthetic construct
DYAMH

SEQ ID NO: 264 - protein, 5 aa, synthetic construct
DYGMH

SEQ ID NO: 265 - protein, 5 aa, synthetic construct
DYGMH

SEQ ID NO: 266 - protein, 5 aa, synthetic construct
DYGMH

SEQ ID NO: 267 - protein, 5 aa, synthetic construct
DYGMH

SEQ ID NO: 268 - protein, 5 aa, synthetic construct
DYGMH

SEQ ID NO: 269 - protein, 5 aa, synthetic construct
DYGMH

SEQ ID NO: 270 - protein, 5 aa, synthetic construct
DYGMH

SEQ ID NO: 271 - protein, 5 aa, synthetic construct
DYGMH

SEQ ID NO: 272 - protein, 5 aa, synthetic construct
DYGMH

SEQ ID NO: 273 - protein, 5 aa, synthetic construct
DYGMH

SEQ ID NO: 274 - protein, 5 aa, synthetic construct
DYGMH

SEQ ID NO: 275 - protein, 5 aa, synthetic construct
DYGMH

SEQ ID NO: 276 - protein, 5 aa, synthetic construct
DYGMH

SEQ ID NO: 277 - protein, 5 aa, synthetic construct
DYGMH

SEQ ID NO: 278 - protein, 5 aa, synthetic construct
DYGMH

SEQ ID NO: 279 - protein, 5 aa, synthetic construct
DYGMH

SEQ ID NO: 280 - protein, 5 aa, synthetic construct
DYGMH

SEQ ID NO: 281 - protein, 5 aa, synthetic construct
DYGMH

SEQ ID NO: 282 - protein, 5 aa, synthetic construct
DYGMH

SEQ ID NO: 283 - protein, 5 aa, synthetic construct
DYGMH

SEQ ID NO: 284 - protein, 5 aa, synthetic construct
NQGIS

SEQ ID NO: 285 - protein, 7 aa, synthetic construct
SGDYSWS

SEQ ID NO: 286 - protein, 7 aa, synthetic construct
SGSHYWS

SEQ ID NO: 287 - protein, 5 aa, synthetic construct
NDLIE

SEQ ID NO: 288 - protein, 5 aa, synthetic construct
NDLIE

SEQ ID NO: 289 - protein, 5 aa, synthetic construct
NYLIE

SEQ ID NO: 290 - protein, 5 aa, synthetic construct
NYLIE

SEQ ID NO: 291 - protein, 16 aa, synthetic construct
AIDRGGSTDYADSVEG

SEQ ID NO: 292 - protein, 16 aa, synthetic construct
AIDRGGSTDYADSVEG

SEQ ID NO: 293 - protein, 16 aa, synthetic construct
AIDAGGSTDYADSVEG

SEQ ID NO: 294 - protein, 16 aa, synthetic construct
AIDRGGATDYADSVEG

SEQ ID NO: 295 - protein, 16 aa, synthetic construct
AIDRGGSTYYADSVEG

SEQ ID NO: 296 - protein, 16 aa, synthetic construct
AIDRGGSTDYADSVKG

SEQ ID NO: 297 - protein, 16 aa, synthetic construct
AIDRGGSTDYADSVEG

SEQ ID NO: 298 - protein, 16 aa, synthetic construct
AIDRGGSTDYADSVEG

SEQ ID NO: 299 - protein, 16 aa, synthetic construct
AIDRGGSTDYADSVEG

SEQ ID NO: 300 - protein, 16 aa, synthetic construct
AIDRGGSTDYADSVEG

SEQ ID NO: 301 - protein, 16 aa, synthetic construct
AIDRGGSTDYADSVEG

SEQ ID NO: 302 - protein, 16 aa, synthetic construct
AIDRGGSTDYADSVEG

SEQ ID NO: 303 - protein, 16 aa, synthetic construct
AIDRGGSTDYADSVEG

SEQ ID NO: 304 - protein, 16 aa, synthetic construct
AIDRGGSTDYADSVEG

SEQ ID NO: 305 - protein, 16 aa, synthetic construct
AIDRGGSTDYADSVEG

SEQ ID NO: 306 - protein, 16 aa, synthetic construct
AIDRGGSTDYADSVEG

SEQ ID NO: 307 - protein, 16 aa, synthetic construct
AIDRGGSTDYADSVEG

SEQ ID NO: 308 - protein, 16 aa, synthetic construct
AIDRGGSTDYADSVEG

SEQ ID NO: 309 - protein, 16 aa, synthetic construct
AIDRGGSTDYADSVEG

SEQ ID NO: 310 - protein, 16 aa, synthetic construct
AIDRGGSTDYADSVEG

SEQ ID NO: 311 - protein, 16 aa, synthetic construct
AIDAGGSTDYADSVEG

SEQ ID NO: 312 - protein, 16 aa, synthetic construct
AIDRGGSTDYADSVEG

SEQ ID NO: 313 - protein, 16 aa, synthetic construct
AIDAGGSTDYADSVEG

SEQ ID NO: 314 - protein, 16 aa, synthetic construct
AIDRGGSTDYADSVEG

SEQ ID NO: 315 - protein, 16 aa, synthetic construct
AIDRGGSTDYADSVEG

SEQ ID NO: 316 - protein, 16 aa, synthetic construct
AIDAGGSTDYADSVEG

SEQ ID NO: 317 - protein, 16 aa, synthetic construct
AIDAGGSTDYADSVEG

SEQ ID NO: 318 - protein, 16 aa, synthetic construct
AIDAGGSTDYADSVEG

SEQ ID NO: 319 - protein, 16 aa, synthetic construct
AISAGGSTDYADSVEG

SEQ ID NO: 320 - protein, 16 aa, synthetic construct
AIDASGSTDYADSVEG

SEQ ID NO: 321 - protein, 16 aa, synthetic construct
AIDAGASTDYADSVEG

SEQ ID NO: 322 - protein, 16 aa, synthetic construct
AIDAGGSADYADSVEG

SEQ ID NO: 323 - protein, 16 aa, synthetic construct
AIDAGGSTAYADSVEG

SEQ ID NO: 324 - protein, 16 aa, synthetic construct
AIDAGGSTGYADSVEG

SEQ ID NO: 325 - protein, 16 aa, synthetic construct
AIDAGGSTDYADSVEG

SEQ ID NO: 326 - protein, 16 aa, synthetic construct
AIDAGGSTDYADSVEG

SEQ ID NO: 327 - protein, 16 aa, synthetic construct
AIDAGGSTDYADSVEG

SEQ ID NO: 328 - protein, 16 aa, synthetic construct
AIDAGGSTDYADSVEG

SEQ ID NO: 329 - protein, 16 aa, synthetic construct
AIDAGGSTDYADSVEG

SEQ ID NO: 330 - protein, 16 aa, synthetic construct
AIDAGGSTDYADSVEG

SEQ ID NO: 331 - protein, 16 aa, synthetic construct
AIDAGGSTDYADSVEG

SEQ ID NO: 332 - protein, 16 aa, synthetic construct
AIDAGGSTDYADSVEG

SEQ ID NO: 333 - protein, 16 aa, synthetic construct
AIDAGGSTDYADSVEG

SEQ ID NO: 334 - protein, 16 aa, synthetic construct
AIDAGGSTDYADSVEG

SEQ ID NO: 335 - protein, 16 aa, synthetic construct
AIDAGGSTDYADSVEG

SEQ ID NO: 336 - protein, 16 aa, synthetic construct
AIDAGGSTDYADSVEG

SEQ ID NO: 337 - protein, 16 aa, synthetic construct
AIDAGGSTDYADSVEG

SEQ ID NO: 338 - protein, 16 aa, synthetic construct
AIDAGGSTDYADSVEG

SEQ ID NO: 339 - protein, 17 aa, synthetic construct
GFDPDAGETIYAQKFQG

SEQ ID NO: 340 - protein, 16 aa, synthetic construct
YIEHSGRTDYNPSLKS

SEQ ID NO: 341 - protein, 16 aa, synthetic construct
YMYHSGSADYNPSLKS

SEQ ID NO: 342 - protein, 17 aa, synthetic construct
VINPGSGRTNYNEKFKG

SEQ ID NO: 343 - protein, 17 aa, synthetic construct
VINPGSGRTNYAQKFQG

SEQ ID NO: 344 - protein, 17 aa, synthetic construct
VISPGSGIINYNEKFKG

SEQ ID NO: 345 - protein, 17 aa, synthetic construct
VISPGSGIINYAQKFQG

SEQ ID NO: 346 - protein, 11 aa, synthetic construct
DLESGYYGPDV

SEQ ID NO: 347 - protein, 11 aa, synthetic construct
DLESGYYGPDV

SEQ ID NO: 348 - protein, 11 aa, synthetic construct
DLESGYYGPDV

SEQ ID NO: 349 - protein, 11 aa, synthetic construct
DLESGYYGPDV

SEQ ID NO: 350 - protein, 11 aa, synthetic construct
DLESGYYGPDV

SEQ ID NO: 351 - protein, 11 aa, synthetic construct
DLESGYYGPDV

SEQ ID NO: 352 - protein, 11 aa, synthetic construct
DLASGYYGPDV

SEQ ID NO: 353 - protein, 11 aa, synthetic construct
DLEAGYYGPDV

SEQ ID NO: 354 - protein, 11 aa, synthetic construct
DLESGAYGPDV

SEQ ID NO: 355 - protein, 11 aa, synthetic construct
DLESGYAGPDV

SEQ ID NO: 356 - protein, 11 aa, synthetic construct
DLESGYYGFDV

SEQ ID NO: 357 - protein, 11 aa, synthetic construct
DLESGYYGPDV

SEQ ID NO: 358 - protein, 11 aa, synthetic construct
DLESGYYGPDV

SEQ ID NO: 359 - protein, 11 aa, synthetic construct
DLESGYYGPDV

SEQ ID NO: 360 - protein, 11 aa, synthetic construct
DLESGYYGPDV

SEQ ID NO: 361 - protein, 11 aa, synthetic construct
DLESGYYGPDV

SEQ ID NO: 362 - protein, 11 aa, synthetic construct
DLESGYYGPDV

SEQ ID NO: 363 - protein, 11 aa, synthetic construct
DLESGYYGPDV

SEQ ID NO: 364 - protein, 11 aa, synthetic construct
DLESGYYGPDV

SEQ ID NO: 365 - protein, 11 aa, synthetic construct
DLESGYYGPDV

SEQ ID NO: 366 - protein, 11 aa, synthetic construct
DLESGYYGPDV

SEQ ID NO: 367 - protein, 11 aa, synthetic construct
DLEAGYYGPDV

SEQ ID NO: 368 - protein, 11 aa, synthetic construct
DLEAGYYGPDV

SEQ ID NO: 369 - protein, 11 aa, synthetic construct
DLESGAYGPDV

SEQ ID NO: 370 - protein, 11 aa, synthetic construct
DLESGAYGFDV

SEQ ID NO: 371 - protein, 11 aa, synthetic construct
DLEAGYYGPDV

SEQ ID NO: 372 - protein, 11 aa, synthetic construct
DLEAGYYGPDV

SEQ ID NO: 373 - protein, 11 aa, synthetic construct
DLEAGYYGPDV

SEQ ID NO: 374 - protein, 11 aa, synthetic construct
DLEAGYYGPDV

SEQ ID NO: 375 - protein, 11 aa, synthetic construct
DLEAGYYGPDV

SEQ ID NO: 376 - protein, 11 aa, synthetic construct
DLEAGYYGPDV

SEQ ID NO: 377 - protein, 11 aa, synthetic construct
DLEAGYYGPDV

SEQ ID NO: 378 - protein, 11 aa, synthetic construct
DLEAGYYGPDV

SEQ ID NO: 379 - protein, 11 aa, synthetic construct
DLEAGYYGPDV

SEQ ID NO: 380 - protein, 11 aa, synthetic construct
ALEAGYYGPDV

SEQ ID NO: 381 - protein, 11 aa, synthetic construct
DAEAGYYGPDV

SEQ ID NO: 382 - protein, 11 aa, synthetic construct
DLEAAYYGPDV

SEQ ID NO: 383 - protein, 11 aa, synthetic construct
DLEAGYYAPDV

SEQ ID NO: 384 - protein, 11 aa, synthetic construct
DLEAGYYGPDV

SEQ ID NO: 385 - protein, 11 aa, synthetic construct
DLEAGYYGPDV

SEQ ID NO: 386 - protein, 11 aa, synthetic construct
DLEAGYYGPDV

SEQ ID NO: 387 - protein, 11 aa, synthetic construct
DLEAGYYGPDV

SEQ ID NO: 388 - protein, 11 aa, synthetic construct
DLEAGYYGPDV

SEQ ID NO: 389 - protein, 11 aa, synthetic construct
DLEAGYYGPDV

SEQ ID NO: 390 - protein, 11 aa, synthetic construct
DLEAGYYGPDV

SEQ ID NO: 391 - protein, 11 aa, synthetic construct
DLEAGYYGPDV

SEQ ID NO: 392 - protein, 11 aa, synthetic construct
DLEAGYYGPDV

SEQ ID NO: 393 - protein, 11 aa, synthetic construct
DLEAGYYGPDV

SEQ ID NO: 394 - protein, 15 aa, synthetic construct
GVDSYGYGRYNWFDP

SEQ ID NO: 395 - protein, 13 aa, synthetic construct
ASGWYGVYDAFDV

SEQ ID NO: 396 - protein, 13 aa, synthetic construct
TLGVYGDYDAFDI

SEQ ID NO: 397 - protein, 12 aa, synthetic construct
IYYGPHSYAMDY

SEQ ID NO: 398 - protein, 12 aa, synthetic construct
IYYGPHSYAMDY

SEQ ID NO: 399 - protein, 11 aa, synthetic construct
IDYSGPYAVDD

SEQ ID NO: 400 - protein, 11 aa, synthetic construct
IDYSGPYAVDD

SEQ ID NO: 401 - protein, 214 aa, synthetic construct; identical to SEQ ID NO: 50

SEQ ID NO: 402 - protein, 214 aa, synthetic construct; identical to SEQ ID NO: 50

SEQ ID NO: 403 - protein, 214 aa, synthetic construct; identical to SEQ ID NO: 50

SEQ ID NO: 404 - protein, 214 aa, synthetic construct; identical to SEQ ID NO: 50

SEQ ID NO: 405 - protein, 214 aa, synthetic construct; identical to SEQ ID NO: 50

SEQ ID NO: 406 - protein, 214 aa, synthetic construct; identical to SEQ ID NO: 50

SEQ ID NO: 407 - protein, 214 aa, synthetic construct; identical to SEQ ID NO: 50

SEQ ID NO: 408 - protein, 214 aa, synthetic construct; identical to SEQ ID NO: 50

SEQ ID NO: 409 - protein, 214 aa, synthetic construct; identical to SEQ ID NO: 50

SEQ ID NO: 410 - protein, 214 aa, synthetic construct; identical to SEQ ID NO: 50

SEQ ID NO: 411 - protein, 214 aa, synthetic construct; identical to SEQ ID NO: 50

SEQ ID NO: 412 - protein, 214 aa, synthetic construct
DIQMTQSPSSLSASVGDRVTITCRASQDIGNFLAWYQQKPGKAPKLLIYAVSNLQSGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 413 - protein, 214 aa, synthetic construct
DIQMTQSPSSLSASVGDRVTITCRASQDIGRALAWYQQKPGKAPKLLIYAVSNLQSGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 414 - protein, 214 aa, synthetic construct
DIQMTQSPSSLSASVGDRVTITCRASQDIGRFLAWYQQKPGKAPKLLIYAASNLQSGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 415 - protein, 214 aa, synthetic construct
DIQMTQSPSSLSASVGDRVTITCRASQDIGRFLAWYQQKPGKAPKLLIYAVSTLQSGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 416 - protein, 214 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQASTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 417 - protein, 214 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQYATSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 418 - protein, 214 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQYSASVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 419 - protein, 214 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQYSTAVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 420 - protein, 214 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQYSTSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 421 - protein, 214 aa, synthetic construct; identical to SEQ ID NO: 50

SEQ ID NO: 422 - protein, 214 aa, synthetic construct; identical to SEQ ID NO: 50

SEQ ID NO: 423 - protein, 214 aa, synthetic construct; identical to SEQ ID NO: 50

SEQ ID NO: 424 - protein, 214 aa, synthetic construct; identical to SEQ ID NO: 413

SEQ ID NO: 425 - protein, 214 aa, synthetic construct; identical to SEQ ID NO: 413

SEQ ID NO: 426 - protein, 214 aa, synthetic construct; identical to SEQ ID NO: 50

SEQ ID NO: 427 - protein, 214 aa, synthetic construct; identical to SEQ ID NO: 50

SEQ ID NO: 428 - protein, 214 aa, synthetic construct; identical to SEQ ID NO: 50

SEQ ID NO: 429 - protein, 214 aa, synthetic construct; identical to SEQ ID NO: 50

SEQ ID NO: 430 - protein, 214 aa, synthetic construct; identical to SEQ ID NO: 50

SEQ ID NO: 431 - protein, 214 aa, synthetic construct; identical to SEQ ID NO: 50

SEQ ID NO: 432 - protein, 214 aa, synthetic construct; identical to SEQ ID NO: 50

SEQ ID NO: 433 - protein, 214 aa, synthetic construct; identical to SEQ ID NO: 50

SEQ ID NO: 434 - protein, 214 aa, synthetic construct; identical to SEQ ID NO: 50

SEQ ID NO: 435 - protein, 214 aa, synthetic construct; identical to SEQ ID NO: 50

SEQ ID NO: 436 - protein, 214 aa, synthetic construct; identical to SEQ ID NO: 50

SEQ ID NO: 437 - protein, 214 aa, synthetic construct; identical to SEQ ID NO: 50

SEQ ID NO: 438 - protein, 214 aa, synthetic construct; identical to SEQ ID NO: 50

SEQ ID NO: 439 - protein, 214 aa, synthetic construct
DIQMTQSPSSLSASVGDRVTITCRASQAIGRFLAWYQQKPGKAPKLLIYAVSNLQSGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 440 - protein, 214 aa, synthetic construct
DIQMTQSPSSLSASVGDRVTITCRASQDISRFLAWYQQKPGKAPKLLIYAVSNLQSGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 441 - protein, 214 aa, synthetic construct
DIQMTQSPSSLSASVGDRVTITCRASQDIGSFLAWYQQKPGKAPKLLIYAVSNLQSGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 442 - protein, 214 aa, synthetic construct
DIQMTQSPSSLSASVGDRVTITCRASQDIGRYLAWYQQKPGKAPKLLIYAVSNLQSGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 443 - protein, 214 aa, synthetic construct
DIQMTQSPSSLSASVGDRVTITCRASQDIGRFLAWYQQKPGKAPKLLIYAVANLQSGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 444 - protein, 214 aa, synthetic construct
DIQMTQSPSSLSASVGDRVTITCRASQDIGRFLAWYQQKPGKAPKLLIYAVSALQSGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 445 - protein, 214 aa, synthetic construct
DIQMTQSPSSLSASVGDRVTITCRASQDIGRFLAWYQQKPGKAPKLLIYAVSNAQSGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 446 - protein, 214 aa, synthetic construct
DIQMTQSPSSLSASVGDRVTITCRASQDIGRFLAWYQQKPGKAPKLLIYAVSNLASGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 447 - protein, 214 aa, synthetic construct
DIQMTQSPSSLSASVGDRVTITCRASQDIGRFLAWYQQKPGKAPKLLIYAVSNLQAGVPSRFSGSGSGTDFTLTISSLQPEDVATYYCQQYSTSVYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 448 - protein, 214 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQYSTSAYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 449 - protein, 214 aa, synthetic construct; identical to SEQ ID NO: 53

SEQ ID NO: 450 - protein, 214 aa, synthetic construct
EIVMTQSPATLSLSPGERATLSCRASQSVSHHLAWYQQKPGQAPRLLIYDTFNRAPGIPARFSGSGSGTDFTLTISSLEPEDFAVYYCQHYDTAPFSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 451 - protein, 214 aa, synthetic construct
EIVMTQSPATLSLSPGERATLSCRASQSVSHNLAWYQQKPGQAPRLLIYDTFNRAPGIPARFSGSGSGTDFTLTISSLEPEDFAVYYCQQYDTRPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 452 - protein, 214 aa, synthetic construct
DIVMTQSHKFMSTVVGDRVSITCKASLDVRTAVAWYQQKPGQSPKLLIYSASYRYTGVPDRFTGSGSGTDFTFNIRSVQAEDLAVYYCQQHYGIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 453 - protein, 214 aa, synthetic construct
DIQMTQSPSSLSASVGDRVTITCKASLDVRTAVAWYQQKPGKAPKLLIYSASYRYTGVPSRFSGSGSGTDFTFTISSLQPEDIATYYCQQHYGIPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 454 - protein, 214 aa, synthetic construct
DIVMTQSHKFMSTSVGDRVSVTCKASQAVNTAVAWYQQKPGQSPKLLIYSASYGYTGVPDRFTGSGSGTDFTLTISSVQAEDLAVYYCQHHYGVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 455 - protein, 214 aa, synthetic construct
DIQMTQSPSSLSASVGDRVTITCKASQAVNTAVAWYQQKPGKAPKLLIYSASYGYTGVPSRFSGSGSGTDFTLTISSLQPEDFATYYCQHHYGVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 456 - protein, 107 aa, synthetic construct; identical to SEQ ID NO: 49

SEQ ID NO: 457 - protein, 107 aa, synthetic construct; identical to SEQ ID NO: 49

SEQ ID NO: 458 - protein, 107 aa, synthetic construct; identical to SEQ ID NO: 49

SEQ ID NO: 459 - protein, 107 aa, synthetic construct; identical to SEQ ID NO: 49

SEQ ID NO: 460 - protein, 107 aa, synthetic construct; identical to SEQ ID NO: 49

SEQ ID NO: 461 - protein, 107 aa, synthetic construct; identical to SEQ ID NO: 49

SEQ ID NO: 462 - protein, 107 aa, synthetic construct; identical to SEQ ID NO: 49

SEQ ID NO: 463 - protein, 107 aa, synthetic construct; identical to SEQ ID NO: 49

SEQ ID NO: 464 - protein, 107 aa, synthetic construct; identical to SEQ ID NO: 49

SEQ ID NO: 465 - protein, 107 aa, synthetic construct; identical to SEQ ID NO: 49

SEQ ID NO: 466 - protein, 107 aa, synthetic construct; identical to SEQ ID NO: 49

SEQ ID NO: 467 - protein, 107 aa, synthetic construct
DIQMTQSPSSLSASVGDRVTITCRASQDIGNFLAWYQQKPGKAPKLLIYAVSNLQSGVPSRFSGSGSGTDFTLTISSLQPEDVATYYCQQYSTSVYTFGGGTKVEIK

SEQ ID NO: 468 - protein, 107 aa, synthetic construct
DIQMTQSPSSLSASVGDRVTITCRASQDIGRALAWYQQKPGKAPKLLIYAVSNLQSGVPSRFSGSGSGTDFTLTISSLQPEDVATYYCQQYSTSVYTFGGGTKVEIK

SEQ ID NO: 469 - protein, 107 aa, synthetic construct
DIQMTQSPSSLSASVGDRVTITCRASQDIGRFLAWYQQKPGKAPKLLIYAASNLQSGVPSRFSGSGSGTDFTLTISSLQPEDVATYYCQQYSTSVYTFGGGTKVEIK

SEQ ID NO: 470 - protein, 107 aa, synthetic construct
DIQMTQSPSSLSASVGDRVTITCRASQDIGRFLAWYQQKPGKAPKLLIYAVSTLQSGVPSRFSGSGSGTDFTLTISSLQPEDVATYYCQQYSTSVYTFGGGTKVEIK

SEQ ID NO: 471 - protein, 107 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQASTSVYTFGGGTKVEIK

SEQ ID NO: 472 - protein, 107 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQYATSVYTFGGGTKVEIK

SEQ ID NO: 473 - protein, 107 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQYSASVYTFGGGTKVEIK

SEQ ID NO: 474 - protein, 107 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQYSTAVYTFGGGTKVEIK

SEQ ID NO: 475 - protein, 107 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQYSTSPYTFGGGTKVEIK

SEQ ID NO: 476 - protein, 107 aa, synthetic construct; identical to SEQ ID NO: 49

SEQ ID NO: 477 - protein, 107 aa, synthetic construct; identical to SEQ ID NO: 49

SEQ ID NO: 478 - protein, 107 aa, synthetic construct; identical to SEQ ID NO: 49

SEQ ID NO: 479 - protein, 107 aa, synthetic construct; identical to SEQ ID NO: 468

SEQ ID NO: 480 - protein, 107 aa, synthetic construct; identical to SEQ ID NO: 468

SEQ ID NO: 481 - protein, 107 aa, synthetic construct; identical to SEQ ID NO: 49

SEQ ID NO: 482 - protein, 107 aa, synthetic construct; identical to SEQ ID NO: 49

SEQ ID NO: 483 - protein, 107 aa, synthetic construct; identical to SEQ ID NO: 49

SEQ ID NO: 484 - protein, 107 aa, synthetic construct; identical to SEQ ID NO: 49

SEQ ID NO: 485 - protein, 107 aa, synthetic construct; identical to SEQ ID NO: 49

SEQ ID NO: 486 - protein, 107 aa, synthetic construct; identical to SEQ ID NO: 49

SEQ ID NO: 487 - protein, 107 aa, synthetic construct; identical to SEQ ID NO: 49

SEQ ID NO: 488 - protein, 107 aa, synthetic construct; identical to SEQ ID NO: 49

SEQ ID NO: 489 - protein, 107 aa, synthetic construct; identical to SEQ ID NO: 49

SEQ ID NO: 490 - protein, 107 aa, synthetic construct; identical to SEQ ID NO: 49

SEQ ID NO: 491 - protein, 107 aa, synthetic construct; identical to SEQ ID NO: 49

SEQ ID NO: 492 - protein, 107 aa, synthetic construct; identical to SEQ ID NO: 49

SEQ ID NO: 493 - protein, 107 aa, synthetic construct; identical to SEQ ID NO: 49

SEQ ID NO: 494 - protein, 107 aa, synthetic construct
DIQMTQSPSSLSASVGDRVTITCRASQAIGRFLAWYQQKPGKAPKLLIYAVSNLQSGVPSRFSGSGSGTDFTLTISSLQPEDVATYYCQQYSTSVYTFGGGTKVEIK

SEQ ID NO: 495 - protein, 107 aa, synthetic construct
DIQMTQSPSSLSASVGDRVTITCRASQDISRFLAWYQQKPGKAPKLLIYAVSNLQSGVPSRFSGSGSGTDFTLTISSLQPEDVATYYCQQYSTSVYTFGGGTKVEIK

SEQ ID NO: 496 - protein, 107 aa, synthetic construct
DIQMTQSPSSLSASVGDRVTITCRASQDIGSFLAWYQQKPGKAPKLLIYAVSNLQSGVPSRFSGSGSGTDFTLTISSLQPEDVATYYCQQYSTSVYTFGGGTKVEIK

SEQ ID NO: 497 - protein, 107 aa, synthetic construct
DIQMTQSPSSLSASVGDRVTITCRASQDIGRYLAWYQQKPGKAPKLLIYAVSNLQSGVPSRFSGSGSGTDFTLTISSLQPEDVATYYCQQYSTSVYTFGGGTKVEIK

SEQ ID NO: 498 - protein, 107 aa, synthetic construct
DIQMTQSPSSLSASVGDRVTITCRASQDIGRFLAWYQQKPGKAPKLLIYAVANLQSGVPSRFSGSGSGTDFTLTISSLQPEDVATYYCQQYSTSVYTFGGGTKVEIK

SEQ ID NO: 499 - protein, 107 aa, synthetic construct
DIQMTQSPSSLSASVGDRVTITCRASQDIGRFLAWYQQKPGKAPKLLIYAVSALQSGVPSRFSGSGSGTDFTLTISSLQPEDVATYYCQQYSTSVYTFGGGTKVEIK

SEQ ID NO: 500 - protein, 107 aa, synthetic construct
DIQMTQSPSSLSASVGDRVTITCRASQDIGRFLAWYQQKPGKAPKLLIYAVSNAQSGVPSRFSGSGSGTDFTLTISSLQPEDVATYYCQQYSTSVYTFGGGTKVEIK

SEQ ID NO: 501 - protein, 107 aa, synthetic construct
DIQMTQSPSSLSASVGDRVTITCRASQDIGRFLAWYQQKPGKAPKLLIYAVSNLASGVPSRFSGSGSGTDFTLTISSLQPEDVATYYCQQYSTSVYTFGGGTKVEIK

SEQ ID NO: 502 - protein, 107 aa, synthetic construct
DIQMTQSPSSLSASVGDRVTITCRASQDIGRFLAWYQQKPGKAPKLLIYAVSNLQAGVPSRFSGSGSGTDFTLTISSLQPEDVATYYCQQYSTSVYTFGGGTKVEIK

SEQ ID NO: 503 - protein, 107 aa, synthetic construct
DIQMTQSPSSLSASVGDRVTITCRASQDIGRFLAWYQQKPGKAPKLLIYAVSNLQSGVPSRFSGSGSGTDFTLTISSLQPEDVATYYCQQYSTSAYTFGGGTKVEIK

SEQ ID NO: 504 - protein, 107 aa, synthetic construct; identical to SEQ ID NO: 52

SEQ ID NO: 505 - protein, 107 aa, synthetic construct
EIVMTQSPATLSLSPGERATLSCRASQSVSHHLAWYQQKPGQAPRLLIYDTFNRAPGIPARFSGSGSGTDFTLTISSLEPEDFAVYYCQHYDTAPFSFGGGTKVEIK

SEQ ID NO: 506 - protein, 107 aa, synthetic construct
EIVMTQSPATLSLSPGERATLSCRASQSVSHNLAWYQQKPGQAPRLLIYDTFNRAPGIPARFSGSGSGTDFTLTISSLEPEDFAVYYCQQYDTRPLTFGGGTKVEIK

SEQ ID NO: 507 - protein, 107 aa, synthetic construct
DIVMTQSHKFMSTVVGDRVSITCKASLDVRTAVAWYQQKPGQSPKLLIYSASYRYTGVPDRFTGSGSGTDFTFNIRSVQAEDLAVYYCQQHYGIPWTFGGGTKLEIK

SEQ ID NO: 508 - protein, 107 aa, synthetic construct
DIQMTQSPSSLSASVGDRVTITCKASLDVRTAVAWYQQKPGKAPKLLIYSASYRYTGVPSRFSGSGSGTDFTFTISSLQPEDIATYYCQQHYGIPWTFGQGTKVEIK

SEQ ID NO: 509 - protein, 107 aa, synthetic construct
DIVMTQSHKFMSTSVGDRVSVTCKASQAVNTAVAWYQQKPGQSPKLLIYSASYGYTGVPDRFTGSGSGTDFTLTISSVQAEDLAVYYCQHHYGVPWTFGGGTKLEIK

SEQ ID NO: 510 - protein, 107 aa, synthetic construct
DIQMTQSPSSLSASVGDRVTITCKASQAVNTAVAWYQQKPGKAPKLLIYSASYGYTGVPSRFSGSGSGTDFTLTISSLQPEDFATYYCQHHYGVPWTFGGGTKLEIK

SEQ ID NO: 511 - protein, 11 aa, synthetic construct
RASQDIGRFLA

SEQ ID NO: 512 - protein, 11 aa, synthetic construct
RASQDIGRFLA

SEQ ID NO: 513 - protein, 11 aa, synthetic construct
RASQDIGRFLA

SEQ ID NO: 514 - protein, 11 aa, synthetic construct
RASQDIGRFLA

SEQ ID NO: 515 - protein, 11 aa, synthetic construct
RASQDIGRFLA

SEQ ID NO: 516 - protein, 11 aa, synthetic construct
RASQDIGRFLA

SEQ ID NO: 517 - protein, 11 aa, synthetic construct
RASQDIGRFLA

SEQ ID NO: 518 - protein, 11 aa, synthetic construct
RASQDIGRFLA

SEQ ID NO: 519 - protein, 11 aa, synthetic construct
RASQDIGRFLA

SEQ ID NO: 520 - protein, 11 aa, synthetic construct
RASQDIGRFLA

SEQ ID NO: 521 - protein, 11 aa, synthetic construct
RASQDIGRFLA

SEQ ID NO: 522 - protein, 11 aa, synthetic construct
RASQDIGNFLA

SEQ ID NO: 523 - protein, 11 aa, synthetic construct
RASQDIGRALA

SEQ ID NO: 524 - protein, 11 aa, synthetic construct
RASQDIGRFLA

SEQ ID NO: 525 - protein, 11 aa, synthetic construct
RASQDIGRFLA

SEQ ID NO: 526 - protein, 11 aa, synthetic construct
RASQDIGRFLA

SEQ ID NO: 527 - protein, 11 aa, synthetic construct
RASQDIGRFLA

SEQ ID NO: 528 - protein, 11 aa, synthetic construct
RASQDIGRFLA

SEQ ID NO: 529 - protein, 11 aa, synthetic construct
RASQDIGRFLA

SEQ ID NO: 530 - protein, 11 aa, synthetic construct
RASQDIGRFLA

SEQ ID NO: 531 - protein, 11 aa, synthetic construct
RASQDIGRFLA

SEQ ID NO: 532 - protein, 11 aa, synthetic construct
RASQDIGRFLA

SEQ ID NO: 533 - protein, 11 aa, synthetic construct
RASQDIGRFLA

SEQ ID NO: 534 - protein, 11 aa, synthetic construct
RASQDIGRALA

SEQ ID NO: 535 - protein, 11 aa, synthetic construct
RASQDIGRALA

SEQ ID NO: 536 - protein, 11 aa, synthetic construct
RASQDIGRFLA

SEQ ID NO: 537 - protein, 11 aa, synthetic construct
RASQDIGRFLA

SEQ ID NO: 538 - protein, 11 aa, synthetic construct
RASQDIGRFLA

SEQ ID NO: 539 - protein, 11 aa, synthetic construct
RASQDIGRFLA

SEQ ID NO: 540 - protein, 11 aa, synthetic construct
RASQDIGRFLA

SEQ ID NO: 541 - protein, 11 aa, synthetic construct
RASQDIGRFLA

SEQ ID NO: 542 - protein, 11 aa, synthetic construct
RASQDIGRFLA

SEQ ID NO: 543 - protein, 11 aa, synthetic construct
RASQDIGRFLA

SEQ ID NO: 544 - protein, 11 aa, synthetic construct
RASQDIGRFLA

SEQ ID NO: 545 - protein, 11 aa, synthetic construct
RASQDIGRFLA

SEQ ID NO: 546 - protein, 11 aa, synthetic construct
RASQDIGRFLA

SEQ ID NO: 547 - protein, 11 aa, synthetic construct
RASQDIGRFLA

SEQ ID NO: 548 - protein, 11 aa, synthetic construct
RASQDIGRFLA

SEQ ID NO: 549 - protein, 11 aa, synthetic construct
RASQAIGRFLA

SEQ ID NO: 550 - protein, 11 aa, synthetic construct
RASQDISRFLA

SEQ ID NO: 551 - protein, 11 aa, synthetic construct
RASQDIGSFLA

SEQ ID NO: 552 - protein, 11 aa, synthetic construct
RASQDIGRYLA

SEQ ID NO: 553 - protein, 11 aa, synthetic construct
RASQDIGRFLA

SEQ ID NO: 554 - protein, 11 aa, synthetic construct
RASQDIGRFLA

SEQ ID NO: 555 - protein, 11 aa, synthetic construct
RASQDIGRFLA

SEQ ID NO: 556 - protein, 11 aa, synthetic construct
RASQDIGRFLA

SEQ ID NO: 557 - protein, 11 aa, synthetic construct
RASQDIGRFLA

SEQ ID NO: 558 - protein, 11 aa, synthetic construct
RASQDIGRFLA

SEQ ID NO: 559 - protein, 11 aa, synthetic construct
RASQDIRHYLA

SEQ ID NO: 560 - protein, 11 aa, synthetic construct
RASQSVSHHLA

SEQ ID NO: 561 - protein, 11 aa, synthetic construct
RASQSVSHNLA

SEQ ID NO: 562 - protein, 11 aa, synthetic construct
KASLDVRTAVA

SEQ ID NO: 563 - protein, 11 aa, synthetic construct
KASLDVRTAVA

SEQ ID NO: 564 - protein, 11 aa, synthetic construct
KASQAVNTAVA

SEQ ID NO: 565 - protein, 11 aa, synthetic construct
KASQAVNTAVA

SEQ ID NO: 566 - protein, 7 aa, synthetic construct
AVSNLQS

SEQ ID NO: 567 - protein, 7 aa, synthetic construct
AVSNLQS

SEQ ID NO: 568 - protein, 7 aa, synthetic construct
AVSNLQS

SEQ ID NO: 569 - protein, 7 aa, synthetic construct
AVSNLQS

SEQ ID NO: 570 - protein, 7 aa, synthetic construct
AVSNLQS

SEQ ID NO: 571 - protein, 7 aa, synthetic construct
AVSNLQS

SEQ ID NO: 572 - protein, 7 aa, synthetic construct
AVSNLQS

SEQ ID NO: 573 - protein, 7 aa, synthetic construct
AVSNLQS

SEQ ID NO: 574 - protein, 7 aa, synthetic construct
AVSNLQS

SEQ ID NO: 575 - protein, 7 aa, synthetic construct
AVSNLQS

SEQ ID NO: 576 - protein, 7 aa, synthetic construct
AVSNLQS

SEQ ID NO: 577 - protein, 7 aa, synthetic construct
AVSNLQS

SEQ ID NO: 578 - protein, 7 aa, synthetic construct
AVSNLQS

SEQ ID NO: 579 - protein, 7 aa, synthetic construct
AASNLQS

SEQ ID NO: 580 - protein, 7 aa, synthetic construct
AVSTLQS

SEQ ID NO: 581 - protein, 7 aa, synthetic construct
AVSNLQS

SEQ ID NO: 582 - protein, 7 aa, synthetic construct
AVSNLQS

SEQ ID NO: 583 - protein, 7 aa, synthetic construct
AVSNLQS

SEQ ID NO: 584 - protein, 7 aa, synthetic construct
AVSNLQS

SEQ ID NO: 585 - protein, 7 aa, synthetic construct
AVSNLQS

SEQ ID NO: 586 - protein, 7 aa, synthetic construct
AVSNLQS

SEQ ID NO: 587 - protein, 7 aa, synthetic construct
AVSNLQS

SEQ ID NO: 588 - protein, 7 aa, synthetic construct
AVSNLQS

SEQ ID NO: 589 - protein, 7 aa, synthetic construct
AVSNLQS

SEQ ID NO: 590 - protein, 7 aa, synthetic construct
AVSNLQS

SEQ ID NO: 591 - protein, 7 aa, synthetic construct
AVSNLQS

SEQ ID NO: 592 - protein, 7 aa, synthetic construct
AVSNLQS

SEQ ID NO: 593 - protein, 7 aa, synthetic construct
AVSNLQS

SEQ ID NO: 594 - protein, 7 aa, synthetic construct
AVSNLQS

SEQ ID NO: 595 - protein, 7 aa, synthetic construct
AVSNLQS

SEQ ID NO: 596 - protein, 7 aa, synthetic construct
AVSNLQS

SEQ ID NO: 597 - protein, 7 aa, synthetic construct
AVSNLQS

SEQ ID NO: 598 - protein, 7 aa, synthetic construct
AVSNLQS

SEQ ID NO: 599 - protein, 7 aa, synthetic construct
AVSNLQS

SEQ ID NO: 600 - protein, 7 aa, synthetic construct
AVSNLQS

SEQ ID NO: 601 - protein, 7 aa, synthetic construct
AVSNLQS

SEQ ID NO: 602 - protein, 7 aa, synthetic construct
AVSNLQS

SEQ ID NO: 603 - protein, 7 aa, synthetic construct
AVSNLQS

SEQ ID NO: 604 - protein, 7 aa, synthetic construct
AVSNLQS

SEQ ID NO: 605 - protein, 7 aa, synthetic construct
AVSNLQS

SEQ ID NO: 606 - protein, 7 aa, synthetic construct
AVSNLQS

SEQ ID NO: 607 - protein, 7 aa, synthetic construct
AVSNLQS

SEQ ID NO: 608 - protein, 7 aa, synthetic construct
AVANLQS

SEQ ID NO: 609 - protein, 7 aa, synthetic construct
AVSALQS

SEQ ID NO: 610 - protein, 7 aa, synthetic construct
AVSNAQS

SEQ ID NO: 611 - protein, 7 aa, synthetic construct
AVSNLAS

SEQ ID NO: 612 - protein, 7 aa, synthetic construct
AVSNLQA

SEQ ID NO: 613 - protein, 7 aa, synthetic construct
AVSNLQS

SEQ ID NO: 614 - protein, 7 aa, synthetic construct
DTFNRAT

SEQ ID NO: 615 - protein, 7 aa, synthetic construct
DTFNRAP

SEQ ID NO: 616 - protein, 7 aa, synthetic construct
DTFNRAP

SEQ ID NO: 617 - protein, 7 aa, synthetic construct
SASYRYT

SEQ ID NO: 618 - protein, 7 aa, synthetic construct
SASYRYT

SEQ ID NO: 619 - protein, 7 aa, synthetic construct
SASYGYT

SEQ ID NO: 620 - protein, 7 aa, synthetic construct
SASYGYT

SEQ ID NO: 621 - protein, 9 aa, synthetic construct
QQYSTSVYT

SEQ ID NO: 622 - protein, 9 aa, synthetic construct
QQYSTSVYT

SEQ ID NO: 623 - protein, 9 aa, synthetic construct
QQYSTSVYT

SEQ ID NO: 624 - protein, 9 aa, synthetic construct
QQYSTSVYT

SEQ ID NO: 625 - protein, 9 aa, synthetic construct
QQYSTSVYT

SEQ ID NO: 626 - protein, 9 aa, synthetic construct
QQYSTSVYT

SEQ ID NO: 627 - protein, 9 aa, synthetic construct
QQYSTSVYT

SEQ ID NO: 628 - protein, 9 aa, synthetic construct
QQYSTSVYT

SEQ ID NO: 629 - protein, 9 aa, synthetic construct
QQYSTSVYT

SEQ ID NO: 630 - protein, 9 aa, synthetic construct
QQYSTSVYT

SEQ ID NO: 631 - protein, 9 aa, synthetic construct
QQYSTSVYT

SEQ ID NO: 632 - protein, 9 aa, synthetic construct
QQYSTSVYT

SEQ ID NO: 633 - protein, 9 aa, synthetic construct
QQYSTSVYT

SEQ ID NO: 634 - protein, 9 aa, synthetic construct
QQYSTSVYT

SEQ ID NO: 635 - protein, 9 aa, synthetic construct
QQYSTSVYT

SEQ ID NO: 636 - protein, 9 aa, synthetic construct
QQASTSVYT

SEQ ID NO: 637 - protein, 9 aa, synthetic construct
QQYATSVYT

SEQ ID NO: 638 - protein, 9 aa, synthetic construct
QQYSASVYT

SEQ ID NO: 639 - protein, 9 aa, synthetic construct
QQYSTAVYT

SEQ ID NO: 640 - protein, 9 aa, synthetic construct
QQYSTSPYT

SEQ ID NO: 641 - protein, 9 aa, synthetic construct
QQYSTSVYT

SEQ ID NO: 642 - protein, 9 aa, synthetic construct
QQYSTSVYT

SEQ ID NO: 643 - protein, 9 aa, synthetic construct
QQYSTSVYT

SEQ ID NO: 644 - protein, 9 aa, synthetic construct
QQYSTSVYT

SEQ ID NO: 645 - protein, 9 aa, synthetic construct
QQYSTSVYT

SEQ ID NO: 646 - protein, 9 aa, synthetic construct
QQYSTSVYT

SEQ ID NO: 647 - protein, 9 aa, synthetic construct
QQYSTSVYT

SEQ ID NO: 648 - protein, 9 aa, synthetic construct
QQYSTSVYT

SEQ ID NO: 649 - protein, 9 aa, synthetic construct
QQYSTSVYT

SEQ ID NO: 650 - protein, 9 aa, synthetic construct
QQYSTSVYT

SEQ ID NO: 651 - protein, 9 aa, synthetic construct
QQYSTSVYT

SEQ ID NO: 652 - protein, 9 aa, synthetic construct
QQYSTSVYT

SEQ ID NO: 653 - protein, 9 aa, synthetic construct
QQYSTSVYT

SEQ ID NO: 654 - protein, 9 aa, synthetic construct
QQYSTSVYT

SEQ ID NO: 655 - protein, 9 aa, synthetic construct
QQYSTSVYT

SEQ ID NO: 656 - protein, 9 aa, synthetic construct
QQYSTSVYT

SEQ ID NO: 657 - protein, 9 aa, synthetic construct
QQYSTSVYT

SEQ ID NO: 658 - protein, 9 aa, synthetic construct
QQYSTSVYT

SEQ ID NO: 659 - protein, 9 aa, synthetic construct
QQYSTSVYT

SEQ ID NO: 660 - protein, 9 aa, synthetic construct
QQYSTSVYT

SEQ ID NO: 661 - protein, 9 aa, synthetic construct
QQYSTSVYT

SEQ ID NO: 662 - protein, 9 aa, synthetic construct
QQYSTSVYT

SEQ ID NO: 663 - protein, 9 aa, synthetic construct
QQYSTSVYT

SEQ ID NO: 664 - protein, 9 aa, synthetic construct
QQYSTSVYT

SEQ ID NO: 665 - protein, 9 aa, synthetic construct
QQYSTSVYT

SEQ ID NO: 666 - protein, 9 aa, synthetic construct
QQYSTSVYT

SEQ ID NO: 667 - protein, 9 aa, synthetic construct
QQYSTSVYT

SEQ ID NO: 668 - protein, 9 aa, synthetic construct
QQYSTSAYT

SEQ ID NO: 669 - protein, 9 aa, synthetic construct
QQYHNLPYS

SEQ ID NO: 670 - protein, 9 aa, synthetic construct
QHYDTAPFS

SEQ ID NO: 671 - protein, 9 aa, synthetic construct
QQYDTRPLT

SEQ ID NO: 672 - protein, 9 aa, synthetic construct
QQHYGIPWT

SEQ ID NO: 673 - protein, 9 aa, synthetic construct
QQHYGIPWT

SEQ ID NO: 674 - protein, 9 aa, synthetic construct
QHHYGVPWT

SEQ ID NO: 675 - protein, 9 aa, synthetic construct
QHHYGVPWT

SEQ ID NO: 676 - protein, 449 aa, synthetic construct
EVQLVESGGGLVQPGRSLRLSCAASGFTFSDYGMHWVRQAPGKGLEWVSAIDAGGSTDYADSVEGRFTISRDNAKNSLYLQMNSLRAEDTAVYYCAKDLEAGYYAPDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 677 - protein, 451 aa, synthetic construct
QVQLVQSGAEVKKPGASVKVSCKASGYDFNNDLIEWVRQAPGQCLEWMAVINPGSGRTNYAQKFQGRVTMTADKSTSTVYMELSSLRSEDTAVYYCAMIYYGPHSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 678 - protein, 16 aa, synthetic construct
  VARIANT 4: note = A or R
  VARIANT 7: note = S or A
  VARIANT 8: note = T or A
  VARIANT 9: note = A, D, G, or Y
AIDXGGXXXYADSVEG

SEQ ID NO: 679 - protein, 11 aa, synthetic construct
  VARIANT 3: note = A or E
  VARIANT 4: note = A or S
  VARIANT 6: note = Y or A
  VARIANT 8: note = A or G
DLXXGXYXPDV

SEQ ID NO: 680 - protein, 8 aa, synthetic construct
  VARIANT 2: note = A or V
  VARIANT 3: note = S or A
  VARIANT 4: note = N, T, or A
AXXXNLQS